(19) United States
(12) Patent Application Publication
(10) Pub. No.: US 2024/0247268 A1
(43) Pub. Date: Jul. 25, 2024

(54) Modulators of Complement Factor B

(71) Applicant: Ionis Pharmaceuticals, Inc., Carlsbad, CA (US)

(72) Inventors: Tamar R. Grossman, La Jolla, CA (US); Michael L. McCaleb, La Jolla, CA (US); Andrew T. Watt, San Diego, CA (US); Susan M. Freier, San Diego, CA (US)

(21) Appl. No.: 18/431,253

(22) Filed: Feb. 2, 2024

Related U.S. Application Data
(63) Continuation of application No. 17/465,035, filed on Sep. 2, 2021, now Pat. No. 11,926,830.
Application No. 17/465,035 is a continuation of application No. 16/439,317, filed on Jun. 12, 2019, now abandoned.
Application No. 16/439,317 is a continuation of application No. 15/912,386, filed on Mar. 5, 2018, now abandoned.
Application No. 15/912,386 is a continuation of application No. 15/021,651, filed on Mar. 11, 2016, now abandoned.

(51) Int. Cl. C12N 15/113

(52) U.S. Cl. CPC C12N 15/1137; C12Y 304/21047; C12N 2310/11; C12N 2310/315; C12N 2310/321; C12N 2310/3231; C12N 2310/3341

(57) ABSTRACT

The present embodiments provide methods, compounds, and compositions for treating, preventing, or ameliorating a disease associated with dysregulation of the complement alternative pathway by administering a Complement Factor B (CFB) specific inhibitor to a subject.

### SEQUENCE LISTING

The present application is being filed along with a Sequence Listing in electronic format. The Sequence Listing is provided as a file entitled BIOL0183USC3SEQ_ST25.txt created Aug. 30, 2021, which is 196 kb in size. The information in the electronic format of the sequence listing is incorporated herein by reference in its entirety.

### FIELD

The present embodiments provide methods, compounds, and compositions for treating, preventing, or ameliorating a disease associated with dysregulation of the complement alternative pathway by administering a Complement Factor B (CFB) specific inhibitor to a subject.

### BACKGROUND

The complement system is part of the host innate immune system involved in lysing foreign cells, enhancing phagocytosis of antigens, clumping antigen-bearing agents, and attracting macrophages and neutrophils. The complement system is divided into three initiation pathways—the classical, lectin, and alternative pathways—that converge at component C3 to generate an enzyme complex known as C3 convertase, which cleaves C3 into C3a and C3b. C3b associates with C3 convertase mediated by CFB and results in generation of C5 convertase, which cleaves C5 into C5a and C5b, which initiates the membrane attack pathway resulting in the formation of the membrane attack complex (MAC) comprising components C5b, C6, C7, C8, and C9. The membrane-attack complex (MAC) forms transmembrane channels and disrupts the phospholipid bilayer of target cells, leading to cell lysis.

In the homeostatic state, the alternative pathway is continuously activated at a low “tickover” level as a result of activation of the alternative pathway by spontaneous hydrolysis of C3 and the production of C3b, which generates C5 convertase.

### SUMMARY

The complement system mediates innate immunity and plays an important role in normal inflammatory response to injury, but its dysregulation may cause severe injury. Activation of the alternative complement pathway beyond its constitutive “tickover” level can lead to unrestrained hyperactivity and manifest as diseases of complement dysregulation.

Certain embodiments provided herein relate to methods of treating, preventing, or ameliorating a disease associated with dysregulation of the complement alternative pathway in a subject by administration of a Complement Factor B (CFB) specific inhibitor. Several embodiments provided herein are drawn to a method of inhibiting expression of CFB in a subject having, or at risk of having, a disease associated with dysregulation of the complement alternative pathway by administering a CFB specific inhibitor to the subject. In certain embodiments, a method of reducing or inhibiting accumulation of C3 deposits in the eye of a subject having, or at risk of having, a disease associated with dysregulation of the complement alternative pathway comprises administering a CFB specific inhibitor to the subject. In several embodiments, a method of reducing or inhibiting accumulation of C3 deposits in the kidney of a subject having, or at risk of having, a disease associated with dysregulation of the complement alternative pathway comprises administering a CFB specific inhibitor to the subject.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification at the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” means a nucleoside comprising a substituent at the 2′-position of the furanosyl ring other than H or OH. In certain embodiments, 2′ substituted nucleosides include nucleosides with bicyclic sugar modifications.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5 position. A 5-methylcytosine is a modified nucleobase.

“About” means within +10% of a value. For example, if it is stated, “the compounds affected at least about 70% inhibition of CFB”, it is implied that CFB levels are inhibited within a range of 60% and 80%.

“Administration” or “administering” refers to routes of introducing an antisense compound provided herein to a subject to perform its intended function. An example of a route of administration that can be used includes, but is not limited to parenteral administration, such as subcutaneous, intravenous, or intramuscular injection or infusion.

“Amelioration” refers to a lessening of at least one indicator, sign, or symptom of an associated disease, disorder, or condition. In certain embodiments, amelioration includes a delay or slowing in the progression of one or more indicators of a condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding region or segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

“Bicyclic nucleic acid” or “BNA” or “BNA nucleosides” means nucleic acid monomers having a bridge connecting two carbon atoms between the 4′ and 2 position of the nucleoside sugar unit, thereby forming a bicyclic sugar. Examples of such bicyclic sugar include, but are not limited to A) α-L-Methyleneoxy (4′-CH₂—O—2′) LNA, (B) β-D-Methyleneoxy (4′—CH₂—O—2) LNA, (C) Ethyleneoxy)(4′—(CH₂)₂—O−2° LNA, (D) Aminooxy (4′—CH₂—O—N(R)—2′) LNA and (E) Oxyamino (4′—CH₂—N(R)—O—2′) LNA, as depicted below.

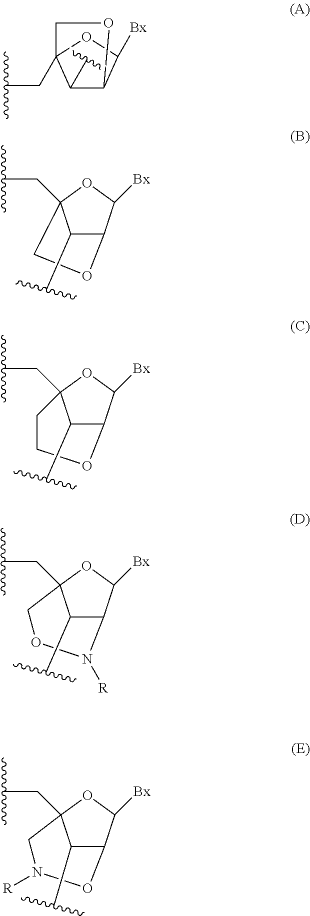

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′—CH₂—2′, 4′—(CH₂)₂-2′, 4′—(CH₂)₃-2′, 4′—CH₂—O—2′, 4′—(CH₂)₂—O-2′, 4′—CH₂—O—N(R₁)-2′ and 4′—CH₂—N(R₁)—O- 2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′—CH₂—O—2′) bridge to form the bicyclic sugar moiety. The bridge can also be a methylene (—CH₂—) group connecting the 2′ oxygen atom and the 4′ carbon atom, for which the term methyleneoxy (4′—CH₂—O-2″) LNA is used. Furthermore; in the case of the bicylic sugar moiety having an ethylene bridging group in this position, the term ethyleneoxy (4′—CH₂CH₂—O-2) LNA is used. α-L-methyleneoxy (4′—CH₂—O—2′), an isomer of methyleneoxy (4—CH₂—O-2) LNA is also encompassed within the definition of LNA, as used herein.

“Cap structure” or “terminal cap moiety” means chemical modifications, which have been incorporated at either terminus of an antisense compound.

“cEt” or “constrained ethyl” means a bicyclic sugar moiety comprising a bridge connecting the 4′-carbon and the 2′-carbon, wherein the bridge has the formula: 4′—CH(CH₃)—O-2″.

“Constrained ethyl nucleoside” (also cEt nucleoside) means a nucleoside comprising a bicyclic sugar moiety comprising a 4—CH(CH₃)—O-2′ bridge.

“Complement Factor B (CFB)” means any nucleic acid or protein of CFB. “CFB nucleic acid” means any nucleic acid encoding CFB. For example, in certain embodiments, a CFB nucleic acid includes a DNA sequence encoding CFB, an RNA sequence transcribed from DNA encoding CFB (including genomic DNA comprising introns and exons), including a non-protein encoding (i.e. non-coding) RNA sequence, and an mRNA sequence encoding CFB. “CFB mRNA” means an mRNA encoding a CFB protein.

“CFB specific inhibitor” refers to any agent capable of specifically inhibiting CFB RNA and/or CFB protein expression or activity at the molecular level. For example, CFB specific inhibitors include nucleic acids (including antisense compounds), peptides, antibodies, small molecules, and other agents capable of inhibiting the expression of CFB RNA and/or CFB protein.

“Chemically distinct region” refers to a region of an antisense compound that is in some way chemically different than another region of the same antisense compound. For example, a region having 2′—O-methoxyethyl nucleotides is chemically distinct from a region having nucleotides without 2′—O-methoxyethyl modifications.

“Chimeric antisense compounds” means antisense compounds that have at least 2 chemically distinct regions, each position having a plurality of subunits.

“Complementarity” means the capacity for pairing between nucleobases of a first nucleic acid and a second nucleic acid.

“Comprise,” “comprises” and “comprising” will be understood to imply the inclusion of a stated step or element or group of steps or elements but not the exclusion of any other step or element or group of steps or elements.

“Contiguous nucleobases” means nucleobases immediately adjacent to each other.

“Deoxyribonucleotide” means a nucleotide having a hydrogen at the 2′ position of the sugar portion of the nucleotide. Deoxyribonucleotides may be modified with any of a variety of substituents.

“Designing” or “Designed to” refer to the process of designing an oligomeric compound that specifically hybridizes with a selected nucleic acid molecule.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Identifying an animal having, or at risk for having, a disease, disorder and/or condition” means identifying an animal having been diagnosed with the disease, disorder and/or condition or identifying an animal predisposed to develop the disease, disorder and/or condition. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Inhibiting the expression or activity” refers to a reduction, blockade of the expression or activity and does not necessarily indicate a total elimination of expression or activity.

“Internucleoside linkage” refers to the chemical bond between nucleosides.

“Lengthened” antisense oligonucleotides are those that have one or more additional nucleosides relative to an antisense oligonucleotide disclosed herein.

“Linked deoxynucleoside” means a nucleic acid base (A, G, C, T, U) substituted by deoxyribose linked by a phosphate ester to form a nucleotide.

“Linked nucleosides” means adjacent nucleosides linked together by an internucleoside linkage.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any change from a natural sugar moiety.

“Modulating” refers to changing or adjusting a feature in a cell, tissue, organ or organism. For example, modulating CFB mRNA can mean to increase or decrease the level of CFB mRNA and/or CFB protein in a cell, tissue, organ or organism. A “modulator” effects the change in the cell, tissue, organ or organism. For example, a CFB antisense compound can be a modulator that decreases the amount of CFB mRNA and/or CFB protein in a cell, tissue, organ or organism.

“Monomer” refers to a single unit of an oligomer. Monomers include, but are not limited to, nucleosides and nucleotides, whether naturally occurring or modified.

“Motif” means the pattern of unmodified and modified nucleosides in an antisense compound.

“Natural sugar moiety” means a sugar moiety found in DNA (2′—H) or RNA (2′—OH).

“Naturally occurring internucleoside linkage” means a 3′ to 5′ phosphodiester linkage.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Nucleotide” means a nucleoside having a phosphate group covalently linked to the sugar portion of the nucleoside.

“Oligomeric compound” means a polymer of linked monomeric subunits which is capable of hybridizing to at least a region of a nucleic acid molecule.

“Oligonucleoside” means an oligonucleotide in which the internucleoside linkages do not contain a phosphorus atom.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutical composition” means a mixture of substances suitable for administering to an individual. For example, a pharmaceutical composition may comprise one or more active pharmaceutical agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound

“Prevent” refers to delaying or forestalling the onset, development or progression of a disease, disorder, or condition for a period of time from minutes to indefinitely. Prevent also means reducing the risk of developing a disease, disorder, or condition.

“Prophylactically effective amount” refers to an amount of a pharmaceutical agent that provides a prophylactic or preventative benefit to an animal.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Segments” are defined as smaller or sub-portions of regions within a target nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Sites,” as used herein, are defined as unique nucleobase positions within a target nucleic acid.

“Slows progression” means decrease in the development of the said disease.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments. “Stringent hybridization conditions” or “stringent conditions” refer to conditions under which an oligomeric compound will hybridize to its target sequence, but to a minimal number of other sequences.

“Subject” means a human or non-human animal selected for treatment or therapy.

“Target” refers to a protein, the modulation of which is desired.

“Target gene” refers to a gene encoding a target.

“Targeting” means the process of design and selection of an antisense compound that will specifically hybridize to a target nucleic acid and induce a desired effect.

“Target nucleic acid,” “target RNA,” “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” refers to administering a pharmaceutical composition to an animal in order to effect an alteration or improvement of a disease, disorder, or condition in the animal. In certain embodiments, one or more pharmaceutical compositions can be administered to the animal.

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Certain embodiments provide methods, compounds and compositions for inhibiting Complement Factor B (CFB) expression.

Certain embodiments provide antisense compounds targeted to a CFB nucleic acid. In certain embodiments, the CFB nucleic acid has the sequence set forth in GENBANK Accession No. NM_001710.5 (incorporated herein as SEQ ID NO: 1), GENBANK Accession No. NT_007592.15 truncated from nucleotides 31852000 to 31861000 (incorporated herein as SEQ ID NO: 2), GENBANK Accession No NW_001116486.1 truncated from nucleotides 536000 to 545000 (incorporated herein as SEQ ID NO: 3), GENBANK Accession No. XM_001113553.2 (incorporated herein as SEQ ID NO: 4), or GENBANK Accession No. NM 008198.2 (incorporated herein as SEQ ID NO: 5).

Certain embodiments provide a compound comprising a modified oligonucleotide consisting of 10 to 30 linked nucleosides and having a nucleobase sequence comprising at least 8 contiguous nucleobases of any of the nucleobase sequences of SEQ ID NOs: 6-808.

Certain embodiments provide a compound comprising a modified oligonucleotide consisting of 10 to 30 linked nucleosides and having a nucleobase sequence comprising at least 9 contiguous nucleobases of any of the nucleobase sequences of SEQ ID NOs: 6-808.

Certain embodiments provide a compound comprising a modified oligonucleotide consisting of 10 to 30 linked nucleosides and having a nucleobase sequence comprising at least 10 contiguous nucleobases of any of the nucleobase sequences of SEQ ID NOs: 6-808.

Certain embodiments provide a compound comprising a modified oligonucleotide consisting of 10 to 30 linked nucleosides and having a nucleobase sequence comprising at least 11 contiguous nucleobases of any of the nucleobase sequences of SEQ ID NOs: 6-808.

Certain embodiments provide a compound comprising a modified oligonucleotide consisting of 10 to 30 linked nucleosides and having a nucleobase sequence comprising at least 12 contiguous nucleobases of any of the nucleobase sequences of SEQ ID NOs: 6-808.

Certain embodiments provide a compound comprising a modified oligonucleotide consisting of 10 to 30 linked nucleosides and having a nucleobase sequence comprising the nucleobase sequence of any one of SEQ ID NOs: 6-808.

Certain embodiments provide a compound comprising a modified oligonucleotide consisting of the nucleobase sequence of any one of SEQ ID NOs: 6-808.

Certain embodiments provide a compound comprising a modified oligonucleotide consisting of 10 to 30 linked nucleosides complementary within nucleobases 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, or 2616-2631 of SEQ ID NO: 1, wherein said modified oligonucleotide is at least 85%, at least 90%, at least 95%, or 100% complementary to SEQ ID NO: 1.

Certain embodiments provide a compound comprising a modified oligonucleotide consisting of 10 to 30 linked nucleosides having a nucleobase sequence comprising a portion of at least 8 contiguous nucleobases complementary to an equal length portion of nucleobases 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, or 2616-2631 of SEQ ID NO: 1, wherein the nucleobase sequence of the modified oligonucleotide is at least 85%, at least 90%, at least 95%, or 100% complementary to SEQ ID NO: 1.

Certain embodiments provide a compound comprising a modified oligonucleotide consisting of 10 to 30 linked nucleosides complementary within nucleobases 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, or 7846-7862 of SEQ ID NO: 2, wherein said modified oligonucleotide is at least 85%, at least 90%, at least 95%, or 100% complementary to SEQ ID NO: 2.

Certain embodiments provide a compound comprising a modified oligonucleotide consisting of 10 to 30 linked nucleosides having a nucleobase sequence comprising a portion of at least 8 contiguous nucleobases complementary to an equal length portion of nucleobases 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838. 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, and 7846-7862 of SEQ ID NO: 2, wherein the nucleobase sequence of the modified oligonucleotide is at least 85%, at least 90%, at least 95%, or 100% complementary to SEQ ID NO: 2.

In certain embodiments, antisense compounds or oligonucleotides target a region of a CFB nucleic acid. In certain embodiments, such compounds or oligonucleotides targeted to a region of a CFB nucleic acid have a contiguous nucleobase portion that is complementary to an equal length nucleobase portion of the region. For example, the portion can be at least an 8, 9, 10, 11, 12, 13, 14, 15, or 16 contiguous nucleobase portion complementary to an equal length portion of a region recited herein. In certain embodiments, such compounds or oligonucleotide target the following nucleotide regions of SEQ ID NO: 1: 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, and 2616-2631.

In certain embodiments, antisense compounds or oligonucleotides target a region of a CFB nucleic acid. In certain embodiments, such compounds or oligonucleotides targeted to a region of a CFB nucleic acid have a contiguous nucleobase portion that is complementary to an equal length nucleobase portion of the region. For example, the portion can be at least an 8, 9, 10, 11, 12, 13, 14, 15, or 16 contiguous nucleobase portion complementary to an equal length portion of a region recited herein. In certain embodiments, such compounds or oligonucleotide target the following nucleotide regions of SEQ ID NO: 2: 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, and 7846-7862.

In certain embodiments, antisense compounds or oligonucleotides target the 3′UTR of a CFB nucleic acid. In certain embodiments, antisense compounds or oligonucleotides target within nucleotides 2574-2626 of a CFB nucleic acid having the nucleobase sequence of SEQ ID NO: 1. In certain embodiments, antisense compounds or oligonucleotides have at least an 8, 9, 10, 11, 12, 13, 14, 15, or 16 contiguous nucleobase portion complementary to an equal length portion within nucleotides 2574-2626 of a CFB nucleic acid having the nucleobase sequence of SEQ ID NO: 1.

In certain embodiments, antisense compounds or oligonucleotides target a region of a CFB nucleic acid having the nucleobase sequence of SEQ ID NO: 1 within nucleobases 2457-2631, 2457-2472, 2457-2474, 2457-2476, 2457-2566, 2457-2570, 2457-2571, 2457-2572, 2457-2573, 2457-2574, 2457-2575, 2457-2576, 2457-2577, 2457-2578, 2457-2579, 2457-2580, 2457-2581, 2457-2582, 2457-2583, 2457-2584, 2457-2585, 2457-2586, 2457-2587, 2457-2588, 2457-2589, 2457-2590, 2457-2591, 2457-2592, 2457-2593, 2457-2594, 2457-2595, 2457-2596, 2457-2597, 2457-2598, 2457-2599, 2457-2600, 2457-2601, 2457-2602, 2457-2603, 2457-2604, 2457-2605, 2457-2606, 2457-2607, 2457-2608, 2457-2609, 2457-2610, 2457-2611, 2457-2612, 2457-2613, 2457-2614, 2457-2615, 2457-2616, 2457-2617, 2457-2618, 2457-2619, 2457-2620, 2457-2621, 2457-2622, 2457-2623, 2457-2624, 2457-2625, 2457-2626, 2457-2627, 2457-2628, 2457-2629, 2457-2630, 2457-2631, 2459-2474, 2459-2476, 2459-2566, 2459-2570, 2459-2571, 2459-2572, 2459-2573, 2459-2574, 2459-2575, 2459-2576, 2459-2577, 2459-2578, 2459-2579, 2459-2580, 2459-2581, 2459-2582, 2459-2583, 2459-2584, 2459-2585, 2459-2586, 2459-2587, 2459-2588, 2459-2589, 2459-2590, 2459-2591, 2459-2592, 2459-2593, 2459-2594, 2459-2595, 2459-2596, 2459-2597, 2459-2598, 2459-2599, 2459-2600, 2459-2601, 2459-2602, 2459-2603, 2459-2604, 2459-2605, 2459-2606, 2459-2607, 2459-2608, 2459-2609, 2459-2610, 2459-2611, 2459-2612, 2459-2613, 2459-2614, 2459-2615, 2459-2616, 2459-2617, 2459-2618, 2459-2619. 2459-2620, 2459-2621, 2459-2622, 2459-2623, 2459-2624, 2459-2625, 2459-2626, 2459-2627, 2459-2628, 2459-2629, 2459-2630, 2459-2631, 2461-2476, 2461-2566, 2461-2570, 2461-2571, 2461-2572, 2461-2573, 2461-2574, 2461-2575, 2461-2576, 2461-2577, 2461-2578, 2461-2579, 2461-2580, 2461-2581, 2461-2582, 2461-2583, 2461-2584, 2461-2585, 2461-2586, 2461-2587, 2461-2588, 2461-2589, 2461-2590, 2461-2591, 2461-2592, 2461-2593, 2461-2594, 2461-2595, 2461-2596, 2461-2597, 2461-2598, 2461-2599, 2461-2600, 2461-2601, 2461-2602, 2461-2603, 2461-2604, 2461-2605, 2461-2606, 2461-2607, 2461-2608, 2461-2609, 2461-2610, 2461-2611, 2461-2612, 2461-2613, 2461-2614, 2461-2615, 2461-2616, 2461-2617, 2461-2618, 2461-2619, 2461-2620, 2461-2621, 2461-2622, 2461-2623, 2461-2624, 2461-2625, 2461-2626, 2461-2627, 2461-2628, 2461-2629, 2461-2630, 2461-2631, 2551-2566, 2551-2570, 2551-2571, 2551-2572, 2551-2573, 2551-2574, 2551-2575, 2551-2576, 2551-2577, 2551-2578, 2551-2579, 2551-2580, 2551-2581, 2551-2582, 2551-2583, 2551-2584, 2551-2585, 2551-2586, 2551-2587, 2551-2588, 2551-2589, 2551-2590, 2551-2591, 2551-2592, 2551-2593, 2551-2594, 2551-2595, 2551-2596, 2551-2597, 2551-2598, 2551-2599, 2551-2600, 2551-2601, 2551-2602, 2551-2603, 2551-2604, 2551-2605, 2551-2606, 2551-2607, 2551-2608, 2551-2609, 2551-2610, 2551-2611, 2551-2612, 2551-2613, 2551-2614, 2551-2615, 2551-2616, 2551-2617, 2551-2618, 2551-2619, 2551-2620, 2551-2621, 2551-2622, 2551-2623, 2551-2624, 2551-2625, 2551-2626, 2551-2627, 2551-2628, 2551-2629, 2551-2630, 2551-2631, 2553-2570, 2553-2571, 2553-2572, 2553-2573, 2553-2574, 2553-2575, 2553-2576, 2553-2577, 2553-2578, 2553-2579, 2553-2580, 2553-2581, 2553-2582, 2553-2583, 2553-2584, 2553-2585, 2553-2586, 2553-2587, 2553-2588, 2553-2589, 2553-2590, 2553-2591, 2553-2592, 2553-2593, 2553-2594, 2553-2595, 2553-2596, 2553-2597, 2553-2598, 2553-2599, 2553-2600, 2553-2601, 2553-2602, 2553-2603, 2553-2604, 2553-2605, 2553-2606, 2553-2607, 2553-2608, 2553-2609, 2553-2610, 2553-2611, 2553-2612, 2553-2613, 2553-2614, 2553-2615, 2553-2616, 2553-2617, 2553-2618, 2553-2619, 2553-2620, 2553-2621, 2553-2622, 2553-2623, 2553-2624, 2553-2625, 2553-2626, 2553-2627, 2553-2628, 2553-2629, 2553-2630, 2553-2631, 2554-2573, 2554-2574, 2554-2575, 2554-2576, 2554-2577, 2554-2578, 2554-2579, 2554-2580, 2554-2581, 2554-2582, 2554-2583, 2554-2584, 2554-2585, 2554-2586, 2554-2587, 2554-2588, 2554-2589, 2554-2590, 2554-2591, 2554-2592, 2554-2593, 2554-2594, 2554-2595, 2554-2596, 2554-2597, 2554-2598, 2554-2599, 2554-2600, 2554-2601, 2554-2602, 2554-2603, 2554-2604, 2554-2605, 2554-2606, 2554-2607, 2554-2608, 2554-2609, 2554-2610, 2554-2611, 2554-2612, 2554-2613, 2554-2614, 2554-2615, 2554-2616, 2554-2617, 2554-2618, 2554-2619, 2554-2620, 2554-2621, 2554-2622, 2554-2623, 2554-2624, 2554-2625, 2554-2626, 2554-2627, 2554-2628, 2554-2629, 2554-2630, 2554-2631, 2555-2572, 2555-2573, 2555-2574, 2555-2575, 2555-2576, 2555-2577, 2555-2578, 2555-2579, 2555-2580, 2555-2581, 2555-2582, 2555-2583, 2555-2584, 2555-2585, 2555-2586, 2555-2587, 2555-2588, 2555-2589, 2555-2590, 2555-2591, 2555-2592, 2555-2593, 2555-2594, 2555-2595, 2555-2596, 2555-2597, 2555-2598, 2555-2599, 2555-2600, 2555-2601, 2555-2602, 2555-2603, 2555-2604, 2555-2605, 2555-2606, 2555-2607, 2555-2608, 2555-2609, 2555-2610, 2555-2611, 2555-2612, 2555-2613, 2555-2614, 2555-2615, 2555-2616, 2555-2617, 2555-2618, 2555-2619, 2555-2620, 2555-2621, 2555-2622, 2555-2623, 2555-2624, 2555-2625, 2555-2626, 2555-2627, 2555-2628, 2555-2629, 2555-2630, 2555-2631, 2556-2573, 2556-2574, 2556-2575, 2556-2576, 2556-2577, 2556-2578, 2556-2579, 2556-2580, 2556-2581, 2556-2582, 2556-2583, 2556-2584, 2556-2585, 2556-2586, 2556-2587, 2556-2588, 2556-2589, 2556-2590, 2556-2591, 2556-2592, 2556-2593, 2556-2594, 2556-2595, 2556-2596, 2556-2597, 2556-2598, 2556-2599, 2556-2600, 2556-2601, 2556-2602, 2556-2603, 2556-2604, 2556-2605, 2556-2606, 2556-2607, 2556-2608, 2556-2609, 2556-2610, 2556-2611, 2556-2612, 2556-2613, 2556-2614, 2556-2615, 2556-2616, 2556-2617, 2556-2618, 2556-2619, 2556-2620, 2556-2621, 2556-2622, 2556-2623, 2556-2624, 2556-2625, 2556-2626, 2556-2627, 2556-2628, 2556-2629, 2556-2630, 2556-2631, 2557-2574, 2557-2575, 2557-2576, 2557-2577, 2557-2578, 2557-2579, 2557-2580, 2557-2581, 2557-2582, 2557-2583, 2557-2584, 2557-2585, 2557-2586, 2557-2587, 2557-2588, 2557-2589, 2557-2590, 2557-2591, 2557-2592, 2557-2593, 2557-2594, 2557-2595, 2557-2596, 2557-2597, 2557-2598, 2557-2599, 2557-2600, 2557-2601, 2557-2602, 2557-2603, 2557-2604, 2557-2605, 2557-2606, 2557-2607, 2557-2608, 2557-2609, 2557-2610, 2557-2611, 2557-2612, 2557-2613, 2557-2614, 2557-2615, 2557-2616, 2557-2617, 2557-2618, 2557-2619, 2557-2620, 2557-2621, 2557-2622, 2557-2623, 2557-2624, 2557-2625, 2557-2626, 2557-2627, 2557-2628, 2557-2629, 2557-2630, 2557-2631, 2558-2575, 2558-2576, 2558-2577, 2558-2578, 2558-2579, 2558-2580, 2558-2581, 2558-2582, 2558-2583, 2558-2584, 2558-2585, 2558-2586, 2558-2587, 2558-2588, 2558-2589, 2558-2590, 2558-2591, 2558-2592, 2558-2593, 2558-2594, 2558-2595, 2558-2596, 2558-2597, 2558-2598, 2558-2599, 2558-2600, 2558-2601, 2558-2602, 2558-2603, 2558-2604, 2558-2605, 2558-2606, 2558-2607, 2558-2608, 2558-2609, 2558-2610, 2558-2611, 2558-2612, 2558-2613, 2558-2614, 2558-2615, 2558-2616, 2558-2617, 2558-2618, 2558-2619, 2558-2620, 2558-2621, 2558-2622, 2558-2623, 2558-2624, 2558-2625, 2558-2626, 2558-2627, 2558-2628, 2558-2629, 2558-2630, 2558-2631, 2559-2576, 2559-2577, 2559-2578, 2559-2579, 2559-2580, 2559-2581, 2559-2582, 2559-2583, 2559-2584, 2559-2585, 2559-2586, 2559-2587, 2559-2588, 2559-2589, 2559-2590, 2559-2591, 2559-2592, 2559-2593, 2559-2594, 2559-2595, 2559-2596, 2559-2597, 2559-2598, 2559-2599, 2559-2600, 2559-2601, 2559-2602, 2559-2603, 2559-2604, 2559-2605, 2559-2606, 2559-2607, 2559-2608, 2559-2609, 2559-2610, 2559-2611, 2559-2612, 2559-2613, 2559-2614, 2559-2615, 2559-2616, 2559-2617, 2559-2618, 2559-2619, 2559-2620, 2559-2621, 2559-2622, 2559-2623, 2559-2624, 2559-2625, 2559-2626, 2559-2627, 2559-2628, 2559-2629, 2559-2630, 2559-2631, 2560-2577, 2560-2578, 2560-2579, 2560-2580, 2560-2581, 2560-2582, 2560-2583, 2560-2584, 2560-2585, 2560-2586, 2560-2587, 2560-2588, 2560-2589, 2560-2590, 2560-2591, 2560-2592, 2560-2593, 2560-2594, 2560-2595, 2560-2596, 2560-2597, 2560-2598, 2560-2599, 2560-2600, 2560-2601, 2560-2602, 2560-2603, 2560-2604, 2560-2605, 2560-2606, 2560-2607, 2560-2608, 2560-2609, 2560-2610, 2560-2611, 2560-2612, 2560-2613, 2560-2614, 2560-2615, 2560-2616, 2560-2617, 2560-2618, 2560-2619, 2560-2620, 2560-2621, 2560-2622, 2560-2623, 2560-2624, 2560-2625, 2560-2626, 2560-2627, 2560-2628, 2560-2629, 2560-2630, 2560-2631, 2561-2578, 2561-2579, 2561-2580, 2561-2581, 2561-2582, 2561-2583, 2561-2584, 2561-2585, 2561-2586, 2561-2587, 2561-2588, 2561-2589, 2561-2590, 2561-2591, 2561-2592, 2561-2593, 2561-2594, 2561-2595, 2561-2596, 2561-2597, 2561-2598, 2561-2599, 2561-2600, 2561-2601, 2561-2602, 2561-2603, 2561-2604, 2561-2605, 2561-2606, 2561-2607, 2561-2608, 2561-2609, 2561-2610, 2561-2611, 2561-2612, 2561-2613, 2561-2614, 2561-2615, 2561-2616, 2561-2617, 2561-2618, 2561-2619, 2561-2620, 2561-2621, 2561-2622, 2561-2623, 2561-2624, 2561-2625, 2561-2626, 2561-2627, 2561-2628, 2561-2629, 2561-2630, 2561-2631, 2562-2577, 2562-2578, 2562-2579, 2562-2580, 2562-2581, 2562-2582, 2562-2583, 2562-2584, 2562-2585, 2562-2586, 2562-2587, 2562-2588, 2562-2589, 2562-2590, 2562-2591, 2562-2592, 2562-2593, 2562-2594, 2562-2595, 2562-2596, 2562-2597, 2562-2598, 2562-2599, 2562-2600, 2562-2601, 2562-2602, 2562-2603, 2562-2604, 2562-2605, 2562-2606, 2562-2607, 2562-2608, 2562-2609, 2562-2610, 2562-2611, 2562-2612, 2562-2613, 2562-2614, 2562-2615, 2562-2616, 2562-2617, 2562-2618, 2562-2619, 2562-2620, 2562-2621, 2562-2622, 2562-2623, 2562-2624, 2562-2625, 2562-2626, 2562-2627, 2562-2628, 2562-2629, 2562-2630, 2562-2631, 2563-2580, 2563-2581, 2563-2582, 2563-2583, 2563-2584, 2563-2585, 2563-2586, 2563-2587, 2563-2588, 2563-2589, 2563-2590, 2563-2591, 2563-2592, 2563-2593, 2563-2594, 2563-2595, 2563-2596, 2563-2597, 2563-2598, 2563-2599, 2563-2600, 2563-2601, 2563-2602, 2563-2603, 2563-2604, 2563-2605, 2563-2606, 2563-2607, 2563-2608, 2563-2609, 2563-2610, 2563-2611, 2563-2612, 2563-2613, 2563-2614, 2563-2615, 2563-2616, 2563-2617, 2563-2618, 2563-2619, 2563-2620, 2563-2621, 2563-2622, 2563-2623, 2563-2624, 2563-2625, 2563-2626, 2563-2627, 2563-2628, 2563-2629, 2563-2630, 2563-2631, 2564-2581, 2564-2582, 2564-2583, 2564-2584, 2564-2585, 2564-2586, 2564-2587, 2564-2588, 2564-2589, 2564-2590, 2564-2591, 2564-2592, 2564-2593, 2564-2594, 2564-2595, 2564-2596, 2564-2597, 2564-2598, 2564-2599, 2564-2600, 2564-2601, 2564-2602, 2564-2603, 2564-2604, 2564-2605, 2564-2606, 2564-2607, 2564-2608, 2564-2609, 2564-2610, 2564-2611, 2564-2612, 2564-2613, 2564-2614, 2564-2615, 2564-2616, 2564-2617, 2564-2618, 2564-2619, 2564-2620, 2564-2621, 2564-2622, 2564-2623, 2564-2624, 2564-2625, 2564-2626, 2564-2627, 2564-2628, 2564-2629, 2564-2630, 2564-2631, 2565-2584, 2565-2585, 2565-2586, 2565-2587, 2565-2588, 2565-2589, 2565-2590, 2565-2591, 2565-2592, 2565-2593, 2565-2594, 2565-2595, 2565-2596, 2565-2597, 2565-2598, 2565-2599, 2565-2600, 2565-2601, 2565-2602, 2565-2603, 2565-2604, 2565-2605, 2565-2606, 2565-2607, 2565-2608, 2565-2609, 2565-2610, 2565-2611, 2565-2612, 2565-2613, 2565-2614, 2565-2615, 2565-2616, 2565-2617, 2565-2618, 2565-2619, 2565-2620, 2565-2621, 2565-2622, 2565-2623, 2565-2624, 2565-2625, 2565-2626, 2565-2627, 2565-2628, 2565-2629, 2565-2630, 2565-2631, 2566-2583, 2566-2584, 2566-2585, 2566-2586, 2566-2587, 2566-2588, 2566-2589, 2566-2590, 2566-2591, 2566-2592, 2566-2593, 2566-2594, 2566-2595, 2566-2596, 2566-2597, 2566-2598, 2566-2599, 2566-2600, 2566-2601, 2566-2602, 2566-2603, 2566-2604, 2566-2605, 2566-2606, 2566-2607, 2566-2608, 2566-2609, 2566-2610, 2566-2611, 2566-2612, 2566-2613, 2566-2614, 2566-2615, 2566-2616, 2566-2617, 2566-2618, 2566-2619, 2566-2620, 2566-2621, 2566-2622, 2566-2623, 2566-2624, 2566-2625, 2566-2626, 2566-2627, 2566-2628, 2566-2629, 2566-2630, 2566-2631, 2567-2584, 2567-2585, 2567-2586, 2567-2587, 2567-2588, 2567-2589, 2567-2590, 2567-2591, 2567-2592, 2567-2593, 2567-2594, 2567-2595, 2567-2596, 2567-2597, 2567-2598, 2567-2599, 2567-2600, 2567-2601, 2567-2602, 2567-2603, 2567-2604, 2567-2605, 2567-2606, 2567-2607, 2567-2608, 2567-2609, 2567-2610, 2567-2611, 2567-2612, 2567-2613, 2567-2614, 2567-2615, 2567-2616, 2567-2617, 2567-2618, 2567-2619, 2567-2620, 2567-2621, 2567-2622, 2567-2623, 2567-2624, 2567-2625, 2567-2626, 2567-2627, 2567-2628, 2567-2629, 2567-2630, 2567-2631, 2568-2585, 2568-2586, 2568-2587, 2568-2588, 2568-2589, 2568-2590, 2568-2591, 2568-2592, 2568-2593, 2568-2594, 2568-2595, 2568-2596, 2568-2597, 2568-2598, 2568-2599, 2568-2600, 2568-2601, 2568-2602, 2568-2603, 2568-2604, 2568-2605, 2568-2606, 2568-2607, 2568-2608, 2568-2609, 2568-2610, 2568-2611, 2568-2612, 2568-2613, 2568-2614, 2568-2615, 2568-2616, 2568-2617, 2568-2618, 2568-2619, 2568-2620, 2568-2621, 2568-2622, 2568-2623, 2568-2624, 2568-2625, 2568-2626, 2568-2627, 2568-2628, 2568-2629, 2568-2630, 2568-2631, 2569-2586, 2569-2587, 2569-2588, 2569-2589, 2569-2590, 2569-2591, 2569-2592, 2569-2593, 2569-2594, 2569-2595, 2569-2596, 2569-2597, 2569-2598, 2569-2599, 2569-2600, 2569-2601, 2569-2602, 2569-2603, 2569-2604, 2569-2605, 2569-2606, 2569-2607, 2569-2608, 2569-2609, 2569-2610, 2569-2611, 2569-2612, 2569-2613, 2569-2614, 2569-2615, 2569-2616, 2569-2617, 2569-2618, 2569-2619, 2569-2620, 2569-2621, 2569-2622, 2569-2623, 2569-2624, 2569-2625, 2569-2626, 2569-2627, 2569-2628, 2569-2629, 2569-2630, 2569-2631, 2569-2586, 2569-2587, 2569-2588, 2569-2589, 2569-2590, 2569-2591, 2569-2592, 2569-2593, 2569-2594, 2569-2595, 2569-2596, 2569-2597, 2569-2598, 2569-2599, 2569-2600, 2569-2601, 2569-2602, 2569-2603, 2569-2604, 2569-2605, 2569-2606, 2569-2607, 2569-2608, 2569-2609, 2569-2610, 2569-2611, 2569-2612, 2569-2613, 2569-2614, 2569-2615, 2569-2616, 2569-2617, 2569-2618, 2569-2619, 2569-2620, 2569-2621, 2569-2622, 2569-2623, 2569-2624, 2569-2625, 2569-2626, 2569-2627, 2569-2628, 2569-2629, 2569-2630, 2569-2631, 2571-2588, 2571-2589, 2571-2590, 2571-2591, 2571-2592, 2571-2593, 2571-2594, 2571-2595, 2571-2596, 2571-2597, 2571-2598, 2571-2599, 2571-2600, 2571-2601, 2571-2602, 2571-2603, 2571-2604, 2571-2605, 2571-2606, 2571-2607, 2571-2608, 2571-2609, 2571-2610, 2571-2611, 2571-2612, 2571-2613, 2571-2614, 2571-2615, 2571-2616, 2571-2617, 2571-2618, 2571-2619, 2571-2620, 2571-2621, 2571-2622, 2571-2623, 2571-2624, 2571-2625, 2571-2626, 2571-2627, 2571-2628, 2571-2629, 2571-2630, 2571-2631, 2572-2589, 2572-2590, 2572-2591, 2572-2592, 2572-2593, 2572-2594, 2572-2595, 2572-2596, 2572-2597, 2572-2598, 2572-2599, 2572-2600, 2572-2601, 2572-2602, 2572-2603, 2572-2604, 2572-2605, 2572-2606, 2572-2607, 2572-2608, 2572-2609, 2572-2610, 2572-2611, 2572-2612, 2572-2613, 2572-2614, 2572-2615, 2572-2616, 2572-2617, 2572-2618, 2572-2619, 2572-2620, 2572-2621, 2572-2622, 2572-2623, 2572-2624, 2572-2625, 2572-2626, 2572-2627, 2572-2628, 2572-2629, 2572-2630, 2572-2631, 2573-2590, 2573-2591, 2573-2592, 2573-2593, 2573-2594, 2573-2595, 2573-2596, 2573-2597, 2573-2598, 2573-2599, 2573-2600, 2573-2601, 2573-2602, 2573-2603, 2573-2604, 2573-2605, 2573-2606, 2573-2607, 2573-2608, 2573-2609, 2573-2610, 2573-2611, 2573-2612, 2573-2613, 2573-2614, 2573-2615, 2573-2616, 2573-2617, 2573-2618, 2573-2619, 2573-2620, 2573-2621, 2573-2622, 2573-2623, 2573-2624, 2573-2625, 2573-2626, 2573-2627, 2573-2628, 2573-2629, 2573-2630, 2573-2631, 2574-2591, 2574-2592, 2574-2593, 2574-2594, 2574-2595, 2574-2596, 2574-2597, 2574-2598, 2574-2599, 2574-2600, 2574-2601, 2574-2602, 2574-2603, 2574-2604, 2574-2605, 2574-2606, 2574-2607, 2574-2608, 2574-2609, 2574-2610, 2574-2611, 2574-2612, 2574-2613, 2574-2614, 2574-2615, 2574-2616, 2574-2617, 2574-2618, 2574-2619, 2574-2620, 2574-2621, 2574-2622, 2574-2623, 2574-2624, 2574-2625, 2574-2626, 2574-2627, 2574-2628, 2574-2629, 2574-2630, 2574-2631, 2575-2592, 2575-2593, 2575-2594, 2575-2595, 2575-2596, 2575-2597, 2575-2598, 2575-2599, 2575-2600, 2575-2601, 2575-2602, 2575-2603, 2575-2604, 2575-2605, 2575-2606, 2575-2607, 2575-2608, 2575-2609, 2575-2610, 2575-2611, 2575-2612, 2575-2613, 2575-2614, 2575-2615, 2575-2616, 2575-2617, 2575-2618, 2575-2619, 2575-2620, 2575-2621, 2575-2622, 2575-2623, 2575-2624, 2575-2625, 2575-2626, 2575-2627, 2575-2628, 2575-2629, 2575-2630, 2575-2631, 2576-2593, 2576-2594, 2576-2595, 2576-2596, 2576-2597, 2576-2598, 2576-2599, 2576-2600, 2576-2601, 2576-2602, 2576-2603, 2576-2604, 2576-2605, 2576-2606, 2576-2607, 2576-2608, 2576-2609, 2576-2610, 2576-2611, 2576-2612, 2576-2613, 2576-2614, 2576-2615, 2576-2616, 2576-2617, 2576-2618, 2576-2619, 2576-2620, 2576-2621, 2576-2622, 2576-2623, 2576-2624, 2576-2625, 2576-2626, 2576-2627, 2576-2628, 2576-2629, 2576-2630, 2576-2631, 2577-2594, 2577-2595, 2577-2596, 2577-2597, 2577-2598, 2577-2599, 2577-2600, 2577-2601, 2577-2602, 2577-2603, 2577-2604, 2577-2605, 2577-2606, 2577-2607, 2577-2608, 2577-2609, 2577-2610, 2577-2611, 2577-2612, 2577-2613, 2577-2614, 2577-2615, 2577-2616, 2577-2617, 2577-2618, 2577-2619, 2577-2620, 2577-2621, 2577-2622, 2577-2623, 2577-2624, 2577-2625, 2577-2626, 2577-2627, 2577-2628, 2577-2629, 2577-2630, 2577-2631, 2578-2597, 2578-2598, 2578-2599, 2578-2600, 2578-2601, 2578-2602, 2578-2603, 2578-2604, 2578-2605, 2578-2606, 2578-2607, 2578-2608, 2578-2609, 2578-2610, 2578-2611, 2578-2612, 2578-2613, 2578-2614, 2578-2615, 2578-2616, 2578-2617, 2578-2618, 2578-2619, 2578-2620, 2578-2621, 2578-2622, 2578-2623, 2578-2624, 2578-2625, 2578-2626, 2578-2627, 2578-2628, 2578-2629, 2578-2630, 2578-2631, 2579-2598, 2579-2599, 2579-2600, 2579-2601, 2579-2602, 2579-2603, 2579-2604, 2579-2605, 2579-2606, 2579-2607, 2579-2608, 2579-2609, 2579-2610, 2579-2611, 2579-2612, 2579-2613, 2579-2614, 2579-2615, 2579-2616, 2579-2617, 2579-2618, 2579-2619, 2579-2620, 2579-2621, 2579-2622, 2579-2623, 2579-2624, 2579-2625, 2579-2626, 2579-2627, 2579-2628, 2579-2629, 2579-2630, 2579-2631, 2580-2598, 2580-2599, 2580-2600, 2580-2601, 2580-2602, 2580-2603, 2580-2604, 2580-2605, 2580-2606, 2580-2607, 2580-2608, 2580-2609, 2580-2610, 2580-2611, 2580-2612, 2580-2613, 2580-2614, 2580-2615, 2580-2616, 2580-2617, 2580-2618, 2580-2619, 2580-2620, 2580-2621, 2580-2622, 2580-2623, 2580-2624, 2580-2625, 2580-2626, 2580-2627, 2580-2628, 2580-2629, 2580-2630, 2580-2631, 2581-2597, 2581-2598, 2581-2599, 2581-2600, 2581-2601, 2581-2602, 2581-2603, 2581-2604, 2581-2605, 2581-2606, 2581-2607, 2581-2608, 2581-2609, 2581-2610, 2581-2611, 2581-2612, 2581-2613, 2581-2614, 2581-2615, 2581-2616, 2581-2617, 2581-2618, 2581-2619, 2581-2620, 2581-2621, 2581-2622, 2581-2623, 2581-2624, 2581-2625, 2581-2626, 2581-2627, 2581-2628, 2581-2629, 2581-2630, 2581-2631, 2582-2600, 2582-2601, 2582-2602, 2582-2603, 2582-2604, 2582-2605, 2582-2606, 2582-2607, 2582-2608, 2582-2609, 2582-2610, 2582-2611, 2582-2612, 2582-2613, 2582-2614, 2582-2615, 2582-2616, 2582-2617, 2582-2618, 2582-2619, 2582-2620, 2582-2621, 2582-2622, 2582-2623, 2582-2624, 2582-2625, 2582-2626, 2582-2627, 2582-2628, 2582-2629, 2582-2630, 2582-2631, 2583-2601, 2583-2602, 2583-2603, 2583-2604, 2583-2605, 2583-2606, 2583-2607, 2583-2608, 2583-2609, 2583-2610, 2583-2611, 2583-2612, 2583-2613, 2583-2614, 2583-2615, 2583-2616, 2583-2617, 2583-2618, 2583-2619, 2583-2620, 2583-2621, 2583-2622, 2583-2623, 2583-2624, 2583-2625, 2583-2626, 2583-2627, 2583-2628, 2583-2629, 2583-2630, 2583-2631, 2585-2603, 2585-2604, 2585-2605, 2585-2606, 2585-2607, 2585-2608, 2585-2609, 2585-2610, 2585-2611, 2585-2612, 2585-2613, 2585-2614, 2585-2615, 2585-2616, 2585-2617, 2585-2618, 2585-2619, 2585-2620, 2585-2621, 2585-2622, 2585-2623, 2585-2624, 2585-2625, 2585-2626, 2585-2627, 2585-2628, 2585-2629, 2585-2630, 2585-2631, 2586-2604, 2586-2605, 2586-2606, 2586-2607, 2586-2608, 2586-2609, 2586-2610, 2586-2611, 2586-2612, 2586-2613, 2586-2614, 2586-2615, 2586-2616, 2586-2617, 2586-2618, 2586-2619, 2586-2620, 2586-2621, 2586-2622, 2586-2623, 2586-2624, 2586-2625, 2586-2626, 2586-2627, 2586-2628, 2586-2629, 2586-2630, 2586-2631, 2587-2605, 2587-2606, 2587-2607, 2587-2608, 2587-2609, 2587-2610, 2587-2611, 2587-2612, 2587-2613, 2587-2614, 2587-2615, 2587-2616, 2587-2617, 2587-2618, 2587-2619, 2587-2620, 2587-2621, 2587-2622, 2587-2623, 2587-2624, 2587-2625, 2587-2626, 2587-2627, 2587-2628, 2587-2629, 2587-2630, 2587-2631, 2588-2606, 2588-2607, 2588-2608, 2588-2609, 2588-2610, 2588-2611, 2588-2612, 2588-2613, 2588-2614, 2588-2615, 2588-2616, 2588-2617, 2588-2618, 2588-2619, 2588-2620, 2588-2621, 2588-2622, 2588-2623, 2588-2624, 2588-2625, 2588-2626, 2588-2627, 2588-2628, 2588-2629, 2588-2630, 2588-2631, 2589-2607, 2589-2608, 2589-2609, 2589-2610, 2589-2611, 2589-2612, 2589-2613, 2589-2614, 2589-2615, 2589-2616, 2589-2617, 2589-2618, 2589-2619, 2589-2620, 2589-2621, 2589-2622, 2589-2623, 2589-2624, 2589-2625, 2589-2626, 2589-2627, 2589-2628, 2589-2629, 2589-2630, 2589-2631, 2590-2606, 2590-2607, 2590-2608, 2590-2609, 2590-2610, 2590-2611, 2590-2612, 2590-2613, 2590-2614, 2590-2615, 2590-2616, 2590-2617, 2590-2618, 2590-2619, 2590-2620, 2590-2621, 2590-2622, 2590-2623, 2590-2624, 2590-2625, 2590-2626, 2590-2627, 2590-2628, 2590-2629, 2590-2630, 2590-2631, 2591-2610, 2591-2611, 2591-2612, 2591-2613, 2591-2614, 2591-2615, 2591-2616, 2591-2617, 2591-2618, 2591-2619, 2591-2620, 2591-2621, 2591-2622, 2591-2623, 2591-2624, 2591-2625, 2591-2626, 2591-2627, 2591-2628, 2591-2629, 2591-2630, 2591-2631, 2592-2611, 2592-2612, 2592-2613, 2592-2614, 2592-2615, 2592-2616, 2592-2617, 2592-2618, 2592-2619, 2592-2620, 2592-2621, 2592-2622, 2592-2623, 2592-2624, 2592-2625, 2592-2626, 2592-2627, 2592-2628, 2592-2629, 2592-2630, 2592-2631, 2593-2608, 2593-2612, 2593-2613, 2593-2614, 2593-2615, 2593-2616, 2593-2617, 2593-2618, 2593-2619, 2593-2620, 2593-2621, 2593-2622, 2593-2623, 2593-2624, 2593-2625, 2593-2626, 2593-2627, 2593-2628, 2593-2629, 2593-2630, 2593-2631, 2594-2612, 2594-2613, 2594-2614, 2594-2615, 2594-2616, 2594-2617, 2594-2618, 2594-2619, 2594-2620, 2594-2621, 2594-2622, 2594-2623, 2594-2624, 2594-2625, 2594-2626, 2594-2627, 2594-2628, 2594-2629, 2594-2630, 2594-2631, 2595-2611, 2595-2612, 2595-2613, 2595-2614, 2595-2615, 2595-2616, 2595-2617, 2595-2618, 2595-2619, 2595-2620, 2595-2621, 2595-2622, 2595-2623, 2595-2624, 2595-2625, 2595-2626, 2595-2627, 2595-2628, 2595-2629, 2595-2630, 2595-2631, 2596-2614, 2596-2615, 2596-2616, 2596-2617, 2596-2618, 2596-2619, 2596-2620, 2596-2621, 2596-2622, 2596-2623, 2596-2624, 2596-2625, 2596-2626, 2596-2627, 2596-2628, 2596-2629, 2596-2630, 2596-2631, 2597-2612, 2597-2613, 2597-2614, 2597-2615, 2597-2616, 2597-2617, 2597-2618, 2597-2619, 2597-2620, 2597-2621, 2597-2622, 2597-2623, 2597-2624, 2597-2625, 2597-2626, 2597-2627, 2597-2628, 2597-2629, 2597-2630, 2597-2631, 2598-2613, 2598-2614, 2598-2615, 2598-2616, 2598-2617, 2598-2618, 2598-2619, 2598-2620, 2598-2621, 2598-2622, 2598-2623, 2598-2624, 2598-2625, 2598-2626, 2598-2627, 2598-2628, 2598-2629, 2598-2630, 2598-2631, 2599-2614, 2599-2615, 2599-2616, 2599-2617, 2599-2618, 2599-2619, 2599-2620, 2599-2621, 2599-2622, 2599-2623, 2599-2624, 2599-2625, 2599-2626, 2599-2627, 2599-2628, 2599-2629, 2599-2630, 2599-2631, 2600-2615, 2600-2616, 2600-2617, 2600-2618, 2600-2619, 2600-2620, 2600-2621, 2600-2622, 2600-2623, 2600-2624, 2600-2625, 2600-2626, 2600-2627, 2600-2628, 2600-2629, 2600-2630, 2600-2631, 2601-2616, 2601-2617, 2601-2618, 2601-2619, 2601-2620, 2601-2621, 2601-2622, 2601-2623, 2601-2624, 2601-2625, 2601-2626, 2601-2627, 2601-2628, 2601-2629, 2601-2630, 2601-2631, 2602-2618, 2602-2619, 2602-2620, 2602-2621, 2602-2622, 2602-2623, 2602-2624, 2602-2625, 2602-2626, 2602-2627, 2602-2628, 2602-2629, 2602-2630, 2602-2631, 2603-2620, 2603-2621, 2603-2622, 2603-2623, 2603-2624, 2603-2625, 2603-2626, 2603-2627, 2603-2628, 2603-2629, 2603-2630, 2603-2631, 2604-2619, 2604-2620, 2604-2621, 2604-2622, 2604-2623, 2604-2624, 2604-2625, 2604-2626, 2604-2627, 2604-2628, 2604-2629, 2604-2630, 2604-2631, 2605-2620, 2605-2621, 2605-2622, 2605-2623, 2605-2624, 2605-2625, 2605-2626, 2605-2627, 2605-2628, 2605-2629, 2605-2630, 2605-2631, 2606-2621, 2606-2622, 2606-2623, 2606-2624, 2606-2625, 2606-2626, 2606-2627, 2606-2628, 2606-2629, 2606-2630, 2606-2631, 2607-2622, 2607-2623, 2607-2624, 2607-2625, 2607-2626, 2607-2627, 2607-2628, 2607-2629, 2607-2630, 2607-2631, 2608-2623, 2608-2624, 2608-2625, 2608-2626, 2608-2627, 2608-2628, 2608-2629, 2608-2630, 2608-2631, 2609-2624, 2609-2625, 2609-2626, 2609-2627, 2609-2628, 2609-2629, 2609-2630, 2609-2631, 2610-2625, 2610-2626, 2610-2627, 2610-2628, 2610-2629, 2610-2630, 2610-2631, 2611-2626, 2611-2627, 2611-2628, 2611-2629, 2611-2630, 2611-2631, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, or 2616-2631. In certain embodiments, antisense compounds or oligonucleotides target at least an 8, 9, 10, 11, 12, 13, 14, 15, or 16 contiguous nucleobases within the aforementioned nucleobase regions.

In certain embodiments, the following nucleotide regions of SEQ ID NO: 1, when targeted by antisense compounds or oligonucleotides, display at least 50% inhibition: 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, and 2616-2631.

In certain embodiments, the following nucleotide regions of SEQ ID NO: 2, when targeted by antisense compounds or oligonucleotides, display at least 50% inhibition: 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, and 7846-7862.

In certain embodiments, the following nucleotide regions of SEQ ID NO: 1, when targeted by antisense compounds or oligonucleotides, display at least 60% inhibition: 48-63, 150-169, 152-171, 154-169, 154-173, 156-171, 156-175, 158-173, 158-177, 600-619, 1135-1154, 1141-1160, 1147-1166, 1153-1172, 1171-1186, 1173-1188, 1175-1190, 1749-1768, 1763-1782, 1763-1782, 1912-1931, 2189-2208, 2191-2210, 2193-2212, 2195-2210, 2195-2214, 2197-2212, 2197-2216, 2223-2238, 2225-2240, 2227-2242, 2238-2257, 2448-2467, 2453-2472, 2455-2474, 2457-2472, 2457-2476, 2459-2474, 2459-2478, 2461-2476, 2461-2480, 2550-2569. 2551-2566, 2552-2571, 2553-2568, 2553-2570, 2553-2571, 2553-2572, 2554-2571, 2554-2572, 2554-2573, 2555-2572, 2555-2574, 2556-2573, 2556-2574, 2556-2575, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2587, 2570-2589, 2571-2588, 2572-2590, 2572-2591, 2573-2590, 2573-2592, 2574-2591, 2574-2593, 2575-2590, 2575-2592, 2575-2594, 2576-2593, 2576-2595, 2577-2594, 2577-2595, 2577-2596, 2578-2594, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2602, 2584-2603, 2585-2601, 2585-2603, 2585-2604, 2586-2602, 2586-2604, 2586-2605, 2587-2603, 2587-2605, 2587-2606, 2588-2603, 2588-2604, 2588-2606, 2588-2607, 2589-2605, 2589-2606, 2589-2607, 2589-2608, 2590-2605, 2590-2606, 2590-2607, 2590-2608, 2590-2609, 2591-2607, 2591-2609, 2591-2610, 2592-2608, 2592-2609, 2592-2611, 2593-2608, 2593-2609, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2630, 2615-2631, 2615-2631, and 2616-2631.

In certain embodiments, the following nucleotide regions of SEQ ID NO: 2, when targeted by antisense compounds or oligonucleotides, display at least 60% inhibition: 1685-1704, 1686-1705, 1769-1784, 1871-1890, 1873-1892, 1875-1890, 1875-1894, 1877-1892, 1877-1896, 1879-1894, 1879-1898, 2808-2827, 3819-3838, 3825-3844, 3831-3850, 3837-3856, 4151-4166, 5890-5909, 5904-5923, 5904-5923, 6406-6425, 6977-6996, 6979-6998, 6981-7000, 6983-6998, 6983-7002, 6985-7000, 6985-7004, 7122-7141, 7683-7702, 7688-7707, 7690-7709, 7692-7707, 7692-7711, 7694-7709, 7696-7711, 7696-7715, 7786-7801, 7787-7806, 7788-7803, 7788-7805, 7788-7806, 7788-7807, 7789-7806, 7789-7807, 7789-7808, 7790-7807, 7790-7809, 7791-7808, 7791-7809, 7791-7810, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7822, 7805-7824, 7806-7823, 7806-7825, 7807-7824, 7807-7825, 7807-7826, 7808-7825, 7808-7827, 7809-7826, 7809-7828, 7810-7825, 7810-7827, 7810-7829, 7811-7828, 7811-7830, 7812-7829, 7812-7830, 7812-7831, 7813-7829, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7837, 7819-7838, 7820-7836, 7820-7838, 7820-7839, 7821-7837, 7821-7839, 7821-7840, 7822-7838, 7822-7840, 7822-7841, 7823-7838, 7823-7839, 7823-7841, 7823-7842, 7824-7840, 7824-7841, 7824-7842, 7824-7843, 7825-7840, 7825-7841, 7825-7842, 7825-7843, 7825-7844, 7826-7842, 7826-7844, 7826-7845, 7827-7843, 7827-7844, 7827-7846, 7828-7843, 7828-7844, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, 7846-7862, and 7847-7862.

In certain embodiments, the following nucleotide regions of SEQ ID NO: 1, when targeted by antisense compounds or oligonucleotides, display at least 70% inhibition: 48-63, 150-169, 152-171, 154-169, 154-173, 156-171, 156-175, 158-173, 158-177, 1135-1154, 1141-1160, 1147-1166, 1171-1186, 1173-1188, 1175-1190, 1749-1768, 1763-1782, 1912-1931, 2193-2212, 2195-2210, 2195-2214, 2197-2212, 2197-2216, 2223-2238, 2225-2240, 2227-2242, 2453-2472, 2455-2474, 2457-2472, 2457-2476, 2459-2474, 2461-2476, 2461-2480, 2550-2569, 2551-2566, 2552-2571, 2553-2570, 2553-2571, 2553-2572, 2554-2571, 2554-2572, 2554-2573, 2554-2573, 2555-2572, 2555-2574, 2555-2574, 2556-2573, 2556-2574, 2556-2575, 2557-2574, 2557-2576, 2558-2575, 2558-2576, 2558-2577, 2559-2576, 2559-2577, 2559-2578, 2560-2577, 2560-2578, 2560-2579, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2585, 2568-2587, 2569-2586, 2569-2588, 2570-2587, 2570-2589, 2571-2588, 2571-2590, 2572-2589, 2572-2591, 2573-2590, 2573-2592, 2574-2591, 2574-2593, 2575-2592, 2575-2594, 2576-2593, 2576-2595, 2577-2594, 2577-2596, 2578-2597, 2579-2598, 2580-2596, 2580-2598, 2580-2599, 2581-2597, 2581-2600, 2582-2598, 2582-2600, 2582-2601, 2583-2599, 2583-2601, 2583-2602, 2584-2600, 2584-2602, 2584-2603, 2585-2601, 2585-2603, 2585-2604, 2586-2605, 2587-2606, 2588-2604, 2588-2606, 2588-2607, 2589-2605, 2589-2606. 2589-2607, 2589-2608, 2590-2605, 2590-2606, 2590-2607, 2590-2609, 2591-2607, 2591-2610, 2592-2611, 2593-2608, 2593-2612, 2594-2609, 2594-2610, 2594-2612, 2594-2613, 2595-2610, 2595-2611, 2595-2612, 2595-2613, 2595-2614, 2596-2611, 2596-2614, 2596-2615, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2630, 2615-2631, and 2616-2631.

In certain embodiments, the following nucleotide regions of SEQ ID NO: 2, when targeted by antisense compounds or oligonucleotides, display at least 70% inhibition: 1685-1704, 1686-1705, 1769-1784, 1871-1890, 1873-1892, 1875-1890, 1875-1894, 1877-1892, 1877-1896, 1879-1894, 1879-1898, 3819-3838, 3825-3844, 3831-3850, 4151-4166, 5890-5909, 5904-5923, 5904-5923, 6406-6425, 6983-6998, 6983-7002, 6985-7000, 6985-7004, 7688-7707, 7690-7709, 7692-7707, 7692-7711, 7694-7709, 7696-7711, 7696-7715, 7786-7801, 7787-7806, 7788-7805, 7788-7806, 7788-7807, 7789-7806, 7789-7807, 7789-7808, 7790-7807, 7790-7809, 7791-7808, 7791-7809, 7791-7810, 7792-7809, 7792-7811, 7793-7810, 7793-7811, 7793-7812, 7794-7811, 7794-7812, 7794-7813, 7795-7812, 7795-7813, 7795-7814, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7820, 7803-7822, 7804-7821, 7804-7823, 7805-7822, 7805-7824, 7806-7823, 7806-7825, 7807-7824, 7807-7826, 7808-7825, 7808-7827, 7809-7826, 7809-7828, 7810-7827, 7811-7828, 7811-7830, 7812-7829, 7812-7831, 7813-7832, 7814-7833, 7815-7831, 7815-7833, 7815-7834, 7816-7832, 7816-7835, 7817-7833, 7817-7835, 7817-7836, 7818-7834, 7818-7836, 7818-7837, 7819-7835, 7819-7837, 7819-7838, 7820-7836, 7820-7838, 7820-7839, 7821-7840, 7822-7841, 7823-7839, 7823-7841, 7823-7842, 7824-7840, 7824-7841, 7824-7842, 7824-7843, 7825-7840, 7825-7841, 7825-7842, 7825-7844, 7826-7842, 7826-7845, 7827-7846, 7828-7843, 7828-7847, 7829-7844, 7829-7845, 7829-7847, 7829-7848, 7830-7845, 7830-7846, 7830-7847, 7830-7848, 7830-7849, 7831-7846, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, 7846-7862, and 7847-7862.

In certain embodiments, the following nucleotide regions of SEQ ID NO: 1, when targeted by antisense compounds or oligonucleotides, display at least 80% inhibition: 152-171, 154-169, 156-171, 158-173, 1135-1154, 1171-1186, 1173-1188, 1175-1190, 1763-1782, 1912-1931, 2197-2212, 2223-2238, 2225-2240, 2227-2242, 2457-2472, 2459-2474, 2461-2476, 2551-2566, 2553-2570, 2553-2571, 2553-2572, 2554-2573, 2555-2572, 2555-2574, 2556-2573, 2556-2574, 2556-2575, 2557-2574, 2557-2576, 2558-2575, 2558-2576, 2559-2577, 2559-2578, 2560-2577, 2560-2578, 2560-2579, 2561-2578, 2561-2579, 2561-2580, 2562-2577, 2562-2579, 2562-2581, 2563-2580, 2563-2582, 2564-2581, 2564-2583, 2565-2584, 2566-2583, 2567-2584, 2567-2586, 2568-2585, 2568-2587, 2569-2586, 2569-2588, 2570-2587, 2571-2588, 2571-2590, 2572-2589, 2572-2591, 2573-2590, 2573-2592, 2574-2591, 2574-2593, 2575-2592, 2576-2593, 2576-2595, 2577-2594, 2577-2596, 2578-2597, 2580-2598, 2580-2599, 2581-2597, 2581-2600, 2582-2601, 2583-2602, 2584-2603, 2585-2604, 2586-2605, 2587-2606, 2588-2607, 2589-2608, 2590-2606, 2590-2607, 2590-2609, 2591-2610, 2592-2611, 2593-2608, 2593-2612, 2594-2613, 2595-2611, 2595-2614, 2596-2615, 2597-2612, 2597-2613, 2597-2614, 2597-2615, 2597-2616, 2598-2613, 2598-2613, 2598-2614, 2598-2615, 2598-2616, 2598-2617, 2599-2614, 2599-2617, 2599-2618, 2600-2615, 2600-2617, 2600-2618, 2600-2619, 2601-2616, 2601-2617, 2601-2619, 2601-2620, 2602-2618, 2602-2621, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2627, 2609-2624, 2609-2626, 2609-2627, 2609-2628, 2610-2625, 2610-2626, 2610-2628, 2610-2629, 2611-2626, 2611-2627, 2611-2629, 2611-2630, 2612-2627, 2612-2628, 2612-2630, 2612-2631, 2613-2628, 2613-2629, 2613-2631, 2614-2629, 2614-2630, 2614-2631, 2615-2630, and 2616-2631.

In certain embodiments, the following nucleotide regions of SEQ ID NO: 2, when targeted by antisense compounds or oligonucleotides, display at least 80% inhibition: 1685-1704, 1686-1705, 1873-1892, 1875-1890, 1877-1892, 1879-1894, 3819-3838, 4151-4166, 5904-5923, 6406-6425, 6985-7000, 7692-7707, 7694-7709, 7696-7711, 7786-7801, 7788-7805, 7788-7806, 7788-7807, 7789-7808, 7790-7807, 7790-7809, 7791-7808, 7791-7809, 7791-7810, 7792-7809, 7792-7811, 7793-7810, 7793-7811, 7794-7812, 7794-7813, 7795-7812, 7795-7813, 7795-7814, 7796-7813, 7796-7814, 7796-7815, 7797-7812, 7797-7814, 7797-7816, 7798-7815, 7798-7817, 7799-7816, 7799-7818, 7800-7819, 7801-7818, 7802-7819, 7802-7821, 7803-7820, 7803-7822, 7804-7821, 7804-7823, 7805-7822, 7806-7823, 7806-7825, 7807-7824, 7807-7826, 7808-7825, 7808-7827, 7809-7826, 7809-7828, 7810-7827, 7811-7828, 7812-7829, 7812-7831, 7813-7832, 7814-7833, 7815-7834, 7816-7832, 7816-7835, 7817-7836, 7818-7837, 7819-7838, 7820-7839, 7821-7840, 7822-7841, 7823-7842, 7824-7843, 7825-7841, 7825-7842, 7825-7844, 7826-7845, 7827-7846, 7828-7843, 7828-7847, 7829-7848, 7830-7846, 7830-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7852, 7834-7853, 7835-7850, 7835-7852, 7835-7853, 7835-7854, 7836-7851, 7836-7852, 7836-7854, 7836-7855, 7837-7853, 7837-7856, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7862, 7844-7859, 7844-7861, 7844-7862, 7845-7860, 7845-7861, 7846-7862, and 7847-7862.

In certain embodiments, the following nucleotide regions of SEQ ID NO: 1, when targeted by antisense compounds or oligonucleotides, display at least 90% inhibition: 154-169, 156-171, 158-173, 1135-1154, 1171-1186, 1173-1188, 1763-1782, 1912-1931, 2223-2238, 2227-2242, 2459-2474, 2461-2476, 2554-2573, 2555-2574, 2560-2577, 2561-2578, 2561-2579, 2562-2581, 2563-2580, 2563-2582, 2564-2581, 2566-2583, 2567-2584, 2568-2585, 2568-2587, 2569-2586, 2570-2587, 2576-2593, 2577-2594, 2577-2596, 2578-2597, 2580-2599, 2581-2600, 2582-2601, 2583-2602, 2584-2603, 2586-2605, 2587-2605, 2587-2606, 2588-2607, 2589-2608, 2590-2607, 2590-2609, 2592-2611, 2595-2614, 2596-2615, 2597-2612, 2597-2613, 2597-2615, 2597-2616, 2598-2613, 2598-2613, 2598-2617, 2599-2614, 2599-2618, 2600-2615, 2600-2619, 2601-2617, 2601-2620, 2602-2621, 2603-2622, 2604-2623, 2605-2621, 2605-2622, 2605-2624, 2606-2625, 2607-2626, 2608-2623, 2608-2625, 2609-2628, 2611-2627, 2611-2630, 2612-2628, 2612-2631, 2613-2629, 2614-2629, 2615-2630, and 2616-2631.

In certain embodiments, the following nucleotide regions of SEQ ID NO: 2, when targeted by antisense compounds or oligonucleotides, display at least 90% inhibition: 1685-1704, 1686-1705, 1875-1890, 1877-1892, 1879-1894, 3819-3838, 5904-5923, 6406-6425, 7694-7709, 7696-7711, 7789-7808, 7790-7809, 7795-7812, 7795-7813, 7796-7813, 7796-7814, 7797-7814, 7797-7816, 7798-7815, 7798-7817, 7799-7816, 7801-7818, 7802-7819, 7803-7820, 7803-7822, 7804-7821, 7805-7822, 7811-7828, 7812-7829, 7812-7831, 7813-7832, 7815-7834, 7818-7837, 7819-7838, 7821-7840, 7822-7840, 7822-7841, 7825-7842, 7832-7847, 7832-7848, 7832-7850, 7833-7848, 7833-7852, 7834-7849, 7834-7853, 7835-7850, 7836-7852, 7836-7855, 7837-7856, 7838-7856, 7839-7857, 7839-7858, 7840-7856, 7840-7857, 7840-7859, 7843-7858, 7843-7860, and 7846-7862.

In certain embodiments, the following antisense compounds or oligonucleotides target a region of a CFB nucleic acid and effect at least a 50% inhibition of a CFB mRNA, ISIS NOs: 516350, 532614, 532632, 532635, 532638, 532639, 532686, 532687, 532688, 532689, 532690, 532691, 532692, 532692, 532693, 532694, 532695, 532696, 532697, 532698, 532699, 532700, 532701, 532702, 532703, 532704, 532705, 532706, 532707, 532770, 532775, 532778, 532780, 532791, 532800, 532809, 532810, 532811, 532917, 532952, 588509, 588510, 588511, 588512, 588513, 588514, 588515, 588516, 588517, 588518, 588519, 588520, 588522, 588523, 588524, 588525, 588527, 588528, 588529, 588530, 588531, 588532, 588533, 588534, 588535, 588536, 588537, 588538, 588539, 588540, 588541, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588566, 588567, 588568, 588569, 588570, 588571, 588572, 588573, 588574, 588575, 588576, 588577, 588580, 588581, 588585, 588586, 588589, 588590, 588599, 588603, 588606, 588608, 588610, 588614, 588616, 588628, 588631, 588632, 588634, 588636, 588638, 588640, 588645, 588646, 588654, 588656, 588658, 588660, 588662, 588664, 588670, 588672, 588676, 588682, 588688, 588696, 588698, 588807, 588808, 588809, 588813, 588814, 588815, 588819, 588820, 588822, 588823, 588838, 588839, 588840, 588841, 588842, 588846, 588847, 588848, 588849, 588850, 588851, 588852, 588853, 588854, 588855, 588856, 588857, 588858, 588859, 588860, 588861, 588862, 588863, 588864, 588865, 588866, 588867, 588868, 588870, 588871, 588872, 588873, 588874, 588875, 588876, 588877, 588878, 588879, 588880, 588881, 588882, 588883, 588884, 598999, 599000, 599001, 599002, 599003, 599004, 599005, 599006, 599007, 599008, 599009, 599010, 599011, 599012, 599013, 599014, 599015, 599018, 599019, 599023, 599024, 599025, 599026, 599027, 599028, 599029, 599030, 599031, 599032, 599033, 599034, 599035, 599058, 599062, 599063, 599064, 599065, 599070, 599071, 599072, 599073, 599074, 599076, 599077, 599078, 599079, 599080, 599081, 599082, 599083, 599084, 599085, 599086, 599087, 599088, 599089, 599090, 599091, 599092, 599093, 599094, 599095, 599096, 599097, 599098, 599102, 599119, 599123, 599124, 599125, 599126, 599127, 599128, 599132, 599133, 599134, 599135, 599136, 599137, 599138, 599139, 599140, 599141, 599142, 599143, 599144, 599145, 599147, 599148, 599149, 599150, 599151, 599152, 599153, 599154, 599155, 599156, 599157, 599158, 599159, 599178, 599179, 599180, 599181, 599182, 599186, 599187, 599188, 599189, 599190, 599191, 599192, 599193, 599194, 599195, 599196, 599197, 599198, 599199, 599200, 599201, 599202, 599203, 599204, 599205, 599206, 599207, 599208, 599209, 599210, 599211, 599212, 599213, 599214, 599215, 599216, 599217, 599218, 599219, 599220, 599221, 599221, 599222, 599223, 599224, 599225, 599226, 599227, 599228, 599229, 599230, 599231, 599232, 599233, 599234, 599235, 599236, 599241, 599247, 599248, 599249, 599255, 599256, 599257, 599258, 599260, 599261, 599262, 599263, 599264, 599265, 599266, 599267, 599268, 599269, 599270, 599271, 599272, 599273, 599274, 599275, 599276, 599277, 599278, 599279, 599280, 599297, 599299, 599306, 599307, 599308, 599309, 599311, 599312, 599313, 599314, 599315, 599316, 599317, 599318, 599319, 599320, 599321, 599322, 599323, 599324, 599325, 599326, 599327, 599328, 599329, 599330, 599338, 599349, 599353, 599354, 599355, 599356, 599357, 599358, 599359, 599360, 599361, 599362, 599363, 599364, 599369, 599371, 599372, 599373, 599376, 599378, 599379, 599382, 599383, 599384, 599385, 599386, 599387, 599388, 599389, 599390, 599391, 599392, 599393, 599394, 599395, 599396, 599397, 599398, 599399, 599400, 599401, 599402, 599403, 599404, 599405, 599406, 599407, 599408, 599409, 599410, 599412, 599413, 599414, 599415, 599416, 599417, 599418, 599419, 599420, 599421, 599422, 599423, 599424, 599425, 599426, 599433, 599434, 599435, 599436, 599437, 599438, 599439, 599440, 599441, 599442, 599443, 599444, 599445, 599446, 599447, 599448, 599450, 599454, 599455, 599456, 599467, 599468, 599469, 599471, 599472, 599473, 599474, 599475, 599476, 599477, 599478, 599479, 599480, 599481, 599482, 599483, 599484, 599485, 599486, 599487, 599488, 599489, 599490, 599491, 599492, 599493, 599494, 599495, 599496, 599497, 599498, 599499, 599500, 599501, 599502, 599503, 599504, 599505, 599506, 599507, 599508, 599509, 599512, 599515, 599518, 599531, 599541, 599541, 599546, 599547, 599548, 599549, 599550, 599552, 599553, 599554, 599555, 599557, 599558, 599561, 599562, 599563, 599564, 599565, 599566, 599567, 599568, 599569, 599570, 599577, 599578, 599579, 599580, 599581, 599581, 599582, 599584, 599585, 599586, 599587, 599588, 599589, 599590, 599591, 599592, 599593, 599594, 599595, 601321, 601322, 601323, 601325, 601327, 601328, 601329, 601330, 601332, 601333, 601334, 601335, 601336, 601337, 601338, 601339, 601341, 601342, 601343, 601344, 601345, 601346, 601347, 601348, 601349, 601362, 601367, 601368, 601369, 601371, 601372, 601373, 601374, 601375, 601377, 601378, 601380, 601381, 601382, 601383, 601384, 601385, 601386, 601387, and 601388.

In certain embodiments, the following antisense compounds or oligonucleotides target a region of a CFB nucleic acid and effect at least a 50% inhibition of a CFB mRNA, SEQ ID NOs: 12, 30, 33, 36, 37, 84, 85, 86, 87, 88, 89, 90, 90, 91, 92, 93, 94, 95, 96, 97, 98, 99, 100, 101, 102, 103, 104, 105, 198, 203, 206, 208, 219, 228, 237, 238, 239, 317, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 468, 472, 473, 475, 478, 479, 488, 492, 494, 495, 498, 499, 500, 502, 503, 509, 510, 511, 512, 513, 514, 515, 517, 518, 522, 523, 524, 525, 529, 530, 531, 534, 535, 537, 540, 541, 542, 543, 544, 545, 546, 547, 549, 550, 551, 552, 553, 554, 555, 556, 557, 558, 559, 563, 564, 565, 569, 570, 572, 573, 577, 588, 589, 590, 591, 592, 594, 595, 596, 597, 598, 599, 600, 601, 602, 603, 604, 605, 606, 607, 608, 609, 610, 611, 612, 613, 614, 615, 616, 617, 618, 619, 623, 640, 641, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 700, 704, 705, 706, 707, 708, 709, 711, 712, 713, 714, 715, 716, 717, 718, 720, 721, 722, 723, 724, 725, 726, 727, 728, 729, 730, 731, 732, 733, 734, 735, 736, 737, 738, 739, 740, 741, 742, 743, 744, 745, 745, 746, 747, 748, 749, 750, 751, 752, 753, 754, 755, 756, 758, 759, 760, 761, 762, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 813, 833, 834, 841, 846, 849, 850, 867, and 873.

In certain embodiments, the following antisense compounds or oligonucleotides target a region of a CFB nucleic acid and effect at least a 60% inhibition of a CFB mRNA, ISIS NOs: 516350, 532614, 532635, 532686, 532687, 532688, 532689, 532770, 532800, 532809, 532810, 532811, 532917, 532952, 588512, 588513, 588514, 588515, 588516, 588517, 588518, 588519, 588522, 588523, 588524, 588525, 588527, 588528, 588529, 588530, 588531, 588532, 588533, 588534, 588535, 588536, 588537, 588538, 588539, 588540, 588541, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588566, 588567, 588568, 588569, 588570, 588571, 588572, 588573, 588574, 588575, 588576, 588577, 588636, 588638, 588640, 588664, 588676, 588696, 588698, 588807, 588808, 588814, 588815, 588819, 588820, 588840, 588842, 588846, 588847, 588848, 588849, 588850, 588851, 588852, 588853, 588854, 588855, 588856, 588857, 588858, 588859, 588860, 588861, 588862, 588863, 588864, 588866, 588867, 588868, 588870, 588871, 588872, 588873, 588874, 588875, 588876, 588877, 588878, 588879, 588880, 588881, 588882, 588883, 588884, 598999, 599000, 599001, 599002, 599003, 599004, 599005, 599006, 599007, 599008, 599009, 599010, 599011, 599012, 599013, 599014, 599015, 599019, 599024, 599025, 599026, 599027, 599028, 599029, 599030, 599031, 599032, 599033, 599034, 599035, 599064, 599065, 599071, 599072, 599077, 599078, 599079, 599080, 599083, 599084, 599085, 599086, 599087, 599088, 599089, 599090, 599091, 599092, 599093, 599094, 599095, 599096, 599097, 599125, 599126, 599127, 599133, 599134, 599135, 599136, 599138, 599139, 599140, 599141, 599142, 599148, 599149, 599150, 599151, 599152, 599154, 599155, 599156, 599157, 599158, 599159, 599178, 599179, 599180, 599181, 599187, 599188, 599190, 599192, 599193, 599194, 599195, 599196, 599197, 599198, 599199, 599200, 599201, 599202, 599203, 599204, 599205, 599206, 599207, 599208, 599209, 599210, 599211, 599212, 599213, 599214, 599215, 599216, 599217, 599218, 599219, 599220, 599221, 599222, 599223, 599224, 599225, 599226, 599227, 599228, 599229, 599230, 599231, 599232, 599233, 599234, 599235, 599236, 599247, 599255, 599256, 599257, 599263, 599264, 599265, 599266, 599270, 599271, 599272, 599273, 599274, 599275, 599276, 599277, 599278, 599279, 599280, 599306, 599307, 599308, 599311, 599312, 599313, 599314, 599315, 599316, 599317, 599318, 599319, 599320, 599321, 599322, 599323, 599324, 599325, 599327, 599328, 599329, 599330, 599349, 599353, 599355, 599356, 599357, 599358, 599359, 599360, 599361, 599362, 599363, 599364, 599369, 599371, 599372, 599373, 599376, 599378, 599379, 599382, 599384, 599386, 599387, 599388, 599389, 599390, 599391, 599392, 599393, 599394, 599395, 599396, 599397, 599398, 599399, 599400, 599401, 599402, 599403, 599404, 599405, 599406, 599407, 599408, 599409, 599410, 599412, 599413, 599414, 599415, 599416, 599417, 599418, 599419, 599420, 599421, 599422, 599423, 599424, 599425, 599433, 599434, 599435, 599436, 599437, 599438, 599439, 599440, 599441, 599442, 599443, 599444, 599445, 599446, 599447, 599448, 599456, 599467, 599468, 599471, 599472, 599473, 599474, 599475, 599476, 599477, 599478, 599479, 599480, 599481, 599482, 599483, 599484, 599485, 599486, 599487, 599488, 599489, 599490, 599491, 599492, 599493, 599494, 599495, 599496, 599497, 599498, 599499, 599500, 599501, 599502, 599503, 599504, 599505, 599506, 599507, 599508, 599512, 599531, 599547, 599548, 599549, 599552, 599553, 599554, 599555, 599557, 599558, 599562, 599563, 599564, 599565, 599566, 599567, 599568, 599569, 599570, 599577, 599578, 599579, 599580, 599581, 599582, 599584, 599585, 599586, 599587, 599588, 599589, 599590, 599591, 599592, 599593, 599594, 599595, 601323, 601327, 601329, 601332, 601333, 601333, 601334, 601335, 601336, 601338, 601339, 601341, 601342, 601343, 601344, 601345, 601346, 601347, 601348, 601349, 601368, 601369, 601371, 601372, 601374, 601375, 601377, 601378, 601380, 601381, 601382, 601383, 601384, 601385, 601386, 601387, and 601388.

In certain embodiments, the following antisense compounds or oligonucleotides target a region of a CFB nucleic acid and effect at least a 60% inhibition of a CFB mRNA, SEQ ID NOs: 12, 33, 84, 85, 86, 87, 198, 228, 237, 238, 239, 317, 395, 396, 397, 398, 399, 400, 401, 402, 403, 404, 405, 406, 407, 408, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 472, 473, 513, 514, 515, 531, 537, 541, 542, 543, 544, 545, 546, 547, 549, 550, 551, 552, 553, 554, 555, 556, 557, 558, 564, 565, 569, 570, 577, 590, 592, 595, 596, 597, 598, 599, 600, 601, 602, 603, 604, 605, 606, 607, 608, 609, 610, 611, 612, 613, 614, 615, 616, 617, 618, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 682, 683, 684, 685, 686, 687, 688, 689, 700, 704, 706, 707, 708, 709, 711, 712, 713, 714, 715, 716, 717, 720, 721, 722, 723, 724, 725, 726, 727, 727, 728, 729, 730, 731, 732, 733, 734, 736, 737, 738, 739, 740, 741, 742, 743, 744, 745, 745, 746, 747, 748, 749, 750, 751, 752, 753, 754, 755, 756, 758, 759, 760, 761, 767, 768, 770, 772, 773, 774, 775, 775, 776, 776, 777, 777, 778, 779, 780, 781, 782, 783, 783, 784, 784, 785, 786, 787, 788, 789, 790, 791, 792, 793, 794, 795, 796, 797, 798, 799, 813, 833, 834, 841, 846, 849, and 850.

In certain embodiments, the following antisense compounds or oligonucleotides target a region of a CFB nucleic acid and effect at least a 70% inhibition of a CFB mRNA, ISIS NOs: 516350, 532614, 532686, 532687, 532688, 532770, 532800, 532809, 532810, 532811, 532917, 532952, 588512, 588513, 588514, 588515, 588516, 588517, 588518, 588524, 588529, 588530, 588531, 588532, 588533, 588534, 588535, 588536, 588537, 588538, 588539, 588540, 588541, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588568, 588569, 588570, 588571, 588572, 588573, 588574, 588575, 588577, 588636, 588638, 588640, 588696, 588698, 588807, 588814, 588815, 588819, 588842, 588847, 588848, 588849, 588850, 588851, 588852, 588853, 588856, 588857, 588858, 588859, 588860, 588861, 588862, 588863, 588866, 588867, 588870, 588871, 588872, 588873, 588874, 588875, 588876, 588877, 588878, 588879, 588880, 588881, 588882, 588883, 588884, 599000, 599001, 599003, 599004, 599005, 599008, 599009, 599010, 599011, 599014, 599015, 599024, 599025, 599027, 599028, 599029, 599030, 599031, 599032, 599033, 599034, 599072, 599077, 599080, 599085, 599086, 599087, 599088, 599089, 599090, 599091, 599093, 599094, 599095, 599096, 599097, 599125, 599126, 599134, 599138, 599139, 599148, 599149, 599150, 599151, 599152, 599154, 599155, 599156, 599157, 599158, 599187, 599188, 599193, 599195, 599196, 599197, 599198, 599199, 599200, 599201, 599202, 599203, 599204, 599205, 599206, 599207, 599208, 599210, 599211, 599212, 599213, 599214, 599215, 599216, 599217, 599218, 599219, 599220, 599221, 599222, 599223, 599224, 599225, 599226, 599227, 599228, 599229, 599230, 599231, 599232, 599233, 599234, 599235, 599236, 599266, 599272, 599272, 599273, 599274, 599275, 599277, 599278, 599279, 599280, 599280, 599306, 599311, 599312, 599313, 599314, 599315, 599316, 599317, 599318, 599319, 599320, 599321, 599322, 599323, 599325, 599327, 599328, 599329, 599330, 599355, 599357, 599358, 599359, 599360, 599361, 599362, 599363, 599364, 599369, 599371, 599372, 599373, 599378, 599379, 599382, 599384, 599386, 599387, 599388, 599389, 599390, 599391, 599392, 599393, 599394, 599395, 599396, 599397, 599398, 599399, 599400, 599401, 599402, 599403, 599404, 599405, 599406, 599407, 599408, 599409, 599410, 599413, 599414, 599415, 599416, 599417, 599418, 599419, 599420, 599421, 599422, 599423, 599424, 599433, 599434, 599435, 599436, 599437, 599438, 599439, 599440, 599441, 599442, 599443, 599445, 599446, 599447, 599448, 599472, 599473, 599474, 599475, 599476, 599477, 599478, 599479, 599480, 599481, 599482, 599483, 599484, 599485, 599486, 599487, 599488, 599489, 599490, 599491, 599492, 599493, 599494, 599495, 599496, 599497, 599498, 599499, 599500, 599501, 599502, 599503, 599504, 599505, 599506, 599507, 599508, 599512, 599547, 599548, 599552, 599553, 599554, 599555, 599558, 599562, 599563, 599564, 599566, 599567, 599568, 599569, 599570, 599577, 599578, 599579, 599580, 599581, 599582, 599585, 599586, 599587, 599588, 599589, 599590, 599591, 599592, 599593, 599594, 599595, 601332, 601335, 601341, 601343, 601344, 601345, 601346, 601347, 601348, 601349, 601371, 601372, 601380, 601382, 601383, 601384, 601385, 601386, and 601387.

In certain embodiments, the following antisense compounds or oligonucleotides target a region of a CFB nucleic acid and effect at least a 70% inhibition of a CFB mRNA, SEQ ID NOs: 12, 84, 85, 86, 198, 228, 237, 238, 239, 317, 395, 396, 397, 398, 399, 402, 403, 404, 405, 407, 408, 410, 411, 412, 412, 413, 414, 415, 416, 417, 418, 419, 420, 421, 422, 423, 424, 425, 426, 427, 428, 429, 430, 431, 432, 433, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 464, 465, 472, 473, 513, 514, 515, 541, 542, 543, 544, 545, 546, 547, 549, 550, 551, 552, 553, 554, 555, 556, 557, 564, 565, 569, 592, 595, 596, 597, 598, 599, 600, 601, 602, 603, 604, 606, 607, 608, 609, 610, 611, 612, 613, 614, 615, 616, 617, 618, 645, 646, 647, 648, 649, 650, 653, 654, 655, 656, 659, 660, 662, 663, 664, 665, 666, 668, 669, 670, 671, 672, 673, 674, 675, 676, 677, 677, 678, 679, 680, 682, 683, 684, 686, 687, 688, 689, 706, 708, 709, 711, 712, 713, 714, 715, 720, 721, 722, 723, 724, 725, 726, 727, 728, 729, 730, 731, 732, 733, 734, 736, 737, 738, 739, 740, 741, 742, 743, 744, 745, 746, 747, 748, 749, 750, 751, 752, 753, 754, 755, 756, 767, 768, 773, 775, 776, 777, 778, 779, 780, 781, 782, 783, 784, 785, 786, 787, 788, 789, 790, 791, 792, 793, 793, 794, 795, 797, 798, 799, 813, 833, 834, 841, 846, 849, 867, and 873.

In certain embodiments, the following antisense compounds or oligonucleotides target a region of a CFB nucleic acid and effect at least an 80% inhibition of a CFB mRNA, ISIS NOs: 532686, 532809, 532810, 532811, 532917, 532952, 588512, 588517, 588518, 588533, 588534, 588535, 588536, 588537, 588538, 588539, 588540, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588571, 588638, 588640, 588696, 588698, 588807, 588814, 588849, 588850, 588851, 588853, 588857, 588858, 588859, 588860, 588861, 588862, 588863, 588866, 588867, 588871, 588872, 588873, 588874, 588875, 588876, 588877, 588878, 588879, 588880, 588881, 588882, 588883, 599001, 599024, 599025, 599033, 599086, 599087, 599088, 599089, 599093, 599094, 599095, 599096, 599134, 599139, 599148, 599149, 599151, 599154, 599155, 599156, 599158, 599188, 599195, 599196, 599198, 599201, 599202, 599203, 599204, 599205, 599206, 599207, 599212, 599213, 599215, 599216, 599217, 599218, 599219, 599220, 599221, 599222, 599223, 599224, 599225, 599226, 599227, 599228, 599229, 599230, 599231, 599232, 599233, 599234, 599235, 599236, 599272, 599273, 599275, 599277, 599278, 599279, 599280, 599311, 599313, 599314, 599316, 599317, 599318, 599320, 599321, 599322, 599323, 599327, 599328, 599329, 599330, 599355, 599357, 599358, 599359, 599360, 599361, 599362, 599363, 599364, 599371, 599372, 599373, 599378, 599379, 599382, 599384, 599386, 599387, 599388, 599389, 599390, 599391, 599392, 599393, 599397, 599398, 599399, 599400, 599401, 599403, 599404, 599405, 599407, 599408, 599409, 599410, 599413, 599414, 599415, 599416, 599417, 599418, 599419, 599420, 599421, 599422, 599423, 599424, 599433, 599434, 599435, 599436, 599437, 599438, 599439, 599440, 599441, 599445, 599446, 599447, 599448, 599474, 599476, 599477, 599479, 599481, 599482, 599483, 599485, 599486, 599487, 599488, 599489, 599490, 599491, 599492, 599494, 599495, 599496, 599497, 599498, 599499, 599500, 599502, 599503, 599504, 599505, 599506, 599507, 599508, 599547, 599552, 599553, 599554, 599558, 599563, 599567, 599568, 599569, 599570, 599577, 599578, 599581, 599582, 599585, 599587, 599588, 599590, 599591, 599592, 599593, 599594, 601332, 601344, 601345, 601382, 601383, and 601385.

In certain embodiments, the following antisense compounds or oligonucleotides target a region of a CFB nucleic acid and effect at least a 80% inhibition of a CFB mRNA, SEQ ID NOs: 84, 237, 238, 239, 317, 395, 397, 411, 412, 413, 414, 415, 417, 418, 419, 420, 421, 422, 423, 425, 426, 427, 429, 430, 431, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 472, 473, 514, 515, 542, 543, 544, 545, 546, 547, 550, 551, 552, 553, 554, 555, 556, 557, 564, 595, 599, 600, 601, 602, 603, 606, 607, 608, 609, 610, 611, 612, 613, 614, 615, 616, 617, 618, 646, 655, 660, 662, 663, 666, 669, 670, 671, 672, 673, 675, 676, 677, 678, 679, 682, 684, 686, 687, 688, 689, 706, 708, 709, 711, 712, 713, 714, 715, 720, 722, 723, 724, 725, 726, 727, 729, 730, 731, 732, 733, 736, 737, 738, 739, 740, 741, 742, 743, 744, 745, 746, 747, 748, 749, 750, 751, 752, 753, 754, 755, 756, 768, 775, 776, 778, 781, 782, 783, 784, 785, 787, 788, 789, 790, 791, 792, 793, 794, 799, 813, 833, 834, 841, 849, 867, and 873.

In certain embodiments, the following antisense compounds or oligonucleotides target a region of a CFB nucleic acid and effect at least a 90% inhibition of a CFB mRNA, ISIS NOs: 532686, 532811, 532917, 588536, 588537, 588538, 588539, 588544, 588545, 588546, 588548, 588551, 588552, 588553, 588554, 588555, 588556, 588557, 588558, 588559, 588560, 588561, 588562, 588564, 588638, 588640, 588696, 588698, 588849, 588850, 588851, 588860, 588866, 588867, 588872, 588873, 588874, 588876, 588877, 588878, 588879, 588881, 588883, 599149, 599188, 599203, 599206, 599220, 599221, 599222, 599223, 599224, 599225, 599226, 599227, 599228, 599229, 599235, 599236, 599279, 599280, 599314, 599321, 599362, 599378, 599390, 599391, 599398, 599399, 599404, 599413, 599414, 599416, 599419, 599420, 599422, 599435, 599437, 599438, 599441, 599483, 599494, 599508, 599552, 599553, 599554, 599568, 599570, 599577, 599581, 599591, 599592, and 599593.

In certain embodiments, the following antisense compounds or oligonucleotides target a region of a CFB nucleic acid and effect at least a 90% inhibition of a CFB mRNA, SEQ ID NOs: 84, 238, 239, 317, 412, 413, 420, 421, 426, 434, 436, 437, 438, 439, 440, 442, 443, 444, 445, 446, 448, 451, 452, 453, 454, 455, 456, 457, 458, 459, 460, 461, 462, 464, 465, 472, 473, 514, 515, 542, 543, 544, 545, 546, 551, 553, 555, 556, 599, 600, 601, 602, 610, 616, 617, 618, 662, 666, 670, 676, 677, 678, 688, 689, 713, 723, 729, 730, 740, 741, 742, 743, 744, 745, 746, 747, 748, 749, 755, 756, 768, 783, 793, 833, and 867.

In certain embodiments, a compound comprises a modified oligonucleotide consisting of 10 to 30 linked nucleosides complementary within nucleotides 2193-2212, 2195-2210, 2457-2476, 2571-2590, 2584-2603, 2588-2607, 2592-2611, 2594-2613, 2597-2616, 2600-2619, or 2596-2611 of SEQ ID NO: 1.

In certain embodiments, a compound comprises a modified oligonucleotide consisting of 10 to 30 linked nucleosides having a nucleobase sequence comprising any one of SEQ ID NOs: 198, 228, 237, 440, 444, 448, 450, 453, 455, 549, and 598.

In certain embodiments, a compound comprises a modified oligonucleotide having a nucleobase sequence consisting of any one of SEQ ID NOs: 198, 228, 237, 440, 444, 448, 450, 453, 455, 549, and 598.

In certain embodiments, any of the foregoing compounds or oligonucleotides comprises at least one modified internucleoside linkage, at least one modified sugar, and/or at least one modified nucleobase.

In certain embodiments, any of the foregoing compounds or oligonucleotides comprises at least one modified sugar. In certain embodiments, at least one modified sugar comprises a 2′—O-methoxyethyl group. In certain embodiments, at least one modified sugar is a bicyclic sugar, such as a 4′—CH(CH₃)—O-2′ group, a 4′—CH₂—O-2′ group, or a 4′—(CH₂)₂—O-2 group.

In certain embodiments, the modified oligonucleotide comprises at least one modified internucleoside linkage, such as a phosphorothioate internucleoside linkage.

In certain embodiments, any of the foregoing compounds or oligonucleotides comprises at least one modified nucleobase, such as 5-methylcytosine.

In certain embodiments, any of the foregoing compounds or oligonucleotides comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar. In certain embodiments, the oligonucleotide consists of 10 to 30 linked nucleosides having a nucleobase sequence comprising the sequence recited in SEQ ID NO: 198, 228, 237, 440, 444, 448, 450, 453, 455, 549, or 598.

In certain embodiments, the modified oligonucleotide has a nucleobase sequence comprising the sequence recited in SEQ ID NO: 198, 228, 237, 440, 444, 448, 450, 453, or 455, wherein the modified oligonucleotide comprises
a gap segment consisting of ten linked deoxynucleosides;
a 5′ wing segment consisting of five linked nucleosides: and
a 3′ wing segment consisting of five linked nucleosides;
wherein the gap segment is positioned between the 5′ wing segment and the 3′ wing segment, wherein each nucleoside of each wing segment comprises a 2′—O-methoxyethyl sugar; wherein each internucleoside linkage is a phosphorothioate linkage and wherein each cytosine is a 5-methylcytosine.

In certain embodiments, a compound comprises or consists of a single-stranded modified oligonucleotide consisting of 20 linked nucleosides having a nucleobase sequence consisting of the sequence recited in SEQ ID NO: 198, 228, 237, 440, 444, 448, 450, 453, or 455, wherein the oligonucleotide comprises:
a gap segment consisting of ten linked deoxynucleosides;
a 5′ wing segment consisting of five linked nucleosides; and
a 3′ wing segment consisting of five linked nucleosides;
wherein the gap segment is positioned between the 5′ wing segment and the 3′ wing segment, wherein each nucleoside of each wing segment comprises a 2′—O-methoxyethyl sugar; wherein each internucleoside linkage is a phosphorothioate linkage: and wherein each cytosine is a 5-methylcytosine.

In certain embodiments, a compound comprises ISIS 588540. In certain embodiments, a compound consists of ISIS 588540. In certain embodiments, ISIS 588540 has the following chemical structure:

In certain embodiments, a compound comprises or consists of a single-stranded modified oligonucleotide consisting of 16 linked nucleosides having a nucleobase sequence consisting of the sequence recited in SEQ ID NO: 549, wherein the modified oligonucleotide comprises
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment; wherein each nucleoside of each wing segment comprises a cEt sugar; wherein each internucleoside linkage is a phosphorothioate linkage; and wherein each cytosine is a 5-methylcytosine.

In certain embodiments, a compound comprises or consists of a single-stranded modified oligonucleotide consisting of 16 linked nucleosides having a nucleobase sequence comprising the sequence recited in SEQ ID NO: 598, wherein the modified oligonucleotide comprises
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment: wherein the 5′ wing segment comprises a 2′—O-methoxyethyl sugar, 2′—O-methoxyethyl sugar, and cEt sugar in the 5′ to 3′ direction: wherein the 3′ wing segment comprises a cEt sugar, cEt sugar, and 2′—O-methoxyethyl sugar in the 5′ to 3′ direction: wherein each internucleoside linkage is a phosphorothioate linkage: and wherein each cytosine is a 5-methylcytosine.

In any of the foregoing embodiments, the compound or oligonucleotide can be at least 85%, at least 90%, at least 95%, at least 98%, at least 99%, or 100% complementary to a nucleic acid encoding CFB.

In any of the foregoing embodiments, the compound or oligonucleotide can be single-stranded.

In certain embodiments, the compounds or compositions as described herein are efficacious by virtue of having at least one of an in vitro IC₅₀ of less than 250 nM, less than 200 nM, less than 150 nM, less than 100 nM, less than 90 nM, less than 80 nM, less than 70 nM, less than 65 nM, less than 60 nM, less than 55 nM, less than 50 nM, less than 45 nM, less than 40 nM, less than 35 nM, less than 30 nM, less than 25 nM, or less than 20 nM.

In certain embodiments, the compounds or compositions as described herein are highly tolerable as demonstrated by having at least one of an increase an ALT or AST value of no more than 4 fold, 3 fold, or 2 fold over saline treated animals or an increase in liver, spleen, or kidney weight of no more than 30%, 20%, 15%, 12%, 10%, 5%, or 2%. In certain embodiments, the compounds or compositions as described herein are highly tolerable as demonstrated by having no increase of ALT or AST over saline treated animals. In certain embodiments, the compounds or compositions as described herein are highly tolerable as demonstrated by having no increase in liver, spleen, or kidney weight over saline treated animals.

Certain embodiments provide a composition comprising the compound of any of the aforementioned embodiments or salt thereof and at least one of a pharmaceutically acceptable carrier or diluent. In certain embodiments, the composition has a viscosity less than about 40 centipoise (cP), less than about 30 centipose (cP), less than about 20 centipose (cP), less than about 15 centipose (cP), or less than about 10 centipose (cP). In certain embodiments, the composition having any of the aforementioned viscosities comprises a compound provided herein at a concentration of about 100 mg/mL, about 125 mg/mL, about 150 mg/mL, about 175 mg/mL, about 200 mg/mL, about 225 mg/mL, about 250 mg/mL, about 275 mg/mL, or about 300 mg/mL. In certain embodiments, the composition having any of the aforementioned viscosities and/or compound concentrations has a temperature of room temperature or about 20° C., about 21° C., about 22° C., about 23° C., about 24° C., about 25° C., about 26° C., about 27° C., about 28° C., about 29° C., or about 30° C.

In certain embodiments, a method of treating, preventing, or ameliorating a disease associated with dysregulation of the complement alternative pathway in a subject comprises administering to the subject a specific inhibitor of Complement Factor B (CFB), thereby treating, preventing, or ameliorating the disease. In certain embodiments, the complement alternative pathway is activated greater than normal. In certain embodiments, the CFB specific inhibitor is an antisense compound targeted to CFB, such as an antisense oligonucleotide targeted to CFB. In certain embodiments, the CFB specific inhibitor is a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598. In certain embodiments, the CFB specific inhibitor is a compound comprising or consisting of ISIS 532770, 532800, 532809, 588540, 588544, 588548, 588550, 588553, 588555, 588848, or 594430. In certain embodiments, the CFB specific inhibitor is a compound comprising or consisting of ISIS 588540, which has the following chemical structure:

In certain embodiments, the disease is macular degeneration, such as age related macular degeneration (AMD), which can be wet AMD or dry AMD. In certain embodiments, dry AMD can be Geographic Atrophy. In certain embodiments, the disease is a kidney disease such as lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

In certain embodiments, a method of treating, preventing, or ameliorating macular degeneration, such as age-related macular degeneration (AMD) in a subject comprises administering to the subject a CFB specific inhibitor, thereby treating, preventing, or ameliorating AMD, such as wet AMD and dry AMD. In certain embodiments, dry AMD can be Geographic Atrophy. Geographic Atrophy is considered an advanced form of dry AMD involving degeneration of the retina. In certain embodiments, the subject has a complement alternative pathway that is activated greater than normal. In certain embodiments, administering the antisense compound reduces or inhibits accumulation of ocular C3 levels, such as C3 protein levels. In certain embodiments, administering the antisense compound reduces the level of ocular C3 deposits or inhibits accumulation of ocular C3 deposits. In certain embodiments, the CFB specific inhibitor is an antisense compound targeted to CFB, such as an antisense oligonucleotide targeted to CFB. In certain embodiments, the CFB specific inhibitor is a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598. In certain embodiments, the CFB specific inhibitor is a compound comprising or consisting of ISIS 532770, 532800, 532809, 588540, 588544, 588548, 588550, 588553, 588555, 588848, or 594430. In certain embodiments, the compound is administered to the subject parenterally.

In certain embodiments, a method of treating, preventing, or ameliorating a kidney disease associated with dysregulation of the complement alternative pathway in a subject comprises administering to the subject a specific inhibitor of Complement Factor B (CFB), thereby treating, preventing, or ameliorating the kidney disease. In certain embodiments, the complement alternative pathway is activated greater than normal. In certain embodiments, the CFB specific inhibitor is an antisense compound targeted to CFB, such as an antisense oligonucleotide targeted to CFB. In certain embodiments, the CFB specific inhibitor is a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598. In certain embodiments, the CFB specific inhibitor is a compound comprising or consisting of ISIS 532770, 532800, 532809, 588540, 588544, 588548, 588550, 588553, 588555, 588848, or 594430. In certain embodiments, the compound is administered to the subject parenterally. In certain embodiments, the kidney disease is lupus nephritis, systemic lupus erythematosus (SLE), dense deposit disease (DDD). C3 glomerulonephritis (C3GN). CFHR5 nephropathy, or atypical hemolytic uremic syndrome (aHUS), or any combination thereof. In certain embodiments, the kidney disease is associated with C3 deposits, such as C3 deposits in the glomerulus. In certain embodiments, the kidney disease is associated with lower than normal circulating C3 levels, such as serum or plasma C3 levels. In certain embodiments, administering the compound reduces or inhibits accumulation of C3 levels in the kidney, such as C3 protein levels. In certain embodiments, administering the compound reduces the level of kidney C3 deposits or inhibits accumulation of kidney C3 deposits, such as C3 levels in the glomerulus. In certain embodiments, the subject is identified as having or at risk of having a disease associated with dysregulation of the complement alternative pathway, for example by detecting complement levels or membrane-attack complex levels in the subject's blood and/or performing a genetic test for gene mutations of complement factors associated with the disease.

In certain embodiments, a method of inhibiting expression of Complement Factor B (CFB) in a subject having, or at risk of having, a disease associated with dysregulation of the complement alternative pathway comprises administering a Complement Factor B (CFB) specific inhibitor to the subject, thereby inhibiting expression of CFB in the subject. In certain embodiments, administering the inhibitor inhibits expression of CFB in the eye. In certain embodiments, the subject has, or is at risk of having, age related macular degeneration (AMD), such as wet AMD and dry AMD. In certain embodiments, dry AMD can be Geographic Atrophy. In certain embodiments, administering the inhibitor inhibits expression of CFB in the kidney, such as in the glomerulus. In certain embodiments, the subject has, or is at risk of having, lupus nephritis, systemic lupus erythematosus (SLE), dense deposit disease (DDD). C3 glomerulonephritis (C3GN). CFHR5 nephropathy, or atypical hemolytic uremic syndrome (aHUS), or any combination thereof. In certain embodiments, the CFB specific inhibitor is a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598. In certain embodiments, the CFB specific inhibitor is a compound comprising or consisting of ISIS 532770, 532800, 532809, 588540, 588544, 588548, 588550, 588553, 588555, 588848, or 594430. In certain embodiments, the compound is administered to the subject parenterally.

In certain embodiments, a method of reducing or inhibiting accumulation of C3 deposits in the eye of a subject having, or at risk of having, a disease associated with dysregulation of the complement alternative pathway comprises administering a Complement Factor B (CFB) specific inhibitor to the subject, thereby reducing or inhibiting accumulation of C3 deposits in the eye of the subject. In certain embodiments, the subject has, or is at risk of having, age related macular degeneration (AMD), such as wet AMD and dry AMD. In certain embodiments, dry AMD can be Geographic Atrophy. In certain embodiments, the inhibitor is an antisense compound targeted to CFB. In certain embodiments, the CFB specific inhibitor is a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598. In certain embodiments, the CFB specific inhibitor is a compound comprising or consisting of ISIS 532770, 532800, 532809, 588540, 588544, 588548, 588550, 588553, 588555, 588848, or 594430. In certain embodiments, the compound is administered to the subject parenterally.

In certain embodiments, a method of reducing or inhibiting accumulation of C3 deposits in the kidney of a subject having, or at risk of having, a disease associated with dysregulation of the complement alternative pathway comprises administering a Complement Factor B (CFB) specific inhibitor to the subject, thereby reducing or inhibiting accumulation of C3 deposits in the kidney of the subject. In certain embodiments, the subject has, or is at risk of having, lupus nephritis, systemic lupus erythematosus (SLE), dense deposit disease (DDD). C3 glomerulonephritis (C3GN). CFHR5 nephropathy, or atypical hemolytic uremic syndrome (aHUS), or any combination thereof. In certain embodiments, the inhibitor is an antisense compound targeted to CFB. In certain embodiments, the CFB specific inhibitor is a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 6-808. In certain embodiments, the CFB specific inhibitor is a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598. In certain embodiments, the CFB specific inhibitor is a compound comprising or consisting of ISIS 532770, 532800, 532809, 588540, 588544, 588548, 588550, 588553, 588555, 588848, or 594430. In certain embodiments, the compound is administered to the subject parenterally.

Certain embodiments are drawn to a compound or composition described herein for use in therapy. Certain embodiments are drawn to a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808 for use in therapy. Certain embodiments are drawn to a compound comprising or consisting of a modified oligonucleotide consisting of 10 to 30 linked nucleosides having a nucleobase sequence comprising or consisting of any one of SEQ ID NOs: 6-808 for use in therapy. Certain embodiments are drawn to a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598, for use in therapy. Certain embodiments are drawn to a compound comprising or consisting of ISIS 532770, 532800, 532809, 588540, 588544, 588548, 588550, 588553, 588555, 588848, or 594430 for use in therapy.

Certain embodiments are drawn to a compound or composition described herein for use in treating a disease associated with dysregulation of the complement alternative pathway. Certain embodiments are drawn to a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808 for use in treating a disease associated with dysregulation of the complement alternative pathway. Certain embodiments are drawn to a compound comprising or consisting of a modified oligonucleotide consisting of 10 to 30 linked nucleosides having a nucleobase sequence comprising or consisting of any one of SEQ ID NOs: 6-808 for use in treating a disease associated with dysregulation of the complement alternative pathway. Certain embodiments are drawn to a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598, for use in treating a disease associated with dysregulation of the complement alternative pathway. Certain embodiments are drawn to a compound comprising or consisting of ISIS 532770, 532800, 532809, 588540, 588544, 588548, 588550, 588553, 588555, 588848, or 594430 for use in treating a disease associated with dysregulation of the complement alternative pathway. In certain embodiments, the complement alternative pathway is activated greater than normal. In certain embodiments, the disease is macular degeneration, such as age related macular degeneration (AMD), which can be wet AMD or dry AMD. In certain embodiments, dry AMD can be Geographic Atrophy. In certain embodiments, the disease is a kidney disease such as lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

Certain embodiments are drawn to a compound comprising or consisting of ISIS 588540, which has the following chemical structure:
, for use in treating a disease associated with dysregulation of the complement alternative pathway. In certain embodiments, the complement alternative pathway is activated greater than normal. In certain embodiments, the disease is macular degeneration, such as age related macular degeneration (AMD), which can be wet AMD or dry AMD. In certain embodiments, dry AMD can be Geographic Atrophy. In certain embodiments, the disease is a kidney disease such as lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

Certain embodiments are drawn to use of a compound or composition described herein for the manufacture of a medicament for treating a disease associated with dysregulation of the complement alternative pathway. Certain embodiments are drawn to use of a compound comprising or consisting of modified oligonucleotide consisting of 10 to 30 linked nucleosides and having a nucleobase sequence comprising at least 8 contiguous nucleobases of any of the nucleobase sequences of SEQ ID NOs: 6-808 for the manufacture of a medicament for treating disease associated with dysregulation of the complement alternative pathway. Certain embodiments are drawn to use of a compound comprising or consisting of a modified oligonucleotide consisting of 10 to 30 linked nucleosides having a nucleobase sequence comprising or consisting of any one of SEQ ID NOs: 6-808 for the manufacture of a medicament for treating a disease associated with dysregulation of the complement alternative pathway. Certain embodiments are drawn to use of a compound comprising or consisting of a modified oligonucleotide consisting of 10 to 30 linked nucleosides having a nucleobase sequence comprising or consisting of any one of SEQ ID NOs: 198, 228, 237, 440, 444, 448, 450, 453, 455, 549, and 598, for the manufacture of a medicament for treating a disease associated with dysregulation of the complement alternative pathway. Certain embodiments are drawn to use of a compound comprising or consisting of ISIS 532770, 532800, 532809, 588540, 588544, 588548, 588550, 588553, 588555, 588848, or 594430 for the manufacture of a medicament for treating a disease associated with dysregulation of the complement alternative pathway. In certain embodiments, the complement alternative pathway is activated greater than normal. In certain embodiments, the disease is macular degeneration, such as age related macular degeneration (AMD), which can be wet AMD or dry AMD. In certain embodiments, dry AMD can be Geographic Atrophy. In certain embodiments, the disease is a kidney disease such as lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

Certain embodiments are drawn to use of a compound comprising or consisting of ISIS 588540, which has the following chemical structure:
, for the manufacture of a medicament for treating a disease associated with dysregulation of the complement alternative pathway. In certain embodiments, the complement alternative pathway is activated greater than normal. In certain embodiments, the disease is macular degeneration, such as age related macular degeneration (AMD), which can be wet AMD or dry AMD. In certain embodiments, dry AMD can be Geographic Atrophy. In certain embodiments, the disease is a kidney disease such as lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

In any of the foregoing embodiments, the CFB specific inhibitor can be an antisense compound targeted to CFB. In certain embodiments, the antisense compound comprises an antisense oligonucleotide, for example an antisense oligonucleotide consisting of 8 to 80 linked nucleosides, 12 to 30 linked nucleosides, or 20 linked nucleosides. In certain embodiments, the antisense oligonucleotide is at least 80%, 85%, 90%, 95% or 100% complementary to any of the nucleobase sequences recited in SEQ ID NOs: 1-5. In certain embodiments, the antisense oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′—O-methoxyethyl, and the modified nucleobase is a 5-methylcytosine. In certain embodiments, the modified oligonucleotide comprises a gap segment consisting of linked deoxynucleosides; a 5′ wing segment consisting of linked nucleosides: and a 3′ wing segment consisting of linked nucleosides, wherein the gap segment is positioned immediately adjacent to and between the 5′ wing segment and the 3′ wing segment and wherein each nucleoside of each wing segment comprises a modified sugar. In certain embodiments, the antisense oligonucleotide is administered parenterally. For example, in certain embodiments the antisense oligonucleotide can be administered through injection or infusion. Parenteral administration includes subcutaneous administration, intravenous administration, intramuscular administration, intraarterial administration, intraperitoneal administration, or intracranial administration, e.g. intrathecal or intracerebroventricular administration.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted.

In certain embodiments, an antisense compound is 10 to 30 subunits in length. In certain embodiments, an antisense compound is 12 to 30 subunits in length. In certain embodiments, an antisense compound is 12 to 22 subunits in length. In certain embodiments, an antisense compound is 14 to 30 subunits in length. In certain embodiments, an antisense compound is 14 to 20 subunits in length. In certain embodiments, an antisense compound is 15 to 30 subunits in length. In certain embodiments, an antisense compound is 15 to 20 subunits in length. In certain embodiments, an antisense compound is 16 to 30 subunits in length. In certain embodiments, an antisense compound is 16 to 20 subunits in length. In certain embodiments, an antisense compound is 17 to 30 subunits in length. In certain embodiments, an antisense compound is 17 to 20 subunits in length. In certain embodiments, an antisense compound is 18 to 30 subunits in length. In certain embodiments, an antisense compound is 18 to 21 subunits in length. In certain embodiments, an antisense compound is 18 to 20 subunits in length. In certain embodiments, an antisense compound is 20 to 30 subunits in length. In other words, such antisense compounds are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, an antisense compound is 14 subunits in length. In certain embodiments, an antisense compound is 16 subunits in length. In certain embodiments, an antisense compound is 17 subunits in length. In certain embodiments, an antisense compound is 18 subunits in length. In certain embodiments, an antisense compound is 19 subunits in length. In certain embodiments, an antisense compound is 20 subunits in length. In other embodiments, the antisense compound is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides.

In certain embodiments antisense oligonucleotides may be shortened or truncated. For example, a single subunit may be deleted from the 5′ end (5′ truncation), or alternatively from the 3′ end (3′ truncation). A shortened or truncated antisense compound targeted to an CFB nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al. (J. Natl. Cancer Inst. 93:463-471, March 2001) demonstrated the ability of an oligonucleotide having 100% complementarity to the bel-2 mRNA and having 3 mismatches to the bcl-xL mRNA to reduce the expression of both bcl-2 and bel-xL in vitro and in vivo. Furthermore, this oligonucleotide demonstrated potent anti-tumor activity in vivo.

Maher and Dolnick (Nuc. Acid. Res. 16:3341-3358,1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Certain Antisense Compound Motifs and Mechanisms

In certain embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which utilize the RISC pathway and include, without limitation, siRNA, ssRNA and microRNA mechanisms: and occupancy based mechanisms. Certain antisense compounds may act through more than one such mechanism and/or through additional mechanisms.

### RNase H-Mediated Antisense

In certain embodiments, antisense activity results at least in part from degradation of target RNA by RNase H. RNase H is a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In certain embodiments, antisense compounds that utilize RNase H comprise one or more modified nucleosides. In certain embodiments, such antisense compounds comprise at least one block of 1-8 modified nucleosides. In certain such embodiments, the modified nucleosides do not support RNase H activity. In certain embodiments, such antisense compounds are gapmers, as described herein. In certain such embodiments, the gap of the gapmer comprises DNA nucleosides. In certain such embodiments, the gap of the gapmer comprises DNA-like nucleosides. In certain such embodiments, the gap of the gapmer comprises DNA nucleosides and DNA-like nucleosides.

Certain antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE and 2′—O—CH₃, among others), and bicyclic sugar modified nucleosides (such bicyclic sugar modified nucleosides may include those having a constrained ethyl). In certain embodiments, nucleosides in the wings may include several modified sugar moieties, including, for example 2′-MOE and bicyclic sugar moieties such as constrained ethyl or LNA. In certain embodiments, wings may include several modified and unmodified sugar moieties. In certain embodiments, wings may include various combinations of 2′-MOE nucleosides, bicyclic sugar moieties such as constrained ethyl nucleosides or LNA nucleosides, and 2′-deoxynucleosides.

Each distinct region may comprise uniform sugar moieties, variant, or alternating sugar moieties. The wing-gap-wing motif is frequently described as “X-Y-Z”, where “X” represents the length of the 5′-wing. “Y” represents the length of the gap, and “Z” represents the length of the 3′-wing. “X” and “Z” may comprise uniform, variant, or alternating sugar moieties. In certain embodiments. “X” and “Y” may include one or more 2′-deoxynucleosides. “Y” may comprise 2′-deoxynucleosides. As used herein, a gapmer described as “X—Y-Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, “Y” is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30 or more nucleosides.

In certain embodiments, the antisense compound targeted to a CFB nucleic acid has a gapmer motif in which the gap consists of 6, 7, 8, 9, 10, 11, 12, 13, 14, 15, or 16 linked nucleosides.

In certain embodiments, the antisense oligonucleotide has a sugar motif described by Formula A as follows: (J)ₘ-(B)ₙ-(J)ₚ-(B)ᵣ-(A)ₜ-(D)_{g}-(A)ᵥ-(B)_{w}-(J)ₓ-(B)_{y}-(J)_{z}
wherein:
each A is independently a 2′-substituted nucleoside;
each B is independently a bicyclic nucleoside;
each J is independently either a 2′-substituted nucleoside or a 2′-deoxynucleoside;
each D is a 2′-deoxynucleoside;
m is 0-4; n is 0-2; p is 0-2; r is 0-2; t is 0-2; v is 0-2; w is 0-4; x is 0-2; y is 0-2; z is 0-4; g is 6-14; provided that:
at least one of m, n, and r is other than 0;
at least one of w and y is other than 0;
the sum of m, n, p, r, and t is from 2 to 5; and
the sum of v, w, x, y, and z is from 2 to 5.

### RNAi Compounds

In certain embodiments, antisense compounds are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). In certain embodiments, antisense compounds comprise modifications that make them particularly suited for such mechanisms.

i. ssRNA Compounds

In certain embodiments, antisense compounds including those particularly suited for use as single-stranded RNAi compounds (ssRNA) comprise a modified 5′-terminal end. In certain such embodiments, the 5′-terminal end comprises a modified phosphate moiety. In certain embodiments, such modified phosphate is stabilized (e.g., resistant to degradation/cleavage compared to unmodified 5′-phosphate). In certain embodiments, such 5′-terminal nucleosides stabilize the 5′-phosphorous moiety. Certain modified 5′-terminal nucleosides may be found in the art, for example in WO/2011/139702.

In certain embodiments, the 5′-nucleoside of an ssRNA compound has Formula IIc:

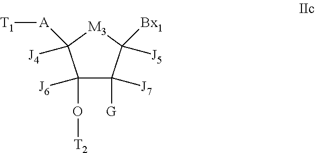

wherein:
T₁ is an optionally protected phosphorus moiety;
T₂ is an internucleoside linking group linking the compound of Formula IIc to the oligomeric compound:

A has one of the formulas:

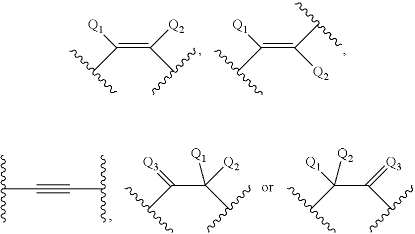

Q₁ and Q₂ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl or N(R₃)(R₄);
Q₃ is O, S, N(R₅) or C(R₆)(R₇);
each R₃, R₄ R₅, R₆ and R₇ is, independently, H, C₁-C₆ alkyl, substituted C₁-C₆ alkyl or C₁-C₆ alkoxy;
M₃ is O, S, NR₁₄, C(R₁₅)(R₁₆), C(R₁₅)(R₁₆)C(R₁₇)(R₁₈), C(R₁₅)═C(R₁₇), OC(R₁₅)(R₁₆) or OC(R₁₅)(Bx₂);
R₁₄ is H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
R₁₅, R₁₆, R₁₇ and R₁₈ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
Bx₁ is a heterocyclic base moiety;
or if Bx₂ is present then Bx₂ is a heterocyclic base moiety and Bx₁ is H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
J₄, J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
or J₄ forms a bridge with one of J₅ or J₇ wherein said bridge comprises from 1 to 3 linked biradical groups selected from O, S, NR₁₉, C(R₂₀)(R₂₁), C(R₂₀)═C(R₂₁), C[═C(R₂₀)(R₂₁)] and C(═O) and the other two of J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
each R₁₉, R₂₀ and R₂₁ is, independently, H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
G is H, OH, halogen or O—[C(R₅)(R₉)]n-[(C═O)ₘ—X₁]ⱼ—Z;
each R₈ and R₉ is, independently, H, halogen, C₁-C₆ alkyl or substituted C₁-C₆ alkyl;
X₁ is O, S or N(E₁);
Z is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to about 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(═X₂)N(J₁)(J₂); X₂ is O, S or NJ₃;
each J₁, J₂ and J₃ is, independently, H or C₁-C₆ alkyl;
when j is 1 then Z is other than halogen or N(E₂)(E₃); and
wherein said oligomeric compound comprises from 8 to 40 monomeric subunits and is hybridizable to at least a portion of a target nucleic acid.

In certain embodiments, M₃ is O, CH═CH, OCH₂ or OC(H)(Bx₂). In certain embodiments, M₃ is O.

In certain embodiments, J₄, J₅, J₆ and J₇ are each H. In certain embodiments, J₄ forms a bridge with one of J₅ or J₇.

In certain embodiments, A has one of the formulas:

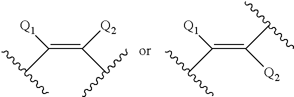

wherein:
Q₁ and Q₂ are each, independently, H, halogen, C₁-C₆ alkyl, substituted C₁-C₆ alkyl, C₁-C₆ alkoxy or substituted C₁-C₆ alkoxy. In certain embodiments, Q₁ and Q₂ are each H. In certain embodiments, Q¹ and Q₂ are each, independently, H or halogen. In certain embodiments, Q₁ and Q₂ is H and the other of Q¹ and Q₂ is F, CH₃ or OCH₃.

In certain embodiments, T₁ has the formula:

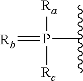

wherein:
Rₐ and R_{c} are each, independently, protected hydroxyl, protected thiol, C₁-C₆ alkyl, substituted C₁-C₆ alkyl, C₁-C₆ alkoxy, substituted C₁-C₆ alkoxy, protected amino or substituted amino; and
R_{b} is O or S. In certain embodiments, R_{b} is O and Rₐ and R_{c} are each, independently, OCH₃, OCH₂CH₃ or CH(CH₃)₂.

In certain embodiments, G is halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₁₀)(R₁₁), O(CH₂)₂—ON(R₁₀)(R₁₁), O(CH₂)₂—O(CH₂)₂—N(R₁₀)(R₁₁), OCH₂C(═O)—N(R₁₀)(R₁₁), OCH₂C(═O)—N(R₁₂)—(CH₂)₂—N(R₁₀)(R₁₁) or O(CH₂)₂—N(R₁₂)—C(═NR₁₃)[N(R₁₀)(R₁₁)] wherein R₁₀, R₁₁, R₁₂ and R₁₃ are each, independently, H or C₁-C₆ alkyl. In certain embodiments, G is halogen, OCH₃, OCF₃, OCH₂CH₃, OCH₂CF₃, OCH₂—CH—CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂ or OCH₂—N(H)—C(═NH)NH₂. In certain embodiments, G is F, OCH₃ or O(CH₂)₂—OCH₃. In certain embodiments, G is O(CH₂)₂—OCH₃.

In certain embodiments, the 5′-terminal nucleoside has Formula Ile:

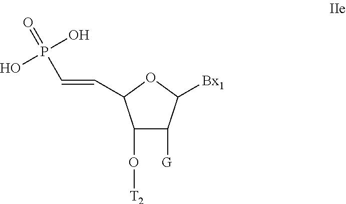

In certain embodiments, antisense compounds, including those particularly suitable for ssRNA comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having uniform sugar modifications. In certain such embodiments, each nucleoside of the region comprises the same RNA-like sugar modification. In certain embodiments, each nucleoside of the region is a 2′—F nucleoside. In certain embodiments, each nucleoside of the region is a 2′—OMe nucleoside. In certain embodiments, each nucleoside of the region is a 2′-MOE nucleoside. In certain embodiments, each nucleoside of the region is a cEt nucleoside. In certain embodiments, each nucleoside of the region is an LNA nucleoside. In certain embodiments, the uniform region constitutes all or essentially all of the oligonucleotide. In certain embodiments, the region constitutes the entire oligonucleotide except for 1-4 terminal nucleosides.

In certain embodiments, oligonucleotides comprise one or more regions of alternating sugar modifications, wherein the nucleosides alternate between nucleotides having a sugar modification of a first type and nucleotides having a sugar modification of a second type. In certain embodiments, nucleosides of both types are RNA-like nucleosides. In certain embodiments the alternating nucleosides are selected from: 2′—OMe, 2′-F, 2′-MOE, LNA, and cEt. In certain embodiments, the alternating modifications are 2′-F and 2′-OMe. Such regions may be contiguous or may be interrupted by differently modified nucleosides or conjugated nucleosides.

In certain embodiments, the alternating region of alternating modifications each consist of a single nucleoside (i.e., the pattern is (AB)ₓA_{y} wherein A is a nucleoside having a sugar modification of a first type and B is a nucleoside having a sugar modification of a second type; x is 1-20 and y is 0 or 1). In certain embodiments, one or more alternating regions in an alternating motif includes more than a single nucleoside of a type. For example, oligonucleotides may include one or more regions of any of the following nucleoside motifs:
AABBAA;
ABBABB;
AABAAB;
ABBABAABB;
ABABAA;
AABABAB;
ABABAA;
ABBAABBABABAA;
BABBAABBABABAA or
ABABBAABBABABAA;
   wherein A is a nucleoside of a first type and B is a nucleoside of a second type. In certain embodiments, A and B are each selected from 2′-F, 2′—OMe, BNA, and MOE.

In certain embodiments, oligonucleotides having such an alternating motif also comprise a modified 5′ terminal nucleoside, such as those of formula IIc or Ile.

In certain embodiments, oligonucleotides comprise a region having a 2-2-3 motif. Such regions comprises the following motif:

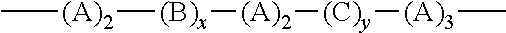

wherein: A is a first type of modifed nucleosde;
B and C, are nucleosides that are differently modified than A, however, B and C may have the same or different modifications as one another;
x and y are from 1 to 15.

In certain embodiments, A is a 2′—OMe modified nucleoside. In certain embodiments, B and C are both 2′-F modified nucleosides. In certain embodiments, A is a 2′—OMe modified nucleoside and B and C are both 2-F modified nucleosides.

In certain embodiments, oligonucleosides have the following sugar motif:

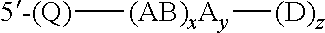

wherein:
Q is a nucleoside comprising a stabilized phosphate moiety. In certain embodiments, Q is a nucleoside having Formula IIc or Ile;
A is a first type of modifed nucleoside;
B is a second type of modified nucleoside;
D is a modified nucleoside comprising a modification different from the nucleoside adjacent to it.

Thus, if y is 0, then D must be differently modified than B and if y is 1, then D must be differently modified than A. In certain embodiments, D differs from both A and B.
X is 5-15;
Y is 0 or 1;
Z is 0-4.

In certain embodiments, oligonucleosides have the following sugar motif:

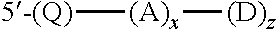

wherein:
Q is a nucleoside comprising a stabilized phosphate moiety. In certain embodiments, Q is a nucleoside having Formula IIc or Ile;
A is a first type of modifed nucleoside;
D is a modified nucleoside comprising a modification different from A.
X is 11-30;
Z is 0-4.

In certain embodiments A, B, C, and D in the above motifs are selected from: 2′—OMe, 2′-F, 2′-MOE, LNA, and cEt. In certain embodiments, D represents terminal nucleosides. In certain embodiments, such terminal nucleosides are not designed to hybridize to the target nucleic acid (though one or more might hybridize by chance). In certain embodiments, the nucleobase of each D nucleoside is adenine, regardless of the identity of the nucleobase at the corresponding position of the target nucleic acid. In certain embodiments the nucleobase of each D nucleoside is thymine.

In certain embodiments, antisense compounds, including those particularly suited for use as ssRNA comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least one block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Oligonucleotides having any of the various sugar motifs described herein, may have any linkage motif. For example, the oligonucleotides, including but not limited to those described above, may have a linkage motif selected from non-limiting the table below:

| 5′ most linkage | Central region | 3′-region |
|---|---|---|
| PS | Alternating PO/PS | 6 PS |
| PS | Alternating PO/PS | 7 PS |
| PS | Alternating PO/PS | 8 PS |

ii. siRNA Compounds

In certain embodiments, antisense compounds are double-stranded RNAi compounds (siRNA). In such embodiments, one or both strands may comprise any modification motif described above for ssRNA. In certain embodiments, ssRNA compounds may be unmodified RNA. In certain embodiments, siRNA compounds may comprise unmodified RNA nucleosides, but modified internucleoside linkages.

Several embodiments relate to double-stranded compositions wherein each strand comprises a motif defined by the location of one or more modified or unmodified nucleosides. In certain embodiments, compositions are provided comprising a first and a second oligomeric compound that are fully or at least partially hybridized to form a duplex region and further comprising a region that is complementary to and hybridizes to a nucleic acid target. It is suitable that such a composition comprise a first oligomeric compound that is an antisense strand having full or partial complementarity to a nucleic acid target and a second oligomeric compound that is a sense strand having one or more regions of complementarity to and forming at least one duplex region with the first oligomeric compound.

The compositions of several embodiments modulate gene expression by hybridizing to a nucleic acid target resulting in loss of its normal function. In certain embodiments, the degradation of the targeted CFB is facilitated by an activated RISC complex that is formed with compositions of the invention.

Several embodiments are directed to double-stranded compositions wherein one of the strands is useful in, for example, influencing the preferential loading of the opposite strand into the RISC (or cleavage) complex. The compositions are useful for targeting selected nucleic acid molecules and modulating the expression of one or more genes. In some embodiments, the compositions of the present invention hybridize to a portion of a target RNA resulting in loss of normal function of the target RNA.

Certain embodiments are drawn to double-stranded compositions wherein both the strands comprises a hemimer motif, a fully modified motif, a positionally modified motif or an alternating motif. Each strand of the compositions of the present invention can be modified to fulfil a particular role in for example the siRNA pathway. Using a different motif in each strand or the same motif with different chemical modifications in each strand permits targeting the antisense strand for the RISC complex while inhibiting the incorporation of the sense strand. Within this model, each strand can be independently modified such that it is enhanced for its particular role. The antisense strand can be modified at the 5′-end to enhance its role in one region of the RISC while the 3′-end can be modified differentially to enhance its role in a different region of the RISC.

The double-stranded oligonucleotide molecules can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The double-stranded oligonucleotide molecules can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double-stranded structure, for example wherein the double-stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the double-stranded oligonucleotide molecule are complementary to the target nucleic acid or a portion thereof). Alternatively, the double-stranded oligonucleotide is assembled from a single oligonucleotide, where the self-complementary sense and antisense regions of the siRNA are linked by means of a nucleic acid based or non-nucleic acid-based linker(s).

The double-stranded oligonucleotide can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The double-stranded oligonucleotide can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi.

In certain embodiments, the double-stranded oligonucleotide comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the double-stranded oligonucleotide comprises nucleotide sequence that is complementary to nucleotide sequence of a target gene. In another embodiment, the double-stranded oligonucleotide interacts with nucleotide sequence of a target gene in a manner that causes inhibition of expression of the target gene.

As used herein, double-stranded oligonucleotides need not be limited to those molecules containing only RNA, but further encompasses chemically modified nucleotides and non-nucleotides. In certain embodiments, the short interfering nucleic acid molecules lack 2′-hydroxy (2′—OH) containing nucleotides. In certain embodiments short interfering nucleic acids optionally do not include any ribonucleotides (e.g., nucleotides having a 2′—OH group). Such double-stranded oligonucleotides that do not require the presence of ribonucleotides within the molecule to support RNAi can however have an attached linker or linkers or other attached or associated groups, moieties, or chains containing one or more nucleotides with 2′—OH groups. Optionally, double-stranded oligonucleotides can comprise ribonucleotides at about 5, 10, 20, 30, 40, or 50% of the nucleotide positions. As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, double-stranded oligonucleotides can be used to epigenetically silence genes at both the post-transcriptional level and the pre-transcriptional level. In a non-limiting example, epigenetic regulation of gene expression by siRNA molecules of the invention can result from siRNA mediated modification of chromatin structure or methylation pattern to alter gene expression (sec. for example. Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

It is contemplated that compounds and compositions of several embodiments provided herein can target CFB by a dsRNA-mediated gene silencing or RNAi mechanism, including, e.g., “hairpin” or stem-loop double-stranded RNA effector molecules in which a single RNA strand with self-complementary sequences is capable of assuming a double-stranded conformation, or duplex dsRNA effector molecules comprising two separate strands of RNA. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA or dsRNA effector molecule may be a single molecule with a region of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may include two different strands that have a region of complementarity to each other.

In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. In certain embodiments, the regions of complementarity are at least 70, 80, 90, 95, 98, or 100% complementary to each other and to a target nucleic acid sequence. In certain embodiments, the region of the dsRNA that is present in a double-stranded conformation includes at least 19, 20, 21, 22, 23, 24, 25, 26, 27, 28, 29, 30, 50, 75, 100, 200, 500, 1000, 2000 or 5000 nucleotides or includes all of the nucleotides in a cDNA or other target nucleic acid sequence being represented in the dsRNA. In some embodiments, the dsRNA does not contain any single stranded regions, such as single stranded ends, or the dsRNA is a hairpin. In other embodiments, the dsRNA has one or more single stranded regions or overhangs. In certain embodiments. RNA/DNA hybrids include a DNA strand or region that is an antisense strand or region (e.g. has at least 70, 80, 90, 95, 98, or 100% complementarity to a target nucleic acid) and an RNA strand or region that is a sense strand or region (e.g. has at least 70, 80, 90, 95, 98, or 100% identity to a target nucleic acid), and vice versa.

In various embodiments, the RNA/DNA hybrid is made in vitro using enzymatic or chemical synthetic methods such as those described herein or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999.) Exemplary circular nucleic acids include lariat structures in which the free 5′ phosphoryl group of a nucleotide becomes linked to the 2′ hydroxyl group of another nucleotide in a loop back fashion.

In other embodiments, the dsRNA includes one or more modified nucleotides in which the 2′ position in the sugar contains a halogen (such as fluorine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. The dsRNAs may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000, or U.S. Ser. No. 60/130,377, filed Apr. 21, 1999.

In other embodiments, the dsRNA can be any of the at least partially dsRNA molecules disclosed in WO 00/63364, as well as any of the dsRNA molecules described in U.S. Provisional Application 60/399,998; and U.S. Provisional Application 60/419,532, and PCT/US2003/033466, the teaching of which is hereby incorporated by reference. Any of the dsRNAs may be expressed in vitro or in vivo using the methods described herein or standard methods, such as those described in WO 00/63364.

### Occupancy

In certain embodiments, antisense compounds are not expected to result in cleavage or the target nucleic acid via RNase H or to result in cleavage or sequestration through the RISC pathway. In certain such embodiments, antisense activity may result from occupancy, wherein the presence of the hybridized antisense compound disrupts the activity of the target nucleic acid. In certain such embodiments, the antisense compound may be uniformly modified or may comprise a mix of modifications and/or modified and unmodified nucleosides.

### Target Nucleic Acids, Target Regions and Nucleotide Sequences

Nucleotide sequences that encode Complement Factor B (CFB) include, without limitation, the following: GENBANK Accession No. NM_001710.5 (incorporated herein as SEQ ID NO: 1), GENBANK Accession No. NT_007592.15 truncated from nucleotides 31852000 to 31861000 (incorporated herein as SEQ ID NO: 2), GENBANK Accession No NW_001116486.1 truncated from nucleotides 536000 to 545000 (incorporated herein as SEQ ID NO: 3), GENBANK Accession No. XM_001113553.2 (incorporated herein as SEQ ID NO: 4), or GENBANK Accession No. NM_008198.2 (incorporated herein as SEQ ID NO: 5).

### Hybridization

In some embodiments, hybridization occurs between an antisense compound disclosed herein and a CFB nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a CFB nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a CFB nucleic acid).

Non-complementary nucleobases between an antisense compound and a CFB nucleic acid may be tolerated provided that the antisense compound remains able to specifically hybridize to a target nucleic acid. Moreover, an antisense compound may hybridize over one or more segments of a CFB nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a CFB nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having four noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package. Version 8 for Unix. Genetics Computer Group. University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound may be fully complementary to a CFB nucleic acid, or a target region, or a target segment or target sequence thereof. As used herein. “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a CFB nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a CFB nucleic acid, or specified portion thereof.

The antisense compounds provided also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds are complementary to at least an 11 nucleobase portion of a target segment. In certain embodiments, the antisense compounds are complementary to at least a 12 nucleobase portion of a target segment. In certain embodiments, the antisense compounds are complementary to at least a 13 nucleobase portion of a target segment. In certain embodiments, the antisense compounds are complementary to at least a 14 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence. SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are, or are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a CFB nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H. C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′ 0,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′—OCH₃, 2′—OCH₂CH₃, 2′—OCH₂CH₂F and 2′—O(CH₂)₂OCH₃ substituent groups. The substituent at the 2′ position can also be selected from allyl, amino, azido, thio, O-allyl. O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each R₁, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′—(CH₂)—O-2′ (LNA); 4′—(CH₂)—S-2′; 4′—(CH₂)₂—O-2′ (ENA); 4′—CH(CH₃)—O-2′ (also referred to as constrained ethyl or cEt) and 4′—CH(CH₂OCH₃)—O-2′ (and analogs thereof see U.S. Pat. No. 7,399,845, issued on Jul. 15, 2008); 4′—C(CH₃)(CH₃)—O-2′ (and analogs thereof see published International Application WO/2009/006478, published Jan. 8, 2009); 4′—CH₂—N(OCH₃)-2′ (and analogs thereof see published International Application WO/2008/150729, published Dec. 11, 2008); 4′—CH₂—O—N(CH₃)-2′ (see published U.S. Patent Application US2004-0171570, published September 2. 2004); 4′—CH₂—N(R)—O-2′, wherein R is H, C₁-C₁₂ alkyl, or a protecting group (see U.S. Pat. No. 7,427,672, issued on Sep. 23, 2008); 4′—CH₂—C(H)(CH₃)-2′ (see Chattopadhyaya et al., J. Org. Chem., 2009, 74, 118-134); and 4′—CH₂—C(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs. 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and ß-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′—CH₂-2′, 4′—(CH₂)₂-2′, 4′—(CH₂)₃-2′, 4′—CH₂—O-2′, 4′—(CH₂)₂—O-2′, 4′—CH₂—O—N(R)-2′ and 4′—CH₂—N(R)—O-2′— wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the ß-D configuration. Previously, α-L-methyleneoxy (4′—CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′—CH₂—O-2′) BNA, (B) β-D-methyleneoxy (4′—CH₂—O-2′) BNA, (C) ethyleneoxy (4′—(CH₂)₂—O-2′) BNA, (D) aminooxy (4′—CH₂—O—N(R)-2′) BNA, (E) oxyamino (4′—CH₂—N(R)—O-2′) BNA, and (F) methyl(methyleneoxy) (4′—CH(CH₃)—O-2′) BNA, (G) methylene-thio (4′—CH₂—S-2′) BNA, (H) methylene-amino (4′—CH₂—N(R)-2′) BNA, (I) methyl carbocyclic (4′—CH₂—CH(CH₃)-2′) BNA, (J) propylene carbocyclic (4′—(CH₂)₃-2′) BNA and (K) vinyl BNA as depicted below:

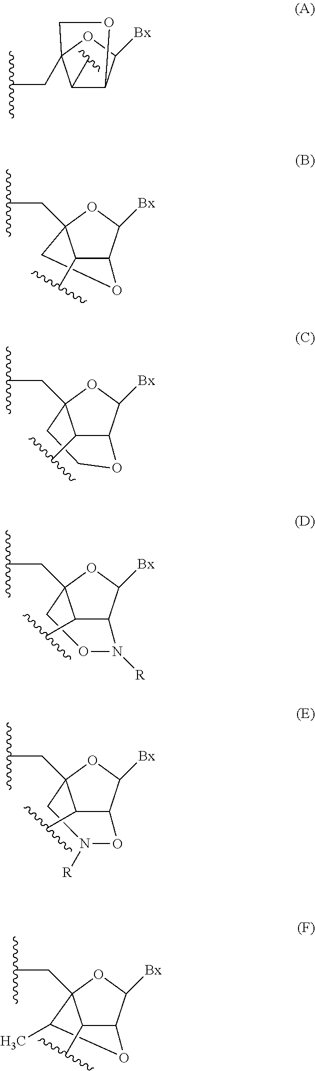

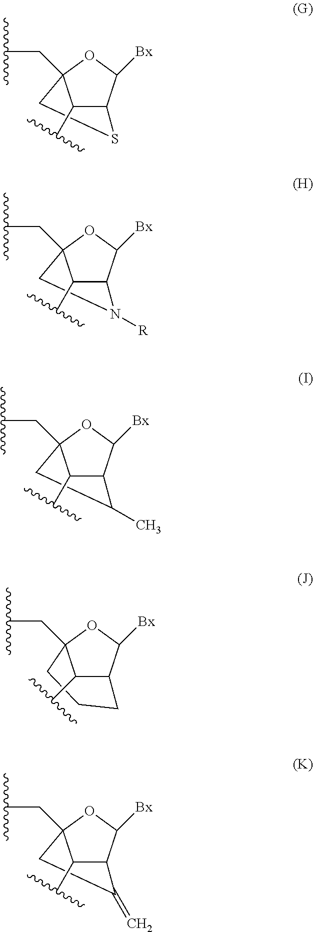

wherein Bx is the base moiety and R is independently H, a protecting group, C₁-C₁₂ alkyl or C₁-C₁₂ alkoxy.

In certain embodiments, bicyclic nucleosides are provided having Formula I:

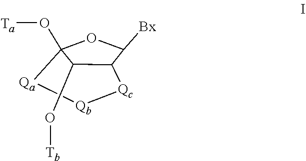

wherein:
Bx is a heterocyclic base moiety;
-Qₐ-Q_{b}-Q_{c}-is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

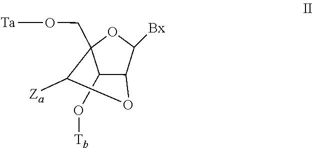

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and J_{c} is, independently, H, C₁-C₆ alkyl, or substituted C₁-C₆ alkyl and X is O or NJ_{c}.

In certain embodiments, bicyclic nucleosides are provided having Formula III:

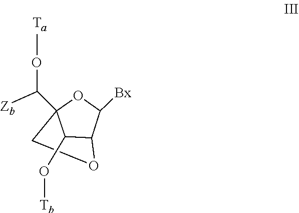

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

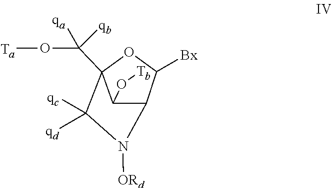

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Rₐ is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

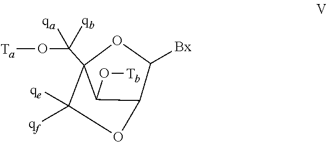

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, q_{c} and qᵣ are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or q_{c} and q_{f} together are ═C(q_{g})(qₕ);
q_{g} and qₕ are each, independently, H, halogen, C₁-C₁₂ alkyl or substituted C₁-C₁₂ alkyl.

The synthesis and preparation of the methyleneoxy (4′—CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′—CH₂—O-2) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

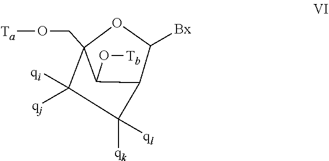

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′—(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, C₁, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modifed nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′—O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854) or fluoro HNA (F—HNA) having a tetrahydropyran ring system as illustrated below:

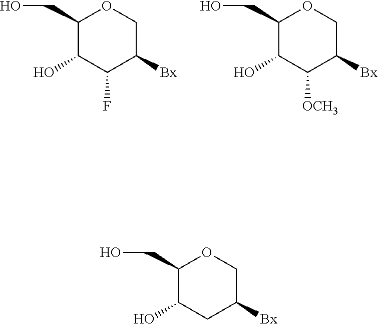

In certain embodiments, sugar surrogates are selected having Formula VII:

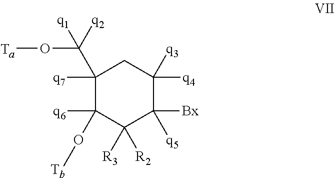

wherein independently for each of said at least one tetrahydropyran nucleoside analog of Formula VII:
Bx is a heterocyclic base moiety;
Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H, R₁ is methoxy and R₂ is H, and R₁ is methoxyethoxy and R₂ is H.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following formula:

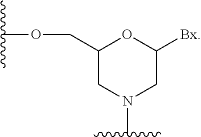

In certain embodiments, morpholinos may be modified, for example by adding or altering various substituent groups from the above morpholino structure. Such sugar surrogates are referred to herein as “modifed morpholinos.”

Combinations of modifications are also provided without limitation, such as 2′-F-5′-methyl substituted nucleosides (see PCT International Application WO 2008/101157 published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′—CH₂—O—2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130(6), 1979-1984; Horváth et al., Tetrahedron Letters. 2007, 48, 3621-3623; Nauwelaerts et al., J. Am. Chem. Soc., 2007, 129(30), 9340-9348; Gu et al., Nucleosides. Nucleotides & Nucleic Acids, 2005, 24(5-7), 993-998; Nauwelaerts et al., Nucleic Acids Research. 2005, 33(8), 2452-2463; Robeyns et al., Acta Crystallographica. Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have Formula X.

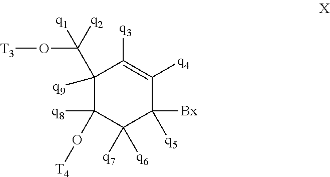

wherein independently for each of said at least one cyclohexenyl nucleoside analog of Formula X: Bx is a heterocyclic base moiety:
T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and
q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2 substituents, such as allyl, amino, azido, thio, O-allyl, O—C₁-C₁₀ alkyl, —OCF₃, O—(CH₂)₂—O—CH₃, 2′—O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modifed nucleosides may further comprise other modifications, for example at other positions of the sugar and/or at the nucleobase.

As used herein, “2”-F″ refers to a nucleoside comprising a sugar comprising a fluoro group at the 2″ position of the sugar ring.

As used herein, “2′—OMe” or “2′—OCH₃” or “2′—O-methyl” each refers to a nucleoside comprising a sugar comprising an —OCH₃ group at the 2′ position of the sugar ring.

As used herein, “MOE” or “2”-MOE″ or “2′—OCH₂CH₂OCH₃” or “2′—O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854). Such ring systems can undergo various additional substitutions to enhance activity.

Methods for the preparations of modified sugars are well known to those skilled in the art. Some representative U.S. patents that teach the preparation of such modified sugars include without limitation, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′—CH(CH₃)—O—2″) bridging group. In certain embodiments, the (4′—CH(CH₃)—O—2′) modified nucleosides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi. Y. S., Crooke. S. T. and Lebleu. B., eds., Antisense Research and Applications. CRC Press. Boca Raton, 1993. pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2. N-6 and 0-6 substituted purines, including 2 aminopropyladenine, 5-propynyluracil and 5-propynylcytosine.

In certain embodiments, antisense compounds targeted to a CFB nucleic acid comprise one or more modified nucleobases. In certain embodiments, shortened or gap-widened antisense oligonucleotides targeted to a CFB nucleic acid comprise one or more modified nucleobases. In certain embodiments, the modified nucleobase is 5-methylcytosine. In certain embodiments, each cytosine is a 5-methylcytosine.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

In certain embodiments, antisense compounds, including, but not limited to those particularly suited for use as ssRNA, are modified by attachment of one or more conjugate groups. In general, conjugate groups modify one or more properties of the attached oligonucleotide, including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA. 1989, 86, 6553-6556), cholic acid (Manoharan et al., Bioorg. Med. Chem. Let., 1994, 4, 1053-1060), a thioether. e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

For additional conjugates including those useful for ssRNA and their placement within antisense compounds, see e.g., U.S. Application No. 61/583,963.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides may be mixed with LIPOFECTIN in OPTI-MEM 1 (Invitrogen, Carlsbad, CA) to achieve the desired final concentration of antisense oligonucleotide and a LIPOFECTIN concentration that may range from 2 to 12 μg/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that may range from 2 to 12 μg/mL per 100 nM antisense oligonucleotide.

Another technique used to introduce antisense oligonucleotides into cultured cells includes electroporation.

Yet another technique used to introduce antisense oligonucleotides into cultured cells includes free uptake of the oligonucleotides by the cells.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, CA) according to the manufacturer's recommended protocols.

### Certain Indications

Certain embodiments provided herein relate to methods of treating, preventing, or ameliorating a disease associated with dysregulation of the complement alternative pathway in a subject by administration of a CFB specific inhibitor, such as an antisense compound targeted to CFB.

Examples of renal diseases associated with dysregulation of the complement alternative pathway treatable, preventable, and/or ameliorable with the methods provided herein include C₃ glomerulopathy, atypical hemolytic uremic syndrome (aHUS), dense deposit disease (DDD; also known as MPGN Type II or C₃Neph), and CFHR5 nephropathy.

Additional renal diseases associated with dysregulation of the complement alternative pathway treatable, preventable, and/or ameliorable with the methods provided herein include IgA nephropathy; mesangiocapillary (membranoproliferative) glomerulonephritis (MPGN); autoimmune disorders including lupus nephritis and systemic lupus erythematosus (SLE); infection-induced glomerulonephritis (also known as Postinfectious glomerulonephritis); and renal ischemia-reperfusion injury, for example post-transplant renal ischemia-reperfusion injury.

Examples of non-renal disorders associated with dysregulation of the complement alternative pathway treatable and/or preventable with the methods provided herein include ocular diseases such as macular degeneration, for example age-related macular degeneration (AMD), including wet AMD and dry AMD, such as Geographic Atrophy; neuromyelitis optica; corneal disease, such as corneal inflammation; autoimmune uveitis; and diabetic retinopathy. It has been reported that complement system is involved in ocular diseases. Jha P, et al., Mol Immunol (2007) 44(16): 3901-3908. Additional examples of non-renal disorders associated with dysregulation of the complement alternative pathway treatable and/or preventable with the methods provided herein include ANCA-assocaited vasculitis, antiphospholipid syndrome (also known as antiphospholipid antibody syndrome (APS)), asthma, rheumatoid arthritis, Myasthenia Gravis, and multiple sclerosis.

Certain embodiments provided herein relate to methods of treating, preventing, or ameliorating a renal disease associated with dysregulation of the complement alternative pathway in a subject by administration of a CFB specific inhibitor, such as an antisense compound targeted to CFB. In certain embodiments, the renal disease is lupus nephritis, systemic lupus erythematosus (SLE), dense deposit disease (DDD). C₃ glomerulonephritis (C₃GN), CFHR5 nephropathy, or atypical hemolytic uremic syndrome (aHUS), or any combination thereof.

Certain embodiments provided herein relate to methods of treating, preventing, or ameliorating macular degeneration, such as age-related macular degeneration (AMD), in a subject by administration of a CFB specific inhibitor, such as an antisense compound targeted to CFB. In certain embodiments, the AMD is wet AMD or dry AMD. In certain embodiments, dry AMD can be Geographic Atrophy. Studies have demonstrated the association of complement alternative pathway dysregulation and AMD. Complement components are common constituents of ocular drusen, the extracellular material that accumulates in the macula of AMD patients. Furthermore, it has been reported that CFH and CFB variants account for nearly 75% of AMD cases in northern Europe and North America. It has also been found that a specific CFB polymorphism confers protection against AMD. Patel. N. et al., Eye (2008) 22(6): 768-76. Additionally. CFB homozygous null mice have lower complement pathway activity, exhibit smaller ocular lesions, and choroidal neovascularization (CNV) after laser photocoagulation. Rohrer. B. et al., Invest Ophthalmol Vis Sci. (2009) 50(7):3056-64. Furthermore. CFB siRNA treatment protects mice from laser induced CNV. Bora, N S et al., J Immunol. (2006) 177(3): 1872-8. Studies have also shown that the kidney and eye share developmental pathways and structural features including basement membrane collagen IV protomer composition and vascularity. Savige et al., J Am Soc Nephrol. (2011) 22(8): 1403-15. There is evidence that the complement pathway is involved in renal and ocular diseases. For instance, inherited complement regulatory protein deficiency causes predisposition to atypical hemolytic uremic syndrome and AMD. Richards A et al., Adv Immunol. (2007) 96:141-77. Additionally, chronic kidney disease has been associated with AMD. Nitsch. D. et al., Ophthalmic Epidemiol. (2009) 16(3): 181-6; Choi. J. et al. Ophthalmic Epidemiol. (2011) 18(6):259-63. Dense deposit disease (DDD), a kidney disease associated with dysregulated complement alternative pathway, is characterized by acute nephritic syndrome and ocular drusen. Cruz and Smith, GeneReviews (2007) Jul. 20. Moreover, mice harboring genetic deletion of a component of the complement alternative pathway have coexisting renal and ocular disease phenotypes. It has been reported that CFH homozygous null mice develop DDD and present retinal abnormalities and visual dysfunction. Pickering et al., Nat Genet. (2002) 31(4):424-8. Mouse models of renal diseases associated with dysregulation of the complement alternative pathway are also accepted as models of AMD. Pennesi M E et al., Mol Apects Med (2012) 33:487-509. CFH null mice, for example, are an accepted model for renal diseases, such as DDD, and AMD. Furthermore, it has been reported that AMD is associated with the systemic source of complement factors, which accumulate locally in the eye to drive alternative pathway complement activation. Loyet et al., Invest Ophthalmol Vis Sci. (2012) 53(10):6628-37.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

It is understood that the sequence set forth in each SEQ ID NO in the examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

### Example 1: Antisense Inhibition of Human Complement Factor B (CFB) in HepG2 Cells by MOE Gapmers

Antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 4,500 nM antisense oligonucleotide. After a treatment period of approximately 24 hours, RNA was isolated from the cells and CFB mRNA levels were measured by quantitative real-time PCR. Human primer probe set RTS3459 (forward sequence AGTCTCTGTGGCATGGTTTGG, designated herein as SEQ ID NO: 810; reverse sequence GGGCGAATGACTGAGATCTTG, designated herein as SEQ ID NO: 811; probe sequence TACCGATTACCACAAGCAACCATGGCA, designated herein as SEQ ID NO: 812) was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 5-10-5 MOE gapmers. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 1
Inhibition of CFB mRNA by 5-10-5 MOE
gapmers targeting SEQ ID NO: 1 and 2

| | SEQ | SEQ | | | | SEQ | SEQ | |
|---|---|---|---|---|---|---|---|---|
| | ID | ID | | | | ID | ID | |
| | NO: | NO: | | | % | NO: | NO: | |
| | 1 | 1 | | | inhi- | 2 | 2 | SEQ |
| ISIS | start | stop | Target | | bi- | start | stop | ID |
| NO | site | site | Region | Sequence | tion | site | site | NO: |
| 532608 | 20 | 39 | Exon 1 | GCTGAGCTGC | 36 | 1741 | 1760 | 6 |
| | | | | CAGTCAAGGA | | | | |
| 532609 | 26 | 45 | Exon 1 | GGCCCCGCTG | 16 | 1747 | 1766 | 7 |
| | | | | AGCTGCCAGT | | | | |
| 532610 | 45 | 64 | Exon 1 | CGGAACATCC | 11 | 1766 | 1785 | 8 |
| | | | | AAGCGGGAGG | | | | |
| 532611 | 51 | 70 | Exon 1 | CTTTCCCGGA | 26 | 1772 | 1791 | 9 |
| | | | | ACATCCAAGC | | | | |
| 532612 | 100 | 119 | Exon 1 | ATCTGTGTTC | 25 | 1821 | 1840 | 10 |
| | | | | TGGCACCTGC | | | | |
| 532613 | 148 | 167 | Exon 1 | GTCACATTCC | 39 | 1869 | 1888 | 11 |
| | | | | CTTCCCCTGC | | | | |
| 532614 | 154 | 173 | Exon 1 | GACCTGGTCA | 71 | 1875 | 1894 | 12 |
| | | | | CATTCCCTTC | | | | |
| 532615 | 160 | 179 | Exon 1 | GACCTAGACC | 35 | 1881 | 1900 | 13 |
| | | | | TGGTCACATT | | | | |
| 532616 | 166 | 185 | Exon 1 | ACTCCAGACC | 39 | 1887 | 1906 | 14 |
| | | | | TAGACCTGGT | | | | |
| 532617 | 172 | 191 | Exon 1 | GCTGAAACTC | 27 | 1893 | 1912 | 15 |
| | | | | CAGACCTAGA | | | | |
| 532618 | 178 | 197 | Exon 1 | GTCCAAGCTG | 29 | 1899 | 1918 | 16 |
| | | | | AAACTCCAGA | | | | |
| 532619 | 184 | 203 | Exon 1 | CTCAGTGTCC | 21 | 1905 | 1924 | 17 |
| | | | | AAGCTGAAAC | | | | |
| 532620 | 246 | 265 | Exon 1 | AGGAGAGAAG | 31 | 1967 | 1986 | 18 |
| | | | | CTGGGCCTGG | | | | |
| 532621 | 252 | 271 | Exon 1 | GAAGGCAGGA | 25 | 1973 | 1992 | 19 |
| | | | | GAGAAGCTGG | | | | |
| 532622 | 336 | 355 | Exon | GTGGTGGTCA | 28 | n/a | n/a | 20 |
| | | | 1-2 | CACCTCCAGA | | | | |
| | | | Junc- | | | | | |
| | | | tion | | | | | |
| 532623 | 381 | 400 | Exon 2 | CCCTCCAGAG | 22 | 2189 | 2208 | 21 |
| | | | | AGCAGGATCC | | | | |
| 532624 | 387 | 406 | Exon 2 | TCTACCCCCT | 37 | 2195 | 2214 | 22 |
| | | | | CCAGAGAGCA | | | | |
| 532625 | 393 | 412 | Exon 2 | TTGATCTCTA | 30 | 2201 | 2220 | 23 |
| | | | | CCCCCTCCAG | | | | |
| 532626 | 417 | 436 | Exon 2 | TGGAGAAGTC | 35 | 2225 | 2244 | 24 |
| | | | | GGAAGGAGCC | | | | |
| 532627 | 423 | 442 | Exon 2 | CCCTCTTGGA | 37 | 2231 | 2250 | 25 |
| | | | | GAAGTCGGAA | | | | |
| 532628 | 429 | 448 | Exon 2 | GCCTGGCCCT | 0 | 2237 | 2256 | 26 |
| | | | | CTTGGAGAAG | | | | |
| 532629 | 435 | 454 | Exon 2 | TCCAGTGCCT | 26 | 2243 | 2262 | 27 |
| | | | | GGCCCTCTTG | | | | |
| 532630 | 458 | 477 | Exon 2 | AGAAGCCAGA | 30 | 2266 | 2285 | 28 |
| | | | | AGGACACACG | | | | |
| 532631 | 464 | 483 | Exon 2 | ACGGGTAGAA | 43 | 2272 | 2291 | 29 |
| | | | | GCCAGAAGGA | | | | |
| 532632 | 480 | 499 | Exon 2 | CGTGTCTGCA | 57 | 2288 | 2307 | 30 |
| | | | | CAGGGTACGG | | | | |
| 532633 | 513 | 532 | Exon 2 | AGGGTGCTCC | 27 | 2321 | 2340 | 31 |
| | | | | AGGACCCCGT | | | | |
| 532634 | 560 | 579 | Exon | TTGCTCTGCA | 41 | n/a | n/a | 32 |
| | | | 2-3 | CTCTGCCTTC | | | | |
| | | | Junc- | | | | | |
| | | | tion | | | | | |
| 532635 | 600 | 619 | Exon 3 | TATTCCCCGT | 67 | 2808 | 2827 | 33 |
| | | | | TCTCGAAGTC | | | | |
| 532636 | 626 | 645 | Exon 3 | CATTGTAGTA | 24 | 2834 | 2853 | 34 |
| | | | | GGGAGACCGG | | | | |
| 532637 | 632 | 651 | Exon 3 | CACTCACATT | 49 | 2840 | 2859 | 35 |
| | | | | GTAGTAGGGA | | | | |
| 532638 | 638 | 657 | Exon 3 | TCTCATCACT | 50 | 2846 | 2865 | 36 |
| | | | | CACATTGTAG | | | | |
| 532639 | 644 | 663 | Exon 3 | AAGAGATCTC | 52 | 2852 | 2871 | 37 |
| | | | | ATCACTCACA | | | | |
| 532640 | 650 | 669 | Exon 3 | AGTGGAAAGA | 34 | 2858 | 2877 | 38 |
| | | | | GATCTCATCA | | | | |
| 532641 | 656 | 675 | Exon 3 | CATAGCAGTG | 32 | 2864 | 2883 | 39 |
| | | | | GAAAGAGATC | | | | |
| 532642 | 662 | 681 | Exon 3 | AACCGTCATA | 45 | 2870 | 2889 | 40 |
| | | | | GCAGTGGAAA | | | | |
| 532643 | 668 | 687 | Exon 3 | GAGTGTAACC | 36 | 2876 | 2895 | 41 |
| | | | | GTCATAGCAG | | | | |
| 532644 | 674 | 693 | Exon 3 | CCCGGAGAGT | 30 | 2882 | 2901 | 42 |
| | | | | GTAACCGTCA | | | | |
| 532645 | 680 | 699 | Exon 3 | CAGAGCCCCG | 27 | 2888 | 2907 | 43 |
| | | | | GAGAGTGTAA | | | | |
| 532646 | 686 | 705 | Exon 3 | GATTGGCAGA | 20 | 2894 | 2913 | 44 |
| | | | | GCCCCGGAGA | | | | |
| 532647 | 692 | 711 | Exon 3 | AGGTGCGATT | 28 | 2900 | 2919 | 45 |
| | | | | GGCAGAGCCC | | | | |
| 532648 | 698 | 717 | Exon 3 | CTTGGCAGGT | 24 | 2906 | 2925 | 46 |
| | | | | GCGATTGGCA | | | | |
| 532649 | 704 | 723 | Exon 3 | CATTCACTTG | 28 | 2912 | 2931 | 47 |
| | | | | GCAGGTGCGA | | | | |
| 532650 | 729 | 748 | Exon 3 | ATCGCTGTCT | 44 | 2937 | 2956 | 48 |
| | | | | GCCCACTCCA | | | | |
| 532651 | 735 | 754 | Exon 3 | TCACAGATCG | 44 | 2943 | 2962 | 49 |
| | | | | CTGTCTGCCC | | | | |
| 532652 | 741 | 760 | Exon 3 | CCGTTGTCAC | 27 | 2949 | 2968 | 50 |
| | | | | AGATCGCTGT | | | | |
| 532653 | 747 | 766 | Exon | CCCGCTCCGT | 28 | n/a | n/a | 51 |
| | | | 3-4 | TGTCACAGAT | | | | |
| | | | Junc- | | | | | |
| | | | tion | | | | | |
| 532654 | 753 | 772 | Exon | CAGTACCCCG | 13 | n/a | n/a | 52 |
| | | | 3-4 | CTCCGTTGTC | | | | |
| | | | Junc- | | | | | |
| | | | tion | | | | | |
| 532655 | 759 | 778 | Exon | TTGGAGCAGT | 8 | n/a | n/a | 53 |
| | | | 3-4 | ACCCCGCTCC | | | | |
| | | | Junc- | | | | | |
| | | | tion | | | | | |
| 532656 | 789 | 808 | Exon 4 | ACCTTCCTTG | 40 | 3152 | 3171 | 54 |
| | | | | TGCCAATGGG | | | | |
| 532657 | 795 | 814 | Exon 4 | CTGCCCACCT | 41 | 3158 | 3177 | 55 |
| | | | | TCCTTGTGCC | | | | |
| 532658 | 818 | 837 | Exon 4 | CGCTGTCTTC | 33 | 3181 | 3200 | 56 |
| | | | | AAGGCGGTAC | | | | |
| 532659 | 835 | 854 | Exon 4 | GCTGCAGTGG | 32 | 3198 | 3217 | 57 |
| | | | | TAGGTGACGC | | | | |
| 532660 | 841 | 860 | Exon 4 | CCCCCGGCTG | 17 | 3204 | 3223 | 58 |
| | | | | CAGTGGTAGG | | | | |
| 532661 | 847 | 866 | Exon 4 | GGTAAGCCCC | 28 | 3210 | 3229 | 59 |
| | | | | CGGCTGCAGT | | | | |
| 532662 | 853 | 872 | Exon 4 | ACGCAGGGTA | 13 | 3216 | 3235 | 60 |
| | | | | AGCCCCCGGC | | | | |
| 532663 | 859 | 878 | Exon 4 | GGAGCCACGC | 33 | 3222 | 3241 | 61 |
| | | | | AGGGTAAGCC | | | | |
| 532664 | 866 | 885 | Exon 4 | GCCGCTGGGA | 10 | 3229 | 3248 | 62 |
| | | | | GCCACGCAGG | | | | |
| 532665 | 891 | 910 | Exon 4 | CAAGAGCCAC | 17 | 3254 | 3273 | 63 |
| | | | | CTTCCTGACA | | | | |
| 532666 | 897 | 916 | Exon 4 | CCGCTCCAAG | 25 | 3260 | 3279 | 64 |
| | | | | AGCCACCTTC | | | | |
| 532667 | 903 | 922 | Exon 4 | TCCGTCCCGC | 29 | 3266 | 3285 | 65 |
| | | | | TCCAAGAGCC | | | | |
| 532668 | 909 | 928 | Exon 4 | GAAGGCTCCG | 14 | 3272 | 3291 | 66 |
| | | | | TCCCGCTCCA | | | | |
| 532669 | 915 | 934 | Exon 4 | TGGCAGGAAG | 18 | 3278 | 3297 | 67 |
| | | | | GCTCCGTCCC | | | | |
| 532670 | 921 | 940 | Exon | GAGTCTTGGC | 20 | n/a | n/a | 68 |
| | | | 4-5 | AGGAAGGCTC | | | | |
| | | | Junc- | | | | | |
| | | | tion | | | | | |
| 532671 | 927 | 946 | Exon | ATGAAGGAGT | 14 | n/a | n/a | 69 |
| | | | 4-5 | CTTGGCAGGA | | | | |
| | | | Junc- | | | | | |
| | | | tion | | | | | |
| 532672 | 956 | 975 | Exon 5 | CTTCGGCCAC | 45 | 3539 | 3558 | 70 |
| | | | | CTCTTGAGGG | | | | |
| 532673 | 962 | 981 | Exon 5 | GGAAAGCTTC | 37 | 3545 | 3564 | 71 |
| | | | | GGCCACCTCT | | | | |
| 532674 | 968 | 987 | Exon 5 | AAGACAGGAA | 28 | 3551 | 3570 | 72 |
| | | | | AGCTTCGGCC | | | | |
| 532675 | 974 | 993 | Exon 5 | TCAGGGAAGA | 16 | 3557 | 3576 | 73 |
| | | | | CAGGAAAGCT | | | | |
| 532676 | 996 | 1015 | Exon 5 | TCGACTCCTT | 31 | 3579 | 3598 | 74 |
| | | | | CTATGGTCTC | | | | |
| 532677 | 1033 | 1052 | Exon | CTTCTGTTGT | 36 | n/a | n/a | 75 |
| | | | 5-6 | TCCCCTGGGC | | | | |
| | | | Junc- | | | | | |
| | | | tion | | | | | |
| 532678 | 1068 | 1087 | Exon 6 | TTCATGGAGC | 19 | 3752 | 3771 | 76 |
| | | | | CTGAAGGGTC | | | | |
| 532679 | 1074 | 1093 | Exon 6 | TAGATGTTCA | 24 | 3758 | 3777 | 77 |
| | | | | TGGAGCCTGA | | | | |
| 532680 | 1080 | 1099 | Exon 6 | ACCAGGTAGA | 13 | 3764 | 3783 | 78 |
| | | | | TGTTCATGGA | | | | |
| 532681 | 1086 | 1105 | Exon 6 | TCTAGCACCA | 20 | 3770 | 3789 | 79 |
| | | | | GGTAGATGTT | | | | |
| 532682 | 1092 | 1111 | Exon 6 | GATCCATCTA | 33 | 3776 | 3795 | 80 |
| | | | | GCACCAGGTA | | | | |
| 532683 | 1098 | 1117 | Exon 6 | CTGTCTGATC | 44 | 3782 | 3801 | 81 |
| | | | | CATCTAGCAC | | | | |
| 532684 | 1104 | 1123 | Exon 6 | CCAATGCTGT | 29 | 3788 | 3807 | 82 |
| | | | | CTGATCCATC | | | | |
| 532685 | 1129 | 1148 | Exon 6 | TTTGGCTCCT | 40 | 3813 | 3832 | 83 |
| | | | | GTGAAGTTGC | | | | |

TABLE 2
Inhibition of CFB mRNA by 5-10-5 MOE gapmers
targeting SEQ ID NO: 1 and 2

| | SEQ | SEQ | | | | SEQ | SEQ | |
|---|---|---|---|---|---|---|---|---|
| | ID | ID | | | | ID | ID | |
| | NO: | NO: | | | | NO: | NO: | |
| | 1 | 1 | | | % | 2 | 2 | SEQ |
| ISIS | start | stop | Target | | Inhibi- | start | stop | ID |
| No | site | site | region | Sequence | tion | site | site | NO: |
| 532686 | 1135 | 1154 | Exon 6 | ACACTTTTTG | 91 | 3819 | 3838 | 84 |
| | | | | GCTCCTGTGA | | | | |
| 532687 | 1141 | 1160 | Exon 6 | GACTAGACAC | 77 | 3825 | 3844 | 85 |
| | | | | TTTTTGGCTC | | | | |
| 532688 | 1147 | 1166 | Exon 6 | TAAGTTGACT | 70 | 3831 | 3850 | 86 |
| | | | | AGACACTTTT | | | | |
| 532689 | 1153 | 1172 | Exon 6 | CTCAATTAAG | 61 | 3837 | 3856 | 87 |
| | | | | TTGACTAGAC | | | | |
| 532690 | 1159 | 1178 | Exon | CACCTTCTCA | 57 | 3843 | 3862 | 88 |
| | | | 6-7 | ATTAAGTTGA | | | | |
| | | | Junc- | | | | | |
| | | | tion | | | | | |
| 532691 | 1165 | 1184 | Exon | ACTTGCCACC | 56 | n/a | n/a | 89 |
| | | | 6-7 | TTCTCAATTA | | | | |
| | | | Junc- | | | | | |
| | | | tion | | | | | |
| 532692 | 1171 | 1190 | Exon | ACCATAACTT | 56 | n/a | n/a | 90 |
| | | | 6-7 | GCCACCTTCT | | | | |
| | | | Junc- | | | | | |
| | | | tion | | | | | |
| 532693 | 1177 | 1196 | Exon 7 | CTTCACACCA | 56 | 4153 | 4172 | 91 |
| | | | | TAACTTGCCA | | | | |
| 532694 | 1183 | 1202 | Exon 7 | TCTTGGCTTC | 55 | 4159 | 4178 | 92 |
| | | | | ACACCATAAC | | | | |
| 532695 | 1208 | 1227 | Exon 7 | ATGTGGCATA | 55 | 4184 | 4203 | 93 |
| | | | | TGTCACTAGA | | | | |
| 532696 | 1235 | 1254 | Exon 7 | CAGACACTTT | 55 | 4211 | 4230 | 94 |
| | | | | GACCCAAATT | | | | |
| 532697 | 1298 | 1317 | Exon | GGTCTTCATA | 53 | n/a | n/a | 95 |
| | | | 7-8 | ATTGATTTCA | | | | |
| | | | Junc- | | | | | |
| | | | tion | | | | | |
| 532698 | 1304 | 1323 | Exon | ACTTGTGGTC | 53 | n/a | n/a | 96 |
| | | | 7-8 | TTCATAATTG | | | | |
| | | | Junc- | | | | | |
| | | | tion | | | | | |
| 532699 | 1310 | 1329 | Exon | ACTTCAACTT | 52 | n/a | n/a | 97 |
| | | | 7-8 | GTGGTCTTCA | | | | |
| | | | Junc- | | | | | |
| | | | tion | | | | | |
| 532700 | 1316 | 1335 | Exon 8 | TCCCTGACTT | 52 | 4609 | 4628 | 98 |
| | | | | CAACTTGTGG | | | | |
| 532701 | 1322 | 1341 | Exon 8 | TGTTAGTCCC | 52 | 4615 | 4634 | 99 |
| | | | | TGACTTCAAC | | | | |
| 532702 | 1328 | 1347 | Exon 8 | TCTTGGTGTT | 51 | 4621 | 4640 | 100 |
| | | | | AGTCCCTGAC | | | | |
| 532703 | 1349 | 1368 | Exon 8 | TGTACACTGC | 51 | 4642 | 4661 | 101 |
| | | | | CTGGAGGGCC | | | | |
| 532704 | 1355 | 1374 | Exon 8 | TCATGCTGTA | 51 | 4648 | 4667 | 102 |
| | | | | CACTGCCTGG | | | | |
| 532705 | 1393 | 1412 | Exon 8 | GTTCCAGCCT | 50 | 4686 | 4705 | 103 |
| | | | | TCAGGAGGGA | | | | |
| 532706 | 1399 | 1418 | Exon 8 | GGTGCGGTTC | 50 | 4692 | 4711 | 104 |
| | | | | CAGCCTTCAG | | | | |
| 532707 | 1405 | 1424 | Exon 8 | ATGGCGGGTG | 50 | 4698 | 4717 | 105 |
| | | | | CGGTTCCAGC | | | | |
| 532708 | 1411 | 1430 | Exon 8 | GATGACATGG | 49 | 4704 | 4723 | 106 |
| | | | | CGGGTGCGGT | | | | |
| 532709 | 1417 | 1436 | Exon 8 | GAGGATGATG | 49 | 4710 | 4729 | 107 |
| | | | | ACATGGCGGG | | | | |
| 532710 | 1443 | 1462 | Exon | CCCATGTTGT | 48 | n/a | n/a | 108 |
| | | | 8-9 | GCAATCCATC | | | | |
| | | | Junc- | | | | | |
| | | | tion | | | | | |
| 532711 | 1449 | 1468 | Exon 9 | TCCCCGCCCA | 48 | 5023 | 5042 | 109 |
| | | | | TGTTGTGCAA | | | | |
| 532712 | 1455 | 1474 | Exon 9 | ATTGGGTCCC | 48 | 5029 | 5048 | 110 |
| | | | | CGCCCATGTT | | | | |
| 532713 | 1461 | 1480 | Exon 9 | ACAGTAATTG | 48 | 5035 | 5054 | 111 |
| | | | | GGTCCCCGCC | | | | |
| 532714 | 1467 | 1486 | Exon 9 | TCAATGACAG | 47 | 5041 | 5060 | 112 |
| | | | | TAATTGGGTC | | | | |
| 532715 | 1473 | 1492 | Exon 9 | ATCTCATCAA | 47 | 5047 | 5066 | 113 |
| | | | | TGACAGTAAT | | | | |
| 532716 | 1479 | 1498 | Exon 9 | TCCCGGATCT | 46 | 5053 | 5072 | 114 |
| | | | | CATCAATGAC | | | | |
| 532717 | 1533 | 1552 | Exon | ACATCCAGAT | 46 | n/a | n/a | 115 |
| | | | 9-10 | AATCCTCCCT | | | | |
| | | | Junc- | | | | | |
| | | | tion | | | | | |
| 532718 | 1539 | 1558 | Exon | ACATAGACAT | 46 | n/a | n/a | 116 |
| | | | 9-10 | CCAGATAATC | | | | |
| | | | Junc- | | | | | |
| | | | tion | | | | | |
| 532719 | 1545 | 1564 | Exon | CCAAACACAT | 46 | n/a | n/a | 117 |
| | | | 9-10 | AGACATCCAG | | | | |
| | | | Junc- | | | | | |
| | | | tion | | | | | |
| 532720 | 1582 | 1601 | Exon 10 | AGCATTGATG | 46 | 5231 | 5250 | 118 |
| | | | | TTCACTTGGT | | | | |
| 532721 | 1588 | 1607 | Exon 10 | AGCCAAAGCA | 45 | 5237 | 5256 | 119 |
| | | | | TTGATGTTCA | | | | |
| 532722 | 1594 | 1613 | Exon 10 | CTTGGAAGCC | 45 | 5243 | 5262 | 120 |
| | | | | AAAGCATTGA | | | | |
| 532723 | 1600 | 1619 | Exon 10 | GTCTTTCTTG | 45 | 5249 | 5268 | 121 |
| | | | | GAAGCCAAAG | | | | |
| 532724 | 1606 | 1625 | Exon 10 | CTCATTGTCT | 44 | 5255 | 5274 | 122 |
| | | | | TTCTTGGAAG | | | | |
| 532725 | 1612 | 1631 | Exon 10 | ATGTTGCTCA | 44 | 5261 | 5280 | 123 |
| | | | | TTGTCTTTCT | | | | |
| 532726 | 1618 | 1637 | Exon 10 | GAACACATGT | 44 | 5267 | 5286 | 124 |
| | | | | TGCTCATTGT | | | | |
| 532727 | 1624 | 1643 | Exon 10 | GACTTTGAAC | 43 | 5273 | 5292 | 125 |
| | | | | ACATGTTGCT | | | | |
| 532728 | 1630 | 1649 | Exon 10 | ATCCTTGACT | 43 | 5279 | 5298 | 126 |
| | | | | TTGAACACAT | | | | |
| 532729 | 1636 | 1655 | Exon 10 | TTCCATATCC | 43 | 5285 | 5304 | 127 |
| | | | | TTGACTTTGA | | | | |
| 532730 | 1642 | 1661 | Exon 10 | CAGGTTTTCC | 42 | 5291 | 5310 | 128 |
| | | | | ATATCCTTGA | | | | |
| 532731 | 1686 | 1705 | Exon 11 | CTCAGAGACT | 42 | 5827 | 5846 | 129 |
| | | | | GGCTTTCATC | | | | |
| 532732 | 1692 | 1711 | Exon 11 | CAGAGACTCA | 42 | 5833 | 5852 | 130 |
| | | | | GAGACTGGCT | | | | |
| 516252 | 1698 | 1717 | Exon 11 | ATGCCACAGA | 42 | 5839 | 5858 | 131 |
| | | | | GACTCAGAGA | | | | |
| 532733 | 1704 | 1723 | Exon 11 | CAAACCATGC | 41 | 5845 | 5864 | 132 |
| | | | | CACAGAGACT | | | | |
| 532734 | 1710 | 1729 | Exon 11 | TGTTCCCAAA | 41 | 5851 | 5870 | 133 |
| | | | | CCATGCCACA | | | | |
| 532735 | 1734 | 1753 | Exon 11 | TTGTGGTAAT | 41 | 5875 | 5894 | 134 |
| | | | | CGGTACCCTT | | | | |
| 532736 | 1740 | 1759 | Exon 11 | GGTTGCTTGT | 40 | 5881 | 5900 | 135 |
| | | | | GGTAATCGGT | | | | |
| 532737 | 1746 | 1765 | Exon 11 | TGCCATGGTT | 40 | 5887 | 5906 | 136 |
| | | | | GCTTGTGGTA | | | | |
| 532738 | 1752 | 1771 | Exon 11 | TTGGCCTGCC | 40 | 5893 | 5912 | 137 |
| | | | | ATGGTTGCTT | | | | |
| 532739 | 1758 | 1777 | Exon 11 | GAGATCTTGG | 38 | 5899 | 5918 | 138 |
| | | | | CCTGCCATGG | | | | |
| 532740 | 1803 | 1822 | Exon 12 | ACAGCCCCCA | 38 | 6082 | 6101 | 139 |
| | | | | TACAGCTCTC | | | | |
| 532741 | 1809 | 1828 | Exon 12 | GACACCACAG | 38 | 6088 | 6107 | 140 |
| | | | | CCCCCATACA | | | | |
| 532742 | 1815 | 1834 | Exon 12 | TACTCAGACA | 38 | 6094 | 6113 | 141 |
| | | | | CCACAGCCCC | | | | |
| 532743 | 1821 | 1840 | Exon 12 | ACAAAGTACT | 37 | 6100 | 6119 | 142 |
| | | | | CAGACACCAC | | | | |
| 532744 | 1827 | 1846 | Exon 12 | GTCAGCACAA | 37 | 6106 | 6125 | 143 |
| | | | | AGTACTCAGA | | | | |
| 532745 | 1872 | 1891 | Exon 12 | TTGATTGAGT | 36 | 6151 | 6170 | 144 |
| | | | | GTTCCTTGTC | | | | |
| 532746 | 1878 | 1897 | Exon 12 | CTGACCTTGA | 35 | 6157 | 6176 | 145 |
| | | | | TTGAGTGTTC | | | | |
| 532747 | 1909 | 1928 | Exon 13 | TATCTCCAGG | 35 | 6403 | 6422 | 146 |
| | | | | TCCCGCTTCT | | | | |
| 532748 | 1967 | 1986 | Exon 13 | GAATTCCTGC | 32 | 6461 | 6480 | 147 |
| | | | | TTCTTTTTTC | | | | |
| 532749 | 1973 | 1992 | Exon 13 | ATTCAGGAAT | 32 | 6467 | 6486 | 148 |
| | | | | TCCTGCTTCT | | | | |
| 532750 | 1979 | 1998 | Exon 13 | CATAAAATTC | 32 | 6473 | 6492 | 149 |
| | | | | AGGAATTCCT | | | | |
| 532751 | 1985 | 2004 | Exon 13 | CATAGTCATA | 31 | 6479 | 6498 | 150 |
| | | | | AAATTCAGGA | | | | |
| 532752 | 2006 | 2025 | Exon 13 | TGAGCTTGAT | 30 | 6500 | 6519 | 151 |
| | | | | CAGGGCAACG | | | | |
| 532753 | 2012 | 2031 | Exon 13 | TATTCTTGAG | 30 | 6506 | 6525 | 152 |
| | | | | CTTGATCAGG | | | | |
| 532754 | 2048 | 2067 | Exon | GACAAATGGG | 30 | n/a | n/a | 153 |
| | | | 13-14 | CCTGATAGTC | | | | |
| | | | Junc- | | | | | |
| | | | tion | | | | | |
| 532755 | 2070 | 2089 | Exon 14 | GTTGTTCCCT | 29 | 6659 | 6678 | 154 |
| | | | | CGGTGCAGGG | | | | |
| 532756 | 2076 | 2095 | Exon 14 | GCTCGAGTTG | 28 | 6665 | 6684 | 155 |
| | | | | TTCCCTCGGT | | | | |
| 532757 | 2082 | 2101 | Exon 14 | CTCAAAGCTC | 28 | 6671 | 6690 | 156 |
| | | | | GAGTTGTTCC | | | | |
| 532758 | 2088 | 2107 | Exon 14 | GGAAGCCTCA | 25 | 6677 | 6696 | 157 |
| | | | | AAGCTCGAGT | | | | |
| 532759 | 2094 | 2113 | Exon 14 | GTTGGAGGAA | 23 | 6683 | 6702 | 158 |
| | | | | GCCTCAAAGC | | | | |
| 532760 | 2100 | 2119 | Exon 14 | GTGGTAGTTG | 23 | 6689 | 6708 | 159 |
| | | | | GAGGAAGCCT | | | | |
| 532761 | 2106 | 2125 | Exon 14 | TGGCAAGTGG | 18 | 6695 | 6714 | 160 |
| | | | | TAGTTGGAGG | | | | |
| 532762 | 2112 | 2131 | Exon 14 | TGTTGCTGGC | 14 | 6701 | 6720 | 161 |
| | | | | AAGTGGTAGT | | | | |

TABLE 3
Inhibition of CFB mRNA by
5-10-5 MOE gapmers targeting SEQ ID NO: 1 and 2

| | SEQ | SEQ | | | | SEQ | SEQ | |
|---|---|---|---|---|---|---|---|---|
| | ID | ID | | | | ID | ID | |
| | NO: | NO: | | | % | NO: | NO: | |
| | 1 | 1 | | | inhi- | 2 | 2 | SEQ |
| ISIS | start | stop | Target | | bi- | start | stop | ID |
| NO | site | site | Region | Sequence | tion | site | site | NO: |
| 532812 | n/a | n/a | Exon 1 | TCCAGCTCAC | 19 | 1593 | 1612 | 162 |
| | | | | TCCCCTGTTG | | | | |
| 532813 | n/a | n/a | Exon 1 | TAAGGATCCA | 40 | 1599 | 1618 | 163 |
| | | | | GCTCACTCCC | | | | |
| 532814 | n/a | n/a | Exon 1 | CAGAAATAAG | 39 | 1605 | 1624 | 164 |
| | | | | GATCCAGCTC | | | | |
| 532815 | n/a | n/a | Exon 1 | AGGGACCAGA | 0 | 1611 | 1630 | 165 |
| | | | | AATAAGGATC | | | | |
| 532816 | n/a | n/a | Exon 1 | CCACTTAGGG | 27 | 1617 | 1636 | 166 |
| | | | | ACCAGAAATA | | | | |
| 532817 | n/a | n/a | Exon 1 | TCCAGGACTC | 39 | 1682 | 1701 | 167 |
| | | | | TCCCCTTCAG | | | | |
| 532818 | n/a | n/a | Exon 1 | AAGTCCCACC | 15 | 1707 | 1726 | 168 |
| | | | | CTTTGCTGCC | | | | |
| 532819 | n/a | n/a | Exon 1 | CTGCAGAAGT | 26 | 1713 | 1732 | 169 |
| | | | | CCCACCCTTT | | | | |
| 532820 | n/a | n/a | Exon 1 | CAGAAACTGC | 8 | 1719 | 1738 | 170 |
| | | | | AGAAGTCCCA | | | | |
| 532821 | n/a | n/a | Exon 2 | AACCTCTGCA | 39 | 2368 | 2387 | 171 |
| | | | -Intron | CTCTGCCTTC | | | | |
| | | | 2 | | | | | |
| 532822 | n/a | n/a | Exon 2 | CCCTCAAACC | 3 | 2374 | 2393 | 172 |
| | | | -Intron | TCTGCACTCT | | | | |
| | | | 2 | | | | | |
| 532823 | n/a | n/a | Exon 2 | TCATTGCCCT | 19 | 2380 | 2399 | 173 |
| | | | -Intron | CAAACCTCTG | | | | |
| | | | 2 | | | | | |
| 532824 | n/a | n/a | Intron 2 | CCACACTCAT | 37 | 2386 | 2405 | 174 |
| | | | | TGCCCTCAAA | | | | |
| 532825 | n/a | n/a | Intron 2 | CACTGCCCAC | 23 | 2392 | 2411 | 175 |
| | | | | ACTCATTGCC | | | | |
| 532826 | n/a | n/a | Intron 2 | TTAGGCCACT | 15 | 2398 | 2417 | 176 |
| | | | | GCCCACACTC | | | | |
| 532827 | n/a | n/a | Intron 2 | CTAGTCCTGA | 28 | 2436 | 2455 | 177 |
| | | | | CCTTGCTGCC | | | | |
| 532828 | n/a | n/a | Intron 2 | CTCATCCTAG | 25 | 2442 | 2461 | 178 |
| | | | | TCCTGACCTT | | | | |
| 532829 | n/a | n/a | Intron 2 | CCTAGTCTCA | 23 | 2448 | 2467 | 179 |
| | | | | TCCTAGTCCT | | | | |
| 532830 | n/a | n/a | Intron 2 | ACCCTGCCTA | 30 | 2454 | 2473 | 180 |
| | | | | GTCTCATCCT | | | | |
| 532831 | n/a | n/a | Intron 2 | CTTGTCACCC | 34 | 2460 | 2479 | 181 |
| | | | | TGCCTAGTCT | | | | |
| 532832 | n/a | n/a | Intron 2 | GCCCACCTTG | 36 | 2466 | 2485 | 182 |
| | | | | TCACCCTGCC | | | | |
| 532833 | n/a | n/a | Intron 2 | CCTAAAACTG | 9 | 2492 | 2511 | 183 |
| | | | | CTCCTACTCC | | | | |
| 532834 | n/a | n/a | Intron 4 | GAGTCAGAAA | 19 | 3494 | 3513 | 184 |
| | | | | TGAGGTCAAA | | | | |
| 532835 | n/a | n/a | Intron | CCCTACTCCC | 16 | 5971 | 5990 | 185 |
| | | | 11 | ATTTCACCTT | | | | |
| 532836 | n/a | n/a | Intron 8 | TGTTGTGCAA | 25 | 5013 | 5032 | 186 |
| | | | -Exon | TCCTGCAGAA | | | | |
| | | | 9 | | | | | |
| 532837 | n/a | n/a | Intron 1 | AAAGGCTGAT | 18 | 2123 | 2142 | 187 |
| | | | | GAAGCCTGGC | | | | |
| 532838 | n/a | n/a | Intron 7 | CCTTTGACCA | 21 | 4461 | 4480 | 188 |
| | | | | CAAAGTGGCC | | | | |
| 532839 | n/a | n/a | Intron | AGGTACCACC | 29 | 6362 | 6381 | 189 |
| | | | 12 | TCTTTGTGGG | | | | |
| 532840 | n/a | n/a | Intron 1 | TGGTGGTCAC | 34 | 2143 | 2162 | 190 |
| | | | -Exon | ACCTGAAGAG | | | | |
| | | | 2 | | | | | |
| 532763 | 2133 | 2152 | Exon | GCAGGGAGCA | 40 | n/a | n/a | 191 |
| | | | 14-15 | GCTCTTCCTT | | | | |
| | | | Junc- | | | | | |
| | | | tion | | | | | |
| 532764 | 2139 | 2158 | Exon 15 | TCCTGTGCAG | 28 | 6927 | 6946 | 192 |
| | | | | GGAGCAGCTC | | | | |
| 532765 | 2145 | 2164 | Exon 15 | TTGATATCCT | 41 | 6933 | 6952 | 193 |
| | | | | GTGCAGGGAG | | | | |
| 532766 | 2151 | 2170 | Exon 15 | AGAGCTTTGA | 36 | 6939 | 6958 | 194 |
| | | | | TATCCTGTGC | | | | |
| 532767 | 2157 | 2176 | Exon 15 | ACAAACAGAG | 33 | 6945 | 6964 | 195 |
| | | | | CTTTGATATC | | | | |
| 532768 | 2163 | 2182 | Exon 15 | TCAGACACAA | 41 | 6951 | 6970 | 196 |
| | | | | ACAGAGCTTT | | | | |
| 532769 | 2169 | 2188 | Exon 15 | TCCTCCTCAG | 49 | 6957 | 6976 | 197 |
| | | | | ACACAAACAG | | | | |
| 532770 | 2193 | 2212 | Exon 15 | ACCTCCTTCC | 61 | 6981 | 7000 | 198 |
| | | | | GAGTCAGCTT | | | | |
| 532771 | 2199 | 2218 | Exon 15 | ATGTAGACCT | 39 | 6987 | 7006 | 199 |
| | | | | CCTTCCGAGT | | | | |
| 532772 | 2205 | 2224 | Exon 15 | TTCTTGATGT | 30 | 6993 | 7012 | 200 |
| | | | | AGACCTCCTT | | | | |
| 532773 | 2211 | 2230 | Exon 15 | TCCCCATTCT | 31 | 6999 | 7018 | 201 |
| | | | | TGATGTAGAC | | | | |
| 532774 | 2217 | 2236 | Exon | TTCTTATCCC | 36 | n/a | n/a | 202 |
| | | | 15-16 | CATTCTTGAT | | | | |
| | | | Junc- | | | | | |
| | | | tion | | | | | |
| 532775 | 2223 | 2242 | Exon | CTGCCTTTCT | 56 | n/a | n/a | 203 |
| | | | 15-16 | TATCCCCATT | | | | |
| | | | Junc- | | | | | |
| | | | tion | | | | | |
| 532776 | 2229 | 2248 | Exon | TCACAGCTGC | 33 | n/a | n/a | 204 |
| | | | 15-16 | CTTTCTTATC | | | | |
| | | | Junc- | | | | | |
| | | | tion | | | | | |
| 532777 | 2235 | 2254 | Exon 16 | TCTCTCTCAC | 38 | 7119 | 7138 | 205 |
| | | | | AGCTGCCTTT | | | | |
| 532778 | 2241 | 2260 | Exon 16 | TGAGCATCTC | 51 | 7125 | 7144 | 206 |
| | | | | TCTCACAGCT | | | | |
| 532779 | 2247 | 2266 | Exon 16 | GCATATTGAG | 39 | 7131 | 7150 | 207 |
| | | | | CATCTCTCTC | | | | |
| 532780 | 2267 | 2286 | Exon 16 | TGACTTTGTC | 56 | 7151 | 7170 | 208 |
| | | | | ATAGCCTGGG | | | | |
| 532781 | 2273 | 2292 | Exon 16 | TGTCCTTGAC | 36 | 7157 | 7176 | 209 |
| | | | | TTTGTCATAG | | | | |
| 532782 | 2309 | 2328 | Exon 16 | CAGTACAAAG | 30 | 7193 | 7212 | 210 |
| | | | | GAACCGAGGG | | | | |
| 532783 | 2315 | 2334 | Exon 16 | CTCCTCCAGT | 21 | 7199 | 7218 | 211 |
| | | | | ACAAAGGAAC | | | | |
| 532784 | 2321 | 2340 | Exon 16 | GACTCACTCC | 31 | 7205 | 7224 | 212 |
| | | | | TCCAGTACAA | | | | |
| 532785 | 2327 | 2346 | Exon 16 | CATAGGGACT | 30 | 7211 | 7230 | 213 |
| | | | | CACTCCTCCA | | | | |
| 532786 | 2333 | 2352 | Exon 16 | GGTCAGCATA | 31 | 7217 | 7236 | 214 |
| | | | | GGGACTCACT | | | | |
| 532787 | 2352 | 2371 | Exon | TCACCTCTGC | 42 | 7236 | 7255 | 215 |
| | | | 16-17 | AAGTATTGGG | | | | |
| | | | Junc- | | | | | |
| | | | tion | | | | | |
| 532788 | 2358 | 2377 | Exon | CCAGAATCAC | 32 | n/a | n/a | 216 |
| | | | 16-17 | CTCTGCAAGT | | | | |
| | | | Junc- | | | | | |
| | | | tion | | | | | |
| 532789 | 2364 | 2383 | Exon | GGGCCGCCAG | 35 | n/a | n/a | 217 |
| | | | 16-17 | AATCACCTCT | | | | |
| | | | Junc- | | | | | |
| | | | tion | | | | | |
| 532790 | 2382 | 2401 | Exon 17 | CTCTTGTGAA | 33 | 7347 | 7366 | 218 |
| | | | | CTATCAAGGG | | | | |
| 532791 | 2388 | 2407 | Exon 17 | CGACTTCTCT | 52 | 7353 | 7372 | 219 |
| | | | | TGTGAACTAT | | | | |
| 532792 | 2394 | 2413 | Exon 17 | ATGAAACGAC | 16 | 7359 | 7378 | 220 |
| | | | | TTCTCTTGTG | | | | |
| 532793 | 2400 | 2419 | Exon | ACTTGAATGA | 45 | 7365 | 7384 | 221 |
| | | | 16-17 | AACGACTTCT | | | | |
| | | | Junc- | | | | | |
| | | | tion | | | | | |
| 532794 | 2406 | 2425 | Exon | ACACCAACTT | 18 | n/a | n/a | 222 |
| | | | 16-17 | GAATGAAACG | | | | |
| | | | Junc- | | | | | |
| | | | tion | | | | | |
| 532795 | 2427 | 2446 | Exon 18 | TCCACTACTC | 30 | 7662 | 7681 | 223 |
| | | | | CCCAGCTGAT | | | | |
| 532796 | 2433 | 2452 | Exon 18 | CAGACATCCA | 38 | 7668 | 7687 | 224 |
| | | | | CTACTCCCCA | | | | |
| 532797 | 2439 | 2458 | Exon 18 | TTTTTGCAGA | 35 | 7674 | 7693 | 225 |
| | | | | CATCCACTAC | | | | |
| 532798 | 2445 | 2464 | Exon 18 | TTCTGGTTTT | 45 | 7680 | 7699 | 226 |
| | | | | TGCAGACATC | | | | |
| 532799 | 2451 | 2470 | Exon 18 | TGCCGCTTCT | 47 | 7686 | 7705 | 227 |
| | | | | GGTTTTTGCA | | | | |
| 532800 | 2457 | 2476 | Exon 18 | TGCTTTTGCC | 61 | 7692 | 7711 | 228 |
| | | | | GCTTCTGGTT | | | | |
| 532801 | 2463 | 2482 | Exon 18 | GGTACCTGCT | 47 | 7698 | 7717 | 229 |
| | | | | TTTGCCGCTT | | | | |
| 532802 | 2469 | 2488 | Exon 18 | TGAGCAGGTA | 31 | 7704 | 7723 | 230 |
| | | | | CCTGCTTTTG | | | | |
| 532803 | 2517 | 2536 | Exon 18 | TTCAGCCAGG | 41 | 7752 | 7771 | 231 |
| | | | | GCAGCACTTG | | | | |
| 532804 | 2523 | 2542 | Exon 18 | TTCTCCTTCA | 44 | 7758 | 7777 | 232 |
| | | | | GCCAGGGCAG | | | | |
| 532805 | 2529 | 2548 | Exon 18 | TGGAGTTTCT | 46 | 7764 | 7783 | 233 |
| | | | | CCTTCAGCCA | | | | |
| 532806 | 2535 | 2554 | Exon 18 | TCATCTTGGA | 49 | 7770 | 7789 | 234 |
| | | | | GTTTCTCCTT | | | | |
| 532807 | 2541 | 2560 | Exon 18 | AAATCCTCAT | 30 | 7776 | 7795 | 235 |
| | | | | CTTGGAGTTT | | | | |
| 532808 | 2547 | 2566 | Exon 18 | AAACCCAAAT | 20 | 7782 | 7801 | 236 |
| | | | | CCTCATCTTG | | | | |
| 532809 | 2571 | 2590 | Exon 18 | GTCCAGCAGG | 65 | 7806 | 7825 | 237 |
| | | | | AAACCCCTTA | | | | |
| 532810 | 2577 | 2596 | Exon 18 | GCCCCTGTCC | 74 | 7812 | 7831 | 238 |
| | | | | AGCAGGAAAC | | | | |
| 532811 | 2599 | 2618 | Exon 18 | AGCTGTTTTA | 96 | 7834 | 7853 | 239 |
| | | | | ATTCAATCCC | | | | |

TABLE 4
Inhibition of CFB mRNA by
5-10-5 MOE gapmers targeting SEQ ID NO: 1 and 2

| | SEQ | SEQ | | | SEQ | SEQ | | |
|---|---|---|---|---|---|---|---|---|
| | ID | ID | | | ID | ID | % | |
| | NO: 1 | NO: 1 | | | NO: 2 | NO: 2 | inhi- | SEQ |
| ISIS | start | stop | Target | | start | stop | bi- | ID |
| NO | site | site | region | Sequence | site | site | tion | NO: |
| 532841 | n/a | n/a | Intron | AACTTGCCAC | 4142 | 4161 | 11 | 240 |
| | | | 6- | CTGTGGGTGA | | | | |
| | | | Exon 7 | | | | | |
| 532842 | n/a | n/a | Exon 15- | TCACCTTATC | 7007 | 7026 | 16 | 241 |
| | | | Intron 15 | CCCATTCTTG | | | | |
| 532843 | n/a | n/a | Intron 11 | TCAACTTTCA | 6015 | 6034 | 19 | 242 |
| | | | | CAAACCACCA | | | | |
| 532844 | n/a | n/a | Intron 16 | CCGCCAGAAT | 7326 | 7345 | 33 | 243 |
| | | | -Exon 17 | CACCTGCAAG | | | | |
| 532845 | n/a | n/a | Intron 10 | AGGAGGAATG | 5431 | 5450 | 29 | 244 |
| | | | | AAGAAGGCTT | | | | |
| 532846 | n/a | n/a | Intron 13 | GCCTTTCCTC | 6561 | 6580 | 26 | 245 |
| | | | | AGGGATCTGG | | | | |
| 532847 | n/a | n/a | Intron 4 | AAATGTCTGG | 3477 | 3496 | 18 | 246 |
| | | | | GAGTGTCAGG | | | | |
| 532848 | n/a | n/a | Intron 15 | GCCTAGAGTG | 7038 | 7057 | 20 | 247 |
| | | | | CCTCCTTAGG | | | | |
| 532849 | n/a | n/a | Intron 17 | GGCATCTCCC | 7396 | 7415 | 16 | 248 |
| | | | | CAGATAGGAA | | | | |
| 532850 | n/a | n/a | Intron 6 | AGGGAGCTAG | 3906 | 3925 | 14 | 249 |
| | | | | TCCTGGAAGA | | | | |
| 532851 | n/a | n/a | Intron 1- | ACACCTGAAG | 2135 | 2154 | 6 | 250 |
| | | | Exon 2 | AGAAAGGCTG | | | | |
| 532852 | n/a | n/a | Intron 7 | CCCTTTGACC | 4462 | 4481 | 25 | 251 |
| | | | | ACAAAGTGGC | | | | |
| 532853 | n/a | n/a | Intron 7 | GCCCTCAAGG | 4354 | 4373 | 26 | 252 |
| | | | | TAGTCTCATG | | | | |
| 532854 | n/a | n/a | Intron 6 | AAGGGAAGGA | 3977 | 3996 | 18 | 253 |
| | | | | GGACAGAATA | | | | |
| 532855 | n/a | n/a | Intron 1 | AAAGGCCAAG | 2099 | 2118 | 9 | 254 |
| | | | | GAGGGATGCT | | | | |
| 532856 | n/a | n/a | Exon 8- | AGAGGTCCCT | 4736 | 4755 | 4 | 255 |
| | | | Intron 8 | TCTGACCATC | | | | |
| 532857 | n/a | n/a | Intron 8 | GCTGGGACAG | 4749 | 4768 | 0 | 256 |
| | | | | GAGAGAGGTC | | | | |
| 532858 | n/a | n/a | Intron 4 | TCAAATGTCT | 3479 | 3498 | 13 | 257 |
| | | | | GGGAGTGTCA | | | | |
| 532859 | n/a | n/a | Intron 10 | AGAAGGAGAA | 5801 | 5820 | 20 | 258 |
| | | | | TGTGCTGAAA | | | | |
| 532860 | n/a | n/a | Intron 17 | TGCTGACCAC | 7408 | 7427 | 20 | 259 |
| | | | | TTGGCATCTC | | | | |
| 532861 | n/a | n/a | Intron 11 | CAACTTTCAC | 6014 | 6033 | 18 | 260 |
| | | | | AAACCACCAT | | | | |
| 532862 | n/a | n/a | Intron 10 | AGCTCTGTGA | 5497 | 5516 | 15 | 261 |
| | | | | TTCTAAGGTT | | | | |
| 532863 | n/a | n/a | Intron | CCACCTGTGG | 4136 | 4155 | 16 | 262 |
| | | | 6- | GTGAGGAGAA | | | | |
| | | | Exon 7 | | | | | |
| 532864 | n/a | n/a | Exon 17- | GAGGACTCAC | 7373 | 7392 | 21 | 263 |
| | | | Intron 17 | TTGAATGAAA | | | | |
| 532865 | n/a | n/a | Intron 6 | TGGAATGATC | 3916 | 3935 | 30 | 264 |
| | | | | AGGGAGCTAG | | | | |
| 532866 | n/a | n/a | Intron 5 | GTCCCTTCTC | 3659 | 3678 | 26 | 265 |
| | | | | CATTTTCCCC | | | | |
| 532867 | n/a | n/a | Intron 7 | TCAACTTTTT | 4497 | 4516 | 14 | 266 |
| | | | | AAGTTAATCA | | | | |
| 532868 | n/a | n/a | Intron 6 | GGGTGAGGAG | 4128 | 4147 | 21 | 267 |
| | | | | AACAAGGCGC | | | | |
| 532869 | n/a | n/a | Intron 7 | CTTCCAAGCC | 4553 | 4572 | 5 | 268 |
| | | | | ATCTTTTAAC | | | | |
| 532870 | n/a | n/a | Exon 17- | AGGACTCACT | 7372 | 7391 | 18 | 269 |
| | | | Intron 17 | TGAATGAAAC | | | | |
| 532871 | n/a | n/a | Intron 10 | TTCCAGGCAA | 5412 | 5431 | 15 | 270 |
| | | | | CTAGAGCTTC | | | | |
| 532872 | n/a | n/a | Exon 1 | CAGAGTCCAG | 1557 | 1576 | 13 | 271 |
| | | | | CCACTGTTTG | | | | |
| 532873 | n/a | n/a | Intron 17 | CCAACCTGCA | 7638 | 7657 | 23 | 272 |
| | | | -Exon 18 | GAGGCAGTGG | | | | |
| 532874 | n/a | n/a | Intron 16 | TGCAAGGAGA | 7312 | 7331 | 10 | 273 |
| | | | | GGAGAAGCTG | | | | |
| 532875 | n/a | n/a | Exon 9- | CTAGGCAGGT | 5120 | 5139 | 21 | 274 |
| | | | Intron 9 | TACTCACCCA | | | | |
| 532876 | n/a | n/a | Intron | CACCATAACT | 4148 | 4167 | 41 | 275 |
| | | | 6- | TGCCACCTGT | | | | |
| | | | Exon 7 | | | | | |
| 532877 | n/a | n/a | Intron 12 | TAGGTACCAC | 6363 | 6382 | 27 | 276 |
| | | | | CTCTTTGTGG | | | | |
| 532878 | n/a | n/a | Intron 11 | CTTGACCTCA | 5954 | 5973 | 13 | 277 |
| | | | | CCTCCCCCAA | | | | |
| 532879 | n/a | n/a | Intron 12 | CCACCTCTTT | 6357 | 6376 | 33 | 278 |
| | | | | GTGGGCAGCT | | | | |
| 532880 | n/a | n/a | Intron 11 | TTCACAAACC | 6009 | 6028 | 8 | 279 |
| | | | | ACCATCTCTT | | | | |
| 532881 | n/a | n/a | Exon 3- | TTCTCACCTC | 2958 | 2977 | 17 | 280 |
| | | | Intron 3 | CGTTGTCACA | | | | |
| 532882 | n/a | n/a | Intron 12 | GAAAGTGGGA | 6225 | 6244 | 19 | 281 |
| | | | | GGTGTTGCCT | | | | |
| 532883 | n/a | n/a | Intron 1 | ACAGCAGGAA | 2075 | 2094 | 34 | 282 |
| | | | | GGGAAGGTTA | | | | |
| 532884 | n/a | n/a | Intron 17 | CATGCTGACC | 7410 | 7429 | 18 | 283 |
| | | | | ACTTGGCATC | | | | |
| 532885 | n/a | n/a | Exon 4- | GGTCACCTTG | 3286 | 3305 | 0 | 284 |
| | | | Intron 4 | GCAGGAAGGC | | | | |
| 532886 | n/a | n/a | Intron 8 | GTATAGTGTT | 4804 | 4823 | 13 | 285 |
| | | | | ACAAGTGGAC | | | | |
| 532887 | n/a | n/a | Intron 7 | GGACTTCCCT | 4468 | 4487 | 18 | 286 |
| | | | | TTGACCACAA | | | | |
| 532888 | n/a | n/a | Intron 11 | TCACCTTGAC | 5958 | 5977 | 20 | 287 |
| | | | | CTCACCTCCC | | | | |
| 532889 | n/a | n/a | Intron 15 | TAGAGTGCCT | 7035 | 7054 | 27 | 288 |
| | | | | CCTTAGGATG | | | | |
| 532890 | n/a | n/a | Intron 7 | TGACTTCAAC | 4605 | 4624 | 16 | 289 |
| | | | | TTGTGGTCTG | | | | |
| 532891 | n/a | n/a | Intron 10 | CAGAGAAGGA | 5804 | 5823 | 25 | 290 |
| | | | | GAATGTGCTG | | | | |
| 532892 | n/a | n/a | Intron 14 | AGGGAGCAGC | 6919 | 6938 | 47 | 291 |
| | | | -Exon 15 | TCTTCCTCTG | | | | |
| 532893 | n/a | n/a | Intron 5- | TGTTCCCCTG | 3710 | 3729 | 24 | 292 |
| | | | Exon 6 | GGTGCCAGGA | | | | |
| 532894 | n/a | n/a | Intron 10 | GGCCTGGCTG | 5612 | 5631 | 15 | 293 |
| | | | | TTTTCAAGCC | | | | |
| 532895 | n/a | n/a | Intron 10 | GACTGGCTTT | 5821 | 5840 | 25 | 294 |
| | | | -Exon 11 | CATCTGGCAG | | | | |
| 532896 | n/a | n/a | Intron 10 | GAAGGCTTTC | 5419 | 5438 | 19 | 295 |
| | | | | CAGGCAACTA | | | | |
| 532897 | n/a | n/a | Exon 17- | TCACTTGAAT | 7367 | 7386 | 11 | 296 |
| | | | Intron 17 | GAAACGACTT | | | | |
| 532898 | n/a | n/a | Intron 1 | GGCCCCAAAA | 2106 | 2125 | 5 | 297 |
| | | | | GGCCAAGGAG | | | | |
| 532899 | n/a | n/a | Intron 16 | AATCACCTGC | 7319 | 7338 | 19 | 298 |
| | | | -Exon 17 | AAGGAGAGGA | | | | |
| 532900 | n/a | n/a | Intron 12 | GACCTTCAGT | 6183 | 6202 | 25 | 299 |
| | | | | TGCATCCTTA | | | | |
| 532901 | n/a | n/a | Intron 1 | TGATGAAGCC | 2117 | 2136 | 0 | 300 |
| | | | | TGGCCCCAAA | | | | |
| 532902 | n/a | n/a | Intron 12 | TAGAAAGTGG | 6227 | 6246 | 0 | 301 |
| | | | | GAGGTGTTGC | | | | |
| 532903 | n/a | n/a | Intron 12 | CCCATCCCTG | 6295 | 6314 | 14 | 302 |
| | | | | ACTGGTCTGG | | | | |
| 532904 | n/a | n/a | Intron 8 | CCATGGGTAT | 4810 | 4829 | 13 | 303 |
| | | | | AGTGTTACAA | | | | |
| 532905 | n/a | n/a | Intron 2 | GTGTTCTCTT | 2586 | 2605 | 23 | 304 |
| | | | | GACTTCCAGG | | | | |
| 532906 | n/a | n/a | Intron 13 | GGCCTGCTCC | 6597 | 6616 | 27 | 305 |
| | | | | TCACCCCAGT | | | | |
| 532907 | n/a | n/a | Intron 10 | GAGGCCTGGC | 5614 | 5633 | 32 | 306 |
| | | | | TGTTTTCAAG | | | | |
| 532908 | n/a | n/a | Exon 1 | GACTCTCCCC | 1677 | 1696 | 16 | 307 |
| | | | | TTCAGTACCT | | | | |
| 532909 | n/a | n/a | Intron 8 | CATGGGTATA | 4809 | 4828 | 10 | 308 |
| | | | | GTGTTACAAG | | | | |
| 532910 | n/a | n/a | Intron 10 | GAAGGAGAAT | 5800 | 5819 | 0 | 309 |
| | | | | GTGCTGAAAA | | | | |
| 532911 | n/a | n/a | Intron 7 | TCACCTGGTC | 4562 | 4581 | 0 | 310 |
| | | | | TTCCAAGCCA | | | | |
| 532912 | n/a | n/a | Intron 17 | CTCCCCAGAT | 7391 | 7410 | 0 | 311 |
| | | | | AGGAAAGGGA | | | | |
| 532913 | n/a | n/a | Exon 17- | GGACTCACTT | 7371 | 7390 | 0 | 312 |
| | | | Intron | GAATGAAACG | | | | |
| | | | 17 | | | | | |
| 532914 | n/a | n/a | Intron 16 | GGCCGCCAGA | 7328 | 7347 | 30 | 313 |
| | | | -Exon 17 | ATCACCTGCA | | | | |
| 532915 | n/a | n/a | Exon 17- | CTCACTTGAA | 7368 | 7387 | 22 | 314 |
| | | | Intron 17 | TGAAACGACT | | | | |
| 532916 | n/a | n/a | Intron 13 | CTTTCCCAGC | 6569 | 6588 | 28 | 315 |
| | | | | CTTTCCTCAG | | | | |
| 532918 | n/a | n/a | Intron 12 | AGAAAGTGGG | 6226 | 6245 | 3 | 316 |
| | | | | AGGTGTTGCC | | | | |
| 532917 | 2604 | 2623 | Exon 18 | GTCGCAGCTG | 7839 | 7858 | 90 | 317 |
| | | | | TTTTAATTCA | | | | |

TABLE 5
Inhibition of CFB mRNA by
5-10-5 MOE gapmers targeting SEQ ID NO: 1 and 2

| | SEQ | SEQ | | | SEQ | SEQ | | |
|---|---|---|---|---|---|---|---|---|
| | ID | ID | | | ID | ID | % | |
| | NO: 1 | NO: 1 | | | NO: 2 | NO: 2 | inhi- | SEQ |
| ISIS | start | stop | Target | | start | stop | bi- | ID |
| NO | site | site | region | Sequence | site | site | tion | NO: |
| 532919 | n/a | n/a | Exon 1 | CCAGGACTCT | 1681 | 1700 | 4 | 318 |
| | | | | CCCCTTCAGT | | | | |
| 532920 | n/a | n/a | Intron 6 | AGGGAAGGAG | 3976 | 3995 | 25 | 319 |
| | | | | GACAGAATAG | | | | |
| 532921 | n/a | n/a | Intron 4 | GAAATGAGGT | 3488 | 3507 | 30 | 320 |
| | | | | CAAATGTCTG | | | | |
| 532922 | n/a | n/a | Intron 4 | GGAGAGTCAG | 3497 | 3516 | 25 | 321 |
| | | | | AAATGAGGTC | | | | |
| 532923 | n/a | n/a | Intron 12 | GTAGAAAGTG | 6228 | 6247 | 26 | 322 |
| | | | | GGAGGTGTTG | | | | |
| 532924 | n/a | n/a | Intron 10 | TAGAAAGATC | 5521 | 5540 | 24 | 323 |
| | | | | TCTGAAGTGC | | | | |
| 532925 | n/a | n/a | Intron 13 | CTGCTCCTCA | 6594 | 6613 | 26 | 324 |
| | | | | CCCCAGTCCT | | | | |
| 532926 | n/a | n/a | Intron 11 | CTACTGGGAT | 5927 | 5946 | 30 | 325 |
| | | | | TCTGTGCTTA | | | | |
| 532927 | n/a | n/a | Intron 1 | CCCAAAAGGC | 2103 | 2122 | 13 | 326 |
| | | | | CAAGGAGGGA | | | | |
| 532928 | n/a | n/a | Intron 17 | TGACCACTTG | 7405 | 7424 | 27 | 327 |
| | | | | GCATCTCCCC | | | | |
| 532929 | n/a | n/a | Intron 16- | CCTGCAAGGA | 7314 | 7333 | 29 | 328 |
| | | | Exon 17 | GAGGAGAAGC | | | | |
| 532930 | n/a | n/a | Exon 16- | CTCTCACCTC | 7239 | 7258 | 44 | 329 |
| | | | Intron 16 | TGCAAGTATT | | | | |
| 532931 | n/a | n/a | Intron 1 | CCCCAAAAGG | 2104 | 2123 | 21 | 330 |
| | | | | CCAAGGAGGG | | | | |
| 532932 | n/a | n/a | Intron 7 | GTCTTCCAAG | 4555 | 4574 | 20 | 331 |
| | | | | CCATCTTTTA | | | | |
| 532933 | n/a | n/a | Intron 8 | GTTACAAGTG | 4797 | 4816 | 30 | 332 |
| | | | | GACTTAAGGG | | | | |
| 532934 | n/a | n/a | Intron 8- | CCCATGTTGT | 5017 | 5036 | 30 | 333 |
| | | | Exon 9 | GCAATCCTGC | | | | |
| 532935 | n/a | n/a | Intron 15 | GAGGTGGGAA | 7091 | 7110 | 17 | 334 |
| | | | | GCATGGAGAA | | | | |
| 532936 | n/a | n/a | Intron 14 | TGCTCCCACC | 6874 | 6893 | 21 | 335 |
| | | | | ACTGTCATCT | | | | |
| 532937 | n/a | n/a | Exon 9- | AGGCAGGTTA | 5118 | 5137 | 18 | 336 |
| | | | Intron 9 | CTCACCCAGA | | | | |
| 532938 | n/a | n/a | Intron 11 | TACTGGGATT | 5926 | 5945 | 15 | 337 |
| | | | | CTGTGCTTAC | | | | |
| 532939 | n/a | n/a | Intron 13 | GCCTTTCCCA | 6571 | 6590 | 27 | 338 |
| | | | | GCCTTTCCTC | | | | |
| 532940 | n/a | n/a | Intron 8- | GTGCAATCCT | 5009 | 5028 | 21 | 339 |
| | | | Exon 9 | GCAGAAGAGA | | | | |
| 532941 | n/a | n/a | Intron 8 | ACAGGAGAGA | 4743 | 4762 | 20 | 340 |
| | | | | GGTCCCTTCT | | | | |
| 532942 | n/a | n/a | Intron 10 | CCCAAAAGGA | 5717 | 5736 | 14 | 341 |
| | | | | GAAAGGGAAA | | | | |
| 532943 | n/a | n/a | Intron 2 | AAGCCCAGGG | 2557 | 2576 | 32 | 342 |
| | | | | TAAATGCTTA | | | | |
| 532944 | n/a | n/a | Intron 1 | GATGAAGCCT | 2116 | 2135 | 22 | 343 |
| | | | | GGCCCCAAAA | | | | |
| 532945 | n/a | n/a | Intron 10 | TGGCAGAGAA | 5807 | 5826 | 22 | 344 |
| | | | | GGAGAATGTG | | | | |
| 532946 | n/a | n/a | Intron 13 | TTCCCAGCCT | 6567 | 6586 | 35 | 345 |
| | | | | TTCCTCAGGG | | | | |
| 532947 | n/a | n/a | Intron 10 | GGCAGAGAAG | 5806 | 5825 | 30 | 346 |
| | | | | GAGAATGTGC | | | | |
| 532948 | n/a | n/a | Intron 10 | ACAGTGCCAG | 5471 | 5490 | 25 | 347 |
| | | | | GAAACAAGAA | | | | |
| 532949 | n/a | n/a | Exon 9- | TAGGCAGGTT | 5119 | 5138 | 22 | 348 |
| | | | Intron 9 | ACTCACCCAG | | | | |
| 532950 | n/a | n/a | Intron 2 | TTCTCTTGAC | 2583 | 2602 | 22 | 349 |
| | | | | TTCCAGGGCT | | | | |
| 532951 | n/a | n/a | Intron 13 | CCTGCTCCTC | 6595 | 6614 | 16 | 350 |
| | | | | ACCCCAGTCC | | | | |
| 532953 | n/a | n/a | Intron 7 | TCCCACTAAC | 4422 | 4441 | 14 | 351 |
| | | | | CTCCATTGCC | | | | |
| 532954 | n/a | n/a | Intron 7 | TTCCCTTTGA | 4464 | 4483 | 16 | 352 |
| | | | | CCACAAAGTG | | | | |
| 532955 | n/a | n/a | Intron 9 | CTGGGTCCTA | 5127 | 5146 | 30 | 353 |
| | | | | GGCAGGTTAC | | | | |
| 532956 | n/a | n/a | Intron 10 | TCCAGGCAAC | 5411 | 5430 | 20 | 354 |
| | | | | TAGAGCTTCA | | | | |
| 532957 | n/a | n/a | Intron 8- | GCCCATGTTG | 5018 | 5037 | 45 | 355 |
| | | | Exon 9 | TGCAATCCTG | | | | |
| 532958 | n/a | n/a | Intron 7 | GGTTCCCACT | 4425 | 4444 | 18 | 356 |
| | | | | AACCTCCATT | | | | |
| 532959 | n/a | n/a | Intron 3 | AGGTAGAGAG | 3052 | 3071 | 26 | 357 |
| | | | | CAAGAGTTAC | | | | |
| 532960 | n/a | n/a | Intron 7 | CCACTAACCT | 4420 | 4439 | 10 | 358 |
| | | | | CCATTGCCCA | | | | |
| 532961 | n/a | n/a | Intron 11 | TCACAAACCA | 6008 | 6027 | 40 | 359 |
| | | | | CCATCTCTTA | | | | |
| 532962 | n/a | n/a | Exon 9- | TACTCACCCA | 5110 | 5129 | 27 | 360 |
| | | | Intron 9 | GATAATCCTC | | | | |
| 532963 | n/a | n/a | Intron 13 | TGCTCCTCAC | 6593 | 6612 | 24 | 361 |
| | | | | CCCAGTCCTC | | | | |
| 532964 | n/a | n/a | Intron 15- | TCTCACAGCT | 7115 | 7134 | 25 | 362 |
| | | | Exon 16 | GCCTTTCTGT | | | | |
| 532965 | n/a | n/a | Exon 17- | GAAAGGGAGG | 7379 | 7398 | 11 | 363 |
| | | | Intron 17 | ACTCACTTGA | | | | |
| 532966 | n/a | n/a | Intron 7 | CCATCTTTTA | 4545 | 4564 | 18 | 364 |
| | | | | ACCCCAGAGA | | | | |
| 532967 | n/a | n/a | Intron 13 | TCCTCACCCC | 6590 | 6609 | 27 | 365 |
| | | | | AGTCCTCCAG | | | | |
| 532968 | n/a | n/a | Intron 10 | CTGGCAGAGA | 5808 | 5827 | 15 | 366 |
| | | | | AGGAGAATGT | | | | |
| 532969 | n/a | n/a | Intron 17 | TCTCCCCAGA | 7392 | 7411 | 23 | 367 |
| | | | | TAGGAAAGGG | | | | |
| 532970 | n/a | n/a | Intron 14 | ACTTCAGCTG | 6882 | 6901 | 18 | 368 |
| | | | | CTCCCACCAC | | | | |
| 532971 | n/a | n/a | Intron 1 | GACAGCAGGA | 2076 | 2095 | 13 | 369 |
| | | | | AGGGAAGGTT | | | | |
| 532972 | n/a | n/a | Intron 13- | GGAGACAAAT | 6640 | 6659 | 33 | 370 |
| | | | Exon 14 | GGGCCTATAA | | | | |
| 532973 | n/a | n/a | Intron 14 | CTGCTCCCAC | 6875 | 6894 | 11 | 371 |
| | | | | CACTGTCATC | | | | |
| 532974 | n/a | n/a | Intron 10 | AGGAATGAAG | 5428 | 5447 | 21 | 372 |
| | | | | AAGGCTTTCC | | | | |
| 532975 | n/a | n/a | Intron 14 | GGGATCTCAT | 6741 | 6760 | 31 | 373 |
| | | | | CCTTATCCTC | | | | |
| 532976 | n/a | n/a | Intron 9 | GTGCTGGGTC | 5130 | 5149 | 16 | 374 |
| | | | | CTAGGCAGGT | | | | |
| 532977 | n/a | n/a | Intron 1 | CAAAAGGCCA | 2101 | 2120 | 14 | 375 |
| | | | | AGGAGGGATG | | | | |
| 532978 | n/a | n/a | Intron 17 | CCATGCTGAC | 7411 | 7430 | 20 | 376 |
| | | | | CACTTGGCAT | | | | |
| 532979 | n/a | n/a | Intron 8 | GGAGGCTGGG | 4753 | 4772 | 25 | 377 |
| | | | | ACAGGAGAGA | | | | |
| 532980 | n/a | n/a | Intron 14- | GGAGCAGCTC | 6917 | 6936 | 36 | 378 |
| | | | Exon 15 | TTCCTCTGGA | | | | |
| 532981 | n/a | n/a | Exon 3- | TCTCACCTCC | 2957 | 2976 | 20 | 379 |
| | | | Intron 3 | GTTGTCACAG | | | | |
| 532982 | n/a | n/a | Intron 13 | CAGTCCTCCA | 6581 | 6600 | 21 | 380 |
| | | | | GCCTTTCCCA | | | | |
| 532983 | n/a | n/a | Intron 13 | AGTCCTCCAG | 6580 | 6599 | 22 | 381 |
| | | | | CCTTTCCCAG | | | | |
| 532984 | n/a | n/a | Intron 4- | TGAAGGAGTC | 3509 | 3528 | 12 | 382 |
| | | | Exon 5 | TGGGAGAGTC | | | | |
| 532985 | n/a | n/a | Intron 16- | CAGAATCACC | 7322 | 7341 | 20 | 383 |
| | | | Exon 17 | TGCAAGGAGA | | | | |
| 532986 | n/a | n/a | Exon 17- | TAGGAAAGGG | 7382 | 7401 | 3 | 384 |
| | | | Intron 17 | AGGACTCACT | | | | |
| 532987 | n/a | n/a | Exon 4- | ACCTTGGCAG | 3282 | 3301 | 12 | 385 |
| | | | Intron 4 | GAAGGCTCCG | | | | |
| 532988 | n/a | n/a | Intron 13- | GAGACAAATG | 6639 | 6658 | 15 | 386 |
| | | | Exon 14 | GGCCTATAAA | | | | |
| 532989 | n/a | n/a | Intron 1 | CTGAAGAGAA | 2131 | 2150 | 17 | 387 |
| | | | | AGGCTGATGA | | | | |
| 532990 | n/a | n/a | Intron 6 | AATGATCAGG | 3913 | 3932 | 30 | 388 |
| | | | | GAGCTAGTCC | | | | |
| 532991 | n/a | n/a | Intron 17 | CTTAGCTGAC | 7557 | 7576 | 22 | 389 |
| | | | | CTAAAGGAAT | | | | |
| 532992 | n/a | n/a | Intron 8 | TGGGTATAGT | 4807 | 4826 | 17 | 390 |
| | | | | GTTACAAGTG | | | | |
| 532993 | n/a | n/a | Intron 1 | TGAAGAGAAA | 2130 | 2149 | 19 | 391 |
| | | | | GGCTGATGAA | | | | |
| 532994 | n/a | n/a | Intron 8 | GTGTTACAAG | 4799 | 4818 | 25 | 392 |
| | | | | TGGACTTAAG | | | | |
| 532995 | n/a | n/a | Intron 6 | ACCTGTGGGT | 4134 | 4153 | 24 | 393 |
| | | | | GAGGAGAACA | | | | |
| 532996 | n/a | n/a | Exon 9- | TCACCCAGAT | 5107 | 5126 | 36 | 394 |
| | | | Intron 9 | AATCCTCCCT | | | | |
| 532952 | 2608 | 2627 | Exon 18 | TGTTGTCGCA | 7843 | 7862 | 90 | 395 |
| | | | | GCTGTTTTAA | | | | |

### Example 2: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. Cultured HepG2 cells at a density of 20,000 cells per well were transfected using electroporation with 4,500 nM antisense oligonucleotide. After a treatment period of approximately 24 hours, RNA was isolated from the cells and CFB mRNA levels were measured by quantitative real-time PCR. Human primer probe set RTS3460_MGB (forward sequence CGAAGCAGCTCAATGAAATCAA, designated herein as SEQ ID NO: 813; reverse sequence TGCCTGGAGGGCCTTCTT, designated herein as SEQ ID NO: 814; probe sequence AGACCACAAGTTGAAGTC, designated herein as SEQ ID NO: 815) was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 5-10-5 MOE gapmers. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 6
Inhibition of CFB mRNA by
5-10-5 MOE gapmers targeting SEQ ID NO: 1 and 2

| | SEQ | SEQ | | | | SEQ | SEQ | |
|---|---|---|---|---|---|---|---|---|
| | ID | ID | | | | ID | ID | |
| | NO: | NO: | | | % | NO: | NO: | |
| | 1 | 1 | | | inhi- | 2 | 2 | SEQ |
| ISIS | start | stop | Target | | bi- | start | stop | ID |
| NO | site | site | region | Sequence | tion | site | site | NO: |
| 532686 | 1135 | 1154 | Exon 6 | ACACTTTTTG | 48 | 3819 | 3838 | 84 |
| | | | | GCTCCTGTGA | | | | |
| 532687 | 1141 | 1160 | Exon 6 | GACTAGACAC | 63 | 3825 | 3844 | 85 |
| | | | | TTTTTGGCTC | | | | |
| 532688 | 1147 | 1166 | Exon 6 | TAAGTTGACT | 47 | 3831 | 3850 | 86 |
| | | | | AGACACTTTT | | | | |
| 532689 | 1153 | 1172 | Exon 6 | CTCAATTAAG | 57 | 3837 | 3856 | 87 |
| | | | | TTGACTAGAC | | | | |
| 532690 | 1159 | 1178 | Exon | CACCTTCTCA | 49 | 3843 | 3862 | 88 |
| | | | 6-7 | ATTAAGTTGA | | | | |
| | | | Junc- | | | | | |
| | | | tion | | | | | |
| 532691 | 1165 | 1184 | Exon | ACTTGCCACC | 33 | n/a | n/a | 89 |
| | | | 6-7 | TTCTCAATTA | | | | |
| | | | Junc- | | | | | |
| | | | tion | | | | | |
| 532692 | 1171 | 1190 | Exon | ACCATAACTT | 67 | n/a | n/a | 90 |
| | | | 6-7 | GCCACCTTCT | | | | |
| | | | Junc- | | | | | |
| | | | tion | | | | | |
| 532693 | 1177 | 1196 | Exon 7 | CTTCACACCA | 56 | 4153 | 4172 | 91 |
| | | | | TAACTTGCCA | | | | |
| 532694 | 1183 | 1202 | Exon 7 | TCTTGGCTTC | 50 | 4159 | 4178 | 92 |
| | | | | ACACCATAAC | | | | |
| 532695 | 1208 | 1227 | Exon 7 | ATGTGGCATA | 53 | 4184 | 4203 | 93 |
| | | | | TGTCACTAGA | | | | |
| 532696 | 1235 | 1254 | Exon 7 | CAGACACTTT | 52 | 4211 | 4230 | 94 |
| | | | | GACCCAAATT | | | | |
| 532697 | 1298 | 1317 | Exon | GGTCTTCATA | 59 | n/a | n/a | 95 |
| | | | 7-8 | ATTGATTTCA | | | | |
| | | | Juncion | | | | | |
| 532698 | 1304 | 1323 | Exon | ACTTGTGGTC | 52 | n/a | n/a | 96 |
| | | | 7-8 | TTCATAATTG | | | | |
| | | | Juncion | | | | | |
| 532699 | 1310 | 1329 | Exon | ACTTCAACTT | 85 | n/a | n/a | 97 |
| | | | 7-8 | GTGGTCTTCA | | | | |
| | | | Juncion | | | | | |
| 532700 | 1316 | 1335 | Exon 8 | TCCCTGACTT | 96 | 4609 | 4628 | 98 |
| | | | | CAACTTGTGG | | | | |
| 532701 | 1322 | 1341 | Exon 8 | TGTTAGTCCC | 56 | 4615 | 4634 | 99 |
| | | | | TGACTTCAAC | | | | |
| 532702 | 1328 | 1347 | Exon 8 | TCTTGGTGTT | 86 | 4621 | 4640 | 100 |
| | | | | AGTCCCTGAC | | | | |
| 532703 | 1349 | 1368 | Exon 8 | TGTACACTGC | 35 | 4642 | 4661 | 101 |
| | | | | CTGGAGGGCC | | | | |
| 532704 | 1355 | 1374 | Exon 8 | TCATGCTGTA | 12 | 4648 | 4667 | 102 |
| | | | | CACTGCCTGG | | | | |
| 532705 | 1393 | 1412 | Exon 8 | GTTCCAGCCT | 27 | 4686 | 4705 | 103 |
| | | | | TCAGGAGGGA | | | | |
| 532706 | 1399 | 1418 | Exon 8 | GGTGCGGTTC | 67 | 4692 | 4711 | 104 |
| | | | | CAGCCTTCAG | | | | |
| 532707 | 1405 | 1424 | Exon 8 | ATGGCGGGTG | 26 | 4698 | 4717 | 105 |
| | | | | CGGTTCCAGC | | | | |
| 532708 | 1411 | 1430 | Exon 8 | GATGACATGG | 28 | 4704 | 4723 | 106 |
| | | | | CGGGTGCGGT | | | | |
| 532709 | 1417 | 1436 | Exon 8 | GAGGATGATG | 6 | 4710 | 4729 | 107 |
| | | | | ACATGGCGGG | | | | |
| 532710 | 1443 | 1462 | Exon | CCCATGTTGT | 35 | n/a | n/a | 108 |
| | | | 8-9 | GCAATCCATC | | | | |
| | | | Junc- | | | | | |
| | | | tion | | | | | |
| 532711 | 1449 | 1468 | Exon 9 | TCCCCGCCCA | 28 | 5023 | 5042 | 109 |
| | | | | TGTTGTGCAA | | | | |
| 532712 | 1455 | 1474 | Exon 9 | ATTGGGTCCC | 19 | 5029 | 5048 | 110 |
| | | | | CGCCCATGTT | | | | |
| 532713 | 1461 | 1480 | Exon 9 | ACAGTAATTG | 29 | 5035 | 5054 | 111 |
| | | | | GGTCCCCGCC | | | | |
| 532714 | 1467 | 1486 | Exon 9 | TCAATGACAG | 49 | 5041 | 5060 | 112 |
| | | | | TAATTGGGTC | | | | |
| 532715 | 1473 | 1492 | Exon 9 | ATCTCATCAA | 45 | 5047 | 5066 | 113 |
| | | | | TGACAGTAAT | | | | |
| 532716 | 1479 | 1498 | Exon 9 | TCCCGGATCT | 54 | 5053 | 5072 | 114 |
| | | | | CATCAATGAC | | | | |
| 532717 | 1533 | 1552 | Exon | ACATCCAGAT | 22 | n/a | n/a | 115 |
| | | | 9- | AATCCTCCCT | | | | |
| | | | 10 | | | | | |
| | | | Junc- | | | | | |
| | | | tion | | | | | |
| 532718 | 1539 | 1558 | Exon | ACATAGACAT | 8 | n/a | n/a | 116 |
| | | | 9- | CCAGATAATC | | | | |
| | | | 10 | | | | | |
| | | | Junc- | | | | | |
| | | | tion | | | | | |
| 532719 | 1545 | 1564 | Exon | CCAAACACAT | 30 | n/a | n/a | 117 |
| | | | 9- | AGACATCCAG | | | | |
| | | | 10 | | | | | |
| | | | Junc- | | | | | |
| | | | tion | | | | | |
| 532720 | 1582 | 1601 | Exon 10 | AGCATTGATG | 62 | 5231 | 5250 | 118 |
| | | | | TTCACTTGGT | | | | |
| 532721 | 1588 | 1607 | Exon 10 | AGCCAAAGCA | 46 | 5237 | 5256 | 119 |
| | | | | TTGATGTTCA | | | | |
| 532722 | 1594 | 1613 | Exon 10 | CTTGGAAGCC | 35 | 5243 | 5262 | 120 |
| | | | | AAAGCATTGA | | | | |
| 532723 | 1600 | 1619 | Exon 10 | GTCTTTCTTG | 43 | 5249 | 5268 | 121 |
| | | | | GAAGCCAAAG | | | | |
| 532724 | 1606 | 1625 | Exon 10 | CTCATTGTCT | 40 | 5255 | 5274 | 122 |
| | | | | TTCTTGGAAG | | | | |
| 532725 | 1612 | 1631 | Exon 10 | ATGTTGCTCA | 49 | 5261 | 5280 | 123 |
| | | | | TTGTCTTTCT | | | | |
| 532726 | 1618 | 1637 | Exon 10 | GAACACATGT | 68 | 5267 | 5286 | 124 |
| | | | | TGCTCATTGT | | | | |
| 532727 | 1624 | 1643 | Exon 10 | GACTTTGAAC | 54 | 5273 | 5292 | 125 |
| | | | | ACATGTTGCT | | | | |
| 532728 | 1630 | 1649 | Exon 10 | ATCCTTGACT | 61 | 5279 | 5298 | 126 |
| | | | | TTGAACACAT | | | | |
| 532729 | 1636 | 1655 | Exon 10 | TTCCATATCC | 55 | 5285 | 5304 | 127 |
| | | | | TTGACTTTGA | | | | |
| 532730 | 1642 | 1661 | Exon 10 | CAGGTTTTCC | 51 | 5291 | 5310 | 440 |
| | | | | ATATCCTTGA | | | | |
| 532731 | 1686 | 1705 | Exon 10- | CTCAGAGACT | 41 | 5827 | 5846 | 129 |
| | | | 11 | GGCTTTCATC | | | | |
| | | | Junc- | | | | | |
| | | | tion | | | | | |
| 532732 | 1692 | 1711 | Exon 11 | CAGAGACTCA | 59 | 5833 | 5852 | 130 |
| | | | | GAGACTGGCT | | | | |
| 516252 | 1698 | 1717 | Exon 11 | ATGCCACAGA | 57 | 5839 | 5858 | 131 |
| | | | | GACTCAGAGA | | | | |
| 532733 | 1704 | 1723 | Exon 11 | CAAACCATGC | 34 | 5845 | 5864 | 132 |
| | | | | CACAGAGACT | | | | |
| 532734 | 1710 | 1729 | Exon 11 | TGTTCCCAAA | 51 | 5851 | 5870 | 133 |
| | | | | CCATGCCACA | | | | |
| 532735 | 1734 | 1753 | Exon 11 | TTGTGGTAAT | 50 | 5875 | 5894 | 134 |
| | | | | CGGTACCCTT | | | | |
| 532736 | 1740 | 1759 | Exon 11 | GGTTGCTTGT | 64 | 5881 | 5900 | 135 |
| | | | | GGTAATCGGT | | | | |
| 532737 | 1746 | 1765 | Exon 11 | TGCCATGGTT | 40 | 5887 | 5906 | 136 |
| | | | | GCTTGTGGTA | | | | |
| 532738 | 1752 | 1771 | Exon 11 | TTGGCCTGCC | 49 | 5893 | 5912 | 137 |
| | | | | ATGGTTGCTT | | | | |
| 532739 | 1758 | 1777 | Exon 11 | GAGATCTTGG | 47 | 5899 | 5918 | 138 |
| | | | | CCTGCCATGG | | | | |
| 532740 | 1803 | 1822 | Exon 12 | ACAGCCCCCA | 48 | 6082 | 6101 | 139 |
| | | | | TACAGCTCTC | | | | |
| 532741 | 1809 | 1828 | Exon 12 | GACACCACAG | 40 | 6088 | 6107 | 140 |
| | | | | CCCCCATACA | | | | |
| 532742 | 1815 | 1834 | Exon 12 | TACTCAGACA | 33 | 6094 | 6113 | 141 |
| | | | | CCACAGCCCC | | | | |
| 532743 | 1821 | 1840 | Exon 12 | ACAAAGTACT | 39 | 6100 | 6119 | 142 |
| | | | | CAGACACCAC | | | | |
| 532744 | 1827 | 1846 | Exon 12 | GTCAGCACAA | 45 | 6106 | 6125 | 143 |
| | | | | AGTACTCAGA | | | | |
| 532745 | 1872 | 1891 | Exon 12 | TTGATTGAGT | 42 | 6151 | 6170 | 144 |
| | | | | GTTCCTTGTC | | | | |
| 532746 | 1878 | 1897 | Exon 12 | CTGACCTTGA | 53 | 6157 | 6176 | 145 |
| | | | | TTGAGTGTTC | | | | |
| 532747 | 1909 | 1928 | Exon 13 | TATCTCCAGG | 31 | 6403 | 6422 | 146 |
| | | | | TCCCGCTTCT | | | | |
| 532748 | 1967 | 1986 | Exon 13 | GAATTCCTGC | 30 | 6461 | 6480 | 147 |
| | | | | TTCTTTTTTC | | | | |
| 532749 | 1973 | 1992 | Exon 13 | ATTCAGGAAT | 40 | 6467 | 6486 | 148 |
| | | | | TCCTGCTTCT | | | | |
| 532750 | 1979 | 1998 | Exon 13 | CATAAAATTC | 45 | 6473 | 6492 | 149 |
| | | | | AGGAATTCCT | | | | |
| 532751 | 1985 | 2004 | Exon 13 | CATAGTCATA | 43 | 6479 | 6498 | 150 |
| | | | | AAATTCAGGA | | | | |
| 532752 | 2006 | 2025 | Exon 13 | TGAGCTTGAT | 61 | 6500 | 6519 | 151 |
| | | | | CAGGGCAACG | | | | |
| 532753 | 2012 | 2031 | Exon 13 | TATTCTTGAG | 47 | 6506 | 6525 | 152 |
| | | | | CTTGATCAGG | | | | |
| 532754 | 2048 | 2067 | Exon | GACAAATGGG | 35 | n/a | n/a | 153 |
| | | | 13- | CCTGATAGTC | | | | |
| | | | 14 | | | | | |
| | | | Junc- | | | | | |
| | | | tion | | | | | |
| 532755 | 2070 | 2089 | Exon 14 | GTTGTTCCCT | 43 | 6659 | 6678 | 154 |
| | | | | CGGTGCAGGG | | | | |
| 532756 | 2076 | 2095 | Exon 14 | GCTCGAGTTG | 51 | 6665 | 6684 | 155 |
| | | | | TTCCCTCGGT | | | | |
| 532757 | 2082 | 2101 | Exon 14 | CTCAAAGCTC | 36 | 6671 | 6690 | 156 |
| | | | | GAGTTGTTCC | | | | |
| 532758 | 2088 | 2107 | Exon 14 | GGAAGCCTCA | 54 | 6677 | 6696 | 157 |
| | | | | AAGCTCGAGT | | | | |
| 532759 | 2094 | 2113 | Exon 14 | GTTGGAGGAA | 52 | 6683 | 6702 | 158 |
| | | | | GCCTCAAAGC | | | | |
| 532760 | 2100 | 2119 | Exon 14 | GTGGTAGTTG | 22 | 6689 | 6708 | 159 |
| | | | | GAGGAAGCCT | | | | |
| 532761 | 2106 | 2125 | Exon 14 | TGGCAAGTGG | 34 | 6695 | 6714 | 160 |
| | | | | TAGTTGGAGG | | | | |
| 532762 | 2112 | 2131 | Exon 14 | TGTTGCTGGC | 52 | 6701 | 6720 | 161 |
| | | | | AAGTGGTAGT | | | | |

### Example 3: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 5,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 5-10-5 MOE gapmers. The gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity. In case the sequence alignment for a target gene in a particular table is not shown, it is understood that none of the oligonucleotides presented in that table align with 100% complementarity with that target gene.

TABLE 7
Inhibition of CFB mRNA by 5-10-5
MOE gapmers targeting SEQ ID NO: 1

| | SEQ | SEQ | | | | |
|---|---|---|---|---|---|---|
| | ID | ID | | | % | |
| | NO: | NO: | | | in- | |
| | 1 | 1 | | | hi- | SEQ |
| ISIS | start | stop | Target | | bi- | ID |
| NO | site | site | region | Sequence | tion | NO: |
| 588570 | 150 | 169 | Exon 1 | TGGTCACATT | 54 | 396 |
| | | | | CCCTTCCCCT | | |
| 588571 | 152 | 171 | Exon 1 | CCTGGTCACA | 63 | 397 |
| | | | | TTCCCTTCCC | | |
| 532614 | 154 | 173 | Exon 1 | GACCTGGTCA | 64 | 12 |
| | | | | CATTCCCTTC | | |
| 588572 | 156 | 175 | Exon 1 | TAGACCTGGT | 62 | 398 |
| | | | | CACATTCCCT | | |
| 588573 | 158 | 177 | Exon 1 | CCTAGACCTG | 53 | 399 |
| | | | | GTCACATTCC | | |
| 588566 | 2189 | 2208 | Exon 15 | CCTTCCGAGT | 60 | 400 |
| | | | | CAGCTTTTTC | | |
| 588567 | 2191 | 2210 | Exon 15 | CTCCTTCCGA | 61 | 401 |
| | | | | GTCAGCTTTT | | |
| 532770 | 2193 | 2212 | Exon 15 | ACCTCCTTCC | 77 | 198 |
| | | | | GAGTCAGCTT | | |
| 588568 | 2195 | 2214 | Exon 15 | AGACCTCCTT | 72 | 402 |
| | | | | CCGAGTCAGC | | |
| 588569 | 2197 | 2216 | Exon 15 | GTAGACCTCC | 46 | 403 |
| | | | | TTCCGAGTCA | | |
| 588574 | 2453 | 2472 | Exon 18 | TTTGCCGCTT | 46 | 404 |
| | | | | CTGGTTTTTG | | |
| 588575 | 2455 | 2474 | Exon 18 | CTTTTGCCGC | 41 | 405 |
| | | | | TTCTGGTTTT | | |
| 532800 | 2457 | 2476 | Exon 18 | TGCTTTTGCC | 69 | 228 |
| | | | | GCTTCTGGTT | | |
| 588576 | 2459 | 2478 | Exon 18 | CCTGCTTTTG | 61 | 406 |
| | | | | CCGCTTCTGG | | |
| 588577 | 2461 | 2480 | Exon 18 | TACCTGCTTT | 51 | 407 |
| | | | | TGCCGCTTCT | | |
| 516350 | 2550 | 2569 | Exon 18 | AGAAAACCCA | 71 | 408 |
| | | | | AATCCTCATC | | |
| 588509 | 2551 | 2570 | Exon 18 | TAGAAAACCC | 58 | 409 |
| | | | | AAATCCTCAT | | |
| 588510 | 2552 | 2571 | Exon 18 | ATAGAAAACC | 57 | 410 |
| | | | | CAAATCCTCA | | |
| 588511 | 2553 | 2572 | Exon 18 | TATAGAAAAC | 57 | 411 |
| | | | | CCAAATCCTC | | |
| 588512 | 2554 | 2573 | Exon 18 | TTATAGAAAA | 44 | 412 |
| | | | | CCCAAATCCT | | |
| 588513 | 2555 | 2574 | Exon 18 | CTTATAGAAA | 37 | 413 |
| | | | | ACCCAAATCC | | |
| 588514 | 2556 | 2575 | Exon 18 | CCTTATAGAA | 50 | 414 |
| | | | | AACCCAAATC | | |
| 588515 | 2557 | 2576 | Exon 18 | CCCTTATAGA | 45 | 415 |
| | | | | AAACCCAAAT | | |
| 588516 | 2558 | 2577 | Exon 18 | CCCCTTATAG | 60 | 416 |
| | | | | AAAACCCAAA | | |
| 588517 | 2559 | 2578 | Exon 18 | ACCCCTTATA | 67 | 417 |
| | | | | GAAAACCCAA | | |
| 588518 | 2560 | 2579 | Exon 18 | AACCCCTTAT | 57 | 418 |
| | | | | AGAAAACCCA | | |
| 588519 | 2561 | 2580 | Exon 18 | AAACCCCTTA | 61 | 419 |
| | | | | TAGAAAACCC | | |
| 588520 | 2562 | 2581 | Exon 18 | GAAACCCCTT | 27 | 420 |
| | | | | ATAGAAAACC | | |
| 588521 | 2563 | 2582 | Exon 18 | GGAAACCCCT | 25 | 421 |
| | | | | TATAGAAAAC | | |
| 588522 | 2564 | 2583 | Exon 18 | AGGAAACCCC | 36 | 422 |
| | | | | TTATAGAAAA | | |
| 588523 | 2565 | 2584 | Exon 18 | CAGGAAACCC | 36 | 423 |
| | | | | CTTATAGAAA | | |
| 588524 | 2566 | 2585 | Exon 18 | GCAGGAAACC | 46 | 424 |
| | | | | CCTTATAGAA | | |
| 588525 | 2567 | 2586 | Exon 18 | AGCAGGAAAC | 38 | 425 |
| | | | | CCCTTATAGA | | |
| 588526 | 2568 | 2587 | Exon 18 | CAGCAGGAAA | 47 | 426 |
| | | | | CCCCTTATAG | | |
| 588527 | 2569 | 2588 | Exon 18 | CCAGCAGGAA | 68 | 427 |
| | | | | ACCCCTTATA | | |
| 588528 | 2570 | 2589 | Exon 18 | TCCAGCAGGA | 63 | 428 |
| | | | | AACCCCTTAT | | |
| 532809 | 2571 | 2590 | Exon 18 | GTCCAGCAGG | 85 | 237 |
| | | | | AAACCCCTTA | | |
| 588529 | 2572 | 2591 | Exon 18 | TGTCCAGCAG | 76 | 429 |
| | | | | GAAACCCCTT | | |
| 588530 | 2573 | 2592 | Exon 18 | CTGTCCAGCA | 74 | 430 |
| | | | | GGAAACCCCT | | |
| 588531 | 2574 | 2593 | Exon 18 | CCTGTCCAGC | 75 | 431 |
| | | | | AGGAAACCCC | | |
| 588532 | 2575 | 2594 | Exon 18 | CCCTGTCCAG | 73 | 432 |
| | | | | CAGGAAACCC | | |
| 588533 | 2576 | 2595 | Exon 18 | CCCCTGTCCA | 82 | 433 |
| | | | | GCAGGAAACC | | |
| 532810 | 2577 | 2596 | Exon 18 | GCCCCTGTCC | 88 | 238 |
| | | | | AGCAGGAAAC | | |
| 588534 | 2578 | 2597 | Exon 18 | CGCCCCTGTC | 86 | 434 |
| | | | | CAGCAGGAAA | | |
| 588535 | 2579 | 2598 | Exon 18 | ACGCCCCTGT | 86 | 435 |
| | | | | CCAGCAGGAA | | |
| 588536 | 2580 | 2599 | Exon 18 | CACGCCCCTG | 93 | 436 |
| | | | | TCCAGCAGGA | | |
| 588537 | 2581 | 2600 | Exon 18 | CCACGCCCCT | 92 | 437 |
| | | | | GTCCAGCAGG | | |
| 588538 | 2582 | 2601 | Exon 18 | CCCACGCCCC | 94 | 438 |
| | | | | TGTCCAGCAG | | |
| 588539 | 2583 | 2602 | Exon 18 | TCCCACGCCC | 96 | 439 |
| | | | | CTGTCCAGCA | | |
| 588540 | 2584 | 2603 | Exon 18 | ATCCCACGCC | 88 | 440 |
| | | | | CCTGTCCAGC | | |
| 588541 | 2585 | 2604 | Exon 18 | AATCCCACGC | 79 | 441 |
| | | | | CCCTGTCCAG | | |
| 588542 | 2586 | 2605 | Exon 18 | CAATCCCACG | 83 | 442 |
| | | | | CCCCTGTCCA | | |
| 588543 | 2587 | 2606 | Exon 18 | TCAATCCCAC | 86 | 443 |
| | | | | GCCCCTGTCC | | |
| 588544 | 2588 | 2607 | Exon 18 | TTCAATCCCA | 90 | 444 |
| | | | | CGCCCCTGTC | | |
| 588545 | 2589 | 2608 | Exon 18 | ATTCAATCCC | 92 | 445 |
| | | | | ACGCCCCTGT | | |
| 588546 | 2590 | 2609 | Exon 18 | AATTCAATCC | 92 | 446 |
| | | | | CACGCCCCTG | | |
| 588547 | 2591 | 2610 | Exon 18 | TAATTCAATC | 88 | 447 |
| | | | | CCACGCCCCT | | |
| 588548 | 2592 | 2611 | Exon 18 | TTAATTCAAT | 93 | 448 |
| | | | | CCCACGCCCC | | |
| 588549 | 2593 | 2612 | Exon 18 | TTTAATTCAA | 88 | 449 |
| | | | | TCCCACGCCC | | |
| 588550 | 2594 | 2613 | Exon 18 | TTTTAATTCA | 89 | 450 |
| | | | | ATCCCACGCC | | |
| 588551 | 2595 | 2614 | Exon 18 | GTTTTAATTC | 94 | 451 |
| | | | | AATCCCACGC | | |
| 588552 | 2596 | 2615 | Exon 18 | TGTTTTAATT | 93 | 452 |
| | | | | CAATCCCACG | | |
| 588553 | 2597 | 2616 | Exon 18 | CTGTTTTAAT | 96 | 453 |
| | | | | TCAATCCCAC | | |
| 588554 | 2598 | 2617 | Exon 18 | GCTGTTTTAA | 98 | 454 |
| | | | | TTCAATCCCA | | |
| 532811 | 2599 | 2618 | Exon 18 | AGCTGTTTTA | 97 | 239 |
| | | | | ATTCAATCCC | | |
| 532811 | 2599 | 2618 | Exon 18 | AGCTGTTTTA | 95 | 239 |
| | | | | ATTCAATCCC | | |
| 588555 | 2600 | 2619 | Exon 18 | CAGCTGTTTT | 93 | 455 |
| | | | | AATTCAATCC | | |
| 588556 | 2601 | 2620 | Exon 18 | GCAGCTGTTT | 96 | 456 |
| | | | | TAATTCAATC | | |
| 588557 | 2602 | 2621 | Exon 18 | CGCAGCTGTT | 98 | 457 |
| | | | | TTAATTCAAT | | |
| 588558 | 2603 | 2622 | Exon 18 | TCGCAGCTGT | 95 | 458 |
| | | | | TTTAATTCAA | | |
| 532917 | 2604 | 2623 | Exon 18 | GTCGCAGCTG | 97 | 317 |
| | | | | TTTTAATTCA | | |
| 588559 | 2605 | 2624 | Exon 18 | TGTCGCAGCT | 95 | 459 |
| | | | | GTTTTAATTC | | |
| 588560 | 2606 | 2625 | Exon 18 | TTGTCGCAGC | 92 | 460 |
| | | | | TGTTTTAATT | | |
| 588561 | 2607 | 2626 | Exon 18 | GTTGTCGCAG | 93 | 461 |
| | | | | CTGTTTTAAT | | |
| 532952 | 2608 | 2627 | Exon 18 | TGTTGTCGCA | 88 | 395 |
| | | | | GCTGTTTTAA | | |
| 588562 | 2609 | 2628 | Exon 18/ | TTGTTGTCGC | 90 | 462 |
| | | | Repeat | AGCTGTTTTA | | |
| 588563 | 2610 | 2629 | Exon 18/ | TTTGTTGTCG | 89 | 463 |
| | | | Repeat | CAGCTGTTTT | | |
| 588564 | 2611 | 2630 | Exon 18/ | TTTTGTTGTC | 92 | 464 |
| | | | Repeat | GCAGCTGTTT | | |
| 588565 | 2612 | 2631 | Exon 18/ | TTTTTGTTGT | 88 | 465 |
| | | | Repeat | CGCAGCTGTT | | |

TABLE 8
Inhibition of CFB mRNA by 5-10-5 MOE gapmers
targeting SEQ ID NO: 1 or SEQ ID NO: 2

| | SEQ | SEQ | | | | SEQ | SEQ | |
|---|---|---|---|---|---|---|---|---|
| | ID | ID | | | | ID | ID | |
| | NO: | NO: | | | % | NO: | NO: | |
| | 1 | 1 | | | inhi | 2 | 2 | SEQ |
| ISIS | start | stop | Target | | bi- | start | stop | ID |
| NO | site | site | region | Sequence | tion | site | site | NO: |
| 588685 | n/a | n/a | Exon 1 | GGATCCAGCT | 48 | 1596 | 1615 | 466 |
| | | | | CACTCCCCTG | | | | |
| 588686 | n/a | n/a | Exon 1 | AAATAAGGAT | 29 | 1602 | n/a | 467 |
| | | | | CCAGCTCACT | | | | |
| 588688 | n/a | n/a | Exon 1 | GACCAGAAAT | 58 | 1608 | 1627 | 468 |
| | | | | AAGGATCCAG | | | | |
| 588690 | n/a | n/a | Exon 1 | CTTAGGGACC | 45 | 1614 | 1633 | 469 |
| | | | | AGAAATAAGG | | | | |
| 588692 | n/a | n/a | Exon 1 | CACCCACTTA | 36 | 1620 | 1639 | 470 |
| | | | | GGGACCAGAA | | | | |
| 588694 | n/a | n/a | Exon 1 | ACCACCCACT | 47 | 1622 | 1641 | 471 |
| | | | | TAGGGACCAG | | | | |
| 588696 | n/a | n/a | Exon 1 | AGGTCCAGGA | 96 | 1685 | 1704 | 472 |
| | | | | CTCTCCCCTT | | | | |
| 588698 | n/a | n/a | Exon 1 | AAGGTCCAGG | 96 | 1686 | 1705 | 473 |
| | | | | ACTCTCCCCT | | | | |
| 588700 | n/a | n/a | Exon 1 | AAACTGCAGA | 2 | 1716 | 1735 | 474 |
| | | | | AGTCCCACCC | | | | |
| 588586 | 30 | 49 | Exon 1 | GGAGGGCCCC | 59 | 1751 | 1770 | 475 |
| | | | | GCTGAGCTGC | | | | |
| 588587 | 48 | 67 | Exon 1 | TCCCGGAACA | 45 | 1769 | 1788 | 476 |
| | | | | TCCAAGCGGG | | | | |
| 588588 | 56 | 75 | Exon 1 | CATCACTTTC | 39 | 1777 | n/a | 477 |
| | | | | CCGGAACATC | | | | |
| 588589 | 151 | 170 | Exon 1 | CTGGTCACAT | 29 | 1872 | 1891 | 478 |
| | | | | TCCCTTCCCC | | | | |
| 588590 | 157 | 176 | Exon 1 | CTAGACCTGG | 47 | 1878 | 1897 | 479 |
| | | | | TCACATTCCC | | | | |
| 588591 | 339 | 358 | Exon | GGAGTGGTGG | 44 | n/a | n/a | 480 |
| | | | 1-2 | TCACACCTCC | | | | |
| | | | Junc- | | | | | |
| | | | tion | | | | | |
| 588592 | 384 | 403 | Exon 2 | ACCCCCTCCA | 43 | 2192 | 2211 | 481 |
| | | | | GAGAGCAGGA | | | | |
| 588593 | 390 | 409 | Exon 2 | ATCTCTACCC | 34 | 2198 | 2217 | 482 |
| | | | | CCTCCAGAGA | | | | |
| 588594 | 467 | 486 | Exon 2 | GGTACGGGTA | 17 | 2275 | 2294 | 483 |
| | | | | GAAGCCAGAA | | | | |
| 588595 | 671 | 690 | Exon 3 | GGAGAGTGTA | 37 | 2879 | 2898 | 484 |
| | | | | ACCGTCATAG | | | | |
| 588596 | 689 | 708 | Exon 3 | TGCGATTGGC | 18 | 2897 | 2916 | 485 |
| | | | | AGAGCCCCGG | | | | |
| 588597 | 695 | 714 | Exon 3 | GGCAGGTGCG | 32 | 2903 | 2922 | 486 |
| | | | | ATTGGCAGAG | | | | |
| 588598 | 707 | 726 | Exon 3 | GGCCATTCAC | 45 | 2915 | 2934 | 487 |
| | | | | TTGGCAGGTG | | | | |
| 588599 | 738 | 757 | Exon 3 | TTGTCACAGA | 52 | 2946 | 2965 | 488 |
| | | | | TCGCTGTCTG | | | | |
| 588600 | 924 | 943 | Exon | AAGGAGTCTT | 39 | n/a | n/a | 489 |
| | | | 4-5 | GGCAGGAAGG | | | | |
| | | | Junc- | | | | | |
| | | | tion | | | | | |
| 588601 | 931 | 950 | Exon | GTACATGAAG | 37 | n/a | n/a | 490 |
| | | | 4-5 | GAGTCTTGGC | | | | |
| | | | Junc- | | | | | |
| | | | tion | | | | | |
| 588602 | 959 | 978 | Exon 5 | AAGCTTCGGC | 21 | 3542 | 3561 | 491 |
| | | | | CACCTCTTGA | | | | |
| 588603 | 1089 | 1108 | Exon 6 | CCATCTAGCA | 22 | 3773 | 3792 | 492 |
| | | | | CCAGGTAGAT | | | | |
| 588604 | 1108 | 1127 | Exon 6 | GGCCCCAATG | 21 | 3792 | 3811 | 493 |
| | | | | CTGTCTGATC | | | | |
| 588606 | 1150 | 1169 | Exon 6 | AATTAAGTTG | 56 | 3834 | 3853 | 494 |
| | | | | ACTAGACACT | | | | |
| 588608 | 1162 | 1181 | Exon | TGCCACCTTC | 50 | | 19 | 495 |
| | | | 6-7 | TCAATTAAGT | | | | |
| | | | Junc- | | | | | |
| | | | tion | | | | | |
| 588578 | 1167 | 1186 | Exon | TAACTTGCCA | 23 | n/a | n/a | 496 |
| | | | 6-7 | CCTTCTCAAT | | | | |
| | | | Junc- | | | | | |
| | | | tion | | | | | |
| 588579 | 1169 | 1188 | Exon | CATAACTTGC | 23 | n/a | n/a | 497 |
| | | | 6-7 | CACCTTCTCA | | | | |
| | | | Junc- | | | | | |
| | | | tion | | | | | |
| 532692 | 1171 | 1190 | Exon | ACCATAACTT | 15 | n/a | n/a | 90 |
| | | | 6-7 | GCCACCTTCT | | | | |
| | | | Junc- | | | | | |
| | | | tion | | | | | |
| 588580 | 1173 | 1192 | Exon | ACACCATAAC | 16 | n/a | n/a | 498 |
| | | | 6-7 | TTGCCACCTT | | | | |
| | | | Junc- | | | | | |
| | | | tion | | | | | |
| 588581 | 1175 | 1194 | Exon | TCACACCATA | 14 | 4151 | 4170 | 499 |
| | | | 6-7 | ACTTGCCACC | | | | |
| | | | Junc- | | | | | |
| | | | tion | | | | | |
| 588610 | 1319 | 1338 | Exon 8 | TAGTCCCTGA | 50 | 4612 | 4631 | 500 |
| | | | | CTTCAACTTG | | | | |
| 588612 | 1325 | 1344 | Exon 8 | TGGTGTTAGT | 47 | 4618 | 4637 | 501 |
| | | | | CCCTGACTTC | | | | |
| 588614 | 1396 | 1415 | Exon 8 | GCGGTTCCAG | 47 | 4689 | 4708 | 502 |
| | | | | CCTTCAGGAG | | | | |
| 588616 | 1421 | 1440 | Exon 8 | TCATGAGGAT | 51 | 4714 | 4733 | 503 |
| | | | | GATGACATGG | | | | |
| 588618 | 1446 | 1465 | Exon 9 | CCGCCCATGT | 18 | 5020 | 5039 | 504 |
| | | | | TGTGCAATCC | | | | |
| 588620 | 1458 | 1477 | Exon 9 | GTAATTGGGT | 40 | 5032 | 5051 | 505 |
| | | | | CCCCGCCCAT | | | | |
| 588623 | 1482 | 1501 | Exon 9 | AAGTCCCGGA | 40 | 5056 | 5075 | 506 |
| | | | | TCTCATCAAT | | | | |
| 588624 | 1542 | 1561 | Exon | AACACATAGA | 45 | n/a | n/a | 507 |
| | | | 9- | CATCCAGATA | | | | |
| | | | 10 | | | | | |
| | | | Junc- | | | | | |
| | | | tion | | | | | |
| 588626 | 1585 | 1604 | Exon 10 | CAAAGCATTG | 43 | 5234 | 5253 | 508 |
| | | | | ATGTTCACTT | | | | |
| 588628 | 1621 | 1640 | Exon 10 | TTTGAACACA | 45 | 5270 | 5289 | 509 |
| | | | | TGTTGCTCAT | | | | |
| 588631 | 1646 | 1665 | Exon 10 | CTTCCAGGTT | 53 | 5295 | 5314 | 510 |
| | | | | TTCCATATCC | | | | |
| 588632 | 1647 | 1666 | Exon 10 | TCTTCCAGGT | 56 | 5296 | 5315 | 511 |
| | | | | TTTCCATATC | | | | |
| 588634 | 1689 | 1708 | Exon 11 | AGACTCAGAG | 35 | 5830 | 5849 | 512 |
| | | | | ACTGGCTTTC | | | | |
| 588636 | 1749 | 1768 | Exon 11 | GCCTGCCATG | 55 | 5890 | 5909 | 513 |
| | | | | GTTGCTTGTG | | | | |
| 588638 | 1763 | 1782 | Exon 11 | TGACTGAGAT | 78 | 5904 | 5923 | 514 |
| | | | | CTTGGCCTGC | | | | |
| 588640 | 1912 | 1931 | Exon 13 | TTCTATCTCC | 95 | 6406 | 6425 | 515 |
| | | | | AGGTCCCGCT | | | | |
| 588642 | 1982 | 2001 | Exon 13 | AGTCATAAAA | 44 | 6476 | 6495 | 516 |
| | | | | TTCAGGAATT | | | | |
| 588645 | 2073 | 2092 | Exon 14 | CGAGTTGTTC | 40 | 6662 | 6681 | 517 |
| | | | | CCTCGGTGCA | | | | |
| 588646 | 2085 | 2104 | Exon 14 | AGCCTCAAAG | 57 | 6674 | 6693 | 518 |
| | | | | CTCGAGTTGT | | | | |
| 588648 | 2091 | 2110 | Exon 14 | GGAGGAAGCC | 48 | 6680 | 6699 | 519 |
| | | | | TCAAAGCTCG | | | | |
| 588651 | 2097 | 2116 | Exon 14 | GTAGTTGGAG | 40 | 6686 | 6705 | 520 |
| | | | | GAAGCCTCAA | | | | |
| 588652 | 2103 | 2122 | Exon 14 | CAAGTGGTAG | 43 | 6692 | 6711 | 521 |
| | | | | TTGGAGGAAG | | | | |
| 588654 | 2166 | 2185 | Exon 15 | TCCTCAGACA | 13 | 6954 | 6973 | 522 |
| | | | | CAAACAGAGC | | | | |
| 588656 | 2172 | 2191 | Exon 15 | TTCTCCTCCT | 55 | 6960 | 6979 | 523 |
| | | | | CAGACACAAA | | | | |
| 588658 | 2196 | 2215 | Exon 15 | TAGACCTCCT | 44 | 6984 | 7003 | 524 |
| | | | | TCCGAGTCAG | | | | |
| 588660 | 2202 | 2221 | Exon 15 | TTGATGTAGA | 50 | 6990 | 7009 | 525 |
| | | | | CCTCCTTCCG | | | | |
| 588582 | 2219 | 2238 | Exon | CTTTCTTATC | 19 | n/a | n/a | 526 |
| | | | 15- | CCCATTCTTG | | | | |
| | | | 16 | | | | | |
| | | | Junc- | | | | | |
| | | | tion | | | | | |
| 588583 | 2221 | 2240 | Exon | GCCTTTCTTA | 14 | n/a | n/a | 527 |
| | | | 15- | TCCCCATTCT | | | | |
| | | | 16 | | | | | |
| | | | Junc- | | | | | |
| | | | tion | | | | | |
| 532775 | 2223 | 2242 | Exon | CTGCCTTTCT | 3 | n/a | n/a | 203 |
| | | | 15- | TATCCCCATT | | | | |
| | | | 16 | | | | | |
| | | | Junc- | | | | | |
| | | | tion | | | | | |
| 588584 | 2225 | 2244 | Exon | AGCTGCCTTT | 18 | n/a | n/a | 528 |
| | | | 15- | CTTATCCCCA | | | | |
| | | | 16 | | | | | |
| | | | Junc- | | | | | |
| | | | tion | | | | | |
| 588662 | 2226 | 2245 | Exon | CAGCTGCCTT | 27 | n/a | n/a | 529 |
| | | | 15- | TCTTATCCCC | | | | |
| | | | 16 | | | | | |
| | | | Junc- | | | | | |
| | | | tion | | | | | |
| 588585 | 2227 | 2246 | Exon | ACAGCTGCCT | 59 | n/a | n/a | 530 |
| | | | 15- | TTCTTATCCC | | | | |
| | | | 16 | | | | | |
| | | | Junc- | | | | | |
| | | | tion | | | | | |
| 588664 | 2238 | 2257 | Exon 16 | GCATCTCTCT | 49 | 7122 | 7141 | 531 |
| | | | | CACAGCTGCC | | | | |
| 588666 | 2276 | 2295 | Exon 16 | AGATGTCCTT | 41 | 7160 | 7179 | 532 |
| | | | | GACTTTGTCA | | | | |
| 588668 | 2330 | 2349 | Exon 16 | CAGCATAGGG | 41 | 7214 | 7233 | 533 |
| | | | | ACTCACTCCT | | | | |
| 588670 | 2361 | 2380 | Exon | CCGCCAGAAT | 43 | n/a | n/a | 534 |
| | | | 16- | CACCTCTGCA | | | | |
| | | | 17 | | | | | |
| | | | Junc- | | | | | |
| | | | tion | | | | | |
| 588672 | 2397 | 2416 | Exon 17 | TGAATGAAAC | 52 | 7362 | 7381 | 535 |
| | | | | GACTTCTCTT | | | | |
| 588674 | 2430 | 2449 | Exon 18 | ACATCCACTA | 39 | 7665 | 7684 | 536 |
| | | | | CTCCCCAGCT | | | | |
| 588676 | 2448 | 2467 | Exon 18 | CGCTTCTGGT | 69 | 7683 | 7702 | 537 |
| | | | | TTTTGCAGAC | | | | |
| 588678 | 2454 | 2473 | Exon 18 | TTTTGCCGCT | 46 | 7689 | 7708 | 538 |
| | | | | TCTGGTTTTT | | | | |
| 588680 | 2466 | 2485 | Exon 18 | GCAGGTACCT | 47 | 7701 | 7720 | 539 |
| | | | | GCTTTTGCCG | | | | |
| 588682 | 2532 | 2551 | Exon 18 | TCTTGGAGTT | 58 | 7767 | 7786 | 540 |
| | | | | TCTCCTTCAG | | | | |
| 532811 | 2599 | 2618 | Exon 18 | AGCTGTTTTA | 10 | 7834 | 7853 | 239 |
| | | | | ATTCAATCCC | | | | |
| 532917 | 2604 | 2623 | Exon 18 | GTCGCAGCTG | 11 | 7839 | 7858 | 317 |
| | | | | TTTTAATTCA | | | | |

### Example 4: Antisense Inhibition of Human Complement Factor B (CFB) in HepG2 Cells by MOE Gapmers

Antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 3,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 4-8-5 MOE, 5-9-5 MOE, 5-10-5 MOE, 3-10-4 MOE, 3-10-7 MOE, 6-7-6-MOE, 6-8-6 MOE, or 5-7-5 MOE gapmers, or as deoxy, MOE, and cEt oligonucleotides.

The 4-8-5 MOE gapmers are 17 nucleosides in length, wherein the central gap segment comprises of eight 2′-deoxynucleosides and is flanked by wing segments on the 5′ direction and the 3′ direction comprising four and five nucleosides respectively. The 5-9-5 MOE gapmers are 19 nucleosides in length, wherein the central gap segment comprises of nine 2′-deoxynucleosides and is flanked by wing segments on the 5′ direction and the 3′ direction comprising five nucleosides each. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. The 5-7-5 MOE gapmers are 17 nucleosides in length, wherein the central gap segment comprises of seven 2′-deoxynucleosides and is flanked by wing segments on the 5′ direction and the 3′ direction comprising five nucleosides each. The 3-10-4 MOE gapmers are 17 nucleosides in length, wherein the central gap segment comprises of ten 2′-deoxynucleosides and is flanked by wing segments on the 5′ direction and the 3′ direction comprising three and four nucleosides respectively. The 3-10-7 MOE gapmers are 20 nucleosides in length, wherein the central gap segment comprises of ten 2′-deoxynucleosides and is flanked by wing segments on the 5′ direction and the 3′ direction comprising three and seven nucleosides respectively. The 6-7-6 MOE gapmers are 19 nucleosides in length, wherein the central gap segment comprises of seven 2′-deoxynucleosides and is flanked by wing segments on the 5′ direction and the 3′ direction comprising six nucleosides each. The 6-8-6 MOE gapmers are 20 nucleosides in length, wherein the central gap segment comprises of eight 2′-deoxynucleosides and is flanked by wing segments on the 5′ direction and the 3′ direction comprising six nucleosides each. The internucleoside linkages throughout each gapmer are phosphorothioate (P═S) linkages. All cytosine residues throughout each gapmer are 5-methylcytosines.

The deoxy, MOE and cEt oligonucleotides are 16 nucleosides in length wherein the nucleoside have either a MOE sugar modification, an cEt sugar modification, or a deoxy modification. The ‘Chemistry’ column describes the sugar modifications of each oligonucleotide. ‘k’ indicates an cEt sugar modification; ‘d’ indicates deoxyribose; and ‘e’ indicates a MOE modification.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 9
Inhibition of CFB mRNA by deoxy, MOE and cEt oligonucleotides targeting SEQ ID NO: 1 or
SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | Start | stop | Target | | % in- | Start | Stop | | ID |
| NO | site | site | region | Sequence | hibition | site | site | Motif | NO: |
| 532811 | 2599 | 2618 | Exon 18 | AGCTGTTTTAATTCAATCCC | 10 | 7834 | 7853 | eeeeeddddddddddee | 239 |
| | | | | | | | | eee | |
| 588884 | 48 | 63 | Exon 1 | GGAACATCCAAGCGGG | 79 | 1769 | 1784 | eekddddddddddkke | 541 |
| 588872 | 154 | 169 | Exon 1 | TGGTCACATTCCCTTC | 91 | 1875 | 1890 | eekddddddddddkke | 542 |

| | | | | | | | | | |
|---|---|---|---|---|---|---|---|---|---|
| 588873 | 156 | 171 | Exon 1 | CCTGGTCACATTCCCT | 91 | 1877 | 1892 | eekddddddddddkke | 543 |

| | | | | | | | | | |
|---|---|---|---|---|---|---|---|---|---|
| 588874 | 158 | 173 | Exon 1 | GACCTGGTCACATTCC | 91 | 1879 | 1894 | eekddddddddddkke | 544 |
| 588878 | 1171 | 1186 | Exon 6-7 | TAACTTGCCACCTTCT | 92 | n/a | n/a | eekddddddddddkke | 545 |
| | | | Junction | | | | | | |
| 588879 | 1173 | 1188 | Exon 6-7 | CATAACTTGCCACCTT | 94 | n/a | n/a | eekddddddddddkke | 546 |
| | | | Junction | | | | | | |
| 588880 | 1175 | 1190 | Exon 6-7 | ACCATAACTTGCCACC | 89 | 4151 | 4166 | eekddddddddddkke | 547 |
| | | | Junction | | | | | | |
| 588869 | 2193 | 2208 | Exon 15 | CCTTCCGAGTCAGCTT | 17 | 6981 | 6996 | eekddddddddddkke | 548 |
| 588870 | 2195 | 2210 | Exon 15 | CTCCTTCCGAGTCAGC | 78 | 6983 | 6998 | eekddddddddddkke | 549 |
| 588871 | 2197 | 2212 | Exon 15 | ACCTCCTTCCGAGTCA | 80 | 6985 | 7000 | eekddddddddddkke | 550 |
| 588881 | 2223 | 2238 | Exon 15- | CTTTCTTATCCCCATT | 93 | n/a | n/a | eekddddddddddkke | 551 |
| | | | 16 | | | | | | |
| | | | Junction | | | | | | |
| 588882 | 2225 | 2240 | Exon 15- | GCCTTTCTTATCCCCA | 88 | n/a | n/a | eekddddddddddkke | 552 |
| | | | 16 | | | | | | |
| | | | Junction | | | | | | |
| 588883 | 2227 | 2242 | Exon 15- | CTGCCTTTCTTATCCC | 90 | n/a | n/a | eekddddddddddkke | 553 |
| | | | 16 | | | | | | |
| | | | Junction | | | | | | |
| 588875 | 2457 | 2472 | Exon 18 | TTTGCCGCTTCTGGTT | 81 | 7692 | 7707 | eekddddddddddkke | 554 |
| 588876 | 2459 | 2474 | Exon 18 | CTTTTGCCGCTTCTGG | 95 | 7694 | 7709 | eekddddddddddkke | 555 |
| 588877 | 2461 | 2476 | Exon 18 | TGCTTTTGCCGCTTCT | 91 | 7696 | 7711 | eekddddddddddkke | 556 |
| 588807 | 2551 | 2566 | Exon 18 | AAACCCAAATCCTCAT | 82 | 7786 | 7801 | eekddddddddddkke | 557 |
| 588808 | 2553 | 2568 | Exon 18 | GAAAACCCAAATCCTC | 69 | 7788 | 7803 | eekddddddddddkke | 558 |
| 588809 | 2555 | 2570 | Exon 18 | TAGAAAACCCAAATCC | 51 | 7790 | 7805 | eekddddddddddkke | 559 |
| 588810 | 2556 | 2571 | Exon 18 | ATAGAAAACCCAAATC | 23 | 7791 | 7806 | eekddddddddddkke | 560 |
| 588811 | 2559 | 2574 | Exon 18 | CTTATAGAAAACCCAA | 13 | 7794 | 7809 | eekddddddddddkke | 561 |
| 588812 | 2560 | 2575 | Exon 18 | CCTTATAGAAAACCCA | 29 | 7795 | 7810 | eekddddddddddkke | 562 |
| 588813 | 2561 | 2576 | Exon 18 | CCCTTATAGAAAACCC | 53 | 7796 | 7811 | eekddddddddddkke | 563 |
| 588814 | 2562 | 2577 | Exon 18 | CCCCTTATAGAAAACC | 86 | 7797 | 7812 | eekddddddddddkke | 564 |
| 588815 | 2563 | 2578 | Exon 18 | ACCCCTTATAGAAAAC | 76 | 7798 | 7813 | eekddddddddddkke | 565 |
| 588816 | 2564 | 2579 | Exon 18 | AACCCCTTATAGAAAA | 33 | 7799 | 7814 | eekddddddddddkke | 566 |
| 588817 | 2565 | 2580 | Exon 18 | AAACCCCTTATAGAAA | 48 | 7800 | 7815 | eekddddddddddkke | 567 |
| 588818 | 2566 | 2581 | Exon 18 | GAAACCCCTTATAGAA | 44 | 7801 | 7816 | eekddddddddddkke | 568 |
| 588819 | 2567 | 2582 | Exon 18 | GGAAACCCCTTATAGA | 74 | 7802 | 7817 | eekddddddddddkke | 569 |
| 588820 | 2568 | 2583 | Exon 18 | AGGAAACCCCTTATAG | 68 | 7803 | 7818 | eekddddddddddkke | 570 |
| 588821 | 2569 | 2584 | Exon 18 | CAGGAAACCCCTTATA | 45 | 7804 | 7819 | eekddddddddddkke | 571 |
| 588822 | 2570 | 2585 | Exon 18 | GCAGGAAACCCCTTAT | 50 | 7805 | 7820 | eekddddddddddkke | 572 |
| 588823 | 2571 | 2586 | Exon 18 | AGCAGGAAACCCCTTA | 54 | 7806 | 7821 | eekddddddddddkke | 573 |
| 588824 | 2572 | 2587 | Exon 18 | CAGCAGGAAACCCCTT | 35 | 7807 | 7822 | eekddddddddddkke | 574 |
| 588825 | 2573 | 2588 | Exon 18 | CCAGCAGGAAACCCCT | 11 | 7808 | 7823 | eekddddddddddkke | 575 |
| 588826 | 2574 | 2589 | Exon 18 | TCCAGCAGGAAACCCC | 19 | 7809 | 7824 | eekddddddddddkke | 576 |
| 588827 | 2575 | 2590 | Exon 18 | GTCCAGCAGGAAACCC | 42 | 7810 | 7825 | eekddddddddddkke | 577 |
| 588828 | 2576 | 2591 | Exon 18 | TGTCCAGCAGGAAACC | 0 | 7811 | 7826 | eekddddddddddkke | 578 |
| 588829 | 2577 | 2592 | Exon 18 | CTGTCCAGCAGGAAAC | 49 | 7812 | 7827 | eekddddddddddkke | 579 |
| 588830 | 2578 | 2593 | Exon 18 | CCTGTCCAGCAGGAAA | 11 | 7813 | 7828 | eekddddddddddkke | 580 |
| 588831 | 2579 | 2594 | Exon 18 | CCCTGTCCAGCAGGAA | 20 | 7814 | 7829 | eekddddddddddkke | 581 |
| 588832 | 2580 | 2595 | Exon 18 | CCCCTGTCCAGCAGGA | 19 | 7815 | 7830 | eekddddddddddkke | 582 |
| 588833 | 2581 | 2596 | Exon 18 | GCCCCTGTCCAGCAGG | 12 | 7816 | 7831 | eekddddddddddkke | 583 |
| 588834 | 2582 | 2597 | Exon 18 | CGCCCCTGTCCAGCAG | 10 | 7817 | 7832 | eekddddddddddkke | 584 |
| 588835 | 2583 | 2598 | Exon 18 | ACGCCCCTGTCCAGCA | 13 | 7818 | 7833 | eekddddddddddkke | 585 |
| 588836 | 2584 | 2599 | Exon 18 | CACGCCCCTGTCCAGC | 13 | 7819 | 7834 | eekddddddddddkke | 586 |
| 588837 | 2585 | 2600 | Exon 18 | CCACGCCCCTGTCCAG | 39 | 7820 | 7835 | eekddddddddddkke | 587 |
| 588838 | 2586 | 2601 | Exon 18 | CCCACGCCCCTGTCCA | 54 | 7821 | 7836 | eekddddddddddkke | 588 |
| 588839 | 2587 | 2602 | Exon 18 | TCCCACGCCCCTGTCC | 51 | 7822 | 7837 | eekddddddddddkke | 589 |
| 588840 | 2588 | 2603 | Exon 18 | ATCCCACGCCCCTGTC | 65 | 7823 | 7838 | eekddddddddddkke | 590 |
| 588841 | 2589 | 2604 | Exon 18 | AATCCCACGCCCCTGT | 59 | 7824 | 7839 | eekddddddddddkke | 591 |
| 588842 | 2590 | 2605 | Exon 18 | CAATCCCACGCCCCTG | 70 | 7825 | 7840 | eekddddddddddkke | 592 |
| 588843 | 2591 | 2606 | Exon 18 | TCAATCCCACGCCCCT | 0 | 7826 | 7841 | eekddddddddddkke | 593 |
| 588844 | 2592 | 2607 | Exon 18 | TTCAATCCCACGCCCC | 48 | 7827 | 7842 | eekddddddddddkke | 594 |
| 588845 | 2593 | 2608 | Exon 18 | ATTCAATCCCACGCCC | 46 | 7828 | 7843 | eekddddddddddkke | 595 |
| 588846 | 2594 | 2609 | Exon 18 | AATTCAATCCCACGCC | 67 | 7829 | 7844 | eekddddddddddkke | 596 |
| 588847 | 2595 | 2610 | Exon 18 | TAATTCAATCCCACGC | 75 | 7830 | 7845 | eekddddddddddkke | 597 |
| 588848 | 2596 | 2611 | Exon 18 | TTAATTCAATCCCACG | 76 | 7831 | 7846 | eekddddddddddkke | 598 |
| 588849 | 2597 | 2612 | Exon 18 | TTTAATTCAATCCCAC | 94 | 7832 | 7847 | eekddddddddddkke | 599 |
| 588850 | 2598 | 2613 | Exon 18 | TTTTAATTCAATCCCA | 91 | 7833 | 7848 | eekddddddddddkke | 600 |
| 588851 | 2599 | 2614 | Exon 18 | GTTTTAATTCAATCCC | 91 | 7834 | 7849 | eekddddddddddkke | 601 |
| 588852 | 2600 | 2615 | Exon 18 | TGTTTTAATTCAATCC | 78 | 7835 | 7850 | eekddddddddddkke | 602 |
| 588853 | 2601 | 2616 | Exon 18 | CTGTTTTAATTCAATC | 81 | 7836 | 7851 | eekddddddddddkke | 603 |
| 588854 | 2602 | 2617 | Exon 18 | GCTGTTTTAATTCAAT | 63 | 7837 | 7852 | eekddddddddddkke | 604 |
| 588855 | 2603 | 2618 | Exon 18 | AGCTGTTTTAATTCAA | 65 | 7838 | 7853 | eekddddddddddkke | 605 |
| 588856 | 2604 | 2619 | Exon 18 | CAGCTGTTTTAATTCA | 76 | 7839 | 7854 | eekddddddddddkke | 606 |
| 588857 | 2605 | 2620 | Exon 18 | GCAGCTGTTTTAATTC | 89 | 7840 | 7855 | eekddddddddddkke | 607 |
| 588858 | 2606 | 2621 | Exon 18 | CGCAGCTGTTTTAATT | 89 | 7841 | 7856 | eekddddddddddkke | 608 |
| 588859 | 2607 | 2622 | Exon 18 | TCGCAGCTGTTTTAAT | 89 | 7842 | 7857 | eekddddddddddkke | 609 |
| 588860 | 2608 | 2623 | Exon 18 | GTCGCAGCTGTTTTAA | 76 | 7843 | 7858 | eekddddddddddkke | 610 |
| 588861 | 2609 | 2624 | Exon 18 | TGTCGCAGCTGTTTTA | 87 | 7844 | 7859 | eekddddddddddkke | 611 |
| 588862 | 2610 | 2625 | Exon 18 | TTGTCGCAGCTGTTTT | 85 | 7845 | 7860 | eekddddddddddkke | 612 |
| 588863 | 2611 | 2626 | Exon 18 | GTTGTCGCAGCTGTTT | 87 | 7846 | 7861 | eekddddddddddkke | 613 |
| 588864 | 2612 | 2627 | Exon 18 | TGTTGTCGCAGCTGTT | 67 | 7847 | 7862 | eekddddddddddkke | 614 |
| 588865 | 2613 | 2628 | Exon 18 | TTGTTGTCGCAGCTGT | 51 | n/a | n/a | eekddddddddddkke | 615 |
| 588866 | 2614 | 2629 | Exon 18 | TTTGTTGTCGCAGCTG | 95 | n/a | n/a | eekddddddddddkke | 616 |
| 588867 | 2615 | 2630 | Exon 18 | TTTTGTTGTCGCAGCT | 92 | n/a | n/a | eekddddddddddkke | 617 |
| 588868 | 2616 | 2631 | Exon 18 | TTTTTGTTGTCGCAGC | 66 | n/a | n/a | eekddddddddddkke | 618 |

TABLE 10
Inhibition of CFB mRNA by 5-10-5 MOE gapmers targeting SEQ ID NO: 1 or SEQ ID NO: 2

| | SEQ | SEQ | | | | SEQ | SEQ | |
|---|---|---|---|---|---|---|---|---|
| | ID | ID | | | | ID NO: | ID NO: | |
| | NO: 1 | NO: 1 | | | | 2 | 2 | SEQ |
| ISIS | start | stop | Target | | % in- | start | stop | ID |
| NO | site | site | region | Sequence | hibition | site | site | NO: |
| 588685 | n/a | n/a | Exon 1 | GGATCCAGCTCACTCCCCTG | 14 | 1596 | 1615 | 466 |
| 588686 | n/a | n/a | Exon 1 | AAATAAGGATCCAGCTCACT | 2 | 1602 | 1621 | 467 |
| 588688 | n/a | n/a | Exon 1 | GACCAGAAATAAGGATCCAG | 3 | 1608 | 1627 | 468 |
| 588690 | n/a | n/a | Exon 1 | CTTAGGGACCAGAAATAAGG | 10 | 1614 | 1633 | 469 |
| 588692 | n/a | n/a | Exon 1 | CACCCACTTAGGGACCAGAA | 23 | 1620 | 1639 | 470 |
| 588694 | n/a | n/a | Exon 1 | ACCACCCACTTAGGGACCAG | 23 | 1622 | 1641 | 471 |
| 588696 | n/a | n/a | Exon 1 | AGGTCCAGGACTCTCCCCTT | 15 | 1685 | 1704 | 472 |
| 588698 | n/a | n/a | Exon 1 | AAGGTCCAGGACTCTCCCCT | 19 | 1686 | 1705 | 473 |
| 588700 | n/a | n/a | Exon 1 | AAACTGCAGAAGTCCCACCC | 16 | 1716 | 1735 | 474 |
| 588586 | 30 | 49 | Exon 1 | GGAGGGCCCCGCTGAGCTGC | 11 | 1751 | 1770 | 475 |
| 588587 | 48 | 67 | Exon 1 | TCCCGGAACATCCAAGCGGG | 14 | 1769 | 1788 | 476 |
| 588588 | 56 | 75 | Exon 1 | CATCACTTTCCCGGAACATC | 18 | 1777 | 1796 | 477 |
| 588589 | 151 | 170 | Exon 1 | CTGGTCACATTCCCTTCCCC | 59 | 1872 | 1891 | 478 |
| 588590 | 157 | 176 | Exon 1 | CTAGACCTGGTCACATTCCC | 59 | 1878 | 1897 | 479 |
| 588591 | 339 | 358 | Exon 1-2 | GGAGTGGTGGTCACACCTCC | 45 | n/a | n/a | 480 |
| | | | Junction | | | | | |
| 588592 | 384 | 403 | Exon 2 | ACCCCCTCCAGAGAGCAGGA | 39 | 2192 | 2211 | 481 |
| 588593 | 390 | 409 | Exon 2 | ATCTCTACCCCCTCCAGAGA | 29 | 2198 | 2217 | 482 |
| 588594 | 467 | 486 | Exon 2 | GGTACGGGTAGAAGCCAGAA | 47 | 2275 | 2294 | 483 |
| 588595 | 671 | 690 | Exon 3 | GGAGAGTGTAACCGTCATAG | 44 | 2879 | 2898 | 484 |
| 588596 | 689 | 708 | Exon 3 | TGCGATTGGCAGAGCCCCGG | 43 | 2897 | 2916 | 638 |
| 588597 | 695 | 714 | Exon 3 | GGCAGGTGCGATTGGCAGAG | 34 | 2903 | 2922 | 486 |
| 588598 | 707 | 726 | Exon 3 | GGCCATTCACTTGGCAGGTG | 17 | 2915 | 2934 | 487 |
| 588599 | 738 | 757 | Exon 3 | TTGTCACAGATCGCTGTCTG | 37 | 2946 | 2965 | 488 |
| 588600 | 924 | 943 | Exon 3-4 | AAGGAGTCTTGGCAGGAAGG | 18 | n/a | n/a | 489 |
| | | | Junction | | | | | |
| 588601 | 931 | 950 | Exon 3-4 | GTACATGAAGGAGTCTTGGC | 32 | n/a | n/a | 490 |
| | | | Junction | | | | | |
| 588602 | 959 | 978 | Exon 5 | AAGCTTCGGCCACCTCTTGA | 45 | 3542 | 3561 | 491 |
| 588603 | 1089 | 1108 | Exon 6 | CCATCTAGCACCAGGTAGAT | 52 | 3773 | 3792 | 492 |
| 588604 | 1108 | 1127 | Exon 6 | GGCCCCAATGCTGTCTGATC | 39 | 3792 | 3811 | 493 |
| 588606 | 1150 | 1169 | Exon 6 | AATTAAGTTGACTAGACACT | 37 | 3834 | 3853 | 494 |
| 588608 | 1162 | 1181 | Exon 6-7 | TGCCACCTTCTCAATTAAGT | 21 | n/a | n/a | 648 |
| | | | Junction | | | | | |
| 588578 | 1167 | 1186 | Exon 6-7 | TAACTTGCCACCTTCTCAAT | 22 | n/a | n/a | 496 |
| | | | Junction | | | | | |
| 588579 | 1169 | 1188 | Exon 6-7 | CATAACTTGCCACCTTCTCA | 21 | n/a | n/a | 497 |
| | | | Junction | | | | | |
| 532692 | 1171 | 1190 | Exon 6-7 | ACCATAACTTGCCACCTTCT | 56 | n/a | n/a | 90 |
| | | | Junction | | | | | |
| 588580 | 1173 | 1192 | Exon 6-7 | ACACCATAACTTGCCACCTT | 50 | n/a | n/a | 498 |
| | | | Junction | | | | | |
| 588581 | 1175 | 1194 | Exon 7 | TCACACCATAACTTGCCACC | 50 | 4151 | 4170 | 499 |
| 588610 | 1319 | 1338 | Exon 8 | TAGTCCCTGACTTCAACTTG | 47 | 4612 | 4631 | 500 |
| 588612 | 1325 | 1344 | Exon 8 | TGGTGTTAGTCCCTGACTTC | 47 | 4618 | 4637 | 501 |
| 588614 | 1396 | 1415 | Exon 8 | GCGGTTCCAGCCTTCAGGAG | 51 | 4689 | 4708 | 502 |
| 588616 | 1421 | 1440 | Exon 8 | TCATGAGGATGATGACATGG | 18 | 4714 | 4733 | 503 |
| 588618 | 1446 | 1465 | Exon 9 | CCGCCCATGTTGTGCAATCC | 40 | 5020 | 5039 | 504 |
| 588620 | 1458 | 1477 | Exon 9 | GTAATTGGGTCCCCGCCCAT | 40 | 5032 | 5051 | 505 |
| 588623 | 1482 | 1501 | Exon 9 | AAGTCCCGGATCTCATCAAT | 45 | 5056 | 5075 | 506 |
| 588624 | 1542 | 1561 | Exon 9-10 | AACACATAGACATCCAGATA | 43 | n/a | n/a | 507 |
| | | | Junction | | | | | |
| 588626 | 1585 | 1604 | Exon 10 | CAAAGCATTGATGTTCACTT | 45 | 5234 | 5253 | 508 |
| 588628 | 1621 | 1640 | Exon 10 | TTTGAACACATGTTGCTCAT | 53 | 5270 | 5289 | 509 |
| 588631 | 1646 | 1665 | Exon 10 | CTTCCAGGTTTTCCATATCC | 56 | 5295 | 5314 | 510 |
| 588632 | 1647 | 1666 | Exon 10 | TCTTCCAGGTTTTCCATATC | 35 | 5296 | 5315 | 511 |
| 588634 | 1689 | 1708 | Exon 11 | AGACTCAGAGACTGGCTTTC | 55 | 5830 | 5849 | 512 |
| 588636 | 1749 | 1768 | Exon 11 | GCCTGCCATGGTTGCTTGTG | 78 | 5890 | 5909 | 513 |
| 588638 | 1763 | 1782 | Exon 11 | TGACTGAGATCTTGGCCTGC | 95 | 5904 | 5923 | 514 |
| 588640 | 1912 | 1931 | Exon 13 | TTCTATCTCCAGGTCCCGCT | 44 | 6406 | 6425 | 515 |
| 588642 | 1982 | 2001 | Exon 13 | AGTCATAAAATTCAGGAATT | 40 | 6476 | 6495 | 516 |
| 588645 | 2073 | 2092 | Exon 14 | CGAGTTGTTCCCTCGGTGCA | 57 | 6662 | 6681 | 517 |
| 588646 | 2085 | 2104 | Exon 14 | AGCCTCAAAGCTCGAGTIGT | 48 | 6674 | 6693 | 518 |
| 588648 | 2091 | 2110 | Exon 14 | GGAGGAAGCCTCAAAGCTCG | 40 | 6680 | 6699 | 519 |
| 588651 | 2097 | 2116 | Exon 14 | GTAGTTGGAGGAAGCCTCAA | 43 | 6686 | 6705 | 520 |
| 588652 | 2103 | 2122 | Exon 14 | CAAGTGGTAGTTGGAGGAAG | 13 | 6692 | 6711 | 521 |
| 588654 | 2166 | 2185 | Exon 15 | TCCTCAGACACAAACAGAGC | 55 | 6954 | 6973 | 522 |
| 588656 | 2172 | 2191 | Exon 15 | TTCTCCTCCTCAGACACAAA | 44 | 6960 | 6979 | 523 |
| 588658 | 2196 | 2215 | Exon 15 | TAGACCTCCTTCCGAGTCAG | 50 | 6984 | 7003 | 524 |
| 588660 | 2202 | 2221 | Exon 15 | TTGATGTAGACCTCCTTCCG | 27 | 6990 | 7009 | 525 |
| 588582 | 2219 | 2238 | Exon 15- | CTTTCTTATCCCCATTCTTG | 49 | n/a | n/a | 526 |
| | | | 16 | | | | | |
| | | | Junction | | | | | |
| 588583 | 2221 | 2240 | Exon 15- | GCCTTTCTTATCCCCATTCT | 41 | n/a | n/a | 527 |
| | | | 16 | | | | | |
| | | | Junction | | | | | |
| 532775 | 2223 | 2242 | Exon 15- | CTGCCTTTCTTATCCCCATT | 41 | n/a | n/a | 203 |
| | | | 16 | | | | | |
| | | | Junction | | | | | |
| 588584 | 2225 | 2244 | Exon 15- | AGCTGCCTTTCTTATCCCCA | 43 | n/a | n/a | 528 |
| | | | 16 | | | | | |
| | | | Junction | | | | | |
| 588662 | 2226 | 2245 | Exon 15- | CAGCTGCCTTTCTTATCCCC | 52 | n/a | n/a | 529 |
| | | | 16 | | | | | |
| | | | Junction | | | | | |
| 588585 | 2227 | 2246 | Exon 15- | ACAGCTGCCTTTCTTATCCC | 39 | n/a | n/a | 530 |
| | | | 16 | | | | | |
| | | | Junction | | | | | |
| 588664 | 2238 | 2257 | Exon 16 | GCATCTCTCTCACAGCTGCC | 69 | 7122 | 7141 | 531 |
| 588666 | 2276 | 2295 | Exon 16 | AGATGTCCTTGACTTTGTCA | 46 | 7160 | 7179 | 532 |
| 588668 | 2330 | 2349 | Exon 16 | CAGCATAGGGACTCACTCCT | 47 | 7214 | 7233 | 533 |
| 588670 | 2361 | 2380 | Exon 16- | CCGCCAGAATCACCTCTGCA | 58 | n/a | n/a | 534 |
| | | | 17 | | | | | |
| | | | Junction | | | | | |
| 588672 | 2397 | 2416 | Exon 17 | TGAATGAAACGACTTCTCTT | 48 | 7362 | 7381 | 535 |
| 588674 | 2430 | 2449 | Exon 18 | ACATCCACTACTCCCCAGCT | 29 | 7665 | 7684 | 536 |
| 588676 | 2448 | 2467 | Exon 18 | CGCTTCTGGTTTTTGCAGAC | 58 | 7683 | 7702 | 537 |
| 588678 | 2454 | 2473 | Exon 18 | TTTTGCCGCTTCTGGTTTTT | 45 | 7689 | 7708 | 538 |
| 588680 | 2466 | 2485 | Exon 18 | GCAGGTACCTGCTTTTGCCG | 36 | 7701 | 7720 | 539 |
| 588682 | 2532 | 2551 | Exon 18 | TCTTGGAGTTTCTCCTTCAG | 47 | 7767 | 7786 | 540 |
| 532811 | 2599 | 2618 | Exon 18 | AGCTGTTTTAATTCAATCCC | 96 | 7834 | 7853 | 239 |
| 532917 | 2604 | 2623 | Exon 18 | GTCGCAGCTGTTTTAATTCA | 96 | 7839 | 7858 | 317 |

TABLE 11
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO |
| 598973 | 2552 | 2568 | Exon 18 | GAAAACCCAAATCCTCA | 40 | 7787 | 7803 | 3-10-4 | 619 |
| 599036 | 2552 | 2568 | Exon 18 | GAAAACCCAAATCCTCA | 18 | 7787 | 7803 | 5-7-5 | 619 |
| 598974 | 2553 | 2569 | Exon 18 | AGAAAACCCAAATCCTC | 28 | 7788 | 7804 | 3-10-4 | 620 |
| 599037 | 2553 | 2569 | Exon 18 | AGAAAACCCAAATCCTC | 19 | 7788 | 7804 | 5-7-5 | 620 |
| 598975 | 2554 | 2570 | Exon 18 | TAGAAAACCCAAATCCT | 15 | 7789 | 7805 | 3-10-4 | 621 |
| 599038 | 2554 | 2570 | Exon 18 | TAGAAAACCCAAATCCT | 32 | 7789 | 7805 | 5-7-5 | 621 |
| 598976 | 2555 | 2571 | Exon 18 | ATAGAAAACCCAAATCC | 12 | 7790 | 7806 | 3-10-4 | 622 |
| 599039 | 2555 | 2571 | Exon 18 | ATAGAAAACCCAAATCC | 7 | 7790 | 7806 | 5-7-5 | 622 |
| 598977 | 2557 | 2573 | Exon 18 | TTATAGAAAACCCAAAT | 13 | 7792 | 7808 | 3-10-4 | 623 |
| 599040 | 2557 | 2573 | Exon 18 | TTATAGAAAACCCAAAT | 13 | 7792 | 7808 | 5-7-5 | 623 |
| 598978 | 2558 | 2574 | Exon 18 | CTTATAGAAAACCCAAA | 0 | 7793 | 7809 | 3-10-4 | 624 |
| 599041 | 2558 | 2574 | Exon 18 | CTTATAGAAAACCCAAA | 0 | 7793 | 7809 | 5-7-5 | 624 |
| 598979 | 2559 | 2575 | Exon 18 | CCTTATAGAAAACCCAA | 8 | 7794 | 7810 | 3-10-4 | 625 |
| 599042 | 2559 | 2575 | Exon 18 | CCTTATAGAAAACCCAA | 19 | 7794 | 7810 | 5-7-5 | 625 |
| 598980 | 2560 | 2576 | Exon 18 | CCCTTATAGAAAACCCA | 42 | 7795 | 7811 | 3-10-4 | 626 |
| 599043 | 2560 | 2576 | Exon 18 | CCCTTATAGAAAACCCA | 10 | 7795 | 7811 | 5-7-5 | 626 |
| 598981 | 2561 | 2577 | Exon 18 | CCCCTTATAGAAAACCC | 20 | 7796 | 7812 | 3-10-4 | 627 |
| 599044 | 2561 | 2577 | Exon 18 | CCCCTTATAGAAAACCC | 12 | 7796 | 7812 | 5-7-5 | 627 |
| 598982 | 2562 | 2578 | Exon 18 | ACCCCTTATAGAAAACC | 10 | 7797 | 7813 | 3-10-4 | 628 |
| 599045 | 2562 | 2578 | Exon 18 | ACCCCTTATAGAAAACC | 3 | 7797 | 7813 | 5-7-5 | 628 |
| 598983 | 2563 | 2579 | Exon 18 | AACCCCTTATAGAAAAC | 0 | 7798 | 7814 | 3-10-4 | 629 |
| 599046 | 2563 | 2579 | Exon 18 | AACCCCTTATAGAAAAC | 18 | 7798 | 7814 | 5-7-5 | 629 |
| 598984 | 2564 | 2580 | Exon 18 | AAACCCCTTATAGAAAA | 0 | 7799 | 7815 | 3-10-4 | 630 |
| 599047 | 2564 | 2580 | Exon 18 | AAACCCCTTATAGAAAA | 7 | 7799 | 7815 | 5-7-5 | 630 |
| 598985 | 2565 | 2581 | Exon 18 | GAAACCCCTTATAGAAA | 0 | 7800 | 7816 | 3-10-4 | 631 |
| 599048 | 2565 | 2581 | Exon 18 | GAAACCCCTTATAGAAA | 9 | 7800 | 7816 | 5-7-5 | 631 |
| 598986 | 2566 | 2582 | Exon 18 | GGAAACCCCTTATAGAA | 0 | 7801 | 7817 | 3-10-4 | 632 |
| 599049 | 2566 | 2582 | Exon 18 | GGAAACCCCTTATAGAA | 18 | 7801 | 7817 | 5-7-5 | 632 |
| 598988 | 2567 | 2583 | Exon 18 | AGGAAACCCCTTATAGA | 0 | 7802 | 7818 | 3-10-4 | 633 |
| 599050 | 2567 | 2583 | Exon 18 | AGGAAACCCCTTATAGA | 8 | 7802 | 7818 | 5-7-5 | 633 |
| 598989 | 2568 | 2584 | Exon 18 | CAGGAAACCCCTTATAG | 0 | 7803 | 7819 | 3-10-4 | 634 |
| 598990 | 2569 | 2585 | Exon 18 | GCAGGAAACCCCTTATA | 8 | 7804 | 7820 | 3-10-4 | 635 |
| 598991 | 2570 | 2586 | Exon 18 | AGCAGGAAACCCCTTAT | 25 | 7805 | 7821 | 3-10-4 | 636 |
| 598992 | 2571 | 2587 | Exon 18 | CAGCAGGAAACCCCTTA | 12 | 7806 | 7822 | 3-10-4 | 637 |
| 598993 | 2572 | 2588 | Exon 18 | CCAGCAGGAAACCCCTT | 37 | 7807 | 7823 | 3-10-4 | 638 |
| 598994 | 2573 | 2589 | Exon 18 | TCCAGCAGGAAACCCCT | 29 | 7808 | 7824 | 3-10-4 | 639 |
| 598995 | 2574 | 2590 | Exon 18 | GTCCAGCAGGAAACCCC | 42 | 7809 | 7825 | 3-10-4 | 640 |
| 598996 | 2575 | 2591 | Exon 18 | TGTCCAGCAGGAAACCC | 36 | 7810 | 7826 | 3-10-4 | 641 |
| 598997 | 2576 | 2592 | Exon 18 | CTGTCCAGCAGGAAACC | 18 | 7811 | 7827 | 3-10-4 | 642 |
| 598998 | 2577 | 2593 | Exon 18 | CCTGTCCAGCAGGAAAC | 27 | 7812 | 7828 | 3-10-4 | 643 |
| 598999 | 2578 | 2594 | Exon 18 | CCCTGTCCAGCAGGAAA | 61 | 7813 | 7829 | 3-10-4 | 644 |
| 599000 | 2580 | 2596 | Exon 18 | GCCCCTGTCCAGCAGGA | 71 | 7815 | 7831 | 3-10-4 | 645 |
| 599001 | 2581 | 2597 | Exon 18 | CGCCCCTGTCCAGCAGG | 80 | 7816 | 7832 | 3-10-4 | 646 |
| 599002 | 2582 | 2598 | Exon 18 | ACGCCCCTGTCCAGCAG | 68 | 7817 | 7833 | 3-10-4 | 647 |
| 599003 | 2583 | 2599 | Exon 18 | CACGCCCCTGTCCAGCA | 71 | 7818 | 7834 | 3-10-4 | 648 |
| 599004 | 2584 | 2600 | Exon 18 | CCACGCCCCTGTCCAGC | 76 | 7819 | 7835 | 3-10-4 | 649 |
| 599005 | 2585 | 2601 | Exon 18 | CCCACGCCCCTGTCCAG | 70 | 7820 | 7836 | 3-10-4 | 650 |
| 599006 | 2586 | 2602 | Exon 18 | TCCCACGCCCCTGTCCA | 65 | 7821 | 7837 | 3-10-4 | 651 |
| 599007 | 2587 | 2603 | Exon 18 | ATCCCACGCCCCTGTCC | 60 | 7822 | 7838 | 3-10-4 | 652 |
| 599008 | 2588 | 2604 | Exon 18 | AATCCCACGCCCCTGTC | 72 | 7823 | 7839 | 3-10-4 | 653 |
| 599009 | 2589 | 2605 | Exon 18 | CAATCCCACGCCCCTGT | 79 | 7824 | 7840 | 3-10-4 | 654 |
| 599010 | 2590 | 2606 | Exon 18 | TCAATCCCACGCCCCTG | 73 | 7825 | 7841 | 3-10-4 | 655 |
| 599011 | 2591 | 2607 | Exon 18 | TTCAATCCCACGCCCCT | 79 | 7826 | 7842 | 3-10-4 | 656 |
| 599012 | 2592 | 2608 | Exon 18 | ATTCAATCCCACGCCCC | 67 | 7827 | 7843 | 3-10-4 | 657 |
| 599013 | 2593 | 2609 | Exon 18 | AATTCAATCCCACGCCC | 65 | 7828 | 7844 | 3-10-4 | 658 |
| 599014 | 2594 | 2610 | Exon 18 | TAATTCAATCCCACGCC | 74 | 7829 | 7845 | 3-10-4 | 659 |
| 599015 | 2595 | 2611 | Exon 18 | TTAATTCAATCCCACGC | 71 | 7830 | 7846 | 3-10-4 | 660 |
| 599016 | 2596 | 2612 | Exon 18 | TTTAATTCAATCCCACG | 48 | 7831 | 7847 | 3-10-4 | 661 |
| 599017 | 2597 | 2613 | Exon 18 | TTTTAATTCAATCCCAC | 34 | 7832 | 7848 | 3-10-4 | 662 |
| 599018 | 2598 | 2614 | Exon 18 | GTTTTAATTCAATCCCA | 56 | 7833 | 7849 | 3-10-4 | 663 |
| 599019 | 2599 | 2615 | Exon 18 | TGTTTTAATTCAATCCC | 60 | 7834 | 7850 | 3-10-4 | 664 |
| 599020 | 2600 | 2616 | Exon 18 | CTGTTTTAATTCAATCC | 0 | 7835 | 7851 | 3-10-4 | 665 |
| 599021 | 2601 | 2617 | Exon 18 | GCTGTTTTAATTCAATC | 33 | 7836 | 7852 | 3-10-4 | 666 |
| 599022 | 2602 | 2618 | Exon 18 | AGCTGTTTTAATTCAAT | 17 | 7837 | 7853 | 3-10-4 | 667 |
| 599023 | 2603 | 2619 | Exon 18 | CAGCTGTTTTAATTCAA | 52 | 7838 | 7854 | 3-10-4 | 668 |
| 532917 | 2604 | 2623 | Exon 18 | GTCGCAGCTGTTTTAATTCA | 86 | 7839 | 7858 | 5-10-5 | 317 |
| 599024 | 2604 | 2620 | Exon 18 | GCAGCTGTTTTAATTCA | 88 | 7839 | 7855 | 3-10-4 | 669 |
| 599025 | 2605 | 2621 | Exon 18 | CGCAGCTGTTTTAATTC | 85 | 7840 | 7856 | 3-10-4 | 670 |
| 599026 | 2606 | 2622 | Exon 18 | TCGCAGCTGTTTTAATT | 69 | 7841 | 7857 | 3-10-4 | 671 |
| 599027 | 2607 | 2623 | Exon 18 | GTCGCAGCTGTTTTAAT | 77 | 7842 | 7858 | 3-10-4 | 672 |
| 599028 | 2608 | 2624 | Exon 18 | TGTCGCAGCTGTTTTAA | 73 | 7843 | 7859 | 3-10-4 | 673 |
| 599029 | 2609 | 2625 | Exon 18 | TTGTCGCAGCTGTTTTA | 78 | 7844 | 7860 | 3-10-4 | 674 |
| 599030 | 2610 | 2626 | Exon 18 | GTTGTCGCAGCTGTTTT | 75 | 7845 | 7861 | 3-10-4 | 675 |
| 599031 | 2611 | 2627 | Exon 18 | TGTTGTCGCAGCTGTTT | 77 | 7846 | 7862 | 3-10-4 | 676 |
| 599032 | 2612 | 2628 | Exon 18/ | TTGTTGTCGCAGCTGTT | 79 | n/a | n/a | 3-10-4 | 677 |
| | | | Repeat | | | | | | |
| 599033 | 2613 | 2629 | Exon 18/ | TTTGTTGTCGCAGCTGT | 80 | n/a | n/a | 3-10-4 | 678 |
| | | | Repeat | | | | | | |
| 599034 | 2614 | 2630 | Exon 18/ | TTTTGTTGTCGCAGCTG | 78 | n/a | n/a | 3-10-4 | 679 |
| | | | Repeat | | | | | | |
| 599035 | 2615 | 2631 | Exon 18/ | TTTTTGTTGTCGCAGCT | 63 | n/a | n/a | 3-10-4 | 680 |
| | | | Repeat | | | | | | |

TABLE 12
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 599098 | 2552 | 2568 | Exon 18 | GAAAACCCAAATCCTCA | 57 | 7787 | 7803 | 4-8-5 | 619 |
| 599099 | 2553 | 2569 | Exon 18 | AGAAAACCCAAATCCTC | 33 | 7788 | 7804 | 4-8-5 | 620 |
| 599100 | 2554 | 2570 | Exon 18 | TAGAAAACCCAAATCCT | 32 | 7789 | 7805 | 4-8-5 | 621 |
| 599101 | 2555 | 2571 | Exon 18 | ATAGAAAACCCAAATCC | 47 | 7790 | 7806 | 4-8-5 | 622 |
| 599102 | 2557 | 2573 | Exon 18 | TTATAGAAAACCCAAAT | 59 | 7792 | 7808 | 4-8-5 | 623 |
| 599103 | 2558 | 2574 | Exon 18 | CTTATAGAAAACCCAAA | 10 | 7793 | 7809 | 4-8-5 | 624 |
| 599104 | 2559 | 2575 | Exon 18 | CCTTATAGAAAACCCAA | 3 | 7794 | 7810 | 4-8-5 | 625 |
| 599105 | 2560 | 2576 | Exon 18 | CCCTTATAGAAAACCCA | 45 | 7795 | 7811 | 4-8-5 | 626 |
| 599106 | 2561 | 2577 | Exon 18 | CCCCTTATAGAAAACCC | 49 | 7796 | 7812 | 4-8-5 | 627 |
| 599107 | 2562 | 2578 | Exon 18 | ACCCCTTATAGAAAACC | 35 | 7797 | 7813 | 4-8-5 | 628 |
| 599108 | 2563 | 2579 | Exon 18 | AACCCCTTATAGAAAAC | 17 | 7798 | 7814 | 4-8-5 | 629 |
| 599109 | 2564 | 2580 | Exon 18 | AAACCCCTTATAGAAAA | 36 | 7799 | 7815 | 4-8-5 | 630 |
| 599110 | 2565 | 2581 | Exon 18 | GAAACCCCTTATAGAAA | 20 | 7800 | 7816 | 4-8-5 | 631 |
| 599111 | 2566 | 2582 | Exon 18 | GGAAACCCCTTATAGAA | 20 | 7801 | 7817 | 4-8-5 | 632 |
| 599112 | 2567 | 2583 | Exon 18 | AGGAAACCCCTTATAGA | 15 | 7802 | 7818 | 4-8-5 | 633 |
| 599113 | 2568 | 2584 | Exon 18 | CAGGAAACCCCTTATAG | 19 | 7803 | 7819 | 4-8-5 | 634 |
| 599051 | 2568 | 2584 | Exon 18 | CAGGAAACCCCTTATAG | 26 | 7803 | 7819 | 5-7-5 | 634 |
| 599114 | 2569 | 2585 | Exon 18 | GCAGGAAACCCCTTATA | 18 | 7804 | 7820 | 4-8-5 | 635 |
| 599052 | 2569 | 2585 | Exon 18 | GCAGGAAACCCCTTATA | 21 | 7804 | 7820 | 5-7-5 | 635 |
| 599115 | 2570 | 2586 | Exon 18 | AGCAGGAAACCCCTTAT | 31 | 7805 | 7821 | 4-8-5 | 636 |
| 599053 | 2570 | 2586 | Exon 18 | AGCAGGAAACCCCTTAT | 25 | 7805 | 7821 | 5-7-5 | 636 |
| 599116 | 2571 | 2587 | Exon 18 | CAGCAGGAAACCCCTTA | 39 | 7806 | 7822 | 4-8-5 | 637 |
| 599054 | 2571 | 2587 | Exon 18 | CAGCAGGAAACCCCTTA | 36 | 7806 | 7822 | 5-7-5 | 637 |
| 599117 | 2572 | 2588 | Exon 18 | CCAGCAGGAAACCCCTT | 46 | 7807 | 7823 | 4-8-5 | 638 |
| 599055 | 2572 | 2588 | Exon 18 | CCAGCAGGAAACCCCTT | 22 | 7807 | 7823 | 5-7-5 | 638 |
| 599118 | 2573 | 2589 | Exon 18 | TCCAGCAGGAAACCCCT | 40 | 7808 | 7824 | 4-8-5 | 639 |
| 599056 | 2573 | 2589 | Exon 18 | TCCAGCAGGAAACCCCT | 32 | 7808 | 7824 | 5-7-5 | 639 |
| 599119 | 2574 | 2590 | Exon 18 | GTCCAGCAGGAAACCCC | 50 | 7809 | 7825 | 4-8-5 | 640 |
| 599057 | 2574 | 2590 | Exon 18 | GTCCAGCAGGAAACCCC | 46 | 7809 | 7825 | 5-7-5 | 640 |
| 599120 | 2575 | 2591 | Exon 18 | TGTCCAGCAGGAAACCC | 30 | 7810 | 7826 | 4-8-5 | 641 |
| 599058 | 2575 | 2591 | Exon 18 | TGTCCAGCAGGAAACCC | 52 | 7810 | 7826 | 5-7-5 | 641 |
| 599121 | 2576 | 2592 | Exon 18 | CTGTCCAGCAGGAAACC | 31 | 7811 | 7827 | 4-8-5 | 642 |
| 599059 | 2576 | 2592 | Exon 18 | CTGTCCAGCAGGAAACC | 24 | 7811 | 7827 | 5-7-5 | 642 |
| 599122 | 2577 | 2593 | Exon 18 | CCTGTCCAGCAGGAAAC | 23 | 7812 | 7828 | 4-8-5 | 643 |
| 599060 | 2577 | 2593 | Exon 18 | CCTGTCCAGCAGGAAAC | 37 | 7812 | 7828 | 5-7-5 | 643 |
| 599123 | 2578 | 2594 | Exon 18 | CCCTGTCCAGCAGGAAA | 51 | 7813 | 7829 | 4-8-5 | 644 |
| 599061 | 2578 | 2594 | Exon 18 | CCCTGTCCAGCAGGAAA | 34 | 7813 | 7829 | 5-7-5 | 644 |
| 599124 | 2580 | 2596 | Exon 18 | GCCCCTGTCCAGCAGGA | 56 | 7815 | 7831 | 4-8-5 | 645 |
| 599062 | 2580 | 2596 | Exon 18 | GCCCCTGTCCAGCAGGA | 51 | 7815 | 7831 | 5-7-5 | 645 |
| 599125 | 2581 | 2597 | Exon 18 | CGCCCCTGTCCAGCAGG | 70 | 7816 | 7832 | 4-8-5 | 646 |
| 599063 | 2581 | 2597 | Exon 18 | CGCCCCTGTCCAGCAGG | 56 | 7816 | 7832 | 5-7-5 | 646 |
| 599126 | 2582 | 2598 | Exon 18 | ACGCCCCTGTCCAGCAG | 76 | 7817 | 7833 | 4-8-5 | 647 |
| 599064 | 2582 | 2598 | Exon 18 | ACGCCCCTGTCCAGCAG | 61 | 7817 | 7833 | 5-7-5 | 647 |
| 599127 | 2583 | 2599 | Exon 18 | CACGCCCCTGTCCAGCA | 67 | 7818 | 7834 | 4-8-5 | 648 |
| 599065 | 2583 | 2599 | Exon 18 | CACGCCCCTGTCCAGCA | 64 | 7818 | 7834 | 5-7-5 | 648 |
| 599066 | 2584 | 2600 | Exon 18 | CCACGCCCCTGTCCAGC | 40 | 7819 | 7835 | 5-7-5 | 649 |
| 599067 | 2585 | 2601 | Exon 18 | CCCACGCCCCTGTCCAG | 37 | 7820 | 7836 | 5-7-5 | 650 |
| 599068 | 2586 | 2602 | Exon 18 | TCCCACGCCCCTGTCCA | 31 | 7821 | 7837 | 5-7-5 | 651 |
| 599069 | 2587 | 2603 | Exon 18 | ATCCCACGCCCCTGTCC | 39 | 7822 | 7838 | 5-7-5 | 652 |
| 599070 | 2588 | 2604 | Exon 18 | AATCCCACGCCCCTGTC | 59 | 7823 | 7839 | 5-7-5 | 653 |
| 599071 | 2589 | 2605 | Exon 18 | CAATCCCACGCCCCTGT | 63 | 7824 | 7840 | 5-7-5 | 657 |
| 599072 | 2590 | 2606 | Exon 18 | TCAATCCCACGCCCCTG | 74 | 7825 | 7841 | 5-7-5 | 655 |
| 599073 | 2591 | 2607 | Exon 18 | TTCAATCCCACGCCCCT | 53 | 7826 | 7842 | 5-7-5 | 656 |
| 599074 | 2592 | 2608 | Exon 18 | ATTCAATCCCACGCCCC | 56 | 7827 | 7843 | 5-7-5 | 657 |
| 599075 | 2593 | 2609 | Exon 18 | AATTCAATCCCACGCCC | 49 | 7828 | 7844 | 5-7-5 | 658 |
| 599076 | 2594 | 2610 | Exon 18 | TAATTCAATCCCACGCC | 54 | 7829 | 7845 | 5-7-5 | 659 |
| 599077 | 2595 | 2611 | Exon 18 | TTAATTCAATCCCACGC | 79 | 7830 | 7846 | 5-7-5 | 660 |
| 599078 | 2596 | 2612 | Exon 18 | TTTAATTCAATCCCACG | 67 | 7831 | 7847 | 5-7-5 | 661 |
| 599079 | 2597 | 2613 | Exon 18 | TTTTAATTCAATCCCAC | 69 | 7832 | 7848 | 5-7-5 | 662 |
| 599080 | 2598 | 2614 | Exon 18 | GTTTTAATTCAATCCCA | 79 | 7833 | 7849 | 5-7-5 | 663 |
| 599081 | 2599 | 2615 | Exon 18 | TGTTTTAATTCAATCCC | 57 | 7834 | 7850 | 5-7-5 | 664 |
| 599082 | 2600 | 2616 | Exon 18 | CTGTTTTAATTCAATCC | 50 | 7835 | 7851 | 5-7-5 | 665 |
| 599083 | 2601 | 2617 | Exon 18 | GCTGTTTTAATTCAATC | 67 | 7836 | 7852 | 5-7-5 | 666 |
| 599084 | 2602 | 2618 | Exon 18 | AGCTGTTTTAATTCAAT | 60 | 7837 | 7853 | 5-7-5 | 667 |
| 599085 | 2603 | 2619 | Exon 18 | CAGCTGTTTTAATTCAA | 71 | 7838 | 7854 | 5-7-5 | 668 |
| 532917 | 2604 | 2623 | Exon 18 | GTCGCAGCTGTTTTAATTCA | 82 | 7839 | 7858 | 5-10-5 | 317 |
| 599086 | 2604 | 2620 | Exon 18 | GCAGCTGTTTTAATTCA | 81 | 7839 | 7855 | 5-7-5 | 669 |
| 599087 | 2605 | 2621 | Exon 18 | CGCAGCTGTTTTAATTC | 88 | 7840 | 7856 | 5-7-5 | 670 |
| 599088 | 2606 | 2622 | Exon 18 | TCGCAGCTGTTTTAATT | 84 | 7841 | 7857 | 5-7-5 | 671 |
| 599089 | 2607 | 2623 | Exon 18 | GTCGCAGCTGTTTTAAT | 81 | 7842 | 7858 | 5-7-5 | 672 |
| 599090 | 2608 | 2624 | Exon 18 | TGTCGCAGCTGTTTTAA | 77 | 7843 | 7859 | 5-7-5 | 673 |
| 599091 | 2609 | 2625 | Exon 18 | TTGTCGCAGCTGTTTTA | 74 | 7844 | 7860 | 5-7-5 | 674 |
| 599092 | 2610 | 2626 | Exon 18 | GTTGTCGCAGCTGTTTT | 66 | 7845 | 7861 | 5-7-5 | 675 |
| 599093 | 2611 | 2627 | Exon 18 | TGTTGTCGCAGCTGTTT | 89 | 7846 | 7862 | 5-7-5 | 676 |
| 599094 | 2612 | 2628 | Exon 18/ | TTGTTGTCGCAGCTGTT | 82 | n/a | n/a | 5-7-5 | 677 |
| | | | Repeat | | | | | | |
| 599095 | 2613 | 2629 | Exon 18/ | TTTGTTGTCGCAGCTGT | 87 | n/a | n/a | 5-7-5 | 678 |
| | | | Repeat | | | | | | |
| 599096 | 2614 | 2630 | Exon 18/ | TTTTGTTGTCGCAGCTG | 85 | n/a | n/a | 5-7-5 | 679 |
| | | | Repeat | | | | | | |
| 599097 | 2615 | 2631 | Exon 18/ | TTTTTGTTGTCGCAGCT | 78 | n/a | n/a | 5-7-5 | 680 |
| | | | Repeat | | | | | | |

TABLE 13
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 599510 | 2552 | 2570 | Exon 18 | TAGAAAACCCAAATCCTCA | 45 | 7787 | 7805 | 5-9-5 | 681 |
| 599331 | 2553 | 2571 | Exon 18 | ATAGAAAACCCAAATCCTC | 46 | 7788 | 7806 | 5-9-5 | 682 |
| 599332 | 2554 | 2572 | Exon 18 | TATAGAAAACCCAAATCCT | 38 | 7789 | 7807 | 5-9-5 | 683 |
| 599333 | 2556 | 2574 | Exon 18 | CTTATAGAAAACCCAAATC | 1 | 7791 | 7809 | 5-9-5 | 684 |
| 599334 | 2557 | 2575 | Exon 18 | CCTTATAGAAAACCCAAAT | 5 | 7792 | 7810 | 5-9-5 | 685 |
| 599335 | 2558 | 2576 | Exon 18 | CCCTTATAGAAAACCCAAA | 34 | 7793 | 7811 | 5-9-5 | 686 |
| 599336 | 2559 | 2577 | Exon 18 | CCCCTTATAGAAAACCCAA | 40 | 7794 | 7812 | 5-9-5 | 687 |
| 599337 | 2560 | 2578 | Exon 18 | ACCCCTTATAGAAAACCCA | 39 | 7795 | 7813 | 5-9-5 | 688 |
| 599338 | 2561 | 2579 | Exon 18 | AACCCCTTATAGAAAACCC | 57 | 7796 | 7814 | 5-9-5 | 689 |
| 599339 | 2562 | 2580 | Exon 18 | AAACCCCTTATAGAAAACC | 26 | 7797 | 7815 | 5-9-5 | 690 |
| 599281 | 2562 | 2580 | Exon 18 | AAACCCCTTATAGAAAACC | 15 | 7797 | 7815 | 6-7-6 | 690 |
| 599340 | 2563 | 2581 | Exon 18 | GAAACCCCTTATAGAAAAC | 17 | 7798 | 7816 | 5-9-5 | 691 |
| 599282 | 2563 | 2581 | Exon 18 | GAAACCCCTTATAGAAAAC | 12 | 7798 | 7816 | 6-7-6 | 691 |
| 599341 | 2564 | 2582 | Exon 18 | GGAAACCCCTTATAGAAAA | 23 | 7799 | 7817 | 5-9-5 | 692 |
| 599283 | 2564 | 2582 | Exon 18 | GGAAACCCCTTATAGAAAA | 18 | 7799 | 7817 | 6-7-6 | 692 |
| 599342 | 2565 | 2583 | Exon 18 | AGGAAACCCCTTATAGAAA | 10 | 7800 | 7818 | 5-9-5 | 693 |
| 599284 | 2565 | 2583 | Exon 18 | AGGAAACCCCTTATAGAAA | 14 | 7800 | 7818 | 6-7-6 | 693 |
| 599343 | 2566 | 2584 | Exon 18 | CAGGAAACCCCTTATAGAA | 10 | 7801 | 7819 | 5-9-5 | 694 |
| 599285 | 2566 | 2584 | Exon 18 | CAGGAAACCCCTTATAGAA | 13 | 7801 | 7819 | 6-7-6 | 694 |
| 599344 | 2567 | 2585 | Exon 18 | GCAGGAAACCCCTTATAGA | 22 | 7802 | 7820 | 5-9-5 | 695 |
| 599286 | 2567 | 2585 | Exon 18 | GCAGGAAACCCCTTATAGA | 31 | 7802 | 7820 | 6-7-6 | 695 |
| 599345 | 2568 | 2586 | Exon 18 | AGCAGGAAACCCCTTATAG | 19 | 7803 | 7821 | 5-9-5 | 696 |
| 599287 | 2568 | 2586 | Exon 18 | AGCAGGAAACCCCTTATAG | 12 | 7803 | 7821 | 6-7-6 | 696 |
| 599346 | 2569 | 2587 | Exon 18 | CAGCAGGAAACCCCTTATA | 30 | 7804 | 7822 | 5-9-5 | 697 |
| 599288 | 2569 | 2587 | Exon 18 | CAGCAGGAAACCCCTTATA | 28 | 7804 | 7822 | 6-7-6 | 697 |
| 599347 | 2570 | 2588 | Exon 18 | CCAGCAGGAAACCCCTTAT | 46 | 7805 | 7823 | 5-9-5 | 698 |
| 599289 | 2570 | 2588 | Exon 18 | CCAGCAGGAAACCCCTTAT | 32 | 7805 | 7823 | 6-7-6 | 698 |
| 599348 | 2571 | 2589 | Exon 18 | TCCAGCAGGAAACCCCTTA | 44 | 7806 | 7824 | 5-9-5 | 699 |
| 599290 | 2571 | 2589 | Exon 18 | TCCAGCAGGAAACCCCTTA | 24 | 7806 | 7824 | 6-7-6 | 699 |
| 599349 | 2572 | 2590 | Exon 18 | GTCCAGCAGGAAACCCCTT | 60 | 7807 | 7825 | 5-9-5 | 700 |
| 599291 | 2572 | 2590 | Exon 18 | GTCCAGCAGGAAACCCCTT | 38 | 7807 | 7825 | 6-7-6 | 700 |
| 599350 | 2573 | 2591 | Exon 18 | TGTCCAGCAGGAAACCCCT | 49 | 7808 | 7826 | 5-9-5 | 701 |
| 599292 | 2573 | 2591 | Exon 18 | TGTCCAGCAGGAAACCCCT | 35 | 7808 | 7826 | 6-7-6 | 701 |
| 599351 | 2575 | 2593 | Exon 18 | CCTGTCCAGCAGGAAACCC | 46 | 7810 | 7828 | 5-9-5 | 702 |
| 599293 | 2575 | 2593 | Exon 18 | CCTGTCCAGCAGGAAACCC | 12 | 7810 | 7828 | 6-7-6 | 702 |
| 599352 | 2576 | 2594 | Exon 18 | CCCTGTCCAGCAGGAAACC | 49 | 7811 | 7829 | 5-9-5 | 703 |
| 599294 | 2576 | 2594 | Exon 18 | CCCTGTCCAGCAGGAAACC | 38 | 7811 | 7829 | 6-7-6 | 703 |
| 599353 | 2577 | 2595 | Exon 18 | CCCCTGTCCAGCAGGAAAC | 64 | 7812 | 7830 | 5-9-5 | 704 |
| 599295 | 2577 | 2595 | Exon 18 | CCCCTGTCCAGCAGGAAAC | 33 | 7812 | 7830 | 6-7-6 | 704 |
| 599354 | 2578 | 2596 | Exon 18 | GCCCCTGTCCAGCAGGAAA | 56 | 7813 | 7831 | 5-9-5 | 705 |
| 599296 | 2578 | 2596 | Exon 18 | GCCCCTGTCCAGCAGGAAA | 13 | 7813 | 7831 | 6-7-6 | 705 |
| 599355 | 2580 | 2598 | Exon 18 | ACGCCCCTGTCCAGCAGGA | 81 | 7815 | 7833 | 5-9-5 | 706 |
| 599297 | 2580 | 2598 | Exon 18 | ACGCCCCTGTCCAGCAGGA | 57 | 7815 | 7833 | 6-7-6 | 706 |
| 599356 | 2581 | 2599 | Exon 18 | CACGCCCCTGTCCAGCAGG | 64 | 7816 | 7834 | 5-9-5 | 707 |
| 599298 | 2581 | 2599 | Exon 18 | CACGCCCCTGTCCAGCAGG | 39 | 7816 | 7834 | 6-7-6 | 707 |
| 599299 | 2582 | 2600 | Exon 18 | CCACGCCCCTGTCCAGCAG | 55 | 7817 | 7835 | 6-7-6 | 708 |
| 599300 | 2583 | 2601 | Exon 18 | CCCACGCCCCTGTCCAGCA | 45 | 7818 | 7836 | 6-7-6 | 709 |
| 599301 | 2584 | 2602 | Exon 18 | TCCCACGCCCCTGTCCAGC | 39 | 7819 | 7837 | 6-7-6 | 710 |
| 599302 | 2585 | 2603 | Exon 18 | ATCCCACGCCCCTGTCCAG | 27 | 7820 | 7838 | 6-7-6 | 711 |
| 599303 | 2586 | 2604 | Exon 18 | AATCCCACGCCCCTGTCCA | 35 | 7821 | 7839 | 6-7-6 | 712 |
| 599304 | 2587 | 2605 | Exon 18 | CAATCCCACGCCCCTGTCC | 16 | 7822 | 7840 | 6-7-6 | 713 |
| 599305 | 2588 | 2606 | Exon 18 | TCAATCCCACGCCCCTGTC | 41 | 7823 | 7841 | 6-7-6 | 714 |
| 599306 | 2589 | 2607 | Exon 18 | TTCAATCCCACGCCCCTGT | 70 | 7824 | 7842 | 6-7-6 | 715 |
| 599307 | 2590 | 2608 | Exon 18 | ATTCAATCCCACGCCCCTG | 66 | 7825 | 7843 | 6-7-6 | 716 |
| 599308 | 2591 | 2609 | Exon 18 | AATTCAATCCCACGCCCCT | 68 | 7826 | 7844 | 6-7-6 | 717 |
| 599309 | 2592 | 2610 | Exon 18 | TAATTCAATCCCACGCCCC | 52 | 7827 | 7845 | 6-7-6 | 718 |
| 599310 | 2593 | 2611 | Exon 18 | TTAATTCAATCCCACGCCC | 39 | 7828 | 7846 | 6-7-6 | 719 |
| 599311 | 2594 | 2612 | Exon 18 | TTTAATTCAATCCCACGCC | 83 | 7829 | 7847 | 6-7-6 | 720 |
| 599312 | 2595 | 2613 | Exon 18 | TTTTAATTCAATCCCACGC | 72 | 7830 | 7848 | 6-7-6 | 721 |
| 599313 | 2596 | 2614 | Exon 18 | GTTTTAATTCAATCCCACG | 86 | 7831 | 7849 | 6-7-6 | 722 |
| 599314 | 2597 | 2615 | Exon 18 | TGTTTTAATTCAATCCCAC | 91 | 7832 | 7850 | 6-7-6 | 723 |
| 599315 | 2598 | 2616 | Exon 18 | CTGTTTTAATTCAATCCCA | 71 | 7833 | 7851 | 6-7-6 | 724 |
| 599316 | 2599 | 2617 | Exon 18 | GCTGTTTTAATTCAATCCC | 89 | 7834 | 7852 | 6-7-6 | 725 |
| 599317 | 2600 | 2618 | Exon 18 | AGCTGTTTTAATTCAATCC | 87 | 7835 | 7853 | 6-7-6 | 726 |
| 599318 | 2601 | 2619 | Exon 18 | CAGCTGTTTTAATTCAATC | 81 | 7836 | 7854 | 6-7-6 | 727 |
| 599319 | 2602 | 2620 | Exon 18 | GCAGCTGTTTTAATTCAAT | 75 | 7837 | 7855 | 6-7-6 | 728 |
| 599320 | 2603 | 2621 | Exon 18 | CGCAGCTGTTTTAATTCAA | 84 | 7838 | 7856 | 6-7-6 | 729 |
| 532917 | 2604 | 2623 | Exon 18 | GTCGCAGCTGTTTTAATTCA | 92 | 7839 | 7858 | 5-10-5 | 317 |
| 599321 | 2604 | 2622 | Exon 18 | TCGCAGCTGTTTTAATTCA | 90 | 7839 | 7857 | 6-7-6 | 730 |
| 599322 | 2605 | 2623 | Exon 18 | GTCGCAGCTGTTTTAATTC | 89 | 7840 | 7858 | 6-7-6 | 731 |
| 599323 | 2606 | 2624 | Exon 18 | TGTCGCAGCTGTTTTAATT | 81 | 7841 | 7859 | 6-7-6 | 732 |
| 599324 | 2607 | 2625 | Exon 18 | TTGTCGCAGCTGTTTTAAT | 68 | 7842 | 7860 | 6-7-6 | 733 |
| 599325 | 2608 | 2626 | Exon 18 | GTTGTCGCAGCTGTTTTAA | 71 | 7843 | 7861 | 6-7-6 | 734 |
| 599326 | 2609 | 2627 | Exon 18 | TGTTGTCGCAGCTGTTTTA | 52 | 7844 | 7862 | 6-7-6 | 735 |
| 599327 | 2610 | 2628 | Exon 18/ | TTGTTGTCGCAGCTGTTTT | 88 | n/a | n/a | 6-7-6 | 736 |
| | | | Repeat | | | | | | |
| 599328 | 2611 | 2629 | Exon 18/ | TTTGTTGTCGCAGCTGTTT | 87 | n/a | n/a | 6-7-6 | 737 |
| | | | Repeat | | | | | | |
| 599329 | 2612 | 2630 | Exon 18/ | TTTTGTTGTCGCAGCTGTT | 84 | n/a | n/a | 6-7-6 | 738 |
| | | | Repeat | | | | | | |
| 599330 | 2613 | 2631 | Exon 18/ | TTTTTGTTGTCGCAGCTGT | 87 | n/a | n/a | 6-7-6 | 739 |
| | | | Repeat | | | | | | |

TABLE 14
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 599512 | 2552 | 2571 | Exon 18 | ATAGAAAACCCAAATCCTCA | 74 | 7787 | 7806 | 3-10-7 | 410 |
| 599449 | 2553 | 2572 | Exon 18 | TATAGAAAACCCAAATCCTC | 43 | 7788 | 7807 | 3-10-7 | 411 |
| 599450 | 2554 | 2573 | Exon 18 | TTATAGAAAACCCAAATCCT | 51 | 7789 | 7808 | 3-10-7 | 412 |
| 599451 | 2555 | 2574 | Exon 18 | CTTATAGAAAACCCAAATCC | 35 | 7790 | 7809 | 3-10-7 | 413 |
| 599452 | 2556 | 2575 | Exon 18 | CCTTATAGAAAACCCAAATC | 34 | 7791 | 7810 | 3-10-7 | 414 |
| 599453 | 2557 | 2576 | Exon 18 | CCCTTATAGAAAACCCAAAT | 44 | 7792 | 7811 | 3-10-7 | 415 |
| 599454 | 2558 | 2577 | Exon 18 | CCCCTTATAGAAAACCCAAA | 54 | 7793 | 7812 | 3-10-7 | 416 |
| 599455 | 2559 | 2578 | Exon 18 | ACCCCTTATAGAAAACCCAA | 53 | 7794 | 7813 | 3-10-7 | 417 |
| 599456 | 2560 | 2579 | Exon 18 | AACCCCTTATAGAAAACCCA | 69 | 7795 | 7814 | 3-10-7 | 418 |
| 599457 | 2561 | 2580 | Exon 18 | AAACCCCTTATAGAAAACCC | 46 | 7796 | 7815 | 3-10-7 | 419 |
| 599458 | 2562 | 2581 | Exon 18 | GAAACCCCTTATAGAAAACC | 0 | 7797 | 7816 | 3-10-7 | 420 |
| 599459 | 2563 | 2582 | Exon 18 | GGAAACCCCTTATAGAAAAC | 12 | 7798 | 7817 | 3-10-7 | 421 |
| 599460 | 2564 | 2583 | Exon 18 | AGGAAACCCCTTATAGAAAA | 17 | 7799 | 7818 | 3-10-7 | 422 |
| 599461 | 2565 | 2584 | Exon 18 | CAGGAAACCCCTTATAGAAA | 24 | 7800 | 7819 | 3-10-7 | 423 |
| 599462 | 2566 | 2585 | Exon 18 | GCAGGAAACCCCTTATAGAA | 33 | 7801 | 7820 | 3-10-7 | 424 |
| 599463 | 2567 | 2586 | Exon 18 | AGCAGGAAACCCCTTATAGA | 38 | 7802 | 7821 | 3-10-7 | 425 |
| 599464 | 2568 | 2587 | Exon 18 | CAGCAGGAAACCCCTTATAG | 33 | 7803 | 7822 | 3-10-7 | 426 |
| 599465 | 2569 | 2588 | Exon 18 | CCAGCAGGAAACCCCTTATA | 49 | 7804 | 7823 | 3-10-7 | 427 |
| 599466 | 2570 | 2589 | Exon 18 | TCCAGCAGGAAACCCCTTAT | 45 | 7805 | 7824 | 3-10-7 | 428 |
| 599467 | 2571 | 2590 | Exon 18 | GTCCAGCAGGAAACCCCTTA | 60 | 7806 | 7825 | 3-10-7 | 237 |
| 599468 | 2572 | 2591 | Exon 18 | TGTCCAGCAGGAAACCCCTT | 61 | 7807 | 7826 | 3-10-7 | 429 |
| 599469 | 2573 | 2592 | Exon 18 | CTGTCCAGCAGGAAACCCCT | 52 | 7808 | 7827 | 3-10-7 | 430 |
| 599470 | 2574 | 2593 | Exon 18 | CCTGTCCAGCAGGAAACCCC | 45 | 7809 | 7828 | 3-10-7 | 431 |
| 599471 | 2575 | 2594 | Exon 18 | CCCTGTCCAGCAGGAAACCC | 67 | 7810 | 7829 | 3-10-7 | 432 |
| 599472 | 2576 | 2595 | Exon 18 | CCCCTGTCCAGCAGGAAACC | 79 | 7811 | 7830 | 3-10-7 | 433 |
| 599473 | 2577 | 2596 | Exon 18 | GCCCCTGTCCAGCAGGAAAC | 72 | 7812 | 7831 | 3-10-7 | 238 |
| 599474 | 2578 | 2597 | Exon 18 | CGCCCCTGTCCAGCAGGAAA | 87 | 7813 | 7832 | 3-10-7 | 434 |
| 599475 | 2579 | 2598 | Exon 18 | ACGCCCCTGTCCAGCAGGAA | 76 | 7814 | 7833 | 3-10-7 | 435 |
| 599476 | 2580 | 2599 | Exon 18 | CACGCCCCTGTCCAGCAGGA | 81 | 7815 | 7834 | 3-10-7 | 436 |
| 599477 | 2581 | 2600 | Exon 18 | CCACGCCCCTGTCCAGCAGG | 83 | 7816 | 7835 | 3-10-7 | 437 |
| 599478 | 2582 | 2601 | Exon 18 | CCCACGCCCCTGTCCAGCAG | 72 | 7817 | 7836 | 3-10-7 | 438 |
| 599479 | 2583 | 2602 | Exon 18 | TCCCACGCCCCTGTCCAGCA | 81 | 7818 | 7837 | 3-10-7 | 439 |
| 599480 | 2584 | 2603 | Exon 18 | ATCCCACGCCCCTGTCCAGC | 77 | 7819 | 7838 | 3-10-7 | 440 |
| 599481 | 2585 | 2604 | Exon 18 | AATCCCACGCCCCTGTCCAG | 83 | 7820 | 7839 | 3-10-7 | 441 |
| 599482 | 2586 | 2605 | Exon 18 | CAATCCCACGCCCCTGTCCA | 87 | 7821 | 7840 | 3-10-7 | 442 |
| 599483 | 2587 | 2606 | Exon 18 | TCAATCCCACGCCCCTGTCC | 90 | 7822 | 7841 | 3-10-7 | 443 |
| 599484 | 2588 | 2607 | Exon 18 | TTCAATCCCACGCCCCTGTC | 72 | 7823 | 7842 | 3-10-7 | 444 |
| 599485 | 2589 | 2608 | Exon 18 | ATTCAATCCCACGCCCCTGT | 82 | 7824 | 7843 | 3-10-7 | 445 |
| 599486 | 2590 | 2609 | Exon 18 | AATTCAATCCCACGCCCCTG | 84 | 7825 | 7844 | 3-10-7 | 446 |
| 599487 | 2591 | 2610 | Exon 18 | TAATTCAATCCCACGCCCCT | 84 | 7826 | 7845 | 3-10-7 | 447 |
| 599488 | 2592 | 2611 | Exon 18 | TTAATTCAATCCCACGCCCC | 87 | 7827 | 7846 | 3-10-7 | 448 |
| 599489 | 2593 | 2612 | Exon 18 | TTTAATTCAATCCCACGCCC | 87 | 7828 | 7847 | 3-10-7 | 449 |
| 599490 | 2594 | 2613 | Exon 18 | TTTTAATTCAATCCCACGCC | 86 | 7829 | 7848 | 3-10-7 | 450 |
| 599491 | 2595 | 2614 | Exon 18 | GTTTTAATTCAATCCCACGC | 87 | 7830 | 7849 | 3-10-7 | 451 |
| 599492 | 2596 | 2615 | Exon 18 | TGTTTTAATTCAATCCCACG | 88 | 7831 | 7850 | 3-10-7 | 452 |
| 599493 | 2597 | 2616 | Exon 18 | CTGTTTTAATTCAATCCCAC | 75 | 7832 | 7851 | 3-10-7 | 453 |
| 599433 | 2597 | 2616 | Exon 18 | CTGTTTTAATTCAATCCCAC | 89 | 7832 | 7851 | 6-8-6 | 453 |
| 599494 | 2598 | 2617 | Exon 18 | GCTGTTTTAATTCAATCCCA | 90 | 7833 | 7852 | 3-10-7 | 454 |
| 599434 | 2598 | 2617 | Exon 18 | GCTGTTTTAATTCAATCCCA | 89 | 7833 | 7852 | 6-8-6 | 454 |
| 599495 | 2599 | 2618 | Exon 18 | AGCTGTTTTAATTCAATCCC | 88 | 7834 | 7853 | 3-10-7 | 239 |
| 599435 | 2599 | 2618 | Exon 18 | AGCTGTTTTAATTCAATCCC | 91 | 7834 | 7853 | 6-8-6 | 239 |
| 599496 | 2600 | 2619 | Exon 18 | CAGCTGTTTTAATTCAATCC | 89 | 7835 | 7854 | 3-10-7 | 455 |
| 599436 | 2600 | 2619 | Exon 18 | CAGCTGTTTTAATTCAATCC | 89 | 7835 | 7854 | 6-8-6 | 455 |
| 599497 | 2601 | 2620 | Exon 18 | GCAGCTGTTTTAATTCAATC | 89 | 7836 | 7855 | 3-10-7 | 456 |
| 599437 | 2601 | 2620 | Exon 18 | GCAGCTGTTTTAATTCAATC | 91 | 7836 | 7855 | 6-8-6 | 456 |
| 599498 | 2602 | 2621 | Exon 18 | CGCAGCTGTTTTAATTCAAT | 88 | 7837 | 7856 | 3-10-7 | 457 |
| 599438 | 2602 | 2621 | Exon 18 | CGCAGCTGTTTTAATTCAAT | 90 | 7837 | 7856 | 6-8-6 | 457 |
| 599499 | 2603 | 2622 | Exon 18 | TCGCAGCTGTTTTAATTCAA | 81 | 7838 | 7857 | 3-10-7 | 458 |
| 599439 | 2603 | 2622 | Exon 18 | TCGCAGCTGTTTTAATTCAA | 88 | 7838 | 7857 | 6-8-6 | 458 |
| 532917 | 2604 | 2623 | Exon 18 | GTCGCAGCTGTTTTAATTCA | 90 | 7839 | 7858 | 5-10-5 | 317 |
| 599500 | 2604 | 2623 | Exon 18 | GTCGCAGCTGTTTTAATTCA | 88 | 7839 | 7858 | 3-10-7 | 317 |
| 599440 | 2604 | 2623 | Exon 18 | GTCGCAGCTGTTTTAATTCA | 88 | 7839 | 7858 | 6-8-6 | 317 |
| 599501 | 2605 | 2624 | Exon 18 | TGTCGCAGCTGTTTTAATTC | 78 | 7840 | 7859 | 3-10-7 | 459 |
| 599441 | 2605 | 2624 | Exon 18 | TGTCGCAGCTGTTTTAATTC | 90 | 7840 | 7859 | 6-8-6 | 459 |
| 599502 | 2606 | 2625 | Exon 18 | TTGTCGCAGCTGTTTTAATT | 87 | 7841 | 7860 | 3-10-7 | 460 |
| 599442 | 2606 | 2625 | Exon 18 | TTGTCGCAGCTGTTTTAATT | 76 | 7841 | 7860 | 6-8-6 | 460 |
| 599503 | 2607 | 2626 | Exon 18 | GTTGTCGCAGCTGTTTTAAT | 83 | 7842 | 7861 | 3-10-7 | 461 |
| 599443 | 2607 | 2626 | Exon 18 | GTTGTCGCAGCTGTTTTAAT | 77 | 7842 | 7861 | 6-8-6 | 461 |
| 599504 | 2608 | 2627 | Exon 18 | TGTTGTCGCAGCTGTTTTAA | 89 | 7843 | 7862 | 3-10-7 | 395 |
| 599444 | 2608 | 2627 | Exon 18 | TGTTGTCGCAGCTGTTTTAA | 69 | 7843 | 7862 | 6-8-6 | 395 |
| 599505 | 2609 | 2628 | Exon 19/ | TTGTTGTCGCAGCTGTTTTA | 83 | n/a | n/a | 3-10-7 | 462 |
| | | | Repeat | | | | | | |
| 599445 | 2609 | 2628 | Exon 19/ | TTGTTGTCGCAGCTGTTTTA | 85 | n/a | n/a | 6-8-6 | 462 |
| | | | Repeat | | | | | | |
| 599506 | 2610 | 2629 | Exon 19/ | TTTGTTGTCGCAGCTGTTTT | 89 | n/a | n/a | 3-10-7 | 463 |
| | | | Repeat | | | | | | |
| 599446 | 2610 | 2629 | Exon 19/ | TTTGTTGTCGCAGCTGTTTT | 85 | n/a | n/a | 6-8-6 | 463 |
| | | | Repeat | | | | | | |
| 599507 | 2611 | 2630 | Exon 19/ | TTTTGTTGTCGCAGCTGTTT | 82 | n/a | n/a | 3-10-7 | 464 |
| | | | Repeat | | | | | | |
| 599447 | 2611 | 2630 | Exon 19/ | TTTTGTTGTCGCAGCTGTTT | 83 | n/a | n/a | 6-8-6 | 464 |
| | | | Repeat | | | | | | |
| 599508 | 2612 | 2631 | Exon 19/ | TTTTTGTTGTCGCAGCTGTT | 90 | n/a | n/a | 3-10-7 | 465 |
| | | | Repeat | | | | | | |
| 599448 | 2612 | 2631 | Exon 19/ | TTTTTGTTGTCGCAGCTGTT | 87 | n/a | n/a | 6-8-6 | 465 |
| | | | Repeat | | | | | | |

### Example 5: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 2,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 4-8-5 MOE, 5-8-5 MOE, 5-9-5 MOE, 5-10-5 MOE, 6-7-6-MOE, 3-10-5 MOE, or 6-8-6 MOE gapmers.

The 4-8-5 MOE gapmers are 17 nucleosides in length, wherein the central gap segment comprises of eight 2′-deoxynucleosides and is flanked by wing segments on the 5′ direction and the 3′ direction comprising four and five nucleosides respectively. The 5-8-5 MOE gapmers are 18 nucleosides in length, wherein the central gap segment comprises of eight 2′-deoxynucleosides and is flanked by wing segments on the 5′ direction and the 3′ direction comprising five nucleosides each. The 5-9-5 MOE gapmers are 19 nucleosides in length, wherein the central gap segment comprises of nine 2′-deoxynucleosides and is flanked by wing segments on the 5′ direction and the 3′ direction comprising five nucleosides each. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. The 3-10-5 MOE gapmers are 18 nucleosides in length, wherein the central gap segment comprises of ten 2′-deoxynucleosides and is flanked by wing segments on the 5′ direction and the 3′ direction comprising three and five nucleosides respectively. The 6-7-6 MOE gapmers are 19 nucleosides in length, wherein the central gap segment comprises of seven 2′-deoxynucleosides and is flanked by wing segments on the 5′ direction and the 3′ direction comprising six nucleosides each. The 6-8-6 MOE gapmers are 20 nucleosides in length, wherein the central gap segment comprises of eight 2′-deoxynucleosides and is flanked by wing segments on the 5′ direction and the 3′ direction comprising six nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 15
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 599160 | 2560 | 2577 | Exon 18 | CCCCTTATAGAAAACCCA | 26 | 7795 | 7812 | 5-8-5 | 740 |
| 599161 | 2561 | 2578 | Exon 18 | ACCCCTTATAGAAAACCC | 20 | 7796 | 7813 | 5-8-5 | 741 |
| 599162 | 2562 | 2579 | Exon 18 | AACCCCTTATAGAAAACC | 12 | 7797 | 7814 | 5-8-5 | 742 |
| 599163 | 2563 | 2580 | Exon 18 | AAACCCCTTATAGAAAAC | 11 | 7798 | 7815 | 5-8-5 | 743 |
| 599164 | 2564 | 2581 | Exon 18 | GAAACCCCTTATAGAAAA | 11 | 7799 | 7816 | 5-8-5 | 744 |
| 599165 | 2566 | 2583 | Exon 18 | AGGAAACCCCTTATAGAA | 0 | 7801 | 7818 | 5-8-5 | 745 |
| 599166 | 2567 | 2584 | Exon 18 | CAGGAAACCCCTTATAGA | 12 | 7802 | 7819 | 5-8-5 | 746 |
| 599167 | 2568 | 2585 | Exon 18 | GCAGGAAACCCCTTATAG | 14 | 7803 | 7820 | 5-8-5 | 747 |
| 599168 | 2569 | 2586 | Exon 18 | AGCAGGAAACCCCTTATA | 16 | 7804 | 7821 | 5-8-5 | 748 |
| 599169 | 2570 | 2587 | Exon 18 | CAGCAGGAAACCCCTTAT | 24 | 7805 | 7822 | 5-8-5 | 749 |
| 599170 | 2571 | 2588 | Exon 18 | CCAGCAGGAAACCCCTTA | 37 | 7806 | 7823 | 5-8-5 | 750 |
| 599171 | 2572 | 2589 | Exon 18 | TCCAGCAGGAAACCCCTT | 30 | 7807 | 7824 | 5-8-5 | 751 |
| 599172 | 2573 | 2590 | Exon 18 | GTCCAGCAGGAAACCCCT | 43 | 7808 | 7825 | 5-8-5 | 752 |
| 599173 | 2574 | 2591 | Exon 18 | TGTCCAGCAGGAAACCCC | 47 | 7809 | 7826 | 5-8-5 | 753 |
| 599174 | 2575 | 2592 | Exon 18 | CTGTCCAGCAGGAAACCC | 27 | 7810 | 7827 | 5-8-5 | 754 |
| 599175 | 2576 | 2593 | Exon 18 | CCTGTCCAGCAGGAAACC | 30 | 7811 | 7828 | 5-8-5 | 755 |
| 599176 | 2577 | 2594 | Exon 18 | CCCTGTCCAGCAGGAAAC | 34 | 7812 | 7829 | 5-8-5 | 756 |
| 599177 | 2578 | 2595 | Exon 18 | CCCCTGTCCAGCAGGAAA | 41 | 7813 | 7830 | 5-8-5 | 757 |
| 599178 | 2580 | 2597 | Exon 18 | CGCCCCTGTCCAGCAGGA | 67 | 7815 | 7832 | 5-8-5 | 758 |
| 599179 | 2581 | 2598 | Exon 18 | ACGCCCCTGTCCAGCAGG | 61 | 7816 | 7833 | 5-8-5 | 759 |
| 599180 | 2582 | 2599 | Exon 18 | CACGCCCCTGTCCAGCAG | 62 | 7817 | 7834 | 5-8-5 | 760 |
| 599181 | 2583 | 2600 | Exon 18 | CCACGCCCCTGTCCAGCA | 63 | 7818 | 7835 | 5-8-5 | 761 |
| 599128 | 2584 | 2600 | Exon 18 | CCACGCCCCTGTCCAGC | 55 | 7819 | 7835 | 4-8-5 | 649 |
| 599182 | 2584 | 2601 | Exon 18 | CCCACGCCCCTGTCCAGC | 58 | 7819 | 7836 | 5-8-5 | 762 |
| 599129 | 2585 | 2601 | Exon 18 | CCCACGCCCCTGTCCAG | 41 | 7820 | 7836 | 4-8-5 | 650 |
| 599183 | 2585 | 2602 | Exon 18 | TCCCACGCCCCTGTCCAG | 43 | 7820 | 7837 | 5-8-5 | 763 |
| 599130 | 2586 | 2602 | Exon 18 | TCCCACGCCCCTGTCCA | 46 | 7821 | 7837 | 4-8-5 | 651 |
| 599184 | 2586 | 2603 | Exon 18 | ATCCCACGCCCCTGTCCA | 32 | 7821 | 7838 | 5-8-5 | 764 |
| 599131 | 2587 | 2603 | Exon 18 | ATCCCACGCCCCTGTCC | 30 | 7822 | 7838 | 4-8-5 | 652 |
| 599185 | 2587 | 2604 | Exon 18 | AATCCCACGCCCCTGTCC | 35 | 7822 | 7839 | 5-8-5 | 765 |
| 599132 | 2588 | 2604 | Exon 18 | AATCCCACGCCCCTGTC | 52 | 7823 | 7839 | 4-8-5 | 653 |
| 599186 | 2588 | 2605 | Exon 18 | CAATCCCACGCCCCTGTC | 55 | 7823 | 7840 | 5-8-5 | 766 |
| 599133 | 2589 | 2605 | Exon 18 | CAATCCCACGCCCCTGT | 66 | 7824 | 7840 | 4-8-5 | 654 |
| 599187 | 2589 | 2606 | Exon 18 | TCAATCCCACGCCCCTGT | 72 | 7824 | 7841 | 5-8-5 | 767 |
| 599134 | 2590 | 2606 | Exon 18 | TCAATCCCACGCCCCTG | 80 | 7825 | 7841 | 4-8-5 | 655 |
| 599188 | 2590 | 2607 | Exon 18 | TTCAATCCCACGCCCCTG | 92 | 7825 | 7842 | 5-8-5 | 768 |
| 599135 | 2591 | 2607 | Exon 18 | TTCAATCCCACGCCCCT | 61 | 7826 | 7842 | 4-8-5 | 656 |
| 599189 | 2591 | 2608 | Exon 18 | ATTCAATCCCACGCCCCT | 52 | 7826 | 7843 | 5-8-5 | 769 |
| 599136 | 2592 | 2608 | Exon 18 | ATTCAATCCCACGCCCC | 68 | 7827 | 7843 | 4-8-5 | 657 |
| 599190 | 2592 | 2609 | Exon 18 | AATTCAATCCCACGCCCC | 62 | 7827 | 7844 | 5-8-5 | 770 |
| 599137 | 2593 | 2609 | Exon 18 | AATTCAATCCCACGCCC | 51 | 7828 | 7844 | 4-8-5 | 658 |
| 599191 | 2593 | 2610 | Exon 18 | TAATTCAATCCCACGCCC | 54 | 7828 | 7845 | 5-8-5 | 771 |
| 599138 | 2594 | 2610 | Exon 18 | TAATTCAATCCCACGCC | 71 | 7829 | 7845 | 4-8-5 | 659 |
| 599192 | 2594 | 2611 | Exon 18 | TTAATTCAATCCCACGCC | 66 | 7829 | 7846 | 5-8-5 | 772 |
| 599139 | 2595 | 2611 | Exon 18 | TTAATTCAATCCCACGC | 80 | 7830 | 7846 | 4-8-5 | 660 |
| 599193 | 2595 | 2612 | Exon 18 | TTTAATTCAATCCCACGC | 74 | 7830 | 7847 | 5-8-5 | 773 |
| 599140 | 2596 | 2612 | Exon 18 | TTTAATTCAATCCCACG | 66 | 7831 | 7847 | 4-8-5 | 786 |
| 599194 | 2596 | 2613 | Exon 18 | TTTTAATTCAATCCCACG | 66 | 7831 | 7848 | 5-8-5 | 774 |
| 599141 | 2597 | 2613 | Exon 18 | TTTTAATTCAATCCCAC | 63 | 7832 | 7848 | 4-8-5 | 662 |
| 599195 | 2597 | 2614 | Exon 18 | GTTTTAATTCAATCCCAC | 86 | 7832 | 7849 | 5-8-5 | 775 |
| 599142 | 2598 | 2614 | Exon 18 | GTTTTAATTCAATCCCA | 69 | 7833 | 7849 | 4-8-5 | 663 |
| 599196 | 2598 | 2615 | Exon 18 | TGTTTTAATTCAATCCCA | 82 | 7833 | 7850 | 5-8-5 | 776 |
| 599143 | 2599 | 2615 | Exon 18 | TGTTTTAATTCAATCCC | 59 | 7834 | 7850 | 4-8-5 | 664 |
| 599197 | 2599 | 2616 | Exon 18 | CTGTTTTAATTCAATCCC | 79 | 7834 | 7851 | 5-8-5 | 777 |
| 599144 | 2600 | 2616 | Exon 18 | CTGTTTTAATTCAATCC | 52 | 7835 | 7851 | 4-8-5 | 665 |
| 599198 | 2600 | 2617 | Exon 18 | GCTGTTTTAATTCAATCC | 86 | 7835 | 7852 | 5-8-5 | 778 |
| 599145 | 2601 | 2617 | Exon 18 | GCTGTTTTAATTCAATC | 53 | 7836 | 7852 | 4-8-5 | 666 |
| 599199 | 2601 | 2618 | Exon 18 | AGCTGTTTTAATTCAATC | 72 | 7836 | 7853 | 5-8-5 | 779 |
| 599146 | 2602 | 2618 | Exon 18 | AGCTGTTTTAATTCAAT | 42 | 7837 | 7853 | 4-8-5 | 667 |
| 599200 | 2602 | 2619 | Exon 18 | CAGCTGTTTTAATTCAAT | 76 | 7837 | 7854 | 5-8-5 | 780 |
| 599147 | 2603 | 2619 | Exon 18 | CAGCTGTTTTAATTCAA | 55 | 7838 | 7854 | 4-8-5 | 668 |
| 599201 | 2603 | 2620 | Exon 18 | GCAGCTGTTTTAATTCAA | 87 | 7838 | 7855 | 5-8-5 | 781 |
| 532917 | 2604 | 2623 | Exon 18 | GTCGCAGCTGTTTTAATTCA | 93 | 7839 | 7858 | 5-10-5 | 317 |
| 599148 | 2604 | 2620 | Exon 18 | GCAGCTGTTTTAATTCA | 84 | 7839 | 7855 | 4-8-5 | 669 |
| 599202 | 2604 | 2621 | Exon 18 | CGCAGCTGTTTTAATTCA | 89 | 7839 | 7856 | 5-8-5 | 782 |
| 599149 | 2605 | 2621 | Exon 18 | CGCAGCTGTTTTAATTC | 92 | 7840 | 7856 | 4-8-5 | 670 |
| 599203 | 2605 | 2622 | Exon 18 | TCGCAGCTGTTTTAATTC | 90 | 7840 | 7857 | 5-8-5 | 783 |
| 599150 | 2606 | 2622 | Exon 18 | TCGCAGCTGTTTTAATT | 75 | 7841 | 7857 | 4-8-5 | 671 |
| 599151 | 2607 | 2623 | Exon 18 | GTCGCAGCTGTTTTAAT | 80 | 7842 | 7858 | 4-8-5 | 672 |
| 599152 | 2608 | 2624 | Exon 18 | TGTCGCAGCTGTTTTAA | 76 | 7843 | 7859 | 4-8-5 | 673 |
| 599153 | 2609 | 2625 | Exon 18 | TTGTCGCAGCTGTTTTA | 56 | 7844 | 7860 | 4-8-5 | 674 |
| 599154 | 2610 | 2626 | Exon 18 | GTTGTCGCAGCTGTTTT | 85 | 7845 | 7861 | 4-8-5 | 675 |
| 599155 | 2611 | 2627 | Exon 18 | TGTTGTCGCAGCTGTTT | 89 | 7846 | 7862 | 4-8-5 | 676 |
| 599156 | 2612 | 2628 | Exon 18/ | TTGTTGTCGCAGCTGTT | 83 | n/a | n/a | 4-8-5 | 813 |
| | | | Repeat | | | | | | |
| 599157 | 2613 | 2629 | Exon 18/ | TTTGTTGTCGCAGCTGT | 78 | n/a | n/a | 4-8-5 | 678 |
| | | | Repeat | | | | | | |
| 599158 | 2614 | 2630 | Exon 18/ | TTTTGTTGTCGCAGCTG | 83 | n/a | n/a | 4-8-5 | 679 |
| | | | Repeat | | | | | | |
| 599159 | 2615 | 2631 | Exon 18/ | TTTTTGTTGTCGCAGCT | 65 | n/a | n/a | 4-8-5 | 680 |
| | | | Repeat | | | | | | |
| 599204 | 2606 | 2623 | Exon 18 | GTCGCAGCTGTTTTAATT | 83 | 7841 | 7858 | 5-8-5 | 784 |

TABLE 16
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 599509 | 2552 | 2570 | Exon 18 | TAGAAAACCCAAATCCTCA | 45 | 7787 | 7805 | 6-7-6 | 681 |
| 599213 | 2553 | 2570 | Exon 18 | TAGAAAACCCAAATCCTC | 89 | 7788 | 7805 | 3-10-5 | 785 |
| 599273 | 2553 | 2571 | Exon 18 | ATAGAAAACCCAAATCCTC | 85 | 7788 | 7806 | 6-7-6 | 682 |
| 599214 | 2554 | 2571 | Exon 18 | ATAGAAAACCCAAATCCT | 79 | 7789 | 7806 | 3-10-5 | 786 |
| 599274 | 2554 | 2572 | Exon 18 | TATAGAAAACCCAAATCCT | 75 | 7789 | 7807 | 6-7-6 | 683 |
| 599215 | 2555 | 2572 | Exon 18 | TATAGAAAACCCAAATCC | 81 | 7790 | 7807 | 3-10-5 | 787 |
| 599216 | 2556 | 2573 | Exon 18 | TTATAGAAAACCCAAATC | 87 | 7791 | 7808 | 3-10-5 | 788 |
| 599275 | 2556 | 2574 | Exon 18 | CTTATAGAAAACCCAAATC | 84 | 7791 | 7809 | 6-7-6 | 684 |
| 599217 | 2557 | 2574 | Exon 18 | CTTATAGAAAACCCAAAT | 84 | 7792 | 7809 | 3-10-5 | 789 |
| 599276 | 2557 | 2575 | Exon 18 | CCTTATAGAAAACCCAAAT | 68 | 7792 | 7810 | 6-7-6 | 685 |
| 599218 | 2558 | 2575 | Exon 18 | CCTTATAGAAAACCCAAA | 82 | 7793 | 7810 | 3-10-5 | 790 |
| 599277 | 2558 | 2576 | Exon 18 | CCCTTATAGAAAACCCAAA | 82 | 7793 | 7811 | 6-7-6 | 686 |
| 599219 | 2559 | 2576 | Exon 18 | CCCTTATAGAAAACCCAA | 81 | 7794 | 7811 | 3-10-5 | 791 |
| 599278 | 2559 | 2577 | Exon 18 | CCCCTTATAGAAAACCCAA | 84 | 7794 | 7812 | 6-7-6 | 687 |
| 599220 | 2560 | 2577 | Exon 18 | CCCCTTATAGAAAACCCA | 92 | 7795 | 7812 | 3-10-5 | 740 |
| 599279 | 2560 | 2578 | Exon 18 | ACCCCTTATAGAAAACCCA | 92 | 7795 | 7813 | 6-7-6 | 688 |
| 599221 | 2561 | 2578 | Exon 18 | ACCCCTTATAGAAAACCC | 93 | 7796 | 7813 | 3-10-5 | 741 |
| 599280 | 2561 | 2579 | Exon 18 | AACCCCTTATAGAAAACCC | 90 | 7796 | 7814 | 6-7-6 | 689 |
| 599222 | 2562 | 2579 | Exon 18 | AACCCCTTATAGAAAACC | 95 | 7797 | 7814 | 3-10-5 | 742 |
| 599223 | 2563 | 2580 | Exon 18 | AAACCCCTTATAGAAAAC | 93 | 7798 | 7815 | 3-10-5 | 743 |
| 599224 | 2564 | 2581 | Exon 18 | GAAACCCCTTATAGAAAA | 90 | 7799 | 7816 | 3-10-5 | 744 |
| 599225 | 2566 | 2583 | Exon 18 | AGGAAACCCCTTATAGAA | 93 | 7801 | 7818 | 3-10-5 | 745 |
| 599226 | 2567 | 2584 | Exon 18 | CAGGAAACCCCTTATAGA | 95 | 7802 | 7819 | 3-10-5 | 746 |
| 599227 | 2568 | 2585 | Exon 18 | GCAGGAAACCCCTTATAG | 94 | 7803 | 7820 | 3-10-5 | 747 |
| 599228 | 2569 | 2586 | Exon 18 | AGCAGGAAACCCCTTATA | 96 | 7804 | 7821 | 3-10-5 | 748 |
| 599229 | 2570 | 2587 | Exon 18 | CAGCAGGAAACCCCTTAT | 92 | 7805 | 7822 | 3-10-5 | 749 |
| 599230 | 2571 | 2588 | Exon 18 | CCAGCAGGAAACCCCTTA | 88 | 7806 | 7823 | 3-10-5 | 750 |
| 599231 | 2572 | 2589 | Exon 18 | TCCAGCAGGAAACCCCTT | 83 | 7807 | 7824 | 3-10-5 | 751 |
| 599232 | 2573 | 2590 | Exon 18 | GTCCAGCAGGAAACCCCT | 89 | 7808 | 7825 | 3-10-5 | 752 |
| 599233 | 2574 | 2591 | Exon 18 | TGTCCAGCAGGAAACCCC | 83 | 7809 | 7826 | 3-10-5 | 753 |
| 599234 | 2575 | 2592 | Exon 18 | CTGTCCAGCAGGAAACCC | 88 | 7810 | 7827 | 3-10-5 | 754 |
| 599235 | 2576 | 2593 | Exon 18 | CCTGTCCAGCAGGAAACC | 91 | 7811 | 7828 | 3-10-5 | 755 |
| 599236 | 2577 | 2594 | Exon 18 | CCCTGTCCAGCAGGAAAC | 90 | 7812 | 7829 | 3-10-5 | 756 |
| 599237 | 2578 | 2595 | Exon 18 | CCCCTGTCCAGCAGGAAA | 34 | 7813 | 7830 | 3-10-5 | 757 |
| 599238 | 2580 | 2597 | Exon 18 | CGCCCCTGTCCAGCAGGA | 14 | 7815 | 7832 | 3-10-5 | 758 |
| 599239 | 2581 | 2598 | Exon 18 | ACGCCCCTGTCCAGCAGG | 10 | 7816 | 7833 | 3-10-5 | 759 |
| 599240 | 2582 | 2599 | Exon 18 | CACGCCCCTGTCCAGCAG | 26 | 7817 | 7834 | 3-10-5 | 760 |
| 599241 | 2583 | 2600 | Exon 18 | CCACGCCCCTGTCCAGCA | 11 | 7818 | 7835 | 3-10-5 | 761 |
| 599242 | 2584 | 2601 | Exon 18 | CCCACGCCCCTGTCCAGC | 24 | 7819 | 7836 | 3-10-5 | 762 |
| 599243 | 2585 | 2602 | Exon 18 | TCCCACGCCCCTGTCCAG | 23 | 7820 | 7837 | 3-10-5 | 763 |
| 599244 | 2586 | 2603 | Exon 18 | ATCCCACGCCCCTGTCCA | 29 | 7821 | 7838 | 3-10-5 | 764 |
| 599245 | 2587 | 2604 | Exon 18 | AATCCCACGCCCCTGTCC | 11 | 7822 | 7839 | 3-10-5 | 765 |
| 599246 | 2588 | 2605 | Exon 18 | CAATCCCACGCCCCTGTC | 0 | 7823 | 7840 | 3-10-5 | 766 |
| 599247 | 2589 | 2606 | Exon 18 | TCAATCCCACGCCCCTGT | 21 | 7824 | 7841 | 3-10-5 | 767 |
| 599248 | 2590 | 2607 | Exon 18 | TTCAATCCCACGCCCCTG | 0 | 7825 | 7842 | 3-10-5 | 768 |
| 599249 | 2591 | 2608 | Exon 18 | ATTCAATCCCACGCCCCT | 9 | 7826 | 7843 | 3-10-5 | 769 |
| 599250 | 2592 | 2609 | Exon 18 | AATTCAATCCCACGCCCC | 4 | 7827 | 7844 | 3-10-5 | 770 |
| 599251 | 2593 | 2610 | Exon 18 | TAATTCAATCCCACGCCC | 12 | 7828 | 7845 | 3-10-5 | 771 |
| 599252 | 2594 | 2611 | Exon 18 | TTAATTCAATCCCACGCC | 2 | 7829 | 7846 | 3-10-5 | 772 |
| 599253 | 2595 | 2612 | Exon 18 | TTTAATTCAATCCCACGC | 28 | 7830 | 7847 | 3-10-5 | 773 |
| 599254 | 2596 | 2613 | Exon 18 | TTTTAATTCAATCCCACG | 27 | 7831 | 7848 | 3-10-5 | 774 |
| 599255 | 2597 | 2614 | Exon 18 | GTTTTAATTCAATCCCAC | 38 | 7832 | 7849 | 3-10-5 | 775 |
| 599256 | 2598 | 2615 | Exon 18 | TGTTTTAATTCAATCCCA | 36 | 7833 | 7850 | 3-10-5 | 776 |
| 599257 | 2599 | 2616 | Exon 18 | CTGTTTTAATTCAATCCC | 48 | 7834 | 7851 | 3-10-5 | 777 |
| 599258 | 2600 | 2617 | Exon 18 | GCTGTTTTAATTCAATCC | 19 | 7835 | 7852 | 3-10-5 | 778 |
| 599259 | 2601 | 2618 | Exon 18 | AGCTGTTTTAATTCAATC | 36 | 7836 | 7853 | 3-10-5 | 779 |
| 599260 | 2602 | 2619 | Exon 18 | CAGCTGTTTTAATTCAAT | 58 | 7837 | 7854 | 3-10-5 | 780 |
| 599261 | 2603 | 2620 | Exon 18 | GCAGCTGTTTTAATTCAA | 35 | 7838 | 7855 | 3-10-5 | 781 |
| 532917 | 2604 | 2623 | Exon 18 | GTCGCAGCTGTTTTAATTCA | 96 | 7839 | 7858 | 5-10-5 | 317 |
| 599262 | 2604 | 2621 | Exon 18 | CGCAGCTGTTTTAATTCA | 52 | 7839 | 7856 | 3-10-5 | 782 |
| 599263 | 2605 | 2622 | Exon 18 | TCGCAGCTGTTTTAATTC | 66 | 7840 | 7857 | 3-10-5 | 783 |
| 599264 | 2606 | 2623 | Exon 18 | GTCGCAGCTGTTTTAATT | 48 | 7841 | 7858 | 3-10-5 | 784 |
| 599265 | 2607 | 2624 | Exon 18 | TGTCGCAGCTGTTTTAAT | 46 | 7842 | 7859 | 3-10-5 | 792 |
| 599205 | 2607 | 2624 | Exon 18 | TGTCGCAGCTGTTTTAAT | 83 | 7842 | 7859 | 5-8-5 | 792 |
| 599266 | 2608 | 2625 | Exon 18 | TTGTCGCAGCTGTTTTAA | 76 | 7843 | 7860 | 3-10-5 | 793 |
| 599206 | 2608 | 2625 | Exon 18 | TTGTCGCAGCTGTTTTAA | 90 | 7843 | 7860 | 5-8-5 | 793 |
| 599267 | 2609 | 2626 | Exon 18 | GTTGTCGCAGCTGTTTTA | 53 | 7844 | 7861 | 3-10-5 | 794 |
| 599207 | 2609 | 2626 | Exon 18 | GTTGTCGCAGCTGTTTTA | 82 | 7844 | 7861 | 5-8-5 | 794 |
| 599268 | 2610 | 2627 | Exon 18 | TGTTGTCGCAGCTGTTTT | 58 | 7845 | 7862 | 3-10-5 | 795 |
| 599208 | 2610 | 2627 | Exon 18 | TGTTGTCGCAGCTGTTTT | 70 | 7845 | 7862 | 5-8-5 | 795 |
| 599269 | 2611 | 2628 | Exon 18/ | TTGTTGTCGCAGCTGTTT | 38 | n/a | n/a | 3-10-5 | 796 |
| | | | Repeat | | | | | | |
| 599209 | 2611 | 2628 | Exon 18/ | TTGTTGTCGCAGCTGTTT | 50 | n/a | n/a | 5-8-5 | 796 |
| | | | Repeat | | | | | | |
| 599270 | 2612 | 2629 | Exon 18/ | TTTGTTGTCGCAGCTGTT | 46 | n/a | n/a | 3-10-5 | 797 |
| | | | Repeat | | | | | | |
| 599210 | 2612 | 2629 | Exon 18/ | TTTGTTGTCGCAGCTGTT | 76 | n/a | n/a | 5-8-5 | 797 |
| | | | Repeat | | | | | | |
| 599271 | 2613 | 2630 | Exon 18/ | TTTTGTTGTCGCAGCTGT | 64 | n/a | n/a | 3-10-5 | 798 |
| | | | Repeat | | | | | | |
| 599211 | 2613 | 2630 | Exon 18/ | TTTTGTTGTCGCAGCTGT | 78 | n/a | n/a | 5-8-5 | 798 |
| | | | Repeat | | | | | | |
| 599272 | 2614 | 2631 | Exon 18/ | TTTTTGTTGTCGCAGCTG | 89 | n/a | n/a | 3-10-5 | 799 |
| | | | Repeat | | | | | | |
| 599212 | 2614 | 2631 | Exon 18/ | TTTTTGTTGTCGCAGCTG | 84 | n/a | n/a | 5-8-5 | 799 |
| | | | Repeat | | | | | | |

TABLE 17
Inhibition of CFB mRNA by MOE gapmers targeting SEQ ID NO: 1 or SEQ ID NO: 2

| | SEQ ID | SEQ ID | | | | SEQ ID | SEQ ID | | |
|---|---|---|---|---|---|---|---|---|---|
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 599511 | 2552 | 2571 | Exon 18 | ATAGAAAACCCAAATCCTCA | 38 | 7787 | 7806 | 6-8-6 | 410 |
| 599389 | 2553 | 2572 | Exon 18 | TATAGAAAACCCAAATCCTC | 80 | 7788 | 7807 | 6-8-6 | 411 |
| 599390 | 2554 | 2573 | Exon 18 | TTATAGAAAACCCAAATCCT | 92 | 7789 | 7808 | 6-8-6 | 412 |
| 599391 | 2555 | 2574 | Exon 18 | CTTATAGAAAACCCAAATCC | 90 | 7790 | 7809 | 6-8-6 | 413 |
| 599392 | 2556 | 2575 | Exon 18 | CCTTATAGAAAACCCAAATC | 87 | 7791 | 7810 | 6-8-6 | 414 |
| 599393 | 2557 | 2576 | Exon 18 | CCCTTATAGAAAACCCAAAT | 87 | 7792 | 7811 | 6-8-6 | 415 |
| 599394 | 2558 | 2577 | Exon 18 | CCCCTTATAGAAAACCCAAA | 74 | 7793 | 7812 | 6-8-6 | 416 |
| 599395 | 2559 | 2578 | Exon 18 | ACCCCTTATAGAAAACCCAA | 78 | 7794 | 7813 | 6-8-6 | 417 |
| 599396 | 2560 | 2579 | Exon 18 | AACCCCTTATAGAAAACCCA | 77 | 7795 | 7814 | 6-8-6 | 418 |
| 599397 | 2561 | 2580 | Exon 18 | AAACCCCTTATAGAAAACCC | 89 | 7796 | 7815 | 6-8-6 | 419 |
| 599398 | 2562 | 2581 | Exon 18 | GAAACCCCTTATAGAAAACC | 90 | 7797 | 7816 | 6-8-6 | 420 |
| 599399 | 2563 | 2582 | Exon 18 | GGAAACCCCTTATAGAAAAC | 91 | 7798 | 7817 | 6-8-6 | 421 |
| 599400 | 2564 | 2583 | Exon 18 | AGGAAACCCCTTATAGAAAA | 88 | 7799 | 7818 | 6-8-6 | 422 |
| 599401 | 2565 | 2584 | Exon 18 | CAGGAAACCCCTTATAGAAA | 85 | 7800 | 7819 | 6-8-6 | 423 |
| 599402 | 2566 | 2585 | Exon 18 | GCAGGAAACCCCTTATAGAA | 77 | 7801 | 7820 | 6-8-6 | 424 |
| 599403 | 2567 | 2586 | Exon 18 | AGCAGGAAACCCCTTATAGA | 85 | 7802 | 7821 | 6-8-6 | 425 |
| 599404 | 2568 | 2587 | Exon 18 | CAGCAGGAAACCCCTTATAG | 90 | 7803 | 7822 | 6-8-6 | 426 |
| 599405 | 2569 | 2588 | Exon 18 | CCAGCAGGAAACCCCTTATA | 89 | 7804 | 7823 | 6-8-6 | 427 |
| 599406 | 2570 | 2589 | Exon 18 | TCCAGCAGGAAACCCCTTAT | 72 | 7805 | 7824 | 6-8-6 | 428 |
| 599407 | 2571 | 2590 | Exon 18 | GTCCAGCAGGAAACCCCTTA | 87 | 7806 | 7825 | 6-8-6 | 237 |
| 599408 | 2572 | 2591 | Exon 18 | TGTCCAGCAGGAAACCCCTT | 87 | 7807 | 7826 | 6-8-6 | 429 |
| 599409 | 2573 | 2592 | Exon 18 | CTGTCCAGCAGGAAACCCCT | 83 | 7808 | 7827 | 6-8-6 | 430 |
| 599410 | 2574 | 2593 | Exon 18 | CCTGTCCAGCAGGAAACCCC | 88 | 7809 | 7828 | 6-8-6 | 431 |
| 599411 | 2575 | 2594 | Exon 18 | CCCTGTCCAGCAGGAAACCC | 45 | 7810 | 7829 | 6-8-6 | 432 |
| 599412 | 2576 | 2595 | Exon 18 | CCCCTGTCCAGCAGGAAACC | 66 | 7811 | 7830 | 6-8-6 | 433 |
| 599413 | 2577 | 2596 | Exon 18 | GCCCCTGTCCAGCAGGAAAC | 92 | 7812 | 7831 | 6-8-6 | 238 |
| 599414 | 2578 | 2597 | Exon 18 | CGCCCCTGTCCAGCAGGAAA | 92 | 7813 | 7832 | 6-8-6 | 434 |
| 599415 | 2579 | 2598 | Exon 18 | ACGCCCCTGTCCAGCAGGAA | 87 | 7814 | 7833 | 6-8-6 | 435 |
| 599416 | 2580 | 2599 | Exon 18 | CACGCCCCTGTCCAGCAGGA | 91 | 7815 | 7834 | 6-8-6 | 436 |
| 599417 | 2581 | 2600 | Exon 18 | CCACGCCCCTGTCCAGCAGG | 84 | 7816 | 7835 | 6-8-6 | 437 |
| 599357 | 2582 | 2600 | Exon 18 | CCACGCCCCTGTCCAGCAG | 88 | 7817 | 7835 | 5-9-5 | 708 |
| 599418 | 2582 | 2601 | Exon 18 | CCCACGCCCCTGTCCAGCAG | 85 | 7817 | 7836 | 6-8-6 | 438 |
| 599358 | 2583 | 2601 | Exon 18 | CCCACGCCCCTGTCCAGCA | 86 | 7818 | 7836 | 5-9-5 | 709 |
| 599419 | 2583 | 2602 | Exon 18 | TCCCACGCCCCTGTCCAGCA | 91 | 7818 | 7837 | 6-8-6 | 833 |
| 599359 | 2584 | 2602 | Exon 18 | TCCCACGCCCCTGTCCAGC | 85 | 7819 | 7837 | 5-9-5 | 834 |
| 599420 | 2584 | 2603 | Exon 18 | ATCCCACGCCCCTGTCCAGC | 91 | 7819 | 7838 | 6-8-6 | 440 |
| 599360 | 2585 | 2603 | Exon 18 | ATCCCACGCCCCTGTCCAG | 89 | 7820 | 7838 | 5-9-5 | 711 |
| 599421 | 2585 | 2604 | Exon 18 | AATCCCACGCCCCTGTCCAG | 87 | 7820 | 7839 | 6-8-6 | 441 |
| 599361 | 2586 | 2604 | Exon 18 | AATCCCACGCCCCTGTCCA | 89 | 7821 | 7839 | 5-9-5 | 712 |
| 599422 | 2586 | 2605 | Exon 18 | CAATCCCACGCCCCTGTCCA | 90 | 7821 | 7840 | 6-8-6 | 442 |
| 599362 | 2587 | 2605 | Exon 18 | CAATCCCACGCCCCTGTCC | 94 | 7822 | 7840 | 5-9-5 | 713 |
| 599423 | 2587 | 2606 | Exon 18 | TCAATCCCACGCCCCTGTCC | 85 | 7822 | 7841 | 6-8-6 | 841 |
| 599363 | 2588 | 2606 | Exon 18 | TCAATCCCACGCCCCTGTC | 88 | 7823 | 7841 | 5-9-5 | 714 |
| 599424 | 2588 | 2607 | Exon 18 | TTCAATCCCACGCCCCTGTC | 88 | 7823 | 7842 | 6-8-6 | 444 |
| 599364 | 2589 | 2607 | Exon 18 | TTCAATCCCACGCCCCTGT | 88 | 7824 | 7842 | 5-9-5 | 715 |
| 599425 | 2589 | 2608 | Exon 18 | ATTCAATCCCACGCCCCTGT | 68 | 7824 | 7843 | 6-8-6 | 445 |
| 599365 | 2590 | 2608 | Exon 18 | ATTCAATCCCACGCCCCTG | 48 | 7825 | 7843 | 5-9-5 | 716 |
| 599426 | 2590 | 2609 | Exon 18 | AATTCAATCCCACGCCCCTG | 55 | 7825 | 7844 | 6-8-6 | 446 |
| 599366 | 2591 | 2609 | Exon 18 | AATTCAATCCCACGCCCCT | 28 | 7826 | 7844 | 5-9-5 | 717 |
| 599427 | 2591 | 2610 | Exon 18 | TAATTCAATCCCACGCCCCT | 13 | 7826 | 7845 | 6-8-6 | 849 |
| 599367 | 2592 | 2610 | Exon 18 | TAATTCAATCCCACGCCCC | 21 | 7827 | 7845 | 5-9-5 | 718 |
| 599428 | 2592 | 2611 | Exon 18 | TTAATTCAATCCCACGCCCC | 39 | 7827 | 7846 | 6-8-6 | 448 |
| 599368 | 2593 | 2611 | Exon 18 | TTAATTCAATCCCACGCCC | 20 | 7828 | 7846 | 5-9-5 | 719 |
| 599429 | 2593 | 2612 | Exon 18 | TTTAATTCAATCCCACGCCC | 18 | 7828 | 7847 | 6-8-6 | 449 |
| 599369 | 2594 | 2612 | Exon 18 | TTTAATTCAATCCCACGCC | 78 | 7829 | 7847 | 5-9-5 | 720 |
| 599430 | 2594 | 2613 | Exon 18 | TTTTAATTCAATCCCACGCC | 24 | 7829 | 7848 | 6-8-6 | 450 |
| 599370 | 2595 | 2613 | Exon 18 | TTTTAATTCAATCCCACGC | 25 | 7830 | 7848 | 5-9-5 | 721 |
| 599431 | 2595 | 2614 | Exon 18 | GTTTTAATTCAATCCCACGC | 30 | 7830 | 7849 | 6-8-6 | 451 |
| 599371 | 2596 | 2614 | Exon 18 | GTTTTAATTCAATCCCACG | 84 | 7831 | 7849 | 5-9-5 | 722 |
| 599432 | 2596 | 2615 | Exon 18 | TGTTTTAATTCAATCCCACG | 29 | 7831 | 7850 | 6-8-6 | 452 |
| 599372 | 2597 | 2615 | Exon 18 | TGTTTTAATTCAATCCCAC | 83 | 7832 | 7850 | 5-9-5 | 723 |
| 599373 | 2598 | 2616 | Exon 18 | CTGTTTTAATTCAATCCCA | 81 | 7833 | 7851 | 5-9-5 | 724 |
| 599374 | 2599 | 2617 | Exon 18 | GCTGTTTTAATTCAATCCC | 26 | 7834 | 7852 | 5-9-5 | 725 |
| 599375 | 2600 | 2618 | Exon 18 | AGCTGTTTTAATTCAATCC | 26 | 7835 | 7853 | 5-9-5 | 726 |
| 599376 | 2601 | 2619 | Exon 18 | CAGCTGTTTTAATTCAATC | 62 | 7836 | 7854 | 5-9-5 | 727 |
| 599377 | 2602 | 2620 | Exon 18 | GCAGCTGTTTTAATTCAAT | 21 | 7837 | 7855 | 5-9-5 | 728 |
| 599378 | 2603 | 2621 | Exon 18 | CGCAGCTGTTTTAATTCAA | 90 | 7838 | 7856 | 5-9-5 | 729 |
| 532917 | 2604 | 2623 | Exon 18 | GTCGCAGCTGTTTTAATTCA | 95 | 7839 | 7858 | 5-10-5 | 867 |
| 599379 | 2604 | 2622 | Exon 18 | TCGCAGCTGTTTTAATTCA | 88 | 7839 | 7857 | 5-9-5 | 730 |
| 599380 | 2605 | 2623 | Exon 18 | GTCGCAGCTGTTTTAATTC | 37 | 7840 | 7858 | 5-9-5 | 869 |
| 599381 | 2606 | 2624 | Exon 18 | TGTCGCAGCTGTTTTAATT | 33 | 7841 | 7859 | 5-9-5 | 732 |
| 599382 | 2607 | 2625 | Exon 18 | TTGTCGCAGCTGTTTTAAT | 81 | 7842 | 7860 | 5-9-5 | 733 |
| 599383 | 2608 | 2626 | Exon 18 | GTTGTCGCAGCTGTTTTAA | 54 | 7843 | 7861 | 5-9-5 | 734 |
| 599384 | 2609 | 2627 | Exon 18 | TGTTGTCGCAGCTGTTTTA | 85 | 7844 | 7862 | 5-9-5 | 873 |
| 599385 | 2610 | 2628 | Exon 18/ | TTGTTGTCGCAGCTGTTTT | 59 | n/a | n/a | 5-9-5 | 736 |
| | | | Repeat | | | | | | |
| 599386 | 2611 | 2629 | Exon 18/ | TTTGTTGTCGCAGCTGTTT | 81 | n/a | n/a | 5-9-5 | 737 |
| | | | Repeat | | | | | | |
| 599387 | 2612 | 2630 | Exon 18/ | TTTTGTTGTCGCAGCTGTT | 80 | n/a | n/a | 5-9-5 | 738 |
| | | | Repeat | | | | | | |
| 599388 | 2613 | 2631 | Exon 18/ | TTTTTGTTGTCGCAGCTGT | 84 | n/a | n/a | 5-9-5 | 739 |
| | | | Repeat | | | | | | |

### Example 6: Antisense Inhibition of Human Complement Factor B (CFB) in HepG2 Cells

Additional antisense oligonucleotides were designed targeting human Complement Factor B (CFB) nucleic acid and were tested for their effects on CFB mRNA in vitro. Cultured HepG2 cells at a density of 20,000 cells per well were transfected using electroporation with 1,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as deoxy, MOE and cEt oligonucleotides. The deoxy, MOE and cEt oligonucleotides are 16 nucleosides in length wherein the nucleoside have either a MOE sugar modification, an cEt sugar modification, or a deoxy modification. The ‘Chemistry’ column describes the sugar modifications of each oligonucleotide. ‘k’ indicates an cEt sugar modification; ‘d’ indicates deoxyribose; and ‘e’ indicates a MOE modification.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 18
Inhibition of CFB mRNA by deoxy, MOE and cEt oligonucleotides targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | | NO: 2 | NO: 2 | | SEQ |
| ISIS | start | stop | Target | | % | start | stop | | ID |
| NO | site | site | region | Sequence | inhibition | site | site | Motif | NO: |
| 599513 | 2551 | 2566 | Exon 18 | AAACCCAAATCCTCAT | 11 | 7786 | 7801 | ekkeekkdddddddkk | 557 |
| 599514 | 2553 | 2568 | Exon 18 | GAAAACCCAAATCCTC | 13 | 7788 | 7803 | ekkeekkdddddddkk | 801 |
| 599515 | 2555 | 2570 | Exon 18 | TAGAAAACCCAAATCC | 54 | 7790 | 7805 | ekkeekkdddddddkk | 559 |
| 599516 | 2559 | 2574 | Exon 18 | CTTATAGAAAACCCAA | 16 | 7794 | 7809 | ekkeekkdddddddkk | 561 |
| 599517 | 2560 | 2575 | Exon 18 | CCTTATAGAAAACCCA | 29 | 7795 | 7810 | ekkeekkdddddddkk | 562 |
| 599518 | 2561 | 2576 | Exon 18 | CCCTTATAGAAAACCC | 55 | 7796 | 7811 | ekkeekkdddddddkk | 563 |
| 599519 | 2562 | 2577 | Exon 18 | CCCCTTATAGAAAACC | 31 | 7797 | 7812 | ekkeekkdddddddkk | 564 |
| 599520 | 2563 | 2578 | Exon 18 | ACCCCTTATAGAAAAC | 14 | 7798 | 7813 | ekkeekkdddddddkk | 565 |
| 599521 | 2564 | 2579 | Exon 18 | AACCCCTTATAGAAAA | 9 | 7799 | 7814 | ekkeekkdddddddkk | 566 |
| 599522 | 2565 | 2580 | Exon 18 | AAACCCCTTATAGAAA | 8 | 7800 | 7815 | ekkeekkdddddddkk | 567 |
| 599523 | 2566 | 2581 | Exon 18 | GAAACCCCTTATAGAA | 6 | 7801 | 7816 | ekkeekkdddddddkk | 568 |
| 599524 | 2567 | 2582 | Exon 18 | GGAAACCCCTTATAGA | 14 | 7802 | 7817 | ekkeekkdddddddkk | 569 |
| 599525 | 2568 | 2583 | Exon 18 | AGGAAACCCCTTATAG | 6 | 7803 | 7818 | ekkeekkdddddddkk | 570 |
| 599526 | 2569 | 2584 | Exon 18 | CAGGAAACCCCTTATA | 16 | 7804 | 7819 | ekkeekkdddddddkk | 571 |
| 599527 | 2570 | 2585 | Exon 18 | GCAGGAAACCCCTTAT | 0 | 7805 | 7820 | ekkeekkdddddddkk | 572 |
| 599528 | 2571 | 2586 | Exon 18 | AGCAGGAAACCCCTTA | 6 | 7806 | 7821 | ekkeekkdddddddkk | 573 |
| 599529 | 2572 | 2587 | Exon 18 | CAGCAGGAAACCCCTT | 6 | 7807 | 7822 | ekkeekkdddddddkk | 574 |
| 599530 | 2574 | 2589 | Exon 18 | TCCAGCAGGAAACCCC | 29 | 7809 | 7824 | ekkeekkdddddddkk | 576 |
| 599531 | 2575 | 2590 | Exon 18 | GTCCAGCAGGAAACCC | 64 | 7810 | 7825 | ekkeekkdddddddkk | 577 |
| 599532 | 2576 | 2591 | Exon 18 | TGTCCAGCAGGAAACC | 43 | 7811 | 7826 | ekkeekkdddddddkk | 578 |
| 599533 | 2577 | 2592 | Exon 18 | CTGTCCAGCAGGAAAC | 25 | 7812 | 7827 | ekkeekkdddddddkk | 820 |
| 599534 | 2578 | 2593 | Exon 18 | CCTGTCCAGCAGGAAA | 12 | 7813 | 7828 | ekkeekkdddddddkk | 580 |
| 599535 | 2580 | 2595 | Exon 18 | CCCCTGTCCAGCAGGA | 16 | 7815 | 7830 | ekkeekkdddddddkk | 582 |
| 599536 | 2582 | 2597 | Exon 18 | CGCCCCTGTCCAGCAG | 27 | 7817 | 7832 | ekkeekkdddddddkk | 584 |
| 599537 | 2583 | 2598 | Exon 18 | ACGCCCCTGTCCAGCA | 35 | 7818 | 7833 | ekkeekkdddddddkk | 585 |
| 599538 | 2584 | 2599 | Exon 18 | CACGCCCCTGTCCAGC | 26 | 7819 | 7834 | ekkeekkdddddddkk | 586 |
| 599539 | 2585 | 2600 | Exon 18 | CCACGCCCCTGTCCAG | 33 | 7820 | 7835 | ekkeekkdddddddkk | 587 |
| 599540 | 2586 | 2601 | Exon 18 | CCCACGCCCCTGTCCA | 27 | 7821 | 7836 | ekkeekkdddddddkk | 588 |
| 599541 | 2587 | 2602 | Exon 18 | TCCCACGCCCCTGTCC | 52 | 7822 | 7837 | ekkeekkdddddddkk | 589 |
| 599542 | 2588 | 2603 | Exon 18 | ATCCCACGCCCCTGTC | 16 | 7823 | 7838 | ekkeekkdddddddkk | 590 |
| 599543 | 2589 | 2604 | Exon 18 | AATCCCACGCCCCTGT | 19 | 7824 | 7839 | ekkeekkdddddddkk | 591 |
| 599544 | 2590 | 2605 | Exon 18 | CAATCCCACGCCCCTG | 33 | 7825 | 7840 | ekkeekkdddddddkk | 831 |
| 599545 | 2591 | 2606 | Exon 18 | TCAATCCCACGCCCCT | 24 | 7826 | 7841 | ekkeekkdddddddkk | 593 |
| 599546 | 2592 | 2607 | Exon 18 | TTCAATCCCACGCCCC | 54 | 7827 | 7842 | ekkeekkdddddddkk | 594 |
| 599547 | 2593 | 2608 | Exon 18 | ATTCAATCCCACGCCC | 87 | 7828 | 7843 | ekkeekkdddddddkk | 595 |
| 599548 | 2594 | 2609 | Exon 18 | AATTCAATCCCACGCC | 79 | 7829 | 7844 | ekkeekkdddddddkk | 596 |
| 599549 | 2595 | 2610 | Exon 18 | TAATTCAATCCCACGC | 62 | 7830 | 7845 | ekkeekkdddddddkk | 597 |
| 599550 | 2596 | 2611 | Exon 18 | TTAATTCAATCCCACG | 52 | 7831 | 7846 | ekkeekkdddddddkk | 598 |
| 599551 | 2597 | 2612 | Exon 18 | TTTAATTCAATCCCAC | 27 | 7832 | 7847 | ekkeekkdddddddkk | 599 |
| 599577 | 2597 | 2613 | Exon 18 | TTTTAATTCAATCCCAC | 90 | 7832 | 7848 | eeekkdddddddkkeee | 662 |
| 599552 | 2598 | 2613 | Exon 18 | TTTTAATTCAATCCCA | 92 | 7833 | 7848 | ekkeekkdddddddkk | 600 |
| 599578 | 2598 | 2614 | Exon 18 | GTTTTAATTCAATCCCA | 88 | 7833 | 7849 | eeekkdddddddkkeee | 663 |
| 599553 | 2599 | 2614 | Exon 18 | GTTTTAATTCAATCCC | 91 | 7834 | 7849 | ekkeekkdddddddkk | 601 |
| 599579 | 2599 | 2615 | Exon 18 | TGTTTTAATTCAATCCC | 79 | 7834 | 7850 | eeekkdddddddkkeee | 664 |
| 599554 | 2600 | 2615 | Exon 18 | TGTTTTAATTCAATCC | 90 | 7835 | 7850 | ekkeekkdddddddkk | 602 |
| 599580 | 2600 | 2616 | Exon 18 | CTGTTTTAATTCAATCC | 79 | 7835 | 7851 | eeekkdddddddkkeee | 665 |
| 599555 | 2601 | 2616 | Exon 18 | CTGTTTTAATTCAATC | 79 | 7836 | 7851 | ekkeekkdddddddkk | 846 |
| 599581 | 2601 | 2617 | Exon 18 | GCTGTTTTAATTCAATC | 90 | 7836 | 7852 | eeekkdddddddkkeee | 666 |
| 599556 | 2602 | 2617 | Exon 18 | GCTGTTTTAATTCAAT | 47 | 7837 | 7852 | ekkeekkdddddddkk | 604 |
| 599582 | 2602 | 2618 | Exon 18 | AGCTGTTTTAATTCAAT | 89 | 7837 | 7853 | eeekkdddddddkkeee | 849 |
| 599557 | 2603 | 2618 | Exon 18 | AGCTGTTTTAATTCAA | 67 | 7838 | 7853 | ekkeekkdddddddkk | 850 |
| 599583 | 2603 | 2619 | Exon 18 | CAGCTGTTTTAATTCAA | 49 | 7838 | 7854 | eeekkdddddddkkeee | 668 |
| 532917 | 2604 | 2623 | Exon 18 | GTCGCAGCTGTTTTAATTCA | 78 | 7839 | 7858 | eeeeeddddddddddeee | 317 |
| | | | | | | | | ee | |
| 599558 | 2604 | 2619 | Exon 18 | CAGCTGTTTTAATTCA | 80 | 7839 | 7854 | ekkeekkdddddddkk | 606 |
| 599584 | 2604 | 2620 | Exon 18 | GCAGCTGTTTTAATTCA | 66 | 7839 | 7855 | eeekkdddddddkkeee | 669 |
| 599559 | 2605 | 2620 | Exon 18 | GCAGCTGTTTTAATTC | 38 | 7840 | 7855 | ekkeekkdddddddkk | 607 |
| 599585 | 2605 | 2621 | Exon 18 | CGCAGCTGTTTTAATTC | 80 | 7840 | 7856 | eeekkdddddddkkeee | 670 |
| 599560 | 2606 | 2621 | Exon 18 | CGCAGCTGTTTTAATT | 16 | 7841 | 7856 | ekkeekkdddddddkk | 608 |
| 599586 | 2606 | 2622 | Exon 18 | TCGCAGCTGTTTTAATT | 78 | 7841 | 7857 | eeekkdddddddkkeee | 671 |
| 599561 | 2607 | 2622 | Exon 18 | TCGCAGCTGTTTTAAT | 58 | 7842 | 7857 | ekkeekkdddddddkk | 609 |
| 599587 | 2607 | 2623 | Exon 18 | GTCGCAGCTGTTTTAAT | 81 | 7842 | 7858 | eeekkdddddddkkeee | 672 |
| 588860 | 2608 | 2623 | Exon 18 | GTCGCAGCTGTTTTAA | 92 | 7843 | 7858 | eekddddddddddkke | 610 |
| 599562 | 2608 | 2623 | Exon 18 | GTCGCAGCTGTTTTAA | 78 | 7843 | 7858 | ekkeekkdddddddkk | 610 |
| 599588 | 2608 | 2624 | Exon 18 | TGTCGCAGCTGTTTTAA | 81 | 7843 | 7859 | eeekkdddddddkkeee | 673 |
| 599563 | 2609 | 2624 | Exon 18 | TGTCGCAGCTGTTTTA | 86 | 7844 | 7859 | ekkeekkdddddddkk | 611 |
| 599589 | 2609 | 2625 | Exon 18 | TTGTCGCAGCTGTTTTA | 75 | 7844 | 7860 | eeekkdddddddkkeee | 674 |
| 599564 | 2610 | 2625 | Exon 18 | TTGTCGCAGCTGTTTT | 75 | 7845 | 7860 | ekkeekkdddddddkk | 612 |
| 599590 | 2610 | 2626 | Exon 18 | GTTGTCGCAGCTGTTTT | 88 | 7845 | 7861 | eeekkdddddddkkeee | 675 |
| 599565 | 2611 | 2626 | Exon 18 | GTTGTCGCAGCTGTTT | 65 | 7846 | 7861 | ekkeekkdddddddkk | 613 |
| 599591 | 2611 | 2627 | Exon 18 | TGTTGTCGCAGCTGTTT | 94 | 7846 | 7862 | eeekkdddddddkkeee | 676 |
| 599566 | 2612 | 2627 | Exon 18 | TGTTGTCGCAGCTGTT | 72 | 7847 | 7862 | ekkeekkdddddddkk | 614 |
| 599592 | 2612 | 2628 | Exon 18/ | TTGTTGTCGCAGCTGTT | 90 | n/a | n/a | eeekkdddddddkkeee | 677 |
| | | | Repeat | | | | | | |
| 599567 | 2613 | 2628 | Exon 18/ | TTGTTGTCGCAGCTGT | 82 | n/a | n/a | ekkeekkdddddddkk | 615 |
| | | | Repeat | | | | | | |
| 599593 | 2613 | 2629 | Exon 18/ | TTTGTTGTCGCAGCTGT | 95 | n/a | n/a | eeekkdddddddkkeee | 678 |
| | | | Repeat | | | | | | |
| 599568 | 2614 | 2629 | Exon 18/ | TTTGTTGTCGCAGCTG | 92 | n/a | n/a | ekkeekkdddddddkk | 616 |
| | | | Repeat | | | | | | |
| 599594 | 2614 | 2630 | Exon 18/ | TTTTGTTGTCGCAGCTG | 86 | n/a | n/a | eeekkdddddddkkeee | 679 |
| | | | Repeat | | | | | | |
| 599569 | 2615 | 2630 | Exon 18/ | TTTTGTTGTCGCAGCT | 89 | n/a | n/a | ekkeekkdddddddkk | 617 |
| | | | Repeat | | | | | | |
| 599595 | 2615 | 2631 | Exon 18/ | TTTTTGTTGTCGCAGCT | 76 | n/a | n/a | eeekkdddddddkkeee | 680 |
| | | | Repeat | | | | | | |
| 599570 | 2616 | 2631 | Exon 18/ | TTTTTGTTGTCGCAGC | 95 | n/a | n/a | ekkeekkdddddddkk | 618 |
| | | | Repeat | | | | | | |

### Example 7: Antisense Inhibition of Human Complement Factor B (CFB) in HepG2 Cells

Additional antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 500 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as deoxy, MOE and cEt oligonucleotides, or as 5-8-5 MOE, 5-9-5 MOE, 5-10-5 MOE, 6-7-6-MOE, 3-10-5 MOE, or 6-8-6 MOE gapmers.

The deoxy, MOE and cEt oligonucleotides are 16 nucleosides in length wherein the nucleoside have either a MOE sugar modification, an cEt sugar modification, or a deoxy modification. The ‘Chemistry’ column describes the sugar modifications of each oligonucleotide. ‘k’ indicates an cEt sugar modification; ‘d’ indicates deoxyribose; and ‘e’ indicates a MOE modification.

The 5-8-5 MOE gapmers are 18 nucleosides in length, wherein the central gap segment comprises of eight 2′-deoxynucleosides and is flanked by wing segments on the 5′ direction and the 3′ direction comprising five nucleosides each. The 5-9-5 MOE gapmers are 19 nucleosides in length, wherein the central gap segment comprises of nine 2′-deoxynucleosides and is flanked by wing segments on the 5′ direction and the 3′ direction comprising five nucleosides each. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. The 3-10-5 MOE gapmers are 18 nucleosides in length, wherein the central gap segment comprises of ten 2′-deoxynucleosides and is flanked by wing segments on the 5′ direction and the 3′ direction comprising three and five nucleosides respectively. The 6-7-6 MOE gapmers are 19 nucleosides in length, wherein the central gap segment comprises of seven 2′-deoxynucleosides and is flanked by wing segments on the 5′ direction and the 3′ direction comprising six nucleosides each. The 6-8-6 MOE gapmers are 20 nucleosides in length, wherein the central gap segment comprises of eight 2′-deoxynucleosides and is flanked by wing segments on the 5′ direction and the 3′ direction comprising six nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 19
Inhibition of CFB mRNA by deoxy, MOE and cEt oligonucleotides targeting SEQ ID NO: 1 or
SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 601152 | 2551 | 2566 | Exon 18 | AAACCCAAATCCTCAT | 22 | 7786 | 7801 | eekkddddddddkkee | 557 |
| 601218 | 2551 | 2566 | Exon 18 | AAACCCAAATCCTCAT | 21 | 7786 | 7801 | ekkkddddddddkeee | 557 |
| 601153 | 2552 | 2567 | Exon 18 | AAAACCCAAATCCTCA | 27 | 7787 | 7802 | eekkddddddddkkee | 800 |
| 601219 | 2552 | 2567 | Exon 18 | AAAACCCAAATCCTCA | 19 | 7787 | 7802 | ekkkddddddddkeee | 800 |
| 601154 | 2553 | 2568 | Exon 18 | GAAAACCCAAATCCTC | 23 | 7788 | 7803 | eekkddddddddkkee | 558 |
| 601220 | 2553 | 2568 | Exon 18 | GAAAACCCAAATCCTC | 24 | 7788 | 7803 | ekkkddddddddkeee | 558 |
| 601155 | 2554 | 2569 | Exon 18 | AGAAAACCCAAATCCT | 20 | 7789 | 7804 | eekkddddddddkkee | 801 |
| 601221 | 2554 | 2569 | Exon 18 | AGAAAACCCAAATCCT | 0 | 7789 | 7804 | ekkkddddddddkeee | 801 |
| 601156 | 2555 | 2570 | Exon 18 | TAGAAAACCCAAATCC | 11 | 7790 | 7805 | eekkddddddddkkee | 559 |
| 601222 | 2555 | 2570 | Exon 18 | TAGAAAACCCAAATCC | 23 | 7790 | 7805 | ekkkddddddddkeee | 559 |
| 601157 | 2556 | 2571 | Exon 18 | ATAGAAAACCCAAATC | 9 | 7791 | 7806 | eekkddddddddkkee | 560 |
| 601223 | 2556 | 2571 | Exon 18 | ATAGAAAACCCAAATC | 0 | 7791 | 7806 | ekkkddddddddkeee | 560 |
| 601158 | 2557 | 2572 | Exon 18 | TATAGAAAACCCAAAT | 0 | 7792 | 7807 | eekkddddddddkkee | 802 |
| 601224 | 2557 | 2572 | Exon 18 | TATAGAAAACCCAAAT | 0 | 7792 | 7807 | ekkkddddddddkeee | 802 |
| 601159 | 2558 | 2573 | Exon 18 | TTATAGAAAACCCAAA | 2 | 7793 | 7808 | eekkddddddddkkee | 803 |
| 601225 | 2558 | 2573 | Exon 18 | TTATAGAAAACCCAAA | 0 | 7793 | 7808 | ekkkddddddddkeee | 803 |
| 601160 | 2559 | 2574 | Exon 18 | CTTATAGAAAACCCAA | 0 | 7794 | 7809 | eekkddddddddkkee | 561 |
| 601226 | 2559 | 2574 | Exon 18 | CTTATAGAAAACCCAA | 0 | 7794 | 7809 | ekkkddddddddkeee | 561 |
| 601161 | 2560 | 2575 | Exon 18 | CCTTATAGAAAACCCA | 1 | 7795 | 7810 | eekkddddddddkkee | 562 |
| 601227 | 2560 | 2575 | Exon 18 | CCTTATAGAAAACCCA | 14 | 7795 | 7810 | ekkkddddddddkeee | 562 |
| 601162 | 2561 | 2576 | Exon 18 | CCCTTATAGAAAACCC | 9 | 7796 | 7811 | eekkddddddddkkee | 563 |
| 601228 | 2561 | 2576 | Exon 18 | CCCTTATAGAAAACCC | 9 | 7796 | 7811 | ekkkddddddddkeee | 563 |
| 601163 | 2562 | 2577 | Exon 18 | CCCCTTATAGAAAACC | 0 | 7797 | 7812 | eekkddddddddkkee | 564 |
| 601164 | 2563 | 2578 | Exon 18 | ACCCCTTATAGAAAAC | 3 | 7798 | 7813 | eekkddddddddkkee | 565 |
| 601165 | 2564 | 2579 | Exon 18 | AACCCCTTATAGAAAA | 0 | 7799 | 7814 | eekkddddddddkkee | 566 |
| 601166 | 2565 | 2580 | Exon 18 | AAACCCCTTATAGAAA | 0 | 7800 | 7815 | eekkddddddddkkee | 567 |
| 601167 | 2566 | 2581 | Exon 18 | GAAACCCCTTATAGAA | 0 | 7801 | 7816 | eekkddddddddkkee | 568 |
| 601168 | 2567 | 2582 | Exon 18 | GGAAACCCCTTATAGA | 0 | 7802 | 7817 | eekkddddddddkkee | 569 |
| 601169 | 2568 | 2583 | Exon 18 | AGGAAACCCCTTATAG | 0 | 7803 | 7818 | eekkddddddddkkee | 570 |
| 601170 | 2569 | 2584 | Exon 18 | CAGGAAACCCCTTATA | 10 | 7804 | 7819 | eekkddddddddkkee | 571 |
| 601171 | 2570 | 2585 | Exon 18 | GCAGGAAACCCCTTAT | 9 | 7805 | 7820 | eekkddddddddkkee | 572 |
| 601172 | 2571 | 2586 | Exon 18 | AGCAGGAAACCCCTTA | 15 | 7806 | 7821 | eekkddddddddkkee | 573 |
| 601173 | 2572 | 2587 | Exon 18 | CAGCAGGAAACCCCTT | 29 | 7807 | 7822 | eekkddddddddkkee | 574 |
| 601174 | 2573 | 2588 | Exon 18 | CCAGCAGGAAACCCCT | 25 | 7808 | 7823 | eekkddddddddkkee | 575 |
| 601175 | 2574 | 2589 | Exon 18 | TCCAGCAGGAAACCCC | 15 | 7809 | 7824 | eekkddddddddkkee | 576 |
| 601176 | 2575 | 2590 | Exon 18 | GTCCAGCAGGAAACCC | 18 | 7810 | 7825 | eekkddddddddkkee | 577 |
| 601177 | 2576 | 2591 | Exon 18 | TGTCCAGCAGGAAACC | 10 | 7811 | 7826 | eekkddddddddkkee | 578 |
| 601178 | 2577 | 2592 | Exon 18 | CTGTCCAGCAGGAAAC | 11 | 7812 | 7827 | eekkddddddddkkee | 579 |
| 601179 | 2578 | 2593 | Exon 18 | CCTGTCCAGCAGGAAA | 19 | 7813 | 7828 | eekkddddddddkkee | 580 |
| 601180 | 2579 | 2594 | Exon 18 | CCCTGTCCAGCAGGAA | 7 | 7814 | 7829 | eekkddddddddkkee | 581 |
| 601181 | 2580 | 2595 | Exon 18 | CCCCTGTCCAGCAGGA | 3 | 7815 | 7830 | eekkddddddddkkee | 582 |
| 601182 | 2581 | 2596 | Exon 18 | GCCCCTGTCCAGCAGG | 0 | 7816 | 7831 | eekkddddddddkkee | 583 |
| 601183 | 2582 | 2597 | Exon 18 | CGCCCCTGTCCAGCAG | 4 | 7817 | 7832 | eekkddddddddkkee | 584 |
| 601184 | 2583 | 2598 | Exon 18 | ACGCCCCTGTCCAGCA | 14 | 7818 | 7833 | eekkddddddddkkee | 585 |
| 601185 | 2584 | 2599 | Exon 18 | CACGCCCCTGTCCAGC | 26 | 7819 | 7834 | eekkddddddddkkee | 586 |
| 601186 | 2585 | 2600 | Exon 18 | CCACGCCCCTGTCCAG | 8 | 7820 | 7835 | eekkddddddddkkee | 587 |
| 601187 | 2586 | 2601 | Exon 18 | CCCACGCCCCTGTCCA | 18 | 7821 | 7836 | eekkddddddddkkee | 588 |
| 601188 | 2587 | 2602 | Exon 18 | TCCCACGCCCCTGTCC | 20 | 7822 | 7837 | eekkddddddddkkee | 589 |
| 601189 | 2588 | 2603 | Exon 18 | ATCCCACGCCCCTGTC | 12 | 7823 | 7838 | eekkddddddddkkee | 590 |
| 601190 | 2589 | 2604 | Exon 18 | AATCCCACGCCCCTGT | 33 | 7824 | 7839 | eekkddddddddkkee | 591 |
| 601191 | 2590 | 2605 | Exon 18 | CAATCCCACGCCCCTG | 52 | 7825 | 7840 | eekkddddddddkkee | 592 |
| 601192 | 2591 | 2606 | Exon 18 | TCAATCCCACGCCCCT | 46 | 7826 | 7841 | eekkddddddddkkee | 593 |
| 601193 | 2592 | 2607 | Exon 18 | TTCAATCCCACGCCCC | 30 | 7827 | 7842 | eekkddddddddkkee | 594 |
| 601194 | 2593 | 2608 | Exon 18 | ATTCAATCCCACGCCC | 41 | 7828 | 7843 | eekkddddddddkkee | 595 |
| 601195 | 2594 | 2609 | Exon 18 | AATTCAATCCCACGCC | 40 | 7829 | 7844 | eekkddddddddkkee | 596 |
| 601196 | 2595 | 2610 | Exon 18 | TAATTCAATCCCACGC | 71 | 7830 | 7845 | eekkddddddddkkee | 597 |
| 601197 | 2596 | 2611 | Exon 18 | TTAATTCAATCCCACG | 42 | 7831 | 7846 | eekkddddddddkkee | 598 |
| 601198 | 2597 | 2612 | Exon 18 | TTTAATTCAATCCCAC | 63 | 7832 | 7847 | eekkddddddddkkee | 599 |
| 601199 | 2598 | 2613 | Exon 18 | TTTTAATTCAATCCCA | 51 | 7833 | 7848 | eekkddddddddkkee | 600 |
| 601200 | 2599 | 2614 | Exon 18 | GTTTTAATTCAATCCC | 65 | 7834 | 7849 | eekkddddddddkkee | 601 |
| 601201 | 2600 | 2615 | Exon 18 | TGTTTTAATTCAATCC | 49 | 7835 | 7850 | eekkddddddddkkee | 602 |
| 601202 | 2601 | 2616 | Exon 18 | CTGTTTTAATTCAATC | 33 | 7836 | 7851 | eekkddddddddkkee | 603 |
| 601203 | 2602 | 2617 | Exon 18 | GCTGTTTTAATTCAAT | 63 | 7837 | 7852 | eekkddddddddkkee | 604 |
| 601204 | 2603 | 2618 | Exon 18 | AGCTGTTTTAATTCAA | 69 | 7838 | 7853 | eekkddddddddkkee | 605 |
| 532917 | 2604 | 2623 | Exon 18 | GTCGCAGCTGTTTTAATT | 73 | 7839 | 7858 | eeeeeddddddddddeeee | 317 |
| | | | | CA | | | | e | |
| 601205 | 2604 | 2619 | Exon 18 | CAGCTGTTTTAATTCA | 51 | 7839 | 7854 | eekkddddddddkkee | 606 |
| 601206 | 2605 | 2620 | Exon 18 | GCAGCTGTTTTAATTC | 43 | 7840 | 7855 | eekkddddddddkkee | 607 |
| 601207 | 2606 | 2621 | Exon 18 | CGCAGCTGTTTTAATT | 52 | 7841 | 7856 | eekkddddddddkkee | 608 |
| 601208 | 2607 | 2622 | Exon 18 | TCGCAGCTGTTTTAAT | 61 | 7842 | 7857 | eekkddddddddkkee | 609 |
| 588860 | 2608 | 2623 | Exon 18 | GTCGCAGCTGTTTTAA | 75 | 7843 | 7858 | eekddddddddddkke | 610 |
| 601209 | 2608 | 2623 | Exon 18 | GTCGCAGCTGTTTTAA | 73 | 7843 | 7858 | eekkddddddddkkee | 610 |
| 601210 | 2609 | 2624 | Exon 18 | TGTCGCAGCTGTTTTA | 80 | 7844 | 7859 | eekkddddddddkkee | 611 |
| 601211 | 2610 | 2625 | Exon 18 | TTGTCGCAGCTGTTTT | 64 | 7845 | 7860 | eekkddddddddkkee | 612 |
| 601212 | 2611 | 2626 | Exon 18 | GTTGTCGCAGCTGTTT | 86 | 7846 | 7861 | eekkddddddddkkee | 613 |
| 601213 | 2612 | 2627 | Exon 18 | TGTTGTCGCAGCTGTT | 87 | 7847 | 7862 | eekkddddddddkkee | 614 |
| 601214 | 2613 | 2628 | Exon 18/ | TTGTTGTCGCAGCTGT | 84 | n/a | n/a | eekkddddddddkkee | 615 |
| | | | Repeat | | | | | | |
| 601215 | 2614 | 2629 | Exon 18/ | TTTGTTGTCGCAGCTG | 78 | n/a | n/a | eekkddddddddkkee | 616 |
| | | | Repeat | | | | | | |
| 601216 | 2615 | 2630 | Exon 18/ | TTTTGTTGTCGCAGCT | 73 | n/a | n/a | eekkddddddddkkee | 617 |
| | | | Repeat | | | | | | |
| 601217 | 2616 | 2631 | Exon 18/ | TTTTTGTTGTCGCAGC | 66 | n/a | n/a | eekkddddddddkkee | 618 |
| | | | Repeat | | | | | | |

TABLE 20
Inhibition of CFB mRNA by deoxy, MOE and cEt oligonucleotides targeting SEQ ID NO: 1 or
SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 601284 | 2551 | 2566 | Exon 18 | AAACCCAAATCCTCAT | 8 | 7786 | 7801 | ekkddddddddkkeee | 557 |
| 601285 | 2552 | 2567 | Exon 18 | AAAACCCAAATCCTCA | 15 | 7787 | 7802 | ekkddddddddkkeee | 800 |
| 601286 | 2553 | 2568 | Exon 18 | GAAAACCCAAATCCTC | 21 | 7788 | 7803 | ekkddddddddkkeee | 558 |
| 601287 | 2554 | 2569 | Exon 18 | AGAAAACCCAAATCCT | 9 | 7789 | 7804 | ekkddddddddkkeee | 801 |
| 601288 | 2555 | 2570 | Exon 18 | TAGAAAACCCAAATCC | 0 | 7790 | 7805 | ekkddddddddkkeee | 559 |
| 601289 | 2556 | 2571 | Exon 18 | ATAGAAAACCCAAATC | 40 | 7791 | 7806 | ekkddddddddkkeee | 560 |
| 601290 | 2557 | 2572 | Exon 18 | TATAGAAAACCCAAAT | 16 | 7792 | 7807 | ekkddddddddkkeee | 802 |
| 601291 | 2558 | 2573 | Exon 18 | TTATAGAAAACCCAAA | 15 | 7793 | 7808 | ekkddddddddkkeee | 803 |
| 601292 | 2559 | 2574 | Exon 18 | CTTATAGAAAACCCAA | 5 | 7794 | 7809 | ekkddddddddkkeee | 561 |
| 601293 | 2560 | 2575 | Exon 18 | CCTTATAGAAAACCCA | 15 | 7795 | 7810 | ekkddddddddkkeee | 562 |
| 601294 | 2561 | 2576 | Exon 18 | CCCTTATAGAAAACCC | 3 | 7796 | 7811 | ekkddddddddkkeee | 563 |
| 601229 | 2562 | 2577 | Exon 18 | CCCCTTATAGAAAACC | 15 | 7797 | 7812 | ekkkddddddddkeee | 564 |
| 601295 | 2562 | 2577 | Exon 18 | CCCCTTATAGAAAACC | 5 | 7797 | 7812 | ekkddddddddkkeee | 564 |
| 601230 | 2563 | 2578 | Exon 18 | ACCCCTTATAGAAAAC | 14 | 7798 | 7813 | ekkkddddddddkeee | 565 |
| 601296 | 2563 | 2578 | Exon 18 | ACCCCTTATAGAAAAC | 0 | 7798 | 7813 | ekkddddddddkkeee | 565 |
| 601231 | 2564 | 2579 | Exon 18 | AACCCCTTATAGAAAA | 14 | 7799 | 7814 | ekkkddddddddkeee | 566 |
| 601297 | 2564 | 2579 | Exon 18 | AACCCCTTATAGAAAA | 14 | 7799 | 7814 | ekkddddddddkkeee | 566 |
| 601232 | 2565 | 2580 | Exon 18 | AAACCCCTTATAGAAA | 15 | 7800 | 7815 | ekkkddddddddkeee | 567 |
| 601298 | 2565 | 2580 | Exon 18 | AAACCCCTTATAGAAA | 7 | 7800 | 7815 | ekkddddddddkkeee | 567 |
| 601233 | 2566 | 2581 | Exon 18 | GAAACCCCTTATAGAA | 0 | 7801 | 7816 | ekkkddddddddkeee | 568 |
| 601299 | 2566 | 2581 | Exon 18 | GAAACCCCTTATAGAA | 0 | 7801 | 7816 | ekkddddddddkkeee | 568 |
| 601234 | 2567 | 2582 | Exon 18 | GGAAACCCCTTATAGA | 0 | 7802 | 7817 | ekkkddddddddkeee | 569 |
| 601300 | 2567 | 2582 | Exon 18 | GGAAACCCCTTATAGA | 9 | 7802 | 7817 | ekkddddddddkkeee | 569 |
| 601235 | 2568 | 2583 | Exon 18 | AGGAAACCCCTTATAG | 3 | 7803 | 7818 | ekkkddddddddkeee | 570 |
| 601301 | 2568 | 2583 | Exon 18 | AGGAAACCCCTTATAG | 14 | 7803 | 7818 | ekkddddddddkkeee | 570 |
| 601236 | 2569 | 2584 | Exon 18 | CAGGAAACCCCTTATA | 0 | 7804 | 7819 | ekkkddddddddkeee | 571 |
| 601302 | 2569 | 2584 | Exon 18 | CAGGAAACCCCTTATA | 0 | 7804 | 7819 | ekkddddddddkkeee | 571 |
| 601237 | 2570 | 2585 | Exon 18 | GCAGGAAACCCCTTAT | 16 | 7805 | 7820 | ekkkddddddddkeee | 572 |
| 601303 | 2570 | 2585 | Exon 18 | GCAGGAAACCCCTTAT | 16 | 7805 | 7820 | ekkddddddddkkeee | 572 |
| 601238 | 2571 | 2586 | Exon 18 | AGCAGGAAACCCCTTA | 11 | 7806 | 7821 | ekkkddddddddkeee | 573 |
| 601304 | 2571 | 2586 | Exon 18 | AGCAGGAAACCCCTTA | 10 | 7806 | 7821 | ekkddddddddkkeee | 573 |
| 601239 | 2572 | 2587 | Exon 18 | CAGCAGGAAACCCCTT | 21 | 7807 | 7822 | ekkkddddddddkeee | 574 |
| 601305 | 2572 | 2587 | Exon 18 | CAGCAGGAAACCCCTT | 7 | 7807 | 7822 | ekkddddddddkkeee | 574 |
| 601240 | 2573 | 2588 | Exon 18 | CCAGCAGGAAACCCCT | 6 | 7808 | 7823 | ekkkddddddddkeee | 575 |
| 601241 | 2574 | 2589 | Exon 18 | TCCAGCAGGAAACCCC | 10 | 7809 | 7824 | ekkkddddddddkeee | 576 |
| 601242 | 2575 | 2590 | Exon 18 | GTCCAGCAGGAAACCC | 19 | 7810 | 7825 | ekkkddddddddkeee | 577 |
| 601243 | 2576 | 2591 | Exon 18 | TGTCCAGCAGGAAACC | 10 | 7811 | 7826 | ekkkddddddddkeee | 578 |
| 601244 | 2577 | 2592 | Exon 18 | CTGTCCAGCAGGAAAC | 28 | 7812 | 7827 | ekkkddddddddkeee | 579 |
| 601245 | 2578 | 2593 | Exon 18 | CCTGTCCAGCAGGAAA | 5 | 7813 | 7828 | ekkkddddddddkeee | 580 |
| 601246 | 2579 | 2594 | Exon 18 | CCCTGTCCAGCAGGAA | 18 | 7814 | 7829 | ekkkddddddddkeee | 581 |
| 601247 | 2580 | 2595 | Exon 18 | CCCCTGTCCAGCAGGA | 4 | 7815 | 7830 | ekkkddddddddkeee | 582 |
| 601248 | 2581 | 2596 | Exon 18 | GCCCCTGTCCAGCAGG | 6 | 7816 | 7831 | ekkkddddddddkeee | 583 |
| 601249 | 2582 | 2597 | Exon 18 | CGCCCCTGTCCAGCAG | 18 | 7817 | 7832 | ekkkddddddddkeee | 584 |
| 601250 | 2583 | 2598 | Exon 18 | ACGCCCCTGTCCAGCA | 26 | 7818 | 7833 | ekkkddddddddkeee | 585 |
| 601251 | 2584 | 2599 | Exon 18 | CACGCCCCTGTCCAGC | 27 | 7819 | 7834 | ekkkddddddddkeee | 586 |
| 601252 | 2585 | 2600 | Exon 18 | CCACGCCCCTGTCCAG | 21 | 7820 | 7835 | ekkkddddddddkeee | 587 |
| 601253 | 2586 | 2601 | Exon 18 | CCCACGCCCCTGTCCA | 0 | 7821 | 7836 | ekkkddddddddkeee | 588 |
| 601254 | 2587 | 2602 | Exon 18 | TCCCACGCCCCTGTCC | 31 | 7822 | 7837 | ekkkddddddddkeee | 589 |
| 601255 | 2588 | 2603 | Exon 18 | ATCCCACGCCCCTGTC | 3 | 7823 | 7838 | ekkkddddddddkeee | 590 |
| 601256 | 2589 | 2604 | Exon 18 | AATCCCACGCCCCTGT | 21 | 7824 | 7839 | ekkkddddddddkeee | 591 |
| 601257 | 2590 | 2605 | Exon 18 | CAATCCCACGCCCCTG | 47 | 7825 | 7840 | ekkkddddddddkeee | 592 |
| 601258 | 2591 | 2606 | Exon 18 | TCAATCCCACGCCCCT | 48 | 7826 | 7841 | ekkkddddddddkeee | 593 |
| 601259 | 2592 | 2607 | Exon 18 | TTCAATCCCACGCCCC | 38 | 7827 | 7842 | ekkkddddddddkeee | 594 |
| 601260 | 2593 | 2608 | Exon 18 | ATTCAATCCCACGCCC | 33 | 7828 | 7843 | ekkkddddddddkeee | 595 |
| 601261 | 2594 | 2609 | Exon 18 | AATTCAATCCCACGCC | 17 | 7829 | 7844 | ekkkddddddddkeee | 596 |
| 601262 | 2595 | 2610 | Exon 18 | TAATTCAATCCCACGC | 40 | 7830 | 7845 | ekkkddddddddkeee | 597 |
| 601263 | 2596 | 2611 | Exon 18 | TTAATTCAATCCCACG | 31 | 7831 | 7846 | ekkkddddddddkeee | 598 |
| 601264 | 2597 | 2612 | Exon 18 | TTTAATTCAATCCCAC | 72 | 7832 | 7847 | ekkkddddddddkeee | 599 |
| 601265 | 2598 | 2613 | Exon 18 | TTTTAATTCAATCCCA | 48 | 7833 | 7848 | ekkkddddddddkeee | 600 |
| 601266 | 2599 | 2614 | Exon 18 | GTTTTAATTCAATCCC | 64 | 7834 | 7849 | ekkkddddddddkeee | 601 |
| 601267 | 2600 | 2615 | Exon 18 | TGTTTTAATTCAATCC | 43 | 7835 | 7850 | ekkkddddddddkeee | 602 |
| 601268 | 2601 | 2616 | Exon 18 | CTGTTTTAATTCAATC | 44 | 7836 | 7851 | ekkkddddddddkeee | 603 |
| 601269 | 2602 | 2617 | Exon 18 | GCTGTTTTAATTCAAT | 66 | 7837 | 7852 | ekkkddddddddkeee | 604 |
| 601270 | 2603 | 2618 | Exon 18 | AGCTGTTTTAATTCAA | 47 | 7838 | 7853 | ekkkddddddddkeee | 605 |
| 532917 | 2604 | 2623 | Exon 18 | GTCGCAGCTGTTTTAATT | 3 | 7839 | 7858 | eeeeeddddddddddeeee | 317 |
| | | | | CA | | | | e | |
| 601271 | 2604 | 2619 | Exon 18 | CAGCTGTTTTAATTCA | 26 | 7839 | 7854 | ekkkddddddddkeee | 606 |
| 601272 | 2605 | 2620 | Exon 18 | GCAGCTGTTTTAATTC | 33 | 7840 | 7855 | ekkkddddddddkeee | 607 |
| 601273 | 2606 | 2621 | Exon 18 | CGCAGCTGTTTTAATT | 34 | 7841 | 7856 | ekkkddddddddkeee | 608 |
| 601274 | 2607 | 2622 | Exon 18 | TCGCAGCTGTTTTAAT | 39 | 7842 | 7857 | ekkkddddddddkeee | 609 |
| 588860 | 2608 | 2623 | Exon 18 | GTCGCAGCTGTTTTAA | 72 | 7843 | 7858 | eekddddddddddkke | 610 |
| 601275 | 2608 | 2623 | Exon 18 | GTCGCAGCTGTTTTAA | 65 | 7843 | 7858 | ekkkddddddddkeee | 610 |
| 601276 | 2609 | 2624 | Exon 18 | TGTCGCAGCTGTTTTA | 65 | 7844 | 7859 | ekkkddddddddkeee | 611 |
| 601277 | 2610 | 2625 | Exon 18 | TTGTCGCAGCTGTTTT | 51 | 7845 | 7860 | ekkkddddddddkeee | 612 |
| 601278 | 2611 | 2626 | Exon 18 | GTTGTCGCAGCTGTTT | 78 | 7846 | 7861 | ekkkddddddddkeee | 613 |
| 601279 | 2612 | 2627 | Exon 18 | TGTTGTCGCAGCTGTT | 79 | 7847 | 7862 | ekkkddddddddkeee | 614 |
| 601280 | 2613 | 2628 | Exon 18/ | TTGTTGTCGCAGCTGT | 70 | n/a | n/a | ekkkddddddddkeee | 615 |
| | | | Repeat | | | | | | |
| 601281 | 2614 | 2629 | Exon 18/ | TTTGTTGTCGCAGCTG | 78 | n/a | n/a | ekkkddddddddkeee | 616 |
| | | | Repeat | | | | | | |
| 601282 | 2615 | 2630 | Exon 18/ | TTTTGTTGTCGCAGCT | 68 | n/a | n/a | ekkkddddddddkeee | 617 |
| | | | Repeat | | | | | | |
| 601283 | 2616 | 2631 | Exon 18/ | TTTTTGTTGTCGCAGC | 61 | n/a | n/a | ekkkddddddddkeee | 618 |
| | | | Repeat | | | | | | |

TABLE 21
Inhibition of CFB mRNA by deoxy, MOE and cEt oligonucleotides tareting SEQ ID NO: 1 or
SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % in- | start | stop | | ID |
| NO | site | site | region | Sequence | hibition | site | site | Motif | NO: |
| 601306 | 2573 | 2588 | Exon 18 | CCAGCAGGAAACCCCT | 22 | 7808 | 7823 | ekkddddddddkkeee | 575 |
| 601307 | 2574 | 2589 | Exon 18 | TCCAGCAGGAAACCCC | 22 | 7809 | 7824 | ekkddddddddkkeee | 576 |
| 601308 | 2575 | 2590 | Exon 18 | GTCCAGCAGGAAACCC | 33 | 7810 | 7825 | ekkddddddddkkeee | 577 |
| 601309 | 2576 | 2591 | Exon 18 | TGTCCAGCAGGAAACC | 33 | 7811 | 7826 | ekkddddddddkkeee | 578 |
| 601310 | 2577 | 2592 | Exon 18 | CTGTCCAGCAGGAAAC | 28 | 7812 | 7827 | ekkddddddddkkeee | 579 |
| 601311 | 2578 | 2593 | Exon 18 | CCTGTCCAGCAGGAAA | 33 | 7813 | 7828 | ekkddddddddkkeee | 580 |
| 601312 | 2579 | 2594 | Exon 18 | CCCTGTCCAGCAGGAA | 13 | 7814 | 7829 | ekkddddddddkkeee | 581 |
| 601313 | 2580 | 2595 | Exon 18 | CCCCTGTCCAGCAGGA | 32 | 7815 | 7830 | ekkddddddddkkeee | 582 |
| 601314 | 2581 | 2596 | Exon 18 | GCCCCTGTCCAGCAGG | 0 | 7816 | 7831 | ekkddddddddkkeee | 583 |
| 601315 | 2582 | 2597 | Exon 18 | CGCCCCTGTCCAGCAG | 36 | 7817 | 7832 | ekkddddddddkkeee | 584 |
| 601316 | 2583 | 2598 | Exon 18 | ACGCCCCTGTCCAGCA | 39 | 7818 | 7833 | ekkddddddddkkeee | 585 |
| 601317 | 2584 | 2599 | Exon 18 | CACGCCCCTGTCCAGC | 33 | 7819 | 7834 | ekkddddddddkkeee | 586 |
| 601356 | 2584 | 2599 | Exon 18 | CACGCCCCTGTCCAGC | 27 | 7819 | 7834 | kkkddddddddkeeee | 586 |
| 601318 | 2585 | 2600 | Exon 18 | CCACGCCCCTGTCCAG | 35 | 7820 | 7835 | ekkddddddddkkeee | 587 |
| 601357 | 2585 | 2600 | Exon 18 | CCACGCCCCTGTCCAG | 26 | 7820 | 7835 | kkkddddddddkeeee | 587 |
| 601319 | 2586 | 2601 | Exon 18 | CCCACGCCCCTGTCCA | 33 | 7821 | 7836 | ekkddddddddkkeee | 588 |
| 601358 | 2586 | 2601 | Exon 18 | CCCACGCCCCTGTCCA | 26 | 7821 | 7836 | kkkddddddddkeeee | 588 |
| 601320 | 2587 | 2602 | Exon 18 | TCCCACGCCCCTGTCC | 25 | 7822 | 7837 | ekkddddddddkkeee | 589 |
| 601359 | 2587 | 2602 | Exon 18 | TCCCACGCCCCTGTCC | 23 | 7822 | 7837 | kkkddddddddkeeee | 589 |
| 601321 | 2588 | 2603 | Exon 18 | ATCCCACGCCCCTGTC | 50 | 7823 | 7838 | ekkddddddddkkeee | 590 |
| 601360 | 2588 | 2603 | Exon 18 | ATCCCACGCCCCTGTC | 33 | 7823 | 7838 | kkkddddddddkeeee | 590 |
| 601322 | 2589 | 2604 | Exon 18 | AATCCCACGCCCCTGT | 52 | 7824 | 7839 | ekkddddddddkkeee | 591 |
| 601361 | 2589 | 2604 | Exon 18 | AATCCCACGCCCCTGT | 48 | 7824 | 7839 | kkkddddddddkeeee | 591 |
| 601323 | 2590 | 2605 | Exon 18 | CAATCCCACGCCCCTG | 67 | 7825 | 7840 | ekkddddddddkkeee | 592 |
| 601362 | 2590 | 2605 | Exon 18 | CAATCCCACGCCCCTG | 51 | 7825 | 7840 | kkkddddddddkeeee | 592 |
| 601324 | 2591 | 2606 | Exon 18 | TCAATCCCACGCCCCT | 42 | 7826 | 7841 | ekkddddddddkkeee | 593 |
| 601363 | 2591 | 2606 | Exon 18 | TCAATCCCACGCCCCT | 42 | 7826 | 7841 | kkkddddddddkeeee | 593 |
| 601325 | 2592 | 2607 | Exon 18 | TTCAATCCCACGCCCC | 52 | 7827 | 7842 | ekkddddddddkkeee | 594 |
| 601364 | 2592 | 2607 | Exon 18 | TTCAATCCCACGCCCC | 48 | 7827 | 7842 | kkkddddddddkeeee | 594 |
| 601326 | 2593 | 2608 | Exon 18 | ATTCAATCCCACGCCC | 27 | 7828 | 7843 | ekkddddddddkkeee | 595 |
| 601365 | 2593 | 2608 | Exon 18 | ATTCAATCCCACGCCC | 36 | 7828 | 7843 | kkkddddddddkeeee | 595 |
| 601327 | 2594 | 2609 | Exon 18 | AATTCAATCCCACGCC | 66 | 7829 | 7844 | ekkddddddddkkeee | 596 |
| 601366 | 2594 | 2609 | Exon 18 | AATTCAATCCCACGCC | 49 | 7829 | 7844 | kkkddddddddkeeee | 596 |
| 601328 | 2595 | 2610 | Exon 18 | TAATTCAATCCCACGC | 55 | 7830 | 7845 | ekkddddddddkkeee | 597 |
| 601367 | 2595 | 2610 | Exon 18 | TAATTCAATCCCACGC | 57 | 7830 | 7845 | kkkddddddddkeeee | 597 |
| 601329 | 2596 | 2611 | Exon 18 | TTAATTCAATCCCACG | 69 | 7831 | 7846 | ekkddddddddkkeee | 598 |
| 601368 | 2596 | 2611 | Exon 18 | TTAATTCAATCCCACG | 68 | 7831 | 7846 | kkkddddddddkeeee | 598 |
| 601330 | 2597 | 2612 | Exon 18 | TTTAATTCAATCCCAC | 58 | 7832 | 7847 | ekkddddddddkkeee | 599 |
| 601369 | 2597 | 2612 | Exon 18 | TTTAATTCAATCCCAC | 65 | 7832 | 7847 | kkkddddddddkeeee | 599 |
| 601331 | 2598 | 2613 | Exon 18 | TTTTAATTCAATCCCA | 45 | 7833 | 7848 | ekkddddddddkkeee | 600 |
| 601370 | 2598 | 2613 | Exon 18 | TTTTAATTCAATCCCA | 42 | 7833 | 7848 | kkkddddddddkeeee | 600 |
| 601332 | 2599 | 2614 | Exon 18 | GTTTTAATTCAATCCC | 84 | 7834 | 7849 | ekkddddddddkkeee | 601 |
| 601371 | 2599 | 2614 | Exon 18 | GTTTTAATTCAATCCC | 79 | 7834 | 7849 | kkkddddddddkeeee | 601 |
| 601333 | 2600 | 2615 | Exon 18 | TGTTTTAATTCAATCC | 61 | 7835 | 7850 | ekkddddddddkkeee | 602 |
| 601372 | 2600 | 2615 | Exon 18 | TGTTTTAATTCAATCC | 71 | 7835 | 7850 | kkkddddddddkeeee | 602 |
| 601334 | 2601 | 2616 | Exon 18 | CTGTTTTAATTCAATC | 61 | 7836 | 7851 | ekkddddddddkkeee | 603 |
| 601373 | 2601 | 2616 | Exon 18 | CTGTTTTAATTCAATC | 57 | 7836 | 7851 | kkkddddddddkeeee | 603 |
| 601335 | 2602 | 2617 | Exon 18 | GCTGTTTTAATTCAAT | 73 | 7837 | 7852 | ekkddddddddkkeee | 604 |
| 601374 | 2602 | 2617 | Exon 18 | GCTGTTTTAATTCAAT | 66 | 7837 | 7852 | kkkddddddddkeeee | 604 |
| 601336 | 2603 | 2618 | Exon 18 | AGCTGTTTTAATTCAA | 64 | 7838 | 7853 | ekkddddddddkkeee | 605 |
| 601375 | 2603 | 2618 | Exon 18 | AGCTGTTTTAATTCAA | 61 | 7838 | 7853 | kkkddddddddkeeee | 605 |
| 532917 | 2604 | 2623 | Exon 18 | GTCGCAGCTGTTTTAATT | 66 | 7839 | 7858 | eeeeedddddddddde | 317 |
| | | | | CA | | | | eeee | |
| 601337 | 2604 | 2619 | Exon 18 | CAGCTGTTTTAATTCA | 53 | 7839 | 7854 | ekkddddddddkkeee | 606 |
| 601376 | 2604 | 2619 | Exon 18 | CAGCTGTTTTAATTCA | 39 | 7839 | 7854 | kkkddddddddkeeee | 606 |
| 601338 | 2605 | 2620 | Exon 18 | GCAGCTGTTTTAATTC | 67 | 7840 | 7855 | ekkddddddddkkeee | 607 |
| 601377 | 2605 | 2620 | Exon 18 | GCAGCTGTTTTAATTC | 67 | 7840 | 7855 | kkkddddddddkeeee | 607 |
| 601339 | 2606 | 2621 | Exon 18 | CGCAGCTGTTTTAATT | 63 | 7841 | 7856 | ekkddddddddkkeee | 608 |
| 601378 | 2606 | 2621 | Exon 18 | CGCAGCTGTTTTAATT | 60 | 7841 | 7856 | kkkddddddddkeeee | 608 |
| 601340 | 2607 | 2622 | Exon 18 | TCGCAGCTGTTTTAAT | 40 | 7842 | 7857 | ekkddddddddkkeee | 609 |
| 601379 | 2607 | 2622 | Exon 18 | TCGCAGCTGTTTTAAT | 36 | 7842 | 7857 | kkkddddddddkeeee | 609 |
| 588860 | 2608 | 2623 | Exon 18 | GTCGCAGCTGTTTTAA | 84 | 7843 | 7858 | eekddddddddddkke | 610 |
| 601341 | 2608 | 2623 | Exon 18 | GTCGCAGCTGTTTTAA | 74 | 7843 | 7858 | ekkddddddddkkeee | 610 |
| 601380 | 2608 | 2623 | Exon 18 | GTCGCAGCTGTTTTAA | 78 | 7843 | 7858 | kkkddddddddkeeee | 610 |
| 601342 | 2609 | 2624 | Exon 18 | TGTCGCAGCTGTTTTA | 68 | 7844 | 7859 | ekkddddddddkkeee | 611 |
| 601381 | 2609 | 2624 | Exon 18 | TGTCGCAGCTGTTTTA | 66 | 7844 | 7859 | kkkddddddddkeeee | 611 |
| 601343 | 2610 | 2625 | Exon 18 | TTGTCGCAGCTGTTTT | 71 | 7845 | 7860 | ekkddddddddkkeee | 612 |
| 601382 | 2610 | 2625 | Exon 18 | TTGTCGCAGCTGTTTT | 84 | 7845 | 7860 | kkkddddddddkeeee | 612 |
| 601344 | 2611 | 2626 | Exon 18 | GTTGTCGCAGCTGTTT | 87 | 7846 | 7861 | ekkddddddddkkeee | 613 |
| 601383 | 2611 | 2626 | Exon 18 | GTTGTCGCAGCTGTTT | 85 | 7846 | 7861 | kkkddddddddkeeee | 613 |
| 601345 | 2612 | 2627 | Exon 18 | TGTTGTCGCAGCTGTT | 82 | 7847 | 7862 | ekkddddddddkkeee | 614 |
| 601384 | 2612 | 2627 | Exon 18 | TGTTGTCGCAGCTGTT | 79 | 7847 | 7862 | kkkddddddddkeeee | 614 |
| 601346 | 2613 | 2628 | Exon 18/ | TTGTTGTCGCAGCTGT | 73 | n/a | n/a | ekkddddddddkkeee | 615 |
| | | | Repeat | | | | | | |
| 601385 | 2613 | 2628 | Exon 18/ | TTGTTGTCGCAGCTGT | 84 | n/a | n/a | kkkddddddddkeeee | 615 |
| | | | Repeat | | | | | | |
| 601347 | 2614 | 2629 | Exon 18/ | TTTGTTGTCGCAGCTG | 70 | n/a | n/a | ekkddddddddkkeee | 616 |
| | | | Repeat | | | | | | |
| 601386 | 2614 | 2629 | Exon 18/ | TTTGTTGTCGCAGCTG | 71 | n/a | n/a | kkkddddddddkeeee | 616 |
| | | | Repeat | | | | | | |
| 601348 | 2615 | 2630 | Exon 18/ | TTTTGTTGTCGCAGCT | 71 | n/a | n/a | ekkddddddddkkeee | 617 |
| | | | Repeat | | | | | | |
| 601387 | 2615 | 2630 | Exon 18/ | TTTTGTTGTCGCAGCT | 76 | n/a | n/a | kkkddddddddkeeee | 617 |
| | | | Repeat | | | | | | |
| 601349 | 2616 | 2631 | Exon 18/ | TTTTTGTTGTCGCAGC | 71 | n/a | n/a | ekkddddddddkkeee | 618 |
| | | | Repeat | | | | | | |
| 601388 | 2616 | 2631 | Exon 18/ | TTTTTGTTGTCGCAGC | 67 | n/a | n/a | kkkddddddddkeeee | 618 |
| | | | Repeat | | | | | | |

TABLE 22
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID | ID | | |
| | NO: 1 | NO: 1 | | | | NO: 2 | NO: 2 | | SEQ |
| ISIS | start | stop | Target | | % | start | stop | | ID |
| NO | site | site | region | Sequence | inhibition | site | site | Motif | NO: |
| 599357 | 2582 | 2600 | Exon 18 | CCACGCCCCTGTCCAGCAG | 26 | 7817 | 7835 | 5-9-5 | 708 |
| 599358 | 2583 | 2601 | Exon 18 | CCCACGCCCCTGTCCAGCA | 22 | 7818 | 7836 | 5-9-5 | 709 |
| 599359 | 2584 | 2602 | Exon 18 | TCCCACGCCCCTGTCCAGC | 13 | 7819 | 7837 | 5-9-5 | 710 |
| 599360 | 2585 | 2603 | Exon 18 | ATCCCACGCCCCTGTCCAG | 7 | 7820 | 7838 | 5-9-5 | 711 |
| 599361 | 2586 | 2604 | Exon 18 | AATCCCACGCCCCTGTCCA | 11 | 7821 | 7839 | 5-9-5 | 712 |
| 599362 | 2587 | 2605 | Exon 18 | CAATCCCACGCCCCTGTCC | 14 | 7822 | 7840 | 5-9-5 | 713 |
| 599363 | 2588 | 2606 | Exon 18 | TCAATCCCACGCCCCTGTC | 17 | 7823 | 7841 | 5-9-5 | 714 |
| 599364 | 2589 | 2607 | Exon 18 | TTCAATCCCACGCCCCTGT | 20 | 7824 | 7842 | 5-9-5 | 715 |
| 599365 | 2590 | 2608 | Exon 18 | ATTCAATCCCACGCCCCTG | 22 | 7825 | 7843 | 5-9-5 | 716 |
| 599366 | 2591 | 2609 | Exon 18 | AATTCAATCCCACGCCCCT | 13 | 7826 | 7844 | 5-9-5 | 717 |
| 599367 | 2592 | 2610 | Exon 18 | TAATTCAATCCCACGCCCC | 11 | 7827 | 7845 | 5-9-5 | 718 |
| 599368 | 2593 | 2611 | Exon 18 | TTAATTCAATCCCACGCCC | 10 | 7828 | 7846 | 5-9-5 | 719 |
| 599369 | 2594 | 2612 | Exon 18 | TTTAATTCAATCCCACGCC | 19 | 7829 | 7847 | 5-9-5 | 720 |
| 599370 | 2595 | 2613 | Exon 18 | TTTTAATTCAATCCCACGC | 23 | 7830 | 7848 | 5-9-5 | 721 |
| 599371 | 2596 | 2614 | Exon 18 | GTTTTAATTCAATCCCACG | 4 | 7831 | 7849 | 5-9-5 | 722 |
| 599372 | 2597 | 2615 | Exon 18 | TGTTTTAATTCAATCCCAC | 16 | 7832 | 7850 | 5-9-5 | 723 |
| 599373 | 2598 | 2616 | Exon 18 | CTGTTTTAATTCAATCCCA | 3 | 7833 | 7851 | 5-9-5 | 724 |
| 599374 | 2599 | 2617 | Exon 18 | GCTGTTTTAATTCAATCCC | 10 | 7834 | 7852 | 5-9-5 | 725 |
| 599375 | 2600 | 2618 | Exon 18 | AGCTGTTTTAATTCAATCC | 17 | 7835 | 7853 | 5-9-5 | 726 |
| 599376 | 2601 | 2619 | Exon 18 | CAGCTGTTTTAATTCAATC | 18 | 7836 | 7854 | 5-9-5 | 727 |
| 599377 | 2602 | 2620 | Exon 18 | GCAGCTGTTTTAATTCAAT | 22 | 7837 | 7855 | 5-9-5 | 728 |
| 599378 | 2603 | 2621 | Exon 18 | CGCAGCTGTTTTAATTCAA | 11 | 7838 | 7856 | 5-9-5 | 729 |
| 599511 | 2552 | 2571 | Exon 18 | ATAGAAAACCCAAATCCTCA | 7 | 7787 | 7806 | 6-8-6 | 410 |
| 599389 | 2553 | 2572 | Exon 18 | TATAGAAAACCCAAATCCTC | 22 | 7788 | 7807 | 6-8-6 | 411 |
| 599390 | 2554 | 2573 | Exon 18 | TTATAGAAAACCCAAATCCT | 21 | 7789 | 7808 | 6-8-6 | 412 |
| 599391 | 2555 | 2574 | Exon 18 | CTTATAGAAAACCCAAATCC | 27 | 7790 | 7809 | 6-8-6 | 413 |
| 599392 | 2556 | 2575 | Exon 18 | CCTTATAGAAAACCCAAATC | 30 | 7791 | 7810 | 6-8-6 | 414 |
| 599393 | 2557 | 2576 | Exon 18 | CCCTTATAGAAAACCCAAAT | 30 | 7792 | 7811 | 6-8-6 | 415 |
| 599394 | 2558 | 2577 | Exon 18 | CCCCTTATAGAAAACCCAAA | 28 | 7793 | 7812 | 6-8-6 | 416 |
| 599395 | 2559 | 2578 | Exon 18 | ACCCCTTATAGAAAACCCAA | 23 | 7794 | 7813 | 6-8-6 | 417 |
| 599396 | 2560 | 2579 | Exon 18 | AACCCCTTATAGAAAACCCA | 53 | 7795 | 7814 | 6-8-6 | 418 |
| 599397 | 2561 | 2580 | Exon 18 | AAACCCCTTATAGAAAACCC | 33 | 7796 | 7815 | 6-8-6 | 419 |
| 599398 | 2562 | 2581 | Exon 18 | GAAACCCCTTATAGAAAACC | 58 | 7797 | 7816 | 6-8-6 | 420 |
| 599399 | 2563 | 2582 | Exon 18 | GGAAACCCCTTATAGAAAAC | 23 | 7798 | 7817 | 6-8-6 | 421 |
| 599400 | 2564 | 2583 | Exon 18 | AGGAAACCCCTTATAGAAAA | 54 | 7799 | 7818 | 6-8-6 | 422 |
| 599401 | 2565 | 2584 | Exon 18 | CAGGAAACCCCTTATAGAAA | 30 | 7800 | 7819 | 6-8-6 | 423 |
| 599402 | 2566 | 2585 | Exon 18 | GCAGGAAACCCCTTATAGAA | 25 | 7801 | 7820 | 6-8-6 | 424 |
| 599403 | 2567 | 2586 | Exon 18 | AGCAGGAAACCCCTTATAGA | 17 | 7802 | 7821 | 6-8-6 | 425 |
| 599404 | 2568 | 2587 | Exon 18 | CAGCAGGAAACCCCTTATAG | 20 | 7803 | 7822 | 6-8-6 | 426 |
| 599405 | 2569 | 2588 | Exon 18 | CCAGCAGGAAACCCCTTATA | 12 | 7804 | 7823 | 6-8-6 | 427 |
| 599406 | 2570 | 2589 | Exon 18 | TCCAGCAGGAAACCCCTTAT | 51 | 7805 | 7824 | 6-8-6 | 428 |
| 599407 | 2571 | 2590 | Exon 18 | GTCCAGCAGGAAACCCCTTA | 39 | 7806 | 7825 | 6-8-6 | 237 |
| 599408 | 2572 | 2591 | Exon 18 | TGTCCAGCAGGAAACCCCTT | 53 | 7807 | 7826 | 6-8-6 | 429 |
| 599409 | 2573 | 2592 | Exon 18 | CTGTCCAGCAGGAAACCCCT | 65 | 7808 | 7827 | 6-8-6 | 430 |
| 599410 | 2574 | 2593 | Exon 18 | CCTGTCCAGCAGGAAACCCC | 56 | 7809 | 7828 | 6-8-6 | 431 |
| 599411 | 2575 | 2594 | Exon 18 | CCCTGTCCAGCAGGAAACCC | 60 | 7810 | 7829 | 6-8-6 | 432 |
| 599412 | 2576 | 2595 | Exon 18 | CCCCTGTCCAGCAGGAAACC | 61 | 7811 | 7830 | 6-8-6 | 433 |
| 599413 | 2577 | 2596 | Exon 18 | GCCCCTGTCCAGCAGGAAAC | 40 | 7812 | 7831 | 6-8-6 | 238 |
| 599414 | 2578 | 2597 | Exon 18 | CGCCCCTGTCCAGCAGGAAA | 41 | 7813 | 7832 | 6-8-6 | 434 |
| 599415 | 2579 | 2598 | Exon 18 | ACGCCCCTGTCCAGCAGGAA | 37 | 7814 | 7833 | 6-8-6 | 435 |
| 599416 | 2580 | 2599 | Exon 18 | CACGCCCCTGTCCAGCAGGA | 54 | 7815 | 7834 | 6-8-6 | 436 |
| 599417 | 2581 | 2600 | Exon 18 | CCACGCCCCTGTCCAGCAGG | 36 | 7816 | 7835 | 6-8-6 | 437 |
| 599418 | 2582 | 2601 | Exon 18 | CCCACGCCCCTGTCCAGCAG | 53 | 7817 | 7836 | 6-8-6 | 438 |
| 599419 | 2583 | 2602 | Exon 18 | TCCCACGCCCCTGTCCAGCA | 54 | 7818 | 7837 | 6-8-6 | 439 |
| 599420 | 2584 | 2603 | Exon 18 | ATCCCACGCCCCTGTCCAGC | 50 | 7819 | 7838 | 6-8-6 | 440 |
| 599421 | 2585 | 2604 | Exon 18 | AATCCCACGCCCCTGTCCAG | 48 | 7820 | 7839 | 6-8-6 | 441 |
| 599422 | 2586 | 2605 | Exon 18 | CAATCCCACGCCCCTGTCCA | 55 | 7821 | 7840 | 6-8-6 | 442 |
| 599423 | 2587 | 2606 | Exon 18 | TCAATCCCACGCCCCTGTCC | 75 | 7822 | 7841 | 6-8-6 | 443 |
| 599424 | 2588 | 2607 | Exon 18 | TTCAATCCCACGCCCCTGTC | 69 | 7823 | 7842 | 6-8-6 | 444 |
| 599425 | 2589 | 2608 | Exon 18 | ATTCAATCCCACGCCCCTGT | 77 | 7824 | 7843 | 6-8-6 | 445 |
| 599426 | 2590 | 2609 | Exon 18 | AATTCAATCCCACGCCCCTG | 60 | 7825 | 7844 | 6-8-6 | 446 |
| 599427 | 2591 | 2610 | Exon 18 | TAATTCAATCCCACGCCCCT | 72 | 7826 | 7845 | 6-8-6 | 447 |
| 599428 | 2592 | 2611 | Exon 18 | TTAATTCAATCCCACGCCCC | 81 | 7827 | 7846 | 6-8-6 | 448 |
| 599429 | 2593 | 2612 | Exon 18 | TTTAATTCAATCCCACGCCC | 68 | 7828 | 7847 | 6-8-6 | 449 |
| 599430 | 2594 | 2613 | Exon 18 | TTTTAATTCAATCCCACGCC | 58 | 7829 | 7848 | 6-8-6 | 450 |
| 599431 | 2595 | 2614 | Exon 18 | GTTTTAATTCAATCCCACGC | 70 | 7830 | 7849 | 6-8-6 | 451 |
| 599432 | 2596 | 2615 | Exon 18 | TGTTTTAATTCAATCCCACG | 85 | 7831 | 7850 | 6-8-6 | 452 |
| 532917 | 2604 | 2623 | Exon 18 | GTCGCAGCTGTTTTAATTCA | 85 | 7839 | 7858 | 5-10-5 | 317 |
| 599379 | 2604 | 2622 | Exon 18 | TCGCAGCTGTTTTAATTCA | 73 | 7839 | 7857 | 5-9-5 | 730 |
| 599380 | 2605 | 2623 | Exon 18 | GTCGCAGCTGTTTTAATTC | 77 | 7840 | 7858 | 5-9-5 | 731 |
| 599381 | 2606 | 2624 | Exon 18 | TGTCGCAGCTGTTTTAATT | 69 | 7841 | 7859 | 5-9-5 | 732 |
| 599382 | 2607 | 2625 | Exon 18 | TTGTCGCAGCTGTTTTAAT | 58 | 7842 | 7860 | 5-9-5 | 733 |
| 599383 | 2608 | 2626 | Exon 18 | GTTGTCGCAGCTGTTTTAA | 52 | 7843 | 7861 | 5-9-5 | 734 |
| 599384 | 2609 | 2627 | Exon 18 | TGTTGTCGCAGCTGTTTTA | 63 | 7844 | 7862 | 5-9-5 | 735 |
| 599385 | 2610 | 2628 | Exon 18/ | TTGTTGTCGCAGCTGTTTT | 53 | n/a | n/a | 5-9-5 | 736 |
| | | | Repeat | | | | | | |
| 599386 | 2611 | 2629 | Exon 18/ | TTTGTTGTCGCAGCTGTTT | 63 | n/a | n/a | 5-9-5 | 737 |
| | | | Repeat | | | | | | |
| 599387 | 2612 | 2630 | Exon 18/ | TTTTGTTGTCGCAGCTGTT | 64 | n/a | n/a | 5-9-5 | 438 |
| | | | Repeat | | | | | | |
| 599388 | 2613 | 2631 | Exon 18/ | TTTTTGTTGTCGCAGCTGT | 66 | n/a | n/a | 5-9-5 | 739 |
| | | | Repeat | | | | | | |

TABLE 23
Inhibition of CFB mRNA by MOE gapmers targeting SEQ ID NO: 1 or SEQ ID NO: 2

| | SEQ | SEQ | | | | SEQ | SEQ | | |
|---|---|---|---|---|---|---|---|---|---|
| | ID | ID | | | | ID NO: | ID NO: | | |
| | NO: 1 | NO: 1 | | | % | 2 | 2 | | SEQ |
| ISIS | start | stop | Target | | in- | start | stop | | ID |
| NO | site | site | region | Sequence | hibition | site | site | Motif | NO: |
| 599213 | 2553 | 2570 | Exon 18 | TAGAAAACCCAAATCCTC | 0 | 7788 | 7805 | 3-10-5 | 785 |
| 599214 | 2554 | 2571 | Exon 18 | ATAGAAAACCCAAATCCT | 0 | 7789 | 7806 | 3-10-5 | 786 |
| 599215 | 2555 | 2572 | Exon 18 | TATAGAAAACCCAAATCC | 36 | 7790 | 7807 | 3-10-5 | 787 |
| 599216 | 2556 | 2573 | Exon 18 | TTATAGAAAACCCAAATC | 8 | 7791 | 7808 | 3-10-5 | 788 |
| 599217 | 2557 | 2574 | Exon 18 | CTTATAGAAAACCCAAAT | 5 | 7792 | 7809 | 3-10-5 | 789 |
| 599218 | 2558 | 2575 | Exon 18 | CCTTATAGAAAACCCAAA | 0 | 7793 | 7810 | 3-10-5 | 790 |
| 599219 | 2559 | 2576 | Exon 18 | CCCTTATAGAAAACCCAA | 8 | 7794 | 7811 | 3-10-5 | 791 |
| 599220 | 2560 | 2577 | Exon 18 | CCCCTTATAGAAAACCCA | 0 | 7795 | 7812 | 3-10-5 | 740 |
| 599221 | 2561 | 2578 | Exon 18 | ACCCCTTATAGAAAACCC | 54 | 7796 | 7813 | 3-10-5 | 741 |
| 599222 | 2562 | 2579 | Exon 18 | AACCCCTTATAGAAAACC | 3 | 7797 | 7814 | 3-10-5 | 742 |
| 599223 | 2563 | 2580 | Exon 18 | AAACCCCTTATAGAAAAC | 0 | 7798 | 7815 | 3-10-5 | 743 |
| 599224 | 2564 | 2581 | Exon 18 | GAAACCCCTTATAGAAAA | 0 | 7799 | 7816 | 3-10-5 | 744 |
| 599225 | 2566 | 2583 | Exon 18 | AGGAAACCCCTTATAGAA | 60 | 7801 | 7818 | 3-10-5 | 745 |
| 599226 | 2567 | 2584 | Exon 18 | CAGGAAACCCCTTATAGA | 0 | 7802 | 7819 | 3-10-5 | 746 |
| 599227 | 2568 | 2585 | Exon 18 | GCAGGAAACCCCTTATAG | 37 | 7803 | 7820 | 3-10-5 | 747 |
| 599228 | 2569 | 2586 | Exon 18 | AGCAGGAAACCCCTTATA | 0 | 7804 | 7821 | 3-10-5 | 748 |
| 599229 | 2570 | 2587 | Exon 18 | CAGCAGGAAACCCCTTAT | 39 | 7805 | 7822 | 3-10-5 | 749 |
| 599230 | 2571 | 2588 | Exon 18 | CCAGCAGGAAACCCCTTA | 10 | 7806 | 7823 | 3-10-5 | 750 |
| 599231 | 2572 | 2589 | Exon 18 | TCCAGCAGGAAACCCCTT | 16 | 7807 | 7824 | 3-10-5 | 751 |
| 599232 | 2573 | 2590 | Exon 18 | GTCCAGCAGGAAACCCCT | 9 | 7808 | 7825 | 3-10-5 | 752 |
| 599233 | 2574 | 2591 | Exon 18 | TGTCCAGCAGGAAACCCC | 44 | 7809 | 7826 | 3-10-5 | 753 |
| 599234 | 2575 | 2592 | Exon 18 | CTGTCCAGCAGGAAACCC | 14 | 7810 | 7827 | 3-10-5 | 754 |
| 599235 | 2576 | 2593 | Exon 18 | CCTGTCCAGCAGGAAACC | 0 | 7811 | 7828 | 3-10-5 | 755 |
| 599236 | 2577 | 2594 | Exon 18 | CCCTGTCCAGCAGGAAAC | 43 | 7812 | 7829 | 3-10-5 | 756 |
| 599237 | 2578 | 2595 | Exon 18 | CCCCTGTCCAGCAGGAAA | 0 | 7813 | 7830 | 3-10-5 | 757 |
| 599238 | 2580 | 2597 | Exon 18 | CGCCCCTGTCCAGCAGGA | 9 | 7815 | 7832 | 3-10-5 | 758 |
| 599239 | 2581 | 2598 | Exon 18 | ACGCCCCTGTCCAGCAGG | 36 | 7816 | 7833 | 3-10-5 | 759 |
| 599240 | 2582 | 2599 | Exon 18 | CACGCCCCTGTCCAGCAG | 11 | 7817 | 7834 | 3-10-5 | 760 |
| 599241 | 2583 | 2600 | Exon 18 | CCACGCCCCTGTCCAGCA | 51 | 7818 | 7835 | 3-10-5 | 761 |
| 599242 | 2584 | 2601 | Exon 18 | CCCACGCCCCTGTCCAGC | 7 | 7819 | 7836 | 3-10-5 | 762 |
| 599243 | 2585 | 2602 | Exon 18 | TCCCACGCCCCTGTCCAG | 47 | 7820 | 7837 | 3-10-5 | 763 |
| 599244 | 2586 | 2603 | Exon 18 | ATCCCACGCCCCTGTCCA | 37 | 7821 | 7838 | 3-10-5 | 764 |
| 599245 | 2587 | 2604 | Exon 18 | AATCCCACGCCCCTGTCC | 35 | 7822 | 7839 | 3-10-5 | 765 |
| 599246 | 2588 | 2605 | Exon 18 | CAATCCCACGCCCCTGTC | 21 | 7823 | 7840 | 3-10-5 | 766 |
| 599247 | 2589 | 2606 | Exon 18 | TCAATCCCACGCCCCTGT | 61 | 7824 | 7841 | 3-10-5 | 767 |
| 599248 | 2590 | 2607 | Exon 18 | TTCAATCCCACGCCCCTG | 51 | 7825 | 7842 | 3-10-5 | 768 |
| 599249 | 2591 | 2608 | Exon 18 | ATTCAATCCCACGCCCCT | 58 | 7826 | 7843 | 3-10-5 | 769 |
| 599250 | 2592 | 2609 | Exon 18 | AATTCAATCCCACGCCCC | 49 | 7827 | 7844 | 3-10-5 | 770 |
| 599251 | 2593 | 2610 | Exon 18 | TAATTCAATCCCACGCCC | 46 | 7828 | 7845 | 3-10-5 | 771 |
| 599252 | 2594 | 2611 | Exon 18 | TTAATTCAATCCCACGCC | 32 | 7829 | 7846 | 3-10-5 | 772 |
| 599253 | 2595 | 2612 | Exon 18 | TTTAATTCAATCCCACGC | 23 | 7830 | 7847 | 3-10-5 | 773 |
| 599254 | 2596 | 2613 | Exon 18 | TTTTAATTCAATCCCACG | 0 | 7831 | 7848 | 3-10-5 | 774 |
| 599255 | 2597 | 2614 | Exon 18 | GTTTTAATTCAATCCCAC | 61 | 7832 | 7849 | 3-10-5 | 775 |
| 599256 | 2598 | 2615 | Exon 18 | TGTTTTAATTCAATCCCA | 64 | 7833 | 7850 | 3-10-5 | 776 |
| 599257 | 2599 | 2616 | Exon 18 | CTGTTTTAATTCAATCCC | 66 | 7834 | 7851 | 3-10-5 | 777 |
| 599258 | 2600 | 2617 | Exon 18 | GCTGTTTTAATTCAATCC | 59 | 7835 | 7852 | 3-10-5 | 778 |
| 599259 | 2601 | 2618 | Exon 18 | AGCTGTTTTAATTCAATC | 40 | 7836 | 7853 | 3-10-5 | 779 |
| 599260 | 2602 | 2619 | Exon 18 | CAGCTGTTTTAATTCAAT | 38 | 7837 | 7854 | 3-10-5 | 780 |
| 599261 | 2603 | 2620 | Exon 18 | GCAGCTGTTTTAATTCAA | 54 | 7838 | 7855 | 3-10-5 | 781 |
| 599509 | 2552 | 2570 | Exon 18 | TAGAAAACCCAAATCCTCA | 54 | 7787 | 7805 | 6-7-6 | 681 |
| 599273 | 2553 | 2571 | Exon 18 | ATAGAAAACCCAAATCCTC | 0 | 7788 | 7806 | 6-7-6 | 682 |
| 599274 | 2554 | 2572 | Exon 18 | TATAGAAAACCCAAATCCT | 57 | 7789 | 7807 | 6-7-6 | 683 |
| 599275 | 2556 | 2574 | Exon 18 | CTTATAGAAAACCCAAATC | 0 | 7791 | 7809 | 6-7-6 | 684 |
| 599276 | 2557 | 2575 | Exon 18 | CCTTATAGAAAACCCAAAT | 44 | 7792 | 7810 | 6-7-6 | 685 |
| 599277 | 2558 | 2576 | Exon 18 | CCCTTATAGAAAACCCAAA | 0 | 7793 | 7811 | 6-7-6 | 686 |
| 599278 | 2559 | 2577 | Exon 18 | CCCCTTATAGAAAACCCAA | 0 | 7794 | 7812 | 6-7-6 | 687 |
| 599279 | 2560 | 2578 | Exon 18 | ACCCCTTATAGAAAACCCA | 20 | 7795 | 7813 | 6-7-6 | 688 |
| 599280 | 2561 | 2579 | Exon 18 | AACCCCTTATAGAAAACCC | 70 | 7796 | 7814 | 6-7-6 | 689 |
| 532917 | 2604 | 2623 | Exon 18 | GTCGCAGCTGTTTTAATTCA | 85 | 7839 | 7858 | 5-10-5 | 317 |
| 599262 | 2604 | 2621 | Exon 18 | CGCAGCTGTTTTAATTCA | 49 | 7839 | 7856 | 3-10-5 | 782 |
| 599263 | 2605 | 2622 | Exon 18 | TCGCAGCTGTTTTAATTC | 49 | 7840 | 7857 | 3-10-5 | 783 |
| 599264 | 2606 | 2623 | Exon 18 | GTCGCAGCTGTTTTAATT | 62 | 7841 | 7858 | 3-10-5 | 784 |
| 599265 | 2607 | 2624 | Exon 18 | TGTCGCAGCTGTTTTAAT | 63 | 7842 | 7859 | 3-10-5 | 792 |
| 599266 | 2608 | 2625 | Exon 18 | TTGTCGCAGCTGTTTTAA | 41 | 7843 | 7860 | 3-10-5 | 793 |
| 599267 | 2609 | 2626 | Exon 18 | GTTGTCGCAGCTGTTTTA | 52 | 7844 | 7861 | 3-10-5 | 794 |
| 599268 | 2610 | 2627 | Exon 18 | TGTTGTCGCAGCTGTTTT | 51 | 7845 | 7862 | 3-10-5 | 795 |
| 599269 | 2611 | 2628 | Exon 18/ | TTGTTGTCGCAGCTGTTT | 58 | n/a | n/a | 3-10-5 | 796 |
| | | | Repeat | | | | | | |
| 599270 | 2612 | 2629 | Exon 18/ | TTTGTTGTCGCAGCTGTT | 69 | n/a | n/a | 3-10-5 | 797 |
| | | | Repeat | | | | | | |
| 599271 | 2613 | 2630 | Exon 18/ | TTTTGTTGTCGCAGCTGT | 69 | n/a | n/a | 3-10-5 | 798 |
| | | | Repeat | | | | | | |
| 599272 | 2614 | 2631 | Exon 18/ | TTTTTGTTGTCGCAGCTG | 72 | n/a | n/a | 3-10-5 | 799 |
| | | | Repeat | | | | | | |
| 599205 | 2607 | 2624 | Exon 18 | TGTCGCAGCTGTTTTAAT | 54 | 7842 | 7859 | 5-8-5 | 792 |
| 599206 | 2608 | 2625 | Exon 18 | TTGTCGCAGCTGTTTTAA | 62 | 7843 | 7860 | 5-8-5 | 793 |
| 599207 | 2609 | 2626 | Exon 18 | GTTGTCGCAGCTGTTTTA | 62 | 7844 | 7861 | 5-8-5 | 794 |
| 599208 | 2610 | 2627 | Exon 18 | TGTTGTCGCAGCTGTTTT | 66 | 7845 | 7862 | 5-8-5 | 795 |
| 599209 | 2611 | 2628 | Exon 18/ | TTGTTGTCGCAGCTGTTT | 60 | n/a | n/a | 5-8-5 | 796 |
| | | | Repeat | | | | | | |
| 599210 | 2612 | 2629 | Exon 18/ | TTTGTTGTCGCAGCTGTT | 62 | n/a | n/a | 5-8-5 | 797 |
| | | | Repeat | | | | | | |
| 599211 | 2613 | 2630 | Exon 18/ | TTTTGTTGTCGCAGCTGT | 65 | n/a | n/a | 5-8-5 | 798 |
| | | | Repeat | | | | | | |
| 599212 | 2614 | 2631 | Exon 18/ | TTTTTGTTGTCGCAGCTG | 67 | n/a | n/a | 5-8-5 | 799 |
| | | | Repeat | | | | | | |

TABLE 24
Inhibition of CFB mRNA by 5-10-5 MOE gapmers targeting SEQ ID NO: 1 or SEQ ID NO: 2

| | SEQ | SEQ | | | | SEQ | SEQ | |
|---|---|---|---|---|---|---|---|---|
| | ID | ID | | | | ID | ID | |
| | NO: 1 | NO: 1 | | | % | NO: 2 | NO: 2 | SEQ |
| ISIS | start | stop | Target | | in- | start | stop | ID |
| NO | site | site | region | Sequence | hibition | site | site | NO: |
| 588570 | 150 | 169 | Exon 1 | TGGTCACATTCCCTTCCCCT | 72 | 1871 | 1890 | 396 |
| 588571 | 152 | 171 | Exon 1 | CCTGGTCACATTCCCTTCCC | 80 | 1873 | 1892 | 397 |
| 532614 | 154 | 173 | Exon 1 | GACCTGGTCACATTCCCTTC | 65 | 1875 | 1894 | 12 |
| 588572 | 156 | 175 | Exon 1 | TAGACCTGGTCACATTCCCT | 74 | 1877 | 1896 | 398 |
| 588573 | 158 | 177 | Exon 1 | CCTAGACCTGGTCACATTCC | 72 | 1879 | 1898 | 399 |
| 588566 | 2189 | 2208 | Exon 15 | CCTTCCGAGTCAGCTTTTTC | 66 | 6977 | 6996 | 400 |
| 588567 | 2191 | 2210 | Exon 15 | CTCCTTCCGAGTCAGCTTTT | 66 | 6979 | 6998 | 401 |
| 532770 | 2193 | 2212 | Exon 15 | ACCTCCTTCCGAGTCAGCTT | 64 | 6981 | 7000 | 198 |
| 588568 | 2195 | 2214 | Exon 15 | AGACCTCCTTCCGAGTCAGC | 78 | 6983 | 7002 | 402 |
| 588569 | 2197 | 2216 | Exon 15 | GTAGACCTCCTTCCGAGTCA | 74 | 6985 | 7004 | 403 |
| 588574 | 2453 | 2472 | Exon 18 | TTTGCCGCTTCTGGTTTTTG | 71 | 7688 | 7707 | 404 |
| 588575 | 2455 | 2474 | Exon 18 | CTTTTGCCGCTTCTGGTTTT | 72 | 7690 | 7709 | 405 |
| 532800 | 2457 | 2476 | Exon 18 | TGCTTTTGCCGCTTCTGGTT | 71 | 7692 | 7711 | 228 |
| 588576 | 2459 | 2478 | Exon 18 | CCTGCTTTTGCCGCTTCTGG | 59 | 7694 | 7713 | 406 |
| 588577 | 2461 | 2480 | Exon 18 | TACCTGCTTTTGCCGCTTCT | 76 | 7696 | 7715 | 407 |
| 516350 | 2550 | 2569 | Exon 18 | AGAAAACCCAAATCCTCATC | 58 | 7785 | 7804 | 408 |
| 588509 | 2551 | 2570 | Exon 18 | TAGAAAACCCAAATCCTCAT | 6 | 7786 | 7805 | 409 |
| 588510 | 2552 | 2571 | Exon 18 | ATAGAAAACCCAAATCCTCA | 10 | 7787 | 7806 | 410 |
| 588511 | 2553 | 2572 | Exon 18 | TATAGAAAACCCAAATCCTC | 9 | 7788 | 7807 | 411 |
| 588512 | 2554 | 2573 | Exon 18 | TTATAGAAAACCCAAATCCT | 80 | 7789 | 7808 | 412 |
| 588513 | 2555 | 2574 | Exon 18 | CTTATAGAAAACCCAAATCC | 70 | 7790 | 7809 | 413 |
| 588514 | 2556 | 2575 | Exon 18 | CCTTATAGAAAACCCAAATC | 71 | 7791 | 7810 | 414 |
| 588515 | 2557 | 2576 | Exon 18 | CCCTTATAGAAAACCCAAAT | 78 | 7792 | 7811 | 415 |
| 588516 | 2558 | 2577 | Exon 18 | CCCCTTATAGAAAACCCAAA | 72 | 7793 | 7812 | 416 |
| 588517 | 2559 | 2578 | Exon 18 | ACCCCTTATAGAAAACCCAA | 80 | 7794 | 7813 | 417 |
| 588518 | 2560 | 2579 | Exon 18 | AACCCCTTATAGAAAACCCA | 80 | 7795 | 7814 | 418 |
| 588519 | 2561 | 2580 | Exon 18 | AAACCCCTTATAGAAAACCC | 62 | 7796 | 7815 | 419 |
| 588520 | 2562 | 2581 | Exon 18 | GAAACCCCTTATAGAAAACC | 59 | 7797 | 7816 | 420 |
| 588521 | 2563 | 2582 | Exon 18 | GGAAACCCCTTATAGAAAAC | 40 | 7798 | 7817 | 421 |
| 588522 | 2564 | 2583 | Exon 18 | AGGAAACCCCTTATAGAAAA | 66 | 7799 | 7818 | 422 |
| 588523 | 2565 | 2584 | Exon 18 | CAGGAAACCCCTTATAGAAA | 63 | 7800 | 7819 | 423 |
| 588524 | 2566 | 2585 | Exon 18 | GCAGGAAACCCCTTATAGAA | 70 | 7801 | 7820 | 424 |
| 588525 | 2567 | 2586 | Exon 18 | AGCAGGAAACCCCTTATAGA | 67 | 7802 | 7821 | 425 |
| 588526 | 2568 | 2587 | Exon 18 | CAGCAGGAAACCCCTTATAG | 0 | 7803 | 7822 | 426 |
| 588527 | 2569 | 2588 | Exon 18 | CCAGCAGGAAACCCCTTATA | 11 | 7804 | 7823 | 427 |
| 588528 | 2570 | 2589 | Exon 18 | TCCAGCAGGAAACCCCTTAT | 15 | 7805 | 7824 | 428 |
| 532809 | 2571 | 2590 | Exon 18 | GTCCAGCAGGAAACCCCTTA | 75 | 7806 | 7825 | 237 |
| 588529 | 2572 | 2591 | Exon 18 | TGTCCAGCAGGAAACCCCTT | 16 | 7807 | 7826 | 429 |
| 588530 | 2573 | 2592 | Exon 18 | CTGTCCAGCAGGAAACCCCT | 16 | 7808 | 7827 | 430 |
| 588531 | 2574 | 2593 | Exon 18 | CCTGTCCAGCAGGAAACCCC | 19 | 7809 | 7828 | 431 |
| 588532 | 2575 | 2594 | Exon 18 | CCCTGTCCAGCAGGAAACCC | 15 | 7810 | 7829 | 432 |
| 588533 | 2576 | 2595 | Exon 18 | CCCCTGTCCAGCAGGAAACC | 29 | 7811 | 7830 | 433 |
| 532810 | 2577 | 2596 | Exon 18 | GCCCCTGTCCAGCAGGAAAC | 74 | 7812 | 7831 | 238 |
| 588534 | 2578 | 2597 | Exon 18 | CGCCCCTGTCCAGCAGGAAA | 21 | 7813 | 7832 | 434 |
| 588535 | 2579 | 2598 | Exon 18 | ACGCCCCTGTCCAGCAGGAA | 16 | 7814 | 7833 | 435 |
| 588536 | 2580 | 2599 | Exon 18 | CACGCCCCTGTCCAGCAGGA | 0 | 7815 | 7834 | 436 |
| 588537 | 2581 | 2600 | Exon 18 | CCACGCCCCTGTCCAGCAGG | 8 | 7816 | 7835 | 437 |
| 588538 | 2582 | 2601 | Exon 18 | CCCACGCCCCTGTCCAGCAG | 10 | 7817 | 7836 | 438 |
| 588539 | 2583 | 2602 | Exon 18 | TCCCACGCCCCTGTCCAGCA | 23 | 7818 | 7837 | 439 |
| 588540 | 2584 | 2603 | Exon 18 | ATCCCACGCCCCTGTCCAGC | 16 | 7819 | 7838 | 440 |
| 588541 | 2585 | 2604 | Exon 18 | AATCCCACGCCCCTGTCCAG | 16 | 7820 | 7839 | 441 |
| 588542 | 2586 | 2605 | Exon 18 | CAATCCCACGCCCCTGTCCA | 12 | 7821 | 7840 | 442 |
| 588543 | 2587 | 2606 | Exon 18 | TCAATCCCACGCCCCTGTCC | 26 | 7822 | 7841 | 443 |
| 588544 | 2588 | 2607 | Exon 18 | TTCAATCCCACGCCCCTGTC | 26 | 7823 | 7842 | 444 |
| 588545 | 2589 | 2608 | Exon 18 | ATTCAATCCCACGCCCCTGT | 31 | 7824 | 7843 | 445 |
| 588546 | 2590 | 2609 | Exon 18 | AATTCAATCCCACGCCCCTG | 22 | 7825 | 7844 | 446 |
| 588547 | 2591 | 2610 | Exon 18 | TAATTCAATCCCACGCCCCT | 12 | 7826 | 7845 | 447 |
| 588548 | 2592 | 2611 | Exon 18 | TTAATTCAATCCCACGCCCC | 20 | 7827 | 7846 | 448 |
| 588549 | 2593 | 2612 | Exon 18 | TTTAATTCAATCCCACGCCC | 26 | 7828 | 7847 | 449 |
| 588550 | 2594 | 2613 | Exon 18 | TTTTAATTCAATCCCACGCC | 32 | 7829 | 7848 | 450 |
| 588551 | 2595 | 2614 | Exon 18 | GTTTTAATTCAATCCCACGC | 48 | 7830 | 7849 | 451 |
| 588552 | 2596 | 2615 | Exon 18 | TGTTTTAATTCAATCCCACG | 57 | 7831 | 7850 | 452 |
| 588553 | 2597 | 2616 | Exon 18 | CTGTTTTAATTCAATCCCAC | 49 | 7832 | 7851 | 453 |
| 588554 | 2598 | 2617 | Exon 18 | GCTGTTTTAATTCAATCCCA | 64 | 7833 | 7852 | 454 |
| 532811 | 2599 | 2618 | Exon 18 | AGCTGTTTTAATTCAATCCC | 78 | 7834 | 7853 | 239 |
| 588555 | 2600 | 2619 | Exon 18 | CAGCTGTTTTAATTCAATCC | 48 | 7835 | 7854 | 455 |
| 588556 | 2601 | 2620 | Exon 18 | GCAGCTGTTTTAATTCAATC | 55 | 7836 | 7855 | 456 |
| 588557 | 2602 | 2621 | Exon 18 | CGCAGCTGTTTTAATTCAAT | 51 | 7837 | 7856 | 457 |
| 588558 | 2603 | 2622 | Exon 18 | TCGCAGCTGTTTTAATTCAA | 51 | 7838 | 7857 | 458 |
| 532917 | 2604 | 2623 | Exon 18 | GTCGCAGCTGTTTTAATTCA | 82 | 7839 | 7858 | 317 |
| 588559 | 2605 | 2624 | Exon 18 | TGTCGCAGCTGTTTTAATTC | 58 | 7840 | 7859 | 459 |
| 588560 | 2606 | 2625 | Exon 18 | TTGTCGCAGCTGTTTTAATT | 72 | 7841 | 7860 | 460 |
| 588561 | 2607 | 2626 | Exon 18 | GTTGTCGCAGCTGTTTTAAT | 75 | 7842 | 7861 | 461 |
| 532952 | 2608 | 2627 | Exon 18 | TGTTGTCGCAGCTGTTTTAA | 39 | 7843 | 7862 | 395 |
| 588562 | 2609 | 2628 | Exon 18/ | TTGTTGTCGCAGCTGTTTTA | 53 | n/a | n/a | 462 |
| | | | Repeat | | | | | |
| 588563 | 2610 | 2629 | Exon 18/ | TTTGTTGTCGCAGCTGTTTT | 62 | n/a | n/a | 463 |
| | | | Repeat | | | | | |
| 588564 | 2611 | 2630 | Exon 18/ | TTTTGTTGTCGCAGCTGTTT | 63 | n/a | n/a | 464 |
| | | | Repeat | | | | | |
| 588565 | 2612 | 2631 | Exon 18/ | TTTTTGTTGTCGCAGCTGTT | 64 | n/a | n/a | 465 |
| | | | Repeat | | | | | |

### Example 8: Dose-Dependent Antisense Inhibition of Human CFB in HepG2 Cells by 5-10-5 MOE Gapmers

Gapmers from studies described above exhibiting in vitro inhibition of CFB mRNA were selected and tested at various doses in HepG2 cells. Cells were plated at a density of 20,000 cells per well and transfected using electroporation with 0.313 μM, 0.625 μM, 1.25 μM, 2.50 UM, 5.00 UM, or 10.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 25

| | 0.313 | 0.625 | 1.25 | 2.50 | 5.00 | 10.00 | IC₅₀ |
|---|---|---|---|---|---|---|---|
| ISIS No | μM | μM | μM | μM | μM | μM | (μM) |
| 532614 | 7 | 13 | 43 | 72 | 65 | 71 | 2.2 |
| 532635 | 12 | 0 | 3 | 28 | 0 | 0 | >10 |
| 532692 | 26 | 0 | 12 | 52 | 55 | 74 | 3.7 |
| 532770 | 21 | 18 | 32 | 73 | 64 | 88 | 1.8 |
| 532775 | 8 | 0 | 26 | 35 | 47 | 59 | 6.2 |
| 532800 | 0 | 5 | 30 | 65 | 50 | 75 | 3.1 |
| 532809 | 12 | 30 | 28 | 40 | 46 | 66 | 4.6 |
| 532810 | 28 | 44 | 32 | 69 | 84 | 95 | 1.2 |
| 532811 | 66 | 83 | 90 | 94 | 97 | 99 | <0.3 |
| 532917 | 64 | 85 | 88 | 96 | 97 | 99 | <0.3 |
| 532952 | 50 | 53 | 68 | 80 | 91 | 94 | 0.4 |

### Example 9: Dose-Dependent Antisense Inhibition of Human CFB in HepG2 Cells

Gapmers from studies described above exhibiting in vitro inhibition of CFB mRNA were selected and tested at various doses in HepG2 cells. The antisense oligonucleotides were tested in a number of experiments with similar culture conditions. The results for each experiment are presented in separate tables shown below. Cells were plated at a density of 20,000 cells per well and transfected using electroporation with 0.08 μM, 0.25 μM, 0.74 M, 2.22 μM, 6.67 μM, and 20.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 26

| | 0.08 | 0.25 | 0.74 | 2.22 | 6.67 | 20.00 | IC₅₀ |
|---|---|---|---|---|---|---|---|
| ISIS No | μM | μM | μM | μM | μM | μM | (μM) |
| 532811 | 19 | 53 | 81 | 87 | 96 | 97 | 0.2 |
| 588834 | 7 | 42 | 64 | 92 | 98 | 98 | 0.5 |
| 588835 | 11 | 30 | 66 | 89 | 97 | 97 | 0.5 |
| 588836 | 14 | 40 | 61 | 91 | 97 | 97 | 0.5 |
| 588837 | 6 | 39 | 67 | 89 | 96 | 97 | 0.5 |
| 588838 | 0 | 27 | 41 | 81 | 87 | 97 | 1.0 |
| 588842 | 17 | 51 | 68 | 86 | 93 | 95 | 0.3 |
| 588843 | 21 | 38 | 72 | 90 | 95 | 96 | 0.4 |
| 588870 | 9 | 31 | 56 | 88 | 95 | 97 | 0.6 |
| 588871 | 14 | 25 | 47 | 79 | 93 | 97 | 0.7 |
| 588872 | 18 | 28 | 59 | 84 | 92 | 97 | 0.6 |

TABLE 27

| | 0.08 | 0.25 | 0.74 | 2.22 | 6.67 | 20.00 | IC₅₀ |
|---|---|---|---|---|---|---|---|
| ISIS No | μM | μM | μM | μM | μM | μM | (μM) |
| 532811 | 31 | 70 | 89 | 94 | 97 | 97 | 0.1 |
| 588844 | 31 | 60 | 77 | 91 | 95 | 96 | 0.1 |
| 588846 | 32 | 52 | 78 | 89 | 95 | 97 | 0.2 |
| 588847 | 22 | 52 | 77 | 91 | 95 | 97 | 0.2 |
| 588848 | 20 | 40 | 73 | 91 | 96 | 98 | 0.3 |
| 588851 | 40 | 52 | 82 | 94 | 97 | 97 | 0.1 |
| 588854 | 17 | 55 | 59 | 84 | 94 | 96 | 0.4 |
| 588855 | 10 | 32 | 56 | 82 | 93 | 96 | 0.6 |
| 588856 | 13 | 46 | 75 | 90 | 96 | 97 | 0.3 |
| 588857 | 11 | 52 | 73 | 94 | 96 | 97 | 0.3 |
| 588858 | 19 | 48 | 75 | 94 | 97 | 98 | 0.3 |

TABLE 28

| | 0.08 | 0.25 | 0.74 | 2.22 | 6.67 | 20.00 | IC₅₀ |
|---|---|---|---|---|---|---|---|
| ISIS No | μM | μM | μM | μM | μM | μM | (μM) |
| 532811 | 42 | 66 | 88 | 96 | 97 | 98 | 0.1 |
| 588859 | 18 | 46 | 66 | 90 | 96 | 97 | 0.4 |
| 588860 | 55 | 80 | 94 | 97 | 97 | 97 | <0.1 |
| 588861 | 24 | 61 | 86 | 93 | 96 | 97 | 0.2 |
| 588862 | 25 | 64 | 85 | 94 | 96 | 98 | 0.1 |
| 588863 | 50 | 73 | 89 | 96 | 96 | 98 | <0.1 |
| 588864 | 52 | 80 | 92 | 96 | 98 | 98 | <0.1 |
| 588865 | 46 | 72 | 91 | 96 | 96 | 99 | <0.1 |
| 588866 | 47 | 76 | 88 | 96 | 97 | 98 | <0.1 |
| 588867 | 43 | 69 | 83 | 92 | 96 | 99 | 0.1 |
| 588868 | 43 | 56 | 65 | 84 | 93 | 97 | 0.1 |

TABLE 29

| | 0.08 | 0.25 | 0.74 | 2.22 | 6.67 | 20.00 | IC₅₀ |
|---|---|---|---|---|---|---|---|
| ISIS No | μM | μM | μM | μM | μM | μM | (μM) |
| 532810 | 0 | 14 | 38 | 72 | 89 | 96 | 1.2 |
| 532811 | 18 | 54 | 79 | 93 | 96 | 97 | 0.3 |
| 532952 | 19 | 34 | 73 | 87 | 94 | 96 | 0.4 |
| 588534 | 17 | 13 | 44 | 77 | 93 | 97 | 0.9 |
| 588544 | 12 | 43 | 69 | 86 | 89 | 93 | 0.4 |
| 588545 | 17 | 55 | 67 | 86 | 91 | 93 | 0.3 |
| 588546 | 10 | 32 | 67 | 85 | 91 | 93 | 0.6 |
| 588552 | 27 | 54 | 76 | 90 | 94 | 97 | 0.2 |
| 588553 | 32 | 68 | 87 | 93 | 95 | 97 | 0.1 |
| 588560 | 16 | 54 | 76 | 90 | 94 | 96 | 0.3 |
| 588561 | 18 | 45 | 68 | 85 | 93 | 96 | 0.4 |

TABLE 30

| | 0.08 | 0.25 | 0.74 | 2.22 | 6.67 | 20.00 | IC₅₀ |
|---|---|---|---|---|---|---|---|
| ISIS No | μM | μM | μM | μM | μM | μM | (μM) |
| 532811 | 22 | 60 | 82 | 94 | 97 | 98 | 0.2 |
| 588536 | 2 | 38 | 65 | 90 | 96 | 97 | 0.6 |
| 588537 | 12 | 38 | 63 | 87 | 94 | 97 | 0.5 |
| 588547 | 19 | 35 | 61 | 86 | 93 | 97 | 0.5 |
| 588548 | 19 | 36 | 75 | 88 | 95 | 96 | 0.4 |
| 588554 | 0 | 76 | 92 | 95 | 97 | 97 | <0.1 |
| 588555 | 31 | 61 | 89 | 96 | 97 | 98 | 0.1 |
| 588556 | 33 | 56 | 82 | 95 | 94 | 97 | 0.1 |
| 588562 | 12 | 39 | 71 | 87 | 94 | 97 | 0.4 |
| 588563 | 25 | 48 | 72 | 86 | 94 | 96 | 0.3 |
| 588564 | 15 | 33 | 63 | 89 | 91 | 97 | 0.5 |

TABLE 31

| | 0.08 | 0.25 | 0.74 | 2.22 | 6.67 | 20.00 | IC₅₀ |
|---|---|---|---|---|---|---|---|
| ISIS No | μM | μM | μM | μM | μM | μM | (μM) |
| 532811 | 39 | 68 | 86 | 96 | 98 | 98 | 0.1 |
| 588538 | 0 | 40 | 82 | 94 | 97 | 98 | 0.3 |
| 588539 | 34 | 65 | 88 | 95 | 98 | 98 | 0.1 |
| 588540 | 30 | 51 | 81 | 91 | 97 | 98 | 0.2 |
| 588549 | 31 | 57 | 82 | 95 | 96 | 98 | 0.1 |
| 588550 | 34 | 65 | 88 | 96 | 98 | 98 | 0.1 |
| 588551 | 47 | 66 | 87 | 96 | 98 | 99 | <0.1 |
| 588557 | 40 | 84 | 95 | 98 | 98 | 98 | <0.1 |
| 588558 | 45 | 73 | 93 | 97 | 98 | 99 | <0.1 |
| 588559 | 51 | 69 | 83 | 96 | 98 | 99 | <0.1 |
| 588565 | 19 | 56 | 81 | 92 | 96 | 98 | 0.2 |

### Example 10: Dose-Dependent Antisense Inhibition of Human CFB in HepG2 Cells

Gapmers from studies described above exhibiting in vitro inhibition of CFB mRNA were selected and tested at various doses in HepG2 cells. The antisense oligonucleotides were tested in a number of experiments with similar culture conditions. The results for each experiment are presented in separate tables shown below. Cells were plated at a density of 20,000 cells per well and transfected using electroporation with 0.06 μM, 0.25 μM, 1.00 μM, and 4.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 32

| ISIS | 0.06 | 0.25 | 1.00 | 4.00 | IC₅₀ |
|---|---|---|---|---|---|
| No | μM | μM | μM | μM | (μM) |
| 532917 | 31 | 58 | 87 | 92 | 0.2 |
| 588860 | 18 | 50 | 79 | 93 | 0.3 |
| 599001 | 16 | 28 | 69 | 90 | 0.5 |
| 599024 | 14 | 32 | 74 | 90 | 0.4 |
| 599025 | 0 | 31 | 56 | 92 | 0.7 |
| 599032 | 28 | 44 | 62 | 88 | 0.3 |
| 599033 | 28 | 46 | 80 | 92 | 0.2 |
| 599077 | 8 | 20 | 59 | 80 | 0.8 |
| 599080 | 9 | 33 | 48 | 76 | 0.9 |
| 599086 | 7 | 22 | 53 | 83 | 0.8 |
| 599087 | 21 | 31 | 74 | 87 | 0.4 |
| 599088 | 13 | 37 | 69 | 82 | 0.5 |
| 599089 | 3 | 36 | 55 | 79 | 0.7 |
| 599093 | 25 | 59 | 79 | 88 | 0.2 |
| 599094 | 19 | 29 | 75 | 89 | 0.4 |
| 599095 | 29 | 43 | 67 | 87 | 0.3 |
| 599096 | 23 | 51 | 70 | 88 | 0.3 |
| 599149 | 20 | 53 | 82 | 92 | 0.3 |
| 599188 | 0 | 21 | 62 | 85 | 0.8 |

TABLE 33

| ISIS | 0.06 | 0.25 | 1.00 | 4.00 | IC₅₀ |
|---|---|---|---|---|---|
| No | μM | μM | μM | μM | (μM) |
| 532917 | 0 | 42 | 81 | 91 | 0.4 |
| 588860 | 17 | 49 | 74 | 92 | 0.3 |
| 599155 | 29 | 52 | 67 | 87 | 0.3 |
| 599198 | 3 | 25 | 64 | 89 | 0.6 |
| 599201 | 13 | 26 | 67 | 91 | 0.5 |
| 599202 | 0 | 44 | 72 | 87 | 0.5 |
| 599203 | 22 | 41 | 75 | 88 | 0.3 |
| 599314 | 12 | 34 | 71 | 84 | 0.5 |
| 599316 | 7 | 37 | 66 | 88 | 0.5 |
| 599317 | 8 | 1 | 54 | 83 | 1.0 |
| 599321 | 8 | 33 | 70 | 85 | 0.5 |
| 599322 | 24 | 38 | 66 | 87 | 0.4 |
| 599327 | 22 | 32 | 66 | 89 | 0.4 |
| 599328 | 0 | 31 | 59 | 88 | 0.7 |
| 599330 | 5 | 43 | 67 | 84 | 0.5 |
| 599374 | 23 | 42 | 80 | 91 | 0.3 |
| 599378 | 21 | 57 | 80 | 93 | 0.2 |
| 599380 | 23 | 56 | 82 | 93 | 0.2 |
| 599432 | 17 | 37 | 73 | 93 | 0.4 |

TABLE 34

| ISIS | 0.06 | 0.25 | 1.00 | 4.00 | IC₅₀ |
|---|---|---|---|---|---|
| No | μM | μM | μM | μM | (μM) |
| 532917 | 23 | 65 | 76 | 93 | 0.2 |
| 588860 | 17 | 60 | 76 | 90 | 0.3 |
| 601282 | 48 | 68 | 81 | 88 | 0.1 |
| 601269 | 18 | 59 | 80 | 94 | 0.2 |
| 601276 | 34 | 64 | 81 | 91 | 0.1 |
| 601275 | 14 | 39 | 78 | 90 | 0.4 |
| 601344 | 52 | 84 | 92 | 94 | <0.06 |
| 601383 | 53 | 81 | 86 | 94 | <0.06 |
| 601382 | 41 | 76 | 88 | 94 | 0.1 |
| 601385 | 52 | 74 | 89 | 91 | <0.06 |
| 601332 | 41 | 69 | 86 | 94 | 0.1 |
| 601345 | 36 | 75 | 86 | 95 | 0.1 |
| 601371 | 34 | 72 | 91 | 93 | 0.1 |
| 601384 | 50 | 78 | 91 | 95 | <0.06 |
| 601380 | 28 | 57 | 83 | 92 | 0.2 |
| 601387 | 48 | 61 | 82 | 88 | 0.1 |
| 601341 | 28 | 65 | 83 | 91 | 0.2 |
| 601346 | 31 | 69 | 82 | 93 | 0.1 |
| 601335 | 24 | 56 | 85 | 92 | 0.2 |

TABLE 35

| ISIS | 0.06 | 0.25 | 1.00 | 4.00 | IC₅₀ |
|---|---|---|---|---|---|
| No | μM | μM | μM | μM | (μM) |
| 532917 | 31 | 66 | 86 | 93 | 0.1 |
| 588860 | 28 | 62 | 85 | 94 | 0.2 |
| 599208 | 24 | 50 | 71 | 89 | 0.3 |
| 599261 | 31 | 49 | 81 | 94 | 0.2 |
| 599267 | 41 | 48 | 80 | 88 | 0.2 |
| 599268 | 28 | 56 | 75 | 92 | 0.2 |
| 599313 | 14 | 24 | 71 | 92 | 0.5 |
| 599441 | 24 | 57 | 80 | 87 | 0.2 |
| 599494 | 13 | 55 | 86 | 94 | 0.3 |
| 599552 | 30 | 69 | 93 | 95 | 0.1 |
| 599553 | 34 | 71 | 93 | 96 | 0.1 |
| 599554 | 30 | 74 | 93 | 96 | 0.1 |
| 599568 | 40 | 77 | 90 | 97 | 0.1 |
| 599570 | 61 | 82 | 93 | 96 | <0.06 |
| 599577 | 18 | 62 | 81 | 93 | 0.2 |
| 599581 | 27 | 60 | 80 | 94 | 0.2 |
| 599591 | 49 | 74 | 93 | 96 | <0.06 |
| 599592 | 46 | 76 | 90 | 94 | 0.1 |
| 599593 | 44 | 72 | 91 | 95 | 0.1 |

TABLE 36

| ISIS | 0.06 | 0.25 | 1.00 | 4.00 | IC₅₀ |
|---|---|---|---|---|---|
| No | μM | μM | μM | μM | (μM) |
| 532917 | 25 | 56 | 84 | 92 | 0.2 |
| 588860 | 11 | 51 | 80 | 92 | 0.3 |
| 599547 | 23 | 60 | 82 | 90 | 0.2 |
| 599569 | 42 | 73 | 85 | 88 | 0.1 |
| 599578 | 29 | 49 | 82 | 89 | 0.2 |
| 599582 | 21 | 56 | 78 | 91 | 0.2 |
| 599590 | 24 | 62 | 80 | 90 | 0.2 |
| 601209 | 21 | 49 | 85 | 88 | 0.3 |
| 601210 | 34 | 64 | 86 | 92 | 0.1 |
| 601212 | 46 | 68 | 88 | 90 | 0.1 |
| 601213 | 54 | 80 | 90 | 92 | <0.06 |
| 601214 | 38 | 77 | 88 | 95 | 0.1 |
| 601215 | 42 | 64 | 85 | 92 | 0.1 |
| 601216 | 45 | 57 | 76 | 89 | 0.1 |
| 601264 | 29 | 58 | 86 | 95 | 0.2 |
| 601278 | 51 | 82 | 83 | 93 | <0.06 |
| 601279 | 44 | 80 | 92 | 96 | 0.1 |
| 601280 | 44 | 73 | 87 | 94 | 0.1 |
| 601281 | 51 | 80 | 91 | 94 | <0.06 |

### Example 11: Dose-Dependent Antisense Inhibition of Human CFB in HepG2 Cells

Gapmers from studies described above exhibiting in vitro inhibition of CFB mRNA were selected and tested at various doses in HepG2 cells. Additionally, a deoxy, MOE and cEt oligonucleotide, ISIS 594430, was designed with the same sequence (CTCCTTCCGAGTCAGC, SEQ ID NO: 549) and target region (target start site 2195 of SEQ ID NO: 1 and target start site 6983 of SED ID NO: 2) as ISIS 588870, another deoxy, MOE, and cEt oligonucleotide. ISIS 594430 is a 3-10-3 cEt gapmer.

Cells were plated at a density of 20,000 cells per well and transfected using electroporation with 0.01 μM, 0.04 μM, 0.12 μM, 0.37 μM, 1.11 μM, 3.33 μM, and 10.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 37

| | 0.01 | 0.04 | 0.12 | 0.37 | 1.11 | 3.33 | 10.00 | IC₅₀ |
|---|---|---|---|---|---|---|---|---|
| ISIS No | μM | μM | μM | μM | μM | μM | μM | (μM) |
| 588536 | 0 | 0 | 0 | 5 | 45 | 73 | 94 | 1.4 |
| 588548 | 0 | 0 | 0 | 19 | 52 | 78 | 90 | 1.2 |
| 588553 | 0 | 0 | 9 | 42 | 76 | 85 | 94 | 0.6 |
| 588555 | 0 | 52 | 23 | 58 | 78 | 83 | 95 | 0.3 |
| 588847 | 4 | 1 | 18 | 45 | 67 | 84 | 96 | 0.5 |
| 588848 | 0 | 3 | 13 | 38 | 67 | 83 | 95 | 0.6 |
| 594430 | 0 | 0 | 10 | 34 | 50 | 55 | 84 | 1.4 |

### Example 12: Tolerability of MOE Gapmers Targeting Human CFB in CD1 Mice

CD1® mice (Charles River, MA) are a multipurpose mouse model, frequently utilized for safety and efficacy testing. The mice were treated with ISIS antisense oligonucleotides selected from studies described above and evaluated for changes in the levels of various plasma chemistry markers.

Study 1 (with 5-10-5 MOE Gapmers)

Groups of seven-week old male CD1 mice were injected subcutaneously once a week for 6 weeks with 100 mg/kg of ISIS oligonucleotide. A group of male CD1 mice was injected subcutaneously once a week for 6 weeks with PBS. One group of mice was injected with subcutaneously once a week for 6 weeks with 100 mg/kg of control oligonucleotide ISIS 141923 (CCTTCCCTGAAGGTTCCTCC, designated herein as SEQ ID NO: 809, 5-10-5 MOE gapmer with no known murine target). Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 38
Plasma chemistry markers in CD1 mice plasma on day 40

| | ALT | AST | BUN |
|---|---|---|---|
| | (IU/L) | (IU/L) | (mg/dL) |
| PBS | 25 | 46 | 20 |
| ISIS 532614 | 513 | 407 | 22 |
| ISIS 532692 | 131 | 130 | 24 |
| ISIS 532770 | 36 | 53 | 25 |
| ISIS 532775 | 193 | 158 | 23 |
| ISIS 532800 | 127 | 110 | 25 |
| ISIS 532809 | 36 | 42 | 22 |
| ISIS 532810 | 229 | 286 | 26 |
| ISIS 532811 | 197 | 183 | 21 |
| ISIS 532917 | 207 | 204 | 27 |
| ISIS 532952 | 246 | 207 | 25 |
| ISIS 141923 | 39 | 67 | 23 |

### Weights

Body weights of the mice were measured on day 40 before sacrificing the mice. Weights of organs, liver, kidney, and spleen were also measured after the mice were sacrificed. The results are presented in the Table below. ISIS oligonucleotides that caused changes in the weights outside the expected range for antisense oligonucleotides were excluded in further studies.

TABLE 39
Weights (g) of CD1 mice on day 40

| | Body | Kidney | Liver | Spleen |
|---|---|---|---|---|
| PBS | 44 | 0.8 | 2.0 | 0.1 |
| ISIS 532614 | 43 | 0.7 | 4.3 | 0.2 |
| ISIS 532692 | 42 | 0.7 | 2.6 | 0.2 |
| ISIS 532770 | 42 | 0.6 | 2.3 | 0.2 |
| ISIS 532775 | 42 | 0.7 | 2.5 | 0.2 |
| ISIS 532800 | 43 | 0.6 | 2.8 | 0.3 |
| ISIS 532809 | 42 | 0.6 | 2.2 | 0.1 |
| ISIS 532810 | 43 | 0.6 | 2.3 | 0.2 |
| ISIS 532811 | 41 | 0.7 | 2.4 | 0.2 |
| ISIS 532917 | 42 | 0.7 | 3.0 | 0.2 |
| ISIS 532952 | 44 | 0.8 | 2.5 | 0.3 |
| ISIS 141923 | 41 | 0.6 | 2.0 | 0.1 |

Study 2 (with 5-10-5 MOE Gapmers)

Groups of six- to eight-week old male CD1 mice were injected subcutaneously once a week for 6 weeks with 100 mg/kg of ISIS oligonucleotide. Two groups of male CD1 mice were injected subcutaneously once a week for 6 weeks with PBS. One group of mice was injected with subcutaneously once a week for 6 weeks with 100 mg/kg of control oligonucleotide ISIS 141923. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 40
Plasma chemistry markers in CD1 mice plasma on day 45

| | ALT | AST | Albumin | BUN |
|---|---|---|---|---|
| | (IU/L) | (IU/L) | (g/dL) | (mg/dL) |
| PBS | 39 | 53 | 2.9 | 29 |
| PBS | 50 | 97 | 2.9 | 30 |
| ISIS 141923 | 163 | 174 | 4.1 | 25 |
| ISIS 532810 | 321 | 297 | 2.5 | 26 |
| ISIS 532952 | 182 | 199 | 2.7 | 27 |
| ISIS 588534 | 276 | 248 | 2.6 | 29 |
| ISIS 588536 | 48 | 60 | 2.9 | 31 |
| ISIS 588537 | 72 | 79 | 4.0 | 25 |
| ISIS 588538 | 63 | 67 | 4.5 | 29 |
| ISIS 588539 | 238 | 177 | 3.9 | 28 |
| ISIS 588545 | 496 | 256 | 4.4 | 24 |
| ISIS 588547 | 323 | 210 | 4.4 | 25 |
| ISIS 588548 | 61 | 63 | 4.2 | 27 |
| ISIS 588549 | 127 | 132 | 4.1 | 23 |
| ISIS 588551 | 302 | 282 | 4.2 | 22 |
| ISIS 588552 | 76 | 98 | 4.0 | 30 |
| ISIS 588558 | 1066 | 521 | 3.9 | 27 |
| ISIS 588559 | 76 | 94 | 4.1 | 26 |
| ISIS 588561 | 502 | 500 | 4.4 | 26 |
| ISIS 588563 | 50 | 99 | 4.4 | 28 |

### Weights

Body weights of the mice were measured on day 42. Weights of organs, liver, kidney, and spleen were also measured after the mice were sacrificed on day 45. The results are presented in the Table below. ISIS oligonucleotides that caused changes in the weights outside the expected range for antisense oligonucleotides were excluded in further studies.

TABLE 41
Weights (g) of CD1 mice on day 40

| | Body | Kidney | Liver | Spleen |
|---|---|---|---|---|
| PBS | 44 | 0.7 | 2.4 | 0.1 |
| PBS | 43 | 0.7 | 2.4 | 0.2 |
| ISIS 141923 | 43 | 0.6 | 2.4 | 0.2 |
| ISIS 532810 | 41 | 0.6 | 1.9 | 0.1 |
| ISIS 532952 | 43 | 0.6 | 2.4 | 0.2 |
| ISIS 588534 | 44 | 0.7 | 2.8 | 0.2 |
| ISIS 588536 | 43 | 0.7 | 2.7 | 0.2 |
| ISIS 588537 | 43 | 0.7 | 2.4 | 0.2 |
| ISIS 588538 | 44 | 0.7 | 2.8 | 0.2 |
| ISIS 588539 | 44 | 0.6 | 2.7 | 0.2 |
| ISIS 588545 | 44 | 0.8 | 3.3 | 0.3 |
| ISIS 588547 | 42 | 0.6 | 3.3 | 0.3 |
| ISIS 588548 | 43 | 0.6 | 2.8 | 0.2 |
| ISIS 588549 | 42 | 0.6 | 2.8 | 0.3 |
| ISIS 588551 | 39 | 0.6 | 2.2 | 0.2 |
| ISIS 588552 | 41 | 0.6 | 2.2 | 0.2 |
| ISIS 588558 | 44 | 0.7 | 3.3 | 0.3 |
| ISIS 588559 | 43 | 0.6 | 2.7 | 0.3 |
| ISIS 588561 | 40 | 0.7 | 2.4 | 0.3 |
| ISIS 588563 | 41 | 0.7 | 2.4 | 0.2 |

Study 3 (with 5-10-5 MOE Gapmers)

Groups of six- to eight-week old male CD1 mice were injected subcutaneously once a week for 6 weeks with 100 mg/kg of ISIS oligonucleotide. Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 42
Plasma chemistry markers in CD1 mice plasma on day 42

| | ALT | AST | Albumin | BUN |
|---|---|---|---|---|
| | (IU/L) | (IU/L) | (g/dL) | (mg/dL) |
| PBS | 37 | 108 | 3.1 | 30 |
| PBS | 45 | 51 | 3.0 | 27 |
| ISIS 588544 | 209 | 168 | 2.9 | 26 |
| ISIS 588546 | 526 | 279 | 3.0 | 22 |
| ISIS 588550 | 82 | 136 | 2.7 | 25 |
| ISIS 588553 | 79 | 105 | 3.0 | 24 |
| ISIS 588554 | 112 | 220 | 3.2 | 19 |
| ISIS 588555 | 95 | 162 | 2.8 | 25 |
| ISIS 588556 | 345 | 236 | 3.0 | 26 |
| ISIS 588557 | 393 | 420 | 2.8 | 24 |
| ISIS 588560 | 109 | 148 | 2.7 | 27 |
| ISIS 588562 | 279 | 284 | 2.8 | 22 |
| ISIS 588564 | 152 | 188 | 3.0 | 23 |
| ISIS 588565 | 247 | 271 | 2.8 | 28 |

### Weights

Body weights of the mice were measured on day 42. Weights of organs, liver, kidney, and spleen were also measured after the mice were sacrificed on day 42. The results are presented in the Table below. ISIS oligonucleotides that caused changes in the weights outside the expected range for antisense oligonucleotides were excluded in further studies.

TABLE 43
Weights (g) of CD1 mice on day 40

| | Body | Kidney | Liver | Spleen |
|---|---|---|---|---|
| PBS | 42 | 0.7 | 2.4 | 0.1 |
| PBS | 41 | 0.7 | 2.4 | 0.2 |
| ISIS 588544 | 44 | 0.6 | 1.9 | 0.1 |
| ISIS 588546 | 43 | 0.6 | 2.4 | 0.2 |
| ISIS 588550 | 41 | 0.7 | 2.8 | 0.2 |
| ISIS 588553 | 44 | 0.7 | 2.7 | 0.2 |
| ISIS 588554 | 40 | 0.7 | 2.4 | 0.2 |
| ISIS 588555 | 44 | 0.7 | 2.8 | 0.2 |
| ISIS 588556 | 39 | 0.6 | 2.7 | 0.2 |
| ISIS 588557 | 41 | 0.8 | 3.3 | 0.3 |
| ISIS 588560 | 38 | 0.6 | 3.2 | 0.3 |
| ISIS 588562 | 41 | 0.6 | 2.8 | 0.2 |
| ISIS 588564 | 40 | 0.6 | 2.8 | 0.3 |
| ISIS 588565 | 39 | 0.6 | 2.2 | 0.2 |

Study 4 (with (S) cEt Gapmers and Deoxy, MOE and cEt Oligonucleotides)

Groups of ten-week old male CD1 mice were injected subcutaneously once a week for 6 weeks with 50 mg/kg of ISIS oligonucleotide from the studies described above. In addition, two oligonucleotides, ISIS 594431 and ISIS 594432, were designed as 3-10-3 cEt gapmers and were also tested in this study. ISIS 594431(ACCTCCTTCCGAGTCA, SEQ ID NO: 550) targets the same region as ISIS 588871, a deoxy, MOE and cEt gapmer (target start site 2197 of SEQ ID NO: 1 and target start site 6985 of SEQ ID NO: 2). ISIS 594432 (TGGTCACATTCCCTTC, SEQ ID NO: 542) targets the same region as ISIS 588872, a deoxy, MOE and cEt gapmer (target start site 154 of SEQ ID NO: 1 and target start site 1875 of SEQ ID NO: 2).

Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 44
Plasma chemistry markers in CD1 mice plasma on day 42

| | | ALT | AST | Albumin | Creatinine | BUN |
|---|---|---|---|---|---|---|
| | Chemistry | (IU/L) | (IU/L) | (g/dL) | (mg/dL) | (mg/dL) |
| PBS | — | 71 | 77 | 2.7 | 0.2 | 29 |
| PBS | — | 30 | 36 | 2.7 | 0.2 | 26 |
| ISIS 588834 | Deoxy, MOE and cEt | 436 | 510 | 2.8 | 0.2 | 25 |
| ISIS 588835 | Deoxy, MOE and cEt | 70 | 98 | 3.0 | 0.2 | 27 |
| ISIS 588836 | Deoxy, MOE and cEt | 442 | 312 | 2.7 | 0.2 | 27 |
| ISIS 588846 | Deoxy, MOE and cEt | 50 | 75 | 2.5 | 0.1 | 28 |
| ISIS 588847 | Deoxy, MOE and cEt | 44 | 71 | 2.6 | 0.1 | 24 |
| ISIS 588848 | Deoxy, MOE and cEt | 47 | 70 | 2.4 | 0.1 | 27 |
| ISIS 588857 | Deoxy, MOE and cEt | 1287 | 655 | 2.7 | 0.2 | 26 |
| ISIS 588858 | Deoxy, MOE and cEt | 1169 | 676 | 2.5 | 0.2 | 26 |
| ISIS 588859 | Deoxy, MOE and cEt | 1036 | 1300 | 3.2 | 0.2 | 25 |
| ISIS 588861 | Deoxy, MOE and cEt | 749 | 466 | 3.1 | 0.1 | 24 |
| ISIS 588862 | Deoxy, MOE and cEt | 1564 | 1283 | 2.9 | 0.2 | 22 |
| ISIS 588863 | Deoxy, MOE and cEt | 477 | 362 | 2.8 | 0.1 | 23 |
| ISIS 588864 | Deoxy, MOE and cEt | 118 | 165 | 2.9 | 0.2 | 27 |
| ISIS 588866 | Deoxy, MOE and cEt | 843 | 784 | 3.2 | 0.2 | 25 |
| ISIS 594430 | 3-10-3 cEt | 89 | 99 | 2.4 | 0.1 | 28 |
| ISIS 594431 | 3-10-3 cEt | 590 | 433 | 3.0 | 0.2 | 24 |
| ISIS 594432 | 3-10-3 cEt | 2595 | 2865 | 2.4 | 0.1 | 25 |

### Weights

Body weights of the mice were measured on day 39. Weights of organs, liver, kidney, and spleen were also measured after the mice were sacrificed on day 42. The results are presented in the Table below. ISIS oligonucleotides that caused changes in the weights outside the expected range for antisense oligonucleotides were excluded in further studies.

TABLE 45
Weights (g) of CD1 mice

| | Chemistry | Body | Kidney | Liver | Spleen |
|---|---|---|---|---|---|
| PBS | — | 37 | 0.6 | 2.1 | 0.1 |
| PBS | — | 45 | 0.7 | 2.5 | 0.2 |
| ISIS 588834 | Deoxy, MOE and cEt | 40 | 0.6 | 3.2 | 0.2 |
| ISIS 588835 | Deoxy, MOE and cEt | 38 | 0.7 | 2.8 | 0.3 |
| ISIS 588836 | Deoxy, MOE and cEt | 41 | 0.7 | 2.3 | 0.2 |
| ISIS 588837 | Deoxy, MOE and cEt | 38 | 0.6 | 2.4 | 0.3 |
| ISIS 588846 | Deoxy, MOE and cEt | 39 | 0.6 | 2.3 | 0.2 |
| ISIS 588847 | Deoxy, MOE and cEt | 40 | 0.7 | 2.5 | 0.2 |
| ISIS 588848 | Deoxy, MOE and cEt | 43 | 0.7 | 2.6 | 0.3 |
| ISIS 588857 | Deoxy, MOE and cEt | 39 | 0.6 | 3.3 | 0.2 |
| ISIS 588858 | Deoxy, MOE and cEt | 37 | 0.6 | 3.4 | 0.2 |
| ISIS 588859 | Deoxy, MOE and cEt | 41 | 0.7 | 2.5 | 0.3 |
| ISIS 588861 | Deoxy, MOE and cEt | 39 | 0.6 | 2.6 | 0.4 |
| ISIS 588862 | Deoxy, MOE and cEt | 34 | 0.6 | 2.5 | 0.4 |
| ISIS 588863 | Deoxy, MOE and cEt | 40 | 0.6 | 2.7 | 0.3 |
| ISIS 588864 | Deoxy, MOE and cEt | 40 | 0.7 | 2.3 | 0.2 |
| ISIS 588866 | Deoxy, MOE and cEt | 45 | 0.7 | 3.0 | 0.2 |
| ISIS 594430 | 3-10-3 cEt | 39 | 0.6 | 2.2 | 0.2 |
| ISIS 594431 | 3-10-3 cEt | 36 | 0.6 | 3.2 | 0.2 |
| ISIS 594432 | 3-10-3 cEt | 31 | 0.4 | 1.9 | 0.1 |

Study 5 (with MOE Gapmers, (S) cEt Gapmers and Deoxy, MOE and cEt Oligonucleotides)

Groups of eight- to nine-week old male CD1 mice were injected subcutaneously once a week for 6 weeks with 50 mg/kg of ISIS oligonucleotide. Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 46
Plasma chemistry markers in CD1 mice plasma on day 42

| | | ALT | AST | Albumin | Creatinine | BUN |
|---|---|---|---|---|---|---|
| | Chemistry | (IU/L) | (IU/L) | (g/dL) | (mg/dL) | (mg/dL) |
| PBS | — | 33 | 84 | 2.9 | 0.2 | 28 |
| PBS | — | 32 | 65 | 2.5 | 0.1 | 27 |
| ISIS 532692 | 5-10-5 MOE | 363 | 281 | 3.0 | 0.2 | 30 |
| ISIS 532770 | 5-10-5 MOE | 69 | 100 | 2.9 | 0.1 | 28 |
| ISIS 532775 | 5-10-5 MOE | 371 | 333 | 2.6 | 0.1 | 29 |
| ISIS 532800 | 5-10-5 MOE | 104 | 106 | 2.7 | 0.1 | 31 |
| ISIS 532809 | 5-10-5 MOE | 69 | 127 | 2.8 | 0.1 | 26 |
| ISIS 588540 | 5-10-5 MOE | 66 | 110 | 2.8 | 0.1 | 26 |
| ISIS 588838 | 3-10-3 cEt | 391 | 330 | 2.9 | 0.1 | 25 |
| ISIS 588842 | Deoxy, MOE and cEt | 224 | 264 | 2.6 | 0.1 | 26 |
| ISIS 588843 | 3-10-3 cEt | 185 | 160 | 2.8 | 0.1 | 24 |
| ISIS 588844 | Deoxy, MOE and cEt | 304 | 204 | 2.7 | 0.1 | 25 |
| ISIS 588851 | Deoxy, MOE and cEt | 186 | 123 | 2.7 | 0.1 | 31 |
| ISIS 588854 | Deoxy, MOE and cEt | 1232 | 925 | 2.7 | 0.1 | 25 |
| ISIS 588855 | Deoxy, MOE and cEt | 425 | 321 | 2.7 | 0.1 | 28 |
| ISIS 588856 | Deoxy, MOE and cEt | 78 | 101 | 2.4 | 0.1 | 31 |
| ISIS 588865 | Deoxy, MOE and cEt | 126 | 145 | 2.5 | 0.1 | 23 |
| ISIS 588867 | Deoxy, MOE and cEt | 108 | 112 | 2.5 | 0.1 | 32 |
| ISIS 588868 | Deoxy, MOE and cEt | 61 | 124 | 2.5 | 0.1 | 28 |
| ISIS 588870 | Deoxy, MOE and cEt | 48 | 69 | 2.4 | 0.1 | 31 |
| ISIS 588871 | Deoxy, MOE and cEt | 723 | 881 | 2.5 | 0.1 | 24 |
| ISIS 588872 | Deoxy, MOE and cEt | 649 | 654 | 2.7 | 0.1 | 26 |

### Weights

Body weights of the mice were measured on day 40. Weights of organs, liver, kidney, and spleen were also measured after the mice were sacrificed on day 42. The results are presented in the Table below. ISIS oligonucleotides that caused changes in the weights outside the expected range for antisense oligonucleotides were excluded in further studies.

TABLE 47
Weights (g) of CD1 mice

| | Chemistry | Body | Kidney | Liver | Spleen |
|---|---|---|---|---|---|
| PBS | — | 46 | 0.7 | 2.3 | 0.2 |
| PBS | — | 44 | 0.7 | 2.3 | 0.2 |
| ISIS 532692 | 5-10-5 MOE | 44 | 0.6 | 2.8 | 0.2 |
| ISIS 532770 | 5-10-5 MOE | 43 | 0.6 | 2.2 | 0.2 |
| ISIS 532775 | 5-10-5 MOE | 43 | 0.6 | 2.8 | 0.2 |
| ISIS 532800 | 5-10-5 MOE | 47 | 0.7 | 2.9 | 0.2 |
| ISIS 532809 | 5-10-5 MOE | 44 | 0.7 | 2.6 | 0.2 |
| ISIS 588540 | 5-10-5 MOE | 44 | 0.7 | 2.5 | 0.2 |
| ISIS 588838 | 3-10-3 cEt | 45 | 0.7 | 3.1 | 0.2 |
| ISIS 588842 | Deoxy, MOE and cEt | 41 | 0.6 | 2.6 | 0.2 |
| ISIS 588843 | 3-10-3 cEt | 43 | 0.7 | 2.9 | 0.2 |
| ISIS 588844 | Deoxy, MOE and cEt | 43 | 0.7 | 2.8 | 0.2 |
| ISIS 588851 | Deoxy, MOE and cEt | 46 | 0.6 | 2.6 | 0.2 |
| ISIS 588854 | Deoxy, MOE and cEt | 45 | 0.7 | 4.1 | 0.2 |
| ISIS 588855 | Deoxy, MOE and cEt | 44 | 0.7 | 2.9 | 0.3 |
| ISIS 588856 | Deoxy, MOE and cEt | 44 | 0.7 | 3.2 | 0.2 |
| ISIS 588865 | Deoxy, MOE and cEt | 45 | 0.7 | 2.6 | 0.3 |
| ISIS 588867 | Deoxy, MOE and cEt | 46 | 0.7 | 3.2 | 0.3 |
| ISIS 588868 | Deoxy, MOE and cEt | 42 | 0.7 | 2.9 | 0.3 |
| ISIS 588870 | Deoxy, MOE and cEt | 43 | 0.6 | 2.2 | 0.2 |
| ISIS 588871 | Deoxy, MOE and cEt | 41 | 0.7 | 3.1 | 0.2 |
| ISIS 588872 | Deoxy, MOE and cEt | 39 | 0.6 | 3.2 | 0.3 |

Study 6 (with Deoxy, MOE and cEt Oligonucleotides)

Groups of eight- to nine-week old male CD1 mice were injected subcutaneously once a week for 6 weeks with 50 mg/kg of deoxy, MOE, and cEt oligonucleotides. Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, bilirubin, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 48
Plasma chemistry markers in CD1 mice plasma on day 45

| | ALT | AST | Albumin | Creatinine | Bilirubin | BUN |
|---|---|---|---|---|---|---|
| | (IU/L) | (IU/L) | (g/dL) | (mg/dL) | (mg/dL) | (mg/dL) |
| PBS | 39 | 78 | 3.4 | 0.2 | 0.2 | 31 |
| PBS | 37 | 59 | 2.9 | 0.1 | 0.2 | 27 |
| ISIS 599552 | 167 | 208 | 3.0 | 0.1 | 0.2 | 32 |
| ISIS 599553 | 43 | 86 | 2.9 | 0.1 | 0.2 | 28 |
| ISIS 599554 | 57 | 101 | 2.2 | 0.2 | 0.2 | 31 |
| ISIS 599569 | 469 | 530 | 3.5 | 0.2 | 0.3 | 27 |
| ISIS 599577 | 37 | 84 | 2.9 | 0.1 | 0.1 | 31 |
| ISIS 599578 | 45 | 104 | 2.8 | 0.1 | 0.2 | 30 |
| ISIS 599581 | 54 | 88 | 3.1 | 0.1 | 0.2 | 31 |
| ISIS 599590 | 1741 | 1466 | 3.1 | 0.1 | 0.3 | 25 |
| ISIS 599591 | 2230 | 1183 | 3.2 | 0.1 | 0.3 | 27 |
| ISIS 601209 | 68 | 104 | 2.9 | 0.1 | 0.2 | 30 |
| ISIS 601212 | 1795 | 968 | 3.2 | 0.1 | 0.3 | 22 |
| ISIS 601215 | 424 | 385 | 3.1 | 0.1 | 0.4 | 25 |
| ISIS 601216 | 90 | 125 | 2.9 | 0.1 | 0.2 | 29 |
| ISIS 601276 | 946 | 366 | 2.9 | 0.1 | 0.5 | 31 |
| ISIS 601282 | 831 | 540 | 3.3 | 0.2 | 0.2 | 32 |

### Weights

Body weights of the mice were measured on day 40. Weights of organs, liver, kidney, and spleen were also measured after the mice were sacrificed on day 45. The results are presented in the Table below. ISIS oligonucleotides that caused changes in the weights outside the expected range for antisense oligonucleotides were excluded in further studies.

TABLE 49
Weights (g) of CD1 mice

| | Body | Kidney | Liver | Spleen |
|---|---|---|---|---|
| PBS | 40 | 0.7 | 2.1 | 0.2 |
| PBS | 42 | 0.8 | 2.3 | 0.2 |
| ISIS 599552 | 38 | 0.6 | 2.3 | 0.2 |
| ISIS 599553 | 39 | 0.7 | 2.2 | 0.2 |
| ISIS 599554 | 39 | 0.7 | 2.4 | 0.2 |
| ISIS 599569 | 39 | 0.7 | 2.2 | 0.2 |
| ISIS 599577 | 41 | 0.7 | 2.5 | 0.2 |
| ISIS 599578 | 37 | 0.6 | 2.0 | 0.2 |
| ISIS 599581 | 40 | 0.6 | 2.5 | 0.2 |
| ISIS 599590 | 34 | 0.6 | 3.5 | 0.2 |
| ISIS 599591 | 38 | 0.8 | 2.7 | 0.2 |
| ISIS 601209 | 42 | 0.7 | 2.6 | 0.3 |
| ISIS 601212 | 38 | 0.6 | 2.9 | 0.2 |
| ISIS 601215 | 36 | 0.7 | 2.6 | 0.2 |
| ISIS 601216 | 42 | 0.6 | 2.7 | 0.2 |
| ISIS 601276 | 42 | 0.7 | 3.2 | 0.2 |
| ISIS 601282 | 38 | 0.7 | 3.2 | 0.2 |

Study 7 (with MOE Gapmers and Deoxy, MOE and cEt Oligonucleotides)

Groups of eight- to nine-week old male CD1 mice were injected subcutaneously once a week for 6 weeks with 100 mg/kg of ISIS oligonucleotides. One group of male CD1 mice was injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 50
Plasma chemistry markers in CD1 mice plasma on day 45

| | | ALT | AST | Albumin | Creatinine | BUN |
|---|---|---|---|---|---|---|
| | Chemistry | (IU/L) | (IU/L) | (g/dL) | (mg/dL) | (mg/dL) |
| PBS | — | 120 | 102 | 2.7 | 0.2 | 26 |
| ISIS 588842 | Deoxy, MOE and cEt | 177 | 164 | 2.7 | 0.1 | 23 |
| ISIS 588843 | Deoxy, MOE and cEt | 98 | 194 | 2.7 | 0.1 | 24 |
| ISIS 588851 | Deoxy, MOE and cEt | 91 | 142 | 2.6 | 0.1 | 23 |
| ISIS 588856 | Deoxy, MOE and cEt | 78 | 110 | 2.7 | 0.1 | 23 |
| ISIS 599024 | 3-10-4 MOE | 91 | 108 | 2.7 | 0.1 | 23 |
| ISIS 599087 | 5-7-5 MOE | 198 | 183 | 2.6 | 0.2 | 28 |
| ISIS 599093 | 5-7-5 MOE | 3285 | 2518 | 2.6 | 0.2 | 24 |
| ISIS 599149 | 4-8-5 MOE | 30 | 64 | 2.9 | 0.2 | 25 |
| ISIS 599155 | 4-8-5 MOE | 145 | 189 | 2.6 | 0.2 | 25 |
| ISIS 599202 | 5-8-5 MOE | 150 | 128 | 2.8 | 0.2 | 23 |
| ISIS 599203 | 5-8-5 MOE | 111 | 127 | 2.8 | 0.2 | 24 |
| ISIS 599208 | 5-8-5 MOE | 146 | 178 | 2.9 | 0.2 | 22 |
| ISIS 599261 | 3-10-5 MOE | 144 | 165 | 2.8 | 0.2 | 26 |
| ISIS 599267 | 3-10-5 MOE | 96 | 132 | 2.6 | 0.2 | 27 |
| ISIS 599268 | 3-10-5 MOE | 87 | 115 | 2.6 | 0.1 | 23 |
| ISIS 599322 | 6-7-6 MOE | 115 | 138 | 2.7 | 0.1 | 22 |
| ISIS 599374 | 5-9-5 MOE | 375 | 271 | 2.6 | 0.1 | 21 |
| ISIS 599378 | 5-9-5 MOE | 77 | 99 | 2.7 | 0.1 | 23 |
| ISIS 599441 | 6-8-6 MOE | 150 | 250 | 2.9 | 0.1 | 23 |

### Weights

Body weights of the mice were measured on day 44. Weights of organs, liver, kidney, and spleen were also measured after the mice were sacrificed on day 49. The results are presented in the Table below. ISIS oligonucleotides that caused changes in the weights outside the expected range for antisense oligonucleotides were excluded in further studies.

TABLE 51
Weights (g) of CD1 mice

| | Chemistry | Body | Kidney | Liver | Spleen |
|---|---|---|---|---|---|
| PBS | — | 39 | 0.6 | 1.9 | 0.1 |
| ISIS 588842 | Deoxy, MOE and cEt | 38 | 0.5 | 2.1 | 0.1 |
| ISIS 588843 | Deoxy, MOE and cEt | 41 | 0.6 | 2.4 | 0.2 |
| ISIS 588851 | Deoxy, MOE and cEt | 42 | 0.6 | 2.2 | 0.2 |
| ISIS 588856 | Deoxy, MOE and cEt | 42 | 0.7 | 2.6 | 0.2 |
| ISIS 599024 | 3-10-4 MOE | 41 | 0.6 | 4.0 | 0.2 |
| ISIS 599087 | 5-7-5 MOE | 44 | 0.8 | 2.6 | 0.3 |
| ISIS 599093 | 5-7-5 MOE | 39 | 0.6 | 2.3 | 0.2 |
| ISIS 599149 | 4-8-5 MOE | 42 | 0.7 | 2.8 | 0.2 |
| ISIS 599155 | 4-8-5 MOE | 41 | 0.7 | 2.1 | 0.2 |
| ISIS 599202 | 5-8-5 MOE | 43 | 0.6 | 2.6 | 0.2 |
| ISIS 599203 | 5-8-5 MOE | 42 | 0.6 | 2.6 | 0.2 |
| ISIS 599208 | 5-8-5 MOE | 40 | 0.6 | 2.1 | 0.2 |
| ISIS 599261 | 3-10-5 MOE | 39 | 0.7 | 3.4 | 0.3 |
| ISIS 599267 | 3-10-5 MOE | 42 | 0.8 | 2.5 | 0.3 |
| ISIS 599268 | 3-10-5 MOE | 41 | 0.7 | 2.1 | 0.2 |
| ISIS 599322 | 6-7-6 MOE | 43 | 0.6 | 2.2 | 0.2 |
| ISIS 599374 | 5-9-5 MOE | 37 | 0.6 | 2.2 | 0.2 |
| ISIS 599378 | 5-9-5 MOE | 43 | 0.7 | 2.7 | 0.2 |
| ISIS 599441 | 6-8-6 MOE | 42 | 0.6 | 2.5 | 0.3 |

Study 8 (with MOE Gapmers, Deoxy, MOE and cEt Oligonucleotides, and cEt Gapmers)

Groups of eight- to nine-week old male CD1 mice were injected subcutaneously once a week for 6 weeks with 100 mg/kg of MOE gapmers, or 50 mg/kg of deoxy, MOE and cEt oligonucleotides or cEt gapmers. One group of male CD1 mice was injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below.

TABLE 52
Plasma chemistry markers in CD1 mice plasma on day 43

| | | Dose | ALT | AST | Albumin | Creatinine | BUN |
|---|---|---|---|---|---|---|---|
| | Chemistry | (mg/kg/wk) | (IU/L) | (IU/L) | (g/dL) | (mg/dL) | (mg/dL) |
| PBS | — | — | 37 | 57 | 2.5 | 0.08 | 26 |
| ISIS 532770 | 5-10-5 MOE | 100 | 57 | 73 | 2.5 | 0.07 | 24 |
| ISIS 532800 | 5-10-5 MOE | 100 | 74 | 126 | 2.8 | 0.10 | 26 |
| ISIS 532809 | 5-10-5 MOE | 100 | 83 | 73 | 2.5 | 0.07 | 23 |
| ISIS 588540 | 5-10-5 MOE | 100 | 106 | 102 | 2.7 | 0.09 | 27 |
| ISIS 588544 | 5-10-5 MOE | 100 | 66 | 62 | 2.6 | 0.10 | 24 |
| ISIS 588548 | 5-10-5 MOE | 100 | 48 | 67 | 2.6 | 0.08 | 23 |
| ISIS 588550 | 5-10-5 MOE | 100 | 65 | 106 | 2.5 | 0.10 | 25 |
| ISIS 588553 | 5-10-5 MOE | 100 | 78 | 90 | 2.6 | 0.09 | 25 |
| ISIS 588555 | 5-10-5 MOE | 100 | 94 | 89 | 2.5 | 0.08 | 23 |
| ISIS 588848 | Deoxy, MOE | 50 | 38 | 54 | 2.3 | 0.07 | 25 |
| | and cEt | | | | | | |
| ISIS 594430 | 3-10-3 cEt | 50 | 63 | 72 | 2.5 | 0.10 | 27 |

### Weights

Body weights of the mice were measured on day 36. Weights of organs, liver, kidney, and spleen were also measured after the mice were sacrificed on day 43. The results for the organ weights were expressed as a ratio to the body weights and normalized to the PBS control ratio.

TABLE 53
Organ Weights/Body weight (BW) of CD1 mice

| | | Dose | Kidney/ | Liver/ | Spleen/ |
|---|---|---|---|---|---|
| | Chemistry | (mg/kg/wk) | BW | BW | BW |
| PBS | — | — | 1.0 | 1.0 | 1.0 |
| ISIS 532770 | 5-10-5 MOE | 100 | 1.4 | 1.1 | 1.0 |
| ISIS 532800 | 5-10-5 MOE | 100 | 1.5 | 1.1 | 0.9 |
| ISIS 532809 | 5-10-5 MOE | 100 | 1.3 | 1.2 | 0.9 |
| ISIS 588540 | 5-10-5 MOE | 100 | 1.3 | 1.2 | 1.0 |
| ISIS 588544 | 5-10-5 MOE | 100 | 1.6 | 1.1 | 1.0 |
| ISIS 588548 | 5-10-5 MOE | 100 | 1.7 | 1.2 | 1.0 |
| ISIS 588550 | 5-10-5 MOE | 100 | 1.5 | 1.2 | 1.0 |
| ISIS 588553 | 5-10-5 MOE | 100 | 1.5 | 1.0 | 0.8 |
| ISIS 588555 | 5-10-5 MOE | 100 | 1.8 | 1.2 | 1.0 |
| ISIS 588848 | Deoxy, MOE | 50 | 1.3 | 1.0 | 0.9 |
| | and cEt | | | | |
| ISIS 594430 | 3-10-3 cEt | 50 | 1.4 | 1.1 | 0.9 |

### Cytokine Assays

Blood obtained from all mice groups were sent to Antech Diagnostics for measurements of the various cytokine levels, such as IL-6, MDC, MIP1β, IP-10, MCP1, MIP-1α, and RANTES. The results are presented in Table 54.

TABLE 54
Cytokine levels (pg/mL) in CD1 mice plasma

| | Chemistry | IL-6 | MDC | MIP1β | IP-10 | MCP1 | MIP-1α | RANTES |
|---|---|---|---|---|---|---|---|---|
| PBS | — | 70 | 16 | 23 | 20 | 17 | 6 | 2 |
| ISIS 532770 | 5-10-5 MOE | 101 | 18 | 146 | 116 | 101 | 24 | 6 |
| ISIS 532800 | 5-10-5 MOE | 78 | 17 | 83 | 53 | 105 | 1 | 3 |
| ISIS 532809 | 5-10-5 MOE | 66 | 19 | 60 | 32 | 55 | 20 | 4 |
| ISIS 588540 | 5-10-5 MOE | 51 | 18 | 126 | 70 | 75 | 4 | 3 |
| ISIS 588544 | 5-10-5 MOE | 157 | 14 | 94 | 34 | 102 | 1 | 3 |
| ISIS 588548 | 5-10-5 MOE | 164 | 12 | 90 | 66 | 84 | 10 | 4 |
| ISIS 588550 | 5-10-5 MOE | 58 | 21 | 222 | 124 | 157 | 3 | 5 |
| ISIS 588553 | 5-10-5 MOE | 62 | 14 | 183 | 60 | 103 | 9 | 4 |
| ISIS 588555 | 5-10-5 MOE | 70 | 19 | 172 | 171 | 178 | 16 | 9 |
| ISIS 588848 | Deoxy, MOE | 59 | 13 | 61 | 27 | 63 | 12 | 4 |
| | and cEt | | | | | | | |
| ISIS 594430 | 3-10-3 cEt | 48 | 14 | 56 | 38 | 85 | 10 | 3 |

Hematology Assays Blood obtained from all mice groups were sent to Antech Diagnostics for measurements of hematocrit (HCT), as well as of the various blood cells, such as WBC, RBC, and platelets, and total hemoglobin (Hb) content. The results are presented in Table 55.

TABLE 55
Hematology markers in CD1 mice plasma

| | | HCT | Hb | WBC | RBC | Platelets |
|---|---|---|---|---|---|---|
| | Chemistry | (%) | (g/dL) | (10³/μL) | (10⁶/μL) | (10³/μL) |
| PBS | — | 46 | 15 | 7 | 9 | 960 |
| ISIS 532770 | 5-10-5 MOE | 45 | 14 | 5 | 9 | 879 |
| ISIS 532800 | 5-10-5 MOE | 45 | 14 | 5 | 9 | 690 |
| ISIS 532809 | 5-10-5 MOE | 46 | 14 | 6 | 9 | 1005 |
| ISIS 588540 | 5-10-5 MOE | 49 | 15 | 6 | 10 | 790 |
| ISIS 588544 | 5-10-5 MOE | 36 | 11 | 7 | 7 | 899 |
| ISIS 588548 | 5-10-5 MOE | 46 | 14 | 6 | 9 | 883 |
| ISIS 588550 | 5-10-5 MOE | 42 | 13 | 8 | 8 | 721 |
| ISIS 588553 | 5-10-5 MOE | 45 | 14 | 6 | 9 | 719 |
| ISIS 588555 | 5-10-5 MOE | 43 | 13 | 8 | 9 | 838 |
| ISIS 588848 | Deoxy, MOE | 40 | 15 | 8 | 10 | 840 |
| | and cEt | | | | | |
| ISIS 594430 | 3-10-3 cEt | 45 | 14 | 8 | 9 | 993 |

### Example 13: Tolerability of Antisense Oligonucleotides Targeting Human CFB in Sprague-Dawley Rats

Sprague-Dawley rats are a multipurpose model used for safety and efficacy evaluations. The rats were treated with ISIS antisense oligonucleotides from the studies described in the Examples above and evaluated for changes in the levels of various plasma chemistry markers.

Study 1 (with 5-10-5 MOE Gapmers)

Male Sprague-Dawley rats, seven- to eight-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of 5-10-5 MOE gapmers. One control group of 6 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 56
Liver function markers in Sprague-Dawley rats

| | ALT | AST |
|---|---|---|
| | (IU/L) | (IU/L) |
| PBS | 66 | 134 |
| ISIS 588544 | 101 | 329 |
| ISIS 588550 | 69 | 157 |
| ISIS 588553 | 88 | 304 |
| ISIS 588554 | 202 | 243 |
| ISIS 588555 | 94 | 113 |
| ISIS 588556 | 102 | 117 |
| ISIS 588560 | 206 | 317 |
| ISIS 588564 | 292 | 594 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 57
Kidney function markers (mg/dL) in Sprague-Dawley rats

| | BUN | Creatinine |
|---|---|---|
| PBS | 18 | 3.5 |
| ISIS 588544 | 21 | 3.1 |
| ISIS 588550 | 21 | 3.0 |
| ISIS 588553 | 22 | 2.8 |
| ISIS 588554 | 23 | 3.0 |
| ISIS 588555 | 22 | 3.5 |
| ISIS 588556 | 21 | 3.2 |
| ISIS 588560 | 26 | 2.4 |
| ISIS 588564 | 24 | 2.7 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 58
Weights (g)

| | Body | Liver | Spleen | Kidney |
|---|---|---|---|---|
| PBS | 422 | 16 | 1.2 | 3.9 |
| ISIS 588544 | 353 | 15 | 1.7 | 2.9 |
| ISIS 588550 | 321 | 14 | 2.1 | 3.2 |
| ISIS 588553 | 313 | 15 | 2.3 | 3.2 |
| ISIS 588554 | 265 | 11 | 1.6 | 2.7 |
| ISIS 588555 | 345 | 14 | 1.4 | 3.3 |
| ISIS 588556 | 328 | 13 | 1.7 | 3.1 |
| ISIS 588560 | 270 | 13 | 2.4 | 3.0 |
| ISIS 588564 | 253 | 12 | 2.9 | 3.0 |

Study 2 (with Deoxy, MOE and cEt Oligonucleotides)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of deoxy, MOE, and cEt oligonucleotides. Two control groups of 3 rats each were injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase), and albumin were measured and the results are presented in the Table below. ISIS oligonucleotides that caused changes in the levels of any markers of liver function outside the expected range for antisense oligonucleotides were excluded in further studies.

TABLE 59
Liver function markers in Sprague-Dawley rats

| | ALT | AST | Albumin |
|---|---|---|---|
| | (IU/L) | (IU/L) | (g/dL) |
| PBS | 55 | 150 | 3.4 |
| PBS | 64 | 91 | 3.5 |
| ISIS 588554 | 52 | 92 | 3.2 |
| ISIS 588835 | 971 | 844 | 4.1 |
| ISIS 588842 | 317 | 359 | 3.8 |
| ISIS 588843 | 327 | 753 | 2.9 |
| ISIS 588846 | 70 | 111 | 3.2 |
| ISIS 588847 | 65 | 100 | 3.0 |
| ISIS 588864 | 91 | 109 | 3.0 |
| ISIS 594430 | 85 | 106 | 3.7 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 60
Kidney function markers (mg/dL) in Sprague-Dawley rats

| | BUN | Creatinine |
|---|---|---|
| PBS | 17 | 0.4 |
| PBS | 21 | 0.4 |
| ISIS 588554 | 20 | 0.4 |
| ISIS 588835 | 23 | 0.5 |
| ISIS 588842 | 22 | 0.4 |
| ISIS 588843 | 51 | 0.4 |
| ISIS 588846 | 25 | 0.5 |
| ISIS 588847 | 23 | 0.5 |
| ISIS 588864 | 23 | 0.4 |
| ISIS 594430 | 22 | 0.5 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 61
Weights (g)

| | Body | Liver | Spleen | Kidney |
|---|---|---|---|---|
| PBS | 466 | 16 | 0.9 | 3.8 |
| PBS | 485 | 16 | 0.9 | 3.6 |
| ISIS 588554 | 393 | 15 | 2.3 | 2.6 |
| ISIS 588835 | 387 | 16 | 1.0 | 3.3 |
| ISIS 588842 | 414 | 22 | 1.5 | 3.7 |
| ISIS 588843 | 427 | 20 | 2.5 | 4.2 |
| ISIS 588846 | 366 | 16 | 2.1 | 3.3 |
| ISIS 588847 | 402 | 15 | 1.6 | 3.1 |
| ISIS 588864 | 364 | 15 | 2.1 | 3.8 |
| ISIS 594430 | 420 | 16 | 1.2 | 3.6 |

Study 3 (with MOE Gapmers)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of MOE gapmers. One control group of 6 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 43 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 62
Liver function markers in Sprague-Dawley rats

| | | ALT | AST | Albumin |
|---|---|---|---|---|
| | Chemistry | (IU/L) | (IU/L) | (g/dL) |
| PBS | — | 52 | 110 | 3.7 |
| ISIS 588563 | 5-10-5 MOE | 175 | 291 | 2.9 |
| ISIS 599024 | 3-10-4 MOE | 139 | 173 | 1.4 |
| ISIS 599093 | 5-7-5 MOE | 116 | 238 | 2.6 |
| ISIS 599149 | 4-8-5 MOE | 232 | 190 | 3.4 |
| ISIS 599155 | 4-8-5 MOE | 108 | 215 | 2.5 |
| ISIS 599202 | 5-8-5 MOE | 65 | 86 | 3.5 |
| ISIS 599203 | 5-8-5 MOE | 71 | 97 | 3.1 |
| ISIS 599208 | 5-8-5 MOE | 257 | 467 | 1.9 |
| ISIS 599261 | 3-10-5 MOE | 387 | 475 | 1.5 |
| ISIS 599267 | 3-10-5 MOE | 201 | 337 | 2.7 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 63
Kidney function markers (mg/dL) in Sprague-Dawley rats

| | Chemistry | BUN | Creatinine |
|---|---|---|---|
| PBS | — | 16 | 0.3 |
| ISIS 588563 | 5-10-5 MOE | 26 | 0.4 |
| ISIS 599024 | 3-10-4 MOE | 135 | 1.2 |
| ISIS 599093 | 5-7-5 MOE | 29 | 0.4 |
| ISIS 599149 | 4-8-5 MOE | 23 | 0.4 |
| ISIS 599155 | 4-8-5 MOE | 29 | 0.4 |
| ISIS 599202 | 5-8-5 MOE | 19 | 0.4 |
| ISIS 599203 | 5-8-5 MOE | 22 | 0.4 |
| ISIS 599208 | 5-8-5 MOE | 26 | 0.3 |
| ISIS 599261 | 3-10-5 MOE | 228 | 1.6 |
| ISIS 599267 | 3-10-5 MOE | 24 | 0.4 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 64
Weights (g)

| | Chemistry | Body | Liver | Spleen | Kidney |
|---|---|---|---|---|---|
| PBS | — | 471 | 16 | 1.0 | 4.1 |
| ISIS 588563 | 5-10-5 MOE | 311 | 16 | 3.4 | 4.1 |
| ISIS 599024 | 3-10-4 MOE | 297 | 11 | 1.0 | 3.5 |
| ISIS 599093 | 5-7-5 MOE | 332 | 18 | 4.1 | 5.0 |
| ISIS 599149 | 4-8-5 MOE | 388 | 16 | 2.3 | 3.7 |
| ISIS 599155 | 4-8-5 MOE | 290 | 15 | 2.9 | 4.5 |
| ISIS 599202 | 5-8-5 MOE | 359 | 13 | 1.3 | 3.2 |
| ISIS 599203 | 5-8-5 MOE | 334 | 14 | 1.8 | 3.3 |
| ISIS 599208 | 5-8-5 MOE | 353 | 29 | 4.7 | 4.6 |
| ISIS 599261 | 3-10-5 MOE | 277 | 10 | 0.9 | 3.2 |
| ISIS 599267 | 3-10-5 MOE | 344 | 21 | 3.9 | 4.7 |

Study 4 (with MOE Gapmers)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of MOE gapmers. One control group of 6 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 65
Liver function markers in Sprague-Dawley rats

| | | ALT | AST | Albumin |
|---|---|---|---|---|
| | Chemistry | (IU/L) | (IU/L) | (g/dL) |
| PBS | — | 48 | 77 | 3.9 |
| ISIS 532800 | 5-10-5 MOE | 72 | 111 | 3.4 |
| ISIS 532809 | 5-10-5 MOE | 59 | 89 | 3.8 |
| ISIS 588540 | 5-10-5 MOE | 146 | 259 | 3.8 |
| ISIS 599268 | 3-10-5 MOE | 175 | 206 | 2.7 |
| ISIS 599322 | 6-7-6 MOE | 523 | 567 | 3.3 |
| ISIS 599374 | 5-9-5 MOE | 114 | 176 | 3.0 |
| ISIS 599378 | 5-9-5 MOE | 124 | 116 | 3.2 |
| ISIS 599380 | 5-9-5 MOE | 148 | 210 | 3.4 |
| ISIS 599441 | 6-8-6 MOE | 51 | 91 | 2.6 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 66
Kidney function markers (mg/dL) in Sprague-Dawley rats

| | Chemistry | BUN | Creatinine |
|---|---|---|---|
| PBS | — | 15 | 0.4 |
| ISIS 532800 | 5-10-5 MOE | 26 | 0.5 |
| ISIS 532809 | 5-10-5 MOE | 18 | 0.5 |
| ISIS 588540 | 5-10-5 MOE | 22 | 0.5 |
| ISIS 599268 | 3-10-5 MOE | 28 | 0.5 |
| ISIS 599322 | 6-7-6 MOE | 24 | 0.5 |
| ISIS 599374 | 5-9-5 MOE | 29 | 0.5 |
| ISIS 599378 | 5-9-5 MOE | 22 | 0.4 |
| ISIS 599380 | 5-9-5 MOE | 26 | 0.5 |
| ISIS 599441 | 6-8-6 MOE | 24 | 0.4 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 67
Weights (g)

| | Chemistry | Body | Liver | Spleen | Kidney |
|---|---|---|---|---|---|
| PBS | — | 502 | 16 | 0.9 | 3.7 |
| ISIS 532800 | 5-10-5 MOE | 376 | 16 | 2.0 | 3.4 |
| ISIS 532809 | 5-10-5 MOE | 430 | 16 | 1.4 | 3.4 |
| ISIS 588540 | 5-10-5 MOE | 391 | 16 | 1.8 | 3.5 |
| ISIS 599268 | 3-10-5 MOE | 332 | 16 | 3.6 | 3.6 |
| ISIS 599322 | 6-7-6 MOE | 348 | 13 | 2.1 | 3.4 |
| ISIS 599374 | 5-9-5 MOE | 302 | 12 | 2.0 | 3.3 |
| ISIS 599378 | 5-9-5 MOE | 332 | 11 | 1.1 | 2.8 |
| ISIS 599380 | 5-9-5 MOE | 350 | 11 | 1.5 | 3.3 |
| ISIS 599441 | 6-8-6 MOE | 368 | 16 | 2.5 | 3.3 |

Study 5 (with MOE Gapmers and Deoxy, MOE and cEt Oligonucleotides)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of MOE gapmer or with 50 mg/kg of deoxy, MOE and cEt oligonucleotides. One control group of 4 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 68
Liver function markers in Sprague-Dawley rats

| | | ALT | AST | Albumin |
|---|---|---|---|---|
| | Chemistry | (IU/L) | (IU/L) | (g/dL) |
| PBS | — | 49 | 74 | 3.3 |
| ISIS 532770 | 5-10-5 MOE | 95 | 132 | 3.3 |
| ISIS 588851 | Deoxy, MOE, and cEt | 47 | 72 | 3.1 |
| ISIS 588856 | Deoxy, MOE, and cEt | 56 | 75 | 3.0 |
| ISIS 588865 | Deoxy, MOE, and cEt | 62 | 84 | 2.9 |
| ISIS 588867 | Deoxy, MOE, and cEt | 73 | 214 | 2.9 |
| ISIS 588868 | Deoxy, MOE, and cEt | 59 | 83 | 3.1 |
| ISIS 588870 | Deoxy, MOE, and cEt | 144 | 144 | 3.4 |

### Kidney Function

To evaluate the effect of ISIS oligonucleotides on kidney function, plasma and urine levels of blood urea nitrogen (BUN) and creatinine were measured using an automated clinical chemistry analyzer (Hitachi Olympus AU400e, Melville, NY). Results are presented in the Tables below, expressed in mg/dL. ISIS oligonucleotides that caused changes in the levels of any of the kidney function markers outside the expected range for antisense oligonucleotides were excluded in further studies.

TABLE 69
Kidney function markers (mg/dL)
in the plasma of Sprague-Dawley rats

| | Chemistry | BUN | Creatinine |
|---|---|---|---|
| PBS | — | 18 | 0.3 |
| ISIS 532770 | 5-10-5 MOE | 20 | 0.4 |
| ISIS 588851 | Deoxy, MOE, and cEt | 20 | 0.4 |
| ISIS 588856 | Deoxy, MOE, and cEt | 22 | 0.4 |
| ISIS 588865 | Deoxy, MOE, and cEt | 24 | 0.5 |
| ISIS 588867 | Deoxy, MOE, and cEt | 22 | 0.4 |
| ISIS 588868 | Deoxy, MOE, and cEt | 19 | 0.4 |
| ISIS 588870 | Deoxy, MOE, and cEt | 20 | 0.5 |

TABLE 70
Kidney function markers (mg/dL)
in the urine of Sprague-Dawley rats

| | | Total | |
|---|---|---|---|
| | Chemistry | protein | Creatinine |
| PBS | — | 80 | 92 |
| ISIS 532770 | 5-10-5 MOE | 466 | 69 |
| ISIS 588851 | Deoxy, MOE, and cEt | 273 | 64 |
| ISIS 588856 | Deoxy, MOE, and cEt | 259 | 68 |
| ISIS 588865 | Deoxy, MOE, and cEt | 277 | 67 |
| ISIS 588867 | Deoxy, MOE, and cEt | 337 | 68 |
| ISIS 588868 | Deoxy, MOE, and cEt | 326 | 75 |
| ISIS 588870 | Deoxy, MOE, and cEt | 388 | 82 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 71
Weights (g)

| | Chemistry | Body | Liver | Spleen | Kidney |
|---|---|---|---|---|---|
| PBS | — | 489 | 16 | 0.9 | 3.5 |
| ISIS 532770 | 5-10-5 MOE | 372 | 15 | 1.7 | 3.1 |
| ISIS 588851 | Deoxy, MOE, and cEt | 285 | 14 | 1.4 | 3.2 |
| ISIS 588856 | Deoxy, MOE, and cEt | 415 | 15 | 1.1 | 3.3 |
| ISIS 588865 | Deoxy, MOE, and cEt | 362 | 14 | 2.0 | 3.3 |
| ISIS 588867 | Deoxy, MOE, and cEt | 406 | 15 | 2.4 | 3.4 |
| ISIS 588868 | Deoxy, MOE, and cEt | 399 | 15 | 1.5 | 3.4 |
| ISIS 588870 | Deoxy, MOE, and cEt | 446 | 14 | 1.4 | 3.3 |

Study 6 (with MOE Gapmers, Deoxy, MOE and cEt Oligonucleotides, and cEt Gapmers)

Male rats were maintained on a 12-hour light/dark cycle and fed ad libitum with Purina normal rat chow, diet 5001. Groups of 4 rats each were injected subcutaneously once a week for 6 weeks with 100 mg/kg of MOE gapmers or with 50 mg/kg of deoxy, MOE and cEt oligonucleotide or cEt gapmer. One control group of 4 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L.

TABLE 72
Liver function markers

| | | Dose | ALT | AST | Albumin |
|---|---|---|---|---|---|
| | Chemistry | (mg/kg/wk) | (IU/L) | (IU/L) | (g/dL) |
| PBS | — | — | 54 | 73 | 4.3 |
| ISIS 532770 | 5-10-5 MOE | 100 | 57 | 114 | 4.4 |
| ISIS 532800 | 5-10-5 MOE | 100 | 176 | 180 | 4.3 |
| ISIS 532809 | 5-10-5 MOE | 100 | 71 | 132 | 4.1 |
| ISIS 588540 | 5-10-5 MOE | 100 | 89 | 202 | 4.4 |
| ISIS 588544 | 5-10-5 MOE | 100 | 75 | 152 | 3.9 |
| ISIS 588548 | 5-10-5 MOE | 100 | 50 | 71 | 4.1 |
| ISIS 588550 | 5-10-5 MOE | 100 | 80 | 133 | 3.6 |
| ISIS 588553 | 5-10-5 MOE | 100 | 59 | 112 | 3.9 |
| ISIS 588555 | 5-10-5 MOE | 100 | 97 | 142 | 3.8 |
| ISIS 588848 | Deoxy, MOE | 50 | 53 | 82 | 3.9 |
| | and cEt | | | | |
| ISIS 594430 | 3-10-3 cEt | 50 | 198 | 172 | 4.4 |

### Kidney Function

To evaluate the effect of ISIS oligonucleotides on kidney function, urine levels of total protein and creatinine were measured using an automated clinical chemistry analyzer (Hitachi Olympus AU400e, Melville, NY). Results are presented in the Table below. ISIS oligonucleotides that caused changes in the levels of any of the kidney function markers outside the expected range for antisense oligonucleotides were excluded in further studies.

TABLE 73
Total protein/creatinine ratio in the urine of rats

| | | Dose | P/C |
|---|---|---|---|
| | Chemistry | (mg/kg/wk) | ratio |
| PBS | — | — | 1.1 |
| ISIS 532770 | 5-10-5 MOE | 100 | 8.3 |
| ISIS 532800 | 5-10-5 MOE | 100 | 6.5 |
| ISIS 532809 | 5-10-5 MOE | 100 | 6.1 |
| ISIS 588540 | 5-10-5 MOE | 100 | 10.1 |
| ISIS 588544 | 5-10-5 MOE | 100 | 7.9 |
| ISIS 588548 | 5-10-5 MOE | 100 | 6.6 |
| ISIS 588550 | 5-10-5 MOE | 100 | 7.6 |
| ISIS 588553 | 5-10-5 MOE | 100 | 7.0 |
| ISIS 588555 | 5-10-5 MOE | 100 | 6.2 |
| ISIS 588848 | Deoxy, MOE and cEt | 50 | 5.2 |
| ISIS 594430 | 3-10-3 cEt | 50 | 5.3 |

### Weights

Body weight measurements were taken on day 39. Liver, heart, spleen and kidney weights were measured at the end of the study on day 42, and are presented in the Table below. The results for the organ weights were expressed as a ratio to the body weights and normalized to the PBS control ratio.

TABLE 74
Organ weights/Body weight (BW) ratios

| | | Dose | Spleen/ | Liver/ | Kidney/ |
|---|---|---|---|---|---|
| | Chemistry | (mg/kg/wk) | BW | BW | BW |
| PBS | — | — | 1.0 | 1.0 | 1.0 |
| ISIS 532770 | 5-10-5 MOE | 100 | 2.0 | 1.2 | 1.0 |
| ISIS 532800 | 5-10-5 MOE | 100 | 2.8 | 1.3 | 1.0 |
| ISIS 532809 | 5-10-5 MOE | 100 | 2.2 | 1.1 | 1.0 |
| ISIS 588540 | 5-10-5 MOE | 100 | 2.2 | 1.4 | 1.0 |
| ISIS 588544 | 5-10-5 MOE | 100 | 2.5 | 1.3 | 1.1 |
| ISIS 588548 | 5-10-5 MOE | 100 | 2.1 | 1.3 | 1.1 |
| ISIS 588550 | 5-10-5 MOE | 100 | 3.9 | 1.4 | 1.1 |
| ISIS 588553 | 5-10-5 MOE | 100 | 4.1 | 1.4 | 1.4 |
| ISIS 588555 | 5-10-5 MOE | 100 | 1.8 | 1.3 | 1.0 |
| ISIS 588848 | Deoxy, MOE | 50 | 3.1 | 1.3 | 1.1 |
| | and cEt | | | | |
| ISIS 594430 | 3-10-3 cEt | 50 | 1.7 | 1.0 | 1.1 |

### Example 14: Efficacy of Antisense Oligonucleotides Against CFB mRNA in hCFB Transgenic Mice

Selected compounds were tested for efficacy in human CFB transgenic mice, founder line #6. The human CFB gene is located on chromosome 6: position 31913721-31919861. A Fosmid (ABC14-50933200C₂₃) containing the CFB sequence was selected to make transgenic mice expressing the human CFB gene. Cla I (31926612) and Age I (31926815) restriction enzymes were used to generate a 22,127 bp fragment containing the CFB gene for pronuclear injection. DNA was confirmed by restriction enzyme analysis using Pvu I. The 22,127 bp DNA fragment was injected into C₅₇BL/6NTac embryos. 6 positive founders were bred. Founder #6 expressed the liver human CFB mRNA and was crossbreed to the 3^{rd} generation. Progeny from 3rd generation mice were used to evaluate human CFB ASOs for human CFB mRNA reduction.

### Treatment

Groups of 3 mice each were injected subcutaneously twice a week for the first week with 50 mg/kg of ISIS oligonucleotides, followed by once a week dosing with 50 mg/kg of ISIS oligonucleotides for an additional three weeks. One control group of 4 mice was injected subcutaneously twice a week for 2 weeks for the first week with PBS for the first week for an additional three weeks. Forty eight hours after the last dose, mice were euthanized and organs and plasma were harvested for further analysis.

### RNA Analysis

At the end of the dosing period, RNA was extracted from the liver and kidney for real-time PCR analysis of CFB mRNA levels. Human CFB mRNA levels were measured using the human primer probe set RTS3459. CFB mRNA levels were normalized to RIBOGREEN®, and also to the housekeeping gene, Cyclophilin. Results were calculated as percent inhibition of CFB mRNA expression compared to the control.

All the antisense oligonucleotides effected inhibition of human CFB mRNA levels in the liver.

TABLE 75
Percent reduction of CFB mRNA levels in hCFB mice

| | Normalized | Normalized |
|---|---|---|
| | to | to |
| ISIS No | RIBOGREEN | Cyclophilin |
| 532770 | 86 | 87 |
| 532800 | 88 | 87 |
| 532809 | 69 | 69 |
| 588540 | 95 | 94 |
| 588544 | 91 | 91 |
| 588548 | 78 | 77 |
| 588550 | 89 | 88 |
| 588553 | 94 | 94 |
| 588555 | 94 | 94 |
| 588848 | 83 | 82 |
| 594430 | 78 | 76 |

### Example 15: In Vivo Antisense Inhibition of Murine CFB

Several antisense oligonucleotides were designed that were targeted to murine CFB mRNA (GENBANK Accession No. NM_008198.2, incorporated herein as SEQ ID NO: 5). The target start sites and sequences of each oligonucleotide are described in the table below. The chimeric antisense oligonucleotides in the table below were designed as 5-10-5 MOE gapmers. The gapmers are 20 nucleosides in length, wherein the central gap segment is comprised of 10 2′-deoxynucleosides and is flanked on both sides (in the 5′ and 3′ directions) by wings comprising 5 nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines.

TABLE 76
Gapmers targeting murine CFB

| | | Target Start | SEQ |
|---|---|---|---|
| | | Site on SEQ | ID |
| ISIS No | Sequence | ID NO: 5 | NO |
| 516269 | GCATAAGAGGGTACCAGCTG | 2593 | 804 |
| 516272 | GTCCTTTAGCCAGGGCAGCA | 2642 | 805 |
| 516323 | TCCACCCATGTTGTGCAAGC | 1568 | 806 |
| 516330 | CCACACCATGCCACAGAGAC | 1826 | 807 |
| 516341 | TTCCGAGTCAGGCTCTTCCC | 2308 | 808 |

### Treatment

Groups of four C₅₇BL/6 mice each were injected with 50 mg/kg of ISIS 516269, ISIS 516272, ISIS 516323, ISIS 516330, or ISIS 516341 administered weekly for 3 weeks. A control group of mice was injected with phosphate buffered saline (PBS) administered weekly for 3 weeks.

### CFB RNA Analysis

At the end of the study, RNA was extracted from liver tissue for real-time PCR analysis of CFB, using primer probe set RTS3430 (forward sequence GGGCAAACAGCAATTTGTGA, designated herein as SEQ ID NO: 816; reverse sequence TGGCTACCCACCTTCCTTGT, designated herein as SEQ ID NO: 817; probe sequence CTGGATACTGTCCCAATCCCGGTATTCCX, designated herein as SEQ ID NO: 818).

The mRNA levels were normalized using RIBOGREEN®. As shown in the Table below, some of the antisense oligonucleotides achieved reduction of murine CFB over the PBS control. Results are presented as percent inhibition of CFB, relative to control.

TABLE 77
Percent inhibition of murine CFB mRNA in C57BL/6 mice

| ISIS No | % |
|---|---|
| 516269 | 29 |
| 516272 | 72 |
| 516323 | 77 |
| 516330 | 62 |
| 516341 | 72 |

### Protein Analysis

CFB protein levels were measured in the kidney, liver, plasma, and in the eye by western Blot using goat anti-CFB antibody (Sigma Aldrich). Results are presented as percent inhibition of CFB, relative to PBS control. ‘n/a’ indicates that measurements were not taken for that sample. As shown in the Table below, antisense inhibition of CFB by ISIS oligonucleotides resulted in a reduction of CFB protein in various tissues. As shown in the Table below, systemic administration of ISIS oligonucleotides was effective in reducing CFB levels in the eye.

TABLE 78
Percent inhibition of murine CFB protein in C57BL/6 mice

| ISIS No | Kidney | Liver | Plasma | Eye |
|---|---|---|---|---|
| 516269 | 20 | 58 | n/a | 70 |
| 516272 | 48 | 74 | n/a | 99 |
| 516323 | 73 | 85 | 90 | 93 |
| 516330 | 77 | 80 | n/a | n/a |
| 516341 | 80 | 88 | 68 | n/a |

### Example 16: Dose-Dependent Antisense Inhibition of Murine CFB

Groups of four C₅₇BL/6 mice each were injected with 25 mg/kg, 50 mg/kg, or 100 mg/kg of ISIS 516272, and ISIS 516323 administered weekly for 6 weeks. Another two groups of mice were injected with 100 mg/kg of ISIS 516330 or ISIS 516341 administered weekly for 6 weeks. Two control groups of mice were injected with phosphate buffered saline (PBS) administered weekly for 6 weeks.

### CFB RNA Analysis

RNA was extracted from liver and kidney tissues for real-time PCR analysis of CFB, using primer probe set RTS3430. The mRNA levels were normalized using RIBOGREEN®. As shown in the Table below, the antisense oligonucleotides achieved dose-dependent reduction of murine CFB over the PBS control. Results are presented as percent inhibition of CFB, relative to control.

TABLE 79
Percent inhibition of murine CFB mRNA in C57BL/6 mice

| | Dose | | |
|---|---|---|---|
| ISIS No | (mg/kg/wk) | Liver | Kidney |
| 516272 | 25 | 39 | 32 |
| | 50 | 73 | 36 |
| | 100 | 87 | 42 |
| 516323 | 25 | 36 | 41 |
| | 50 | 65 | 47 |
| | 100 | 79 | 71 |
| 516330 | 100 | 85 | 45 |
| 516341 | 200 | 89 | 65 |

### Protein Analysis

CFB protein levels were measured in the plasma by western Blot using goat anti-CFB antibody (Sigma Aldrich). As shown in the table below, antisense inhibition of CFB by the ISIS oligonucleotides resulted in a reduction of CFB protein. Results are presented as percent inhibition of CFB, relative to PBS control. ‘n/a’ indicates that measurements were not taken for that sample.

CFB protein levels were also measured in the eye by Western Blot. All treatment groups demonstrated an inhibition of CFB by 95%, with some sample measurements being below detection levels of the assay.

TABLE 80
Percent inhibition of murine CFB protein in C57BL/6 mice

| ISIS No | Dose (mg/kg/wk) | Liver |
|---|---|---|
| 516272 | 25 | 32 |
| | 50 | 70 |
| | 100 | 83 |
| 516323 | 25 | 43 |
| | 50 | 80 |
| | 100 | 90 |
| 516330 | 100 | n/a |
| 516341 | 200 | n/a |

### Example 17: Effect of Antisense Inhibition of CFB in the NZB/W F₁ Mouse Model

The NZB/W F₁ is the oldest classical model of lupus, where the mice develop severe lupus-like phenotypes comparable to that of lupus patients (Theofilopoulos, A. N. and Dixon, F. J. Advances in Immunology, vol. 37, pp. 269-390, 1985). These lupus-like phenotypes include lymphadenopathy, splenomegaly, elevated serum antinuclear autoantibodies (ANA) including anti-dsDNA IgG, a majority of which are IgG2a and IgG3, and immune complex-mediated glomerulonephritis (GN) that becomes apparent at 5-6 months of age, leading to kidney failure and death at 10-12 months of age.

### Study 1

A study was conducted to demonstrate that treatment with antisense oligonucleotides targeting CFB would improve renal pathology in the mouse model. Female NZB/W F1 mice, 17 weeks old, were purchased from Jackson Laboratories. Groups of 16 mice each received doses of 100 μg/kg/week of ISIS 516272 or ISIS 516323 for 20 weeks. Another group of 16 mice received doses of 100 μg/kg/week of control oligonucleotide ISIS 141923 for 20 weeks. Another group of 10 mice received doses of PBS for 20 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. The mRNA levels were normalized using RIBOGREEN®. As shown in the Table below, some of the antisense oligonucleotides achieved reduction of murine CFB over the PBS control. Results are presented as percent inhibition of CFB, relative to control.

TABLE 81
Percent inhibition of murine CFB mRNA in NZB/W Fl mice

| ISIS No | Liver | Kidney |
|---|---|---|
| 516272 | 55 | 25 |
| 516323 | 63 | 43 |
| 141923 | 0 | 0 |

### Proteinuria

Proteinuria is expected in 60% of animals in this mouse model. The cumulative incidence of severe proteinuria was measured by calculating the total protein to creatinine ratio using a clinical analyzer. The results are presented in the table below and demonstrate that treatment with antisense oligonucleotides targeting CFB achieved reduction of proteinuria in the mice compared to the PBS control and the control oligonucleotide treated mice.

TABLE 82
Percent cumulative incidence of
severe proteinuria in NZB/W F1 mice

| | % |
|---|---|
| PBS | 40 |
| ISIS 516272 | 6 |
| ISIS 516323 | 0 |
| ISIS 141923 | 25 |

### Survival

Survival of the mice was monitored by keeping count of the mice at the start of treatment and then again at week 20. The results are presented in the table below and demonstrate that treatment with antisense oligonucleotides targeting CFB increased survival in the mice compared to the PBS control and the control oligonucleotide treated mice.

TABLE 83
Number of surviving mice and % survival

| | Week | Week | % survival at |
|---|---|---|---|
| | 1 | 20 | week 20 |
| PBS | 10 | 6 | 60 |
| ISIS 516272 | 16 | 15 | 94 |
| ISIS 516323 | 16 | 16 | 100 |
| ISIS 141923 | 16 | 12 | 75 |

### Glomerular Deposition

The amount of C₃ deposition, as well as IgG deposition, in the glomeruli of the kidneys was measured by immunohistochemistry with an anti-C₃ antibody. The results are presented in the table below and demonstrate that treatment with antisense oligonucleotides targeting CFB achieved reduction of both C₃ and IgG depositions in the kidney glomeruli compared to the PBS control and the control oligonucleotide treated mice.

TABLE 84
Percent inhibition of glomerula deposition in NZB/W F1 mice

| ISIS No | C3 | IgG |
|---|---|---|
| 516272 | 45 | 20 |
| 516323 | 48 | 2 |
| 141923 | 0 | 0 |

### Study 2

Female NZB/W F1 mice, 16 weeks old, were purchased from Jackson Laboratories. A group of 10 mice received doses of 100 μg/kg/week of ISIS 516323 for 12 weeks. Another group of 10 mice received doses of 100 μg/kg/week of control oligonucleotide ISIS 141923 for 12 weeks. Another group of 10 mice received doses of PBS for 12 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. As shown in the table below, treatment with ISIS 516323 achieved reduction of murine CFB over the PBS control. Results are presented as percent inhibition of CFB, relative to control.

TABLE 85
Percent inhibition of murine CFB mRNA in NZB/W F1 mice

| ISIS No | Liver | Kidney |
|---|---|---|
| 516323 | 75 | 46 |
| 141923 | 0 | 6 |

### Proteinuria

The cumulative incidence of severe proteinuria was assessed by measuring urine total protein to creatinine ratio, as well as by measuring total microalbumin levels. The results are presented in the tables below and demonstrate that treatment with antisense oligonucleotides targeting CFB reduced proteinuria in the mice compared to the PBS control and the control oligonucleotide treated mice.

TABLE 86
Proteinuria in NZB/W F1 mice measured as
urine microalbumin levels (mg/dl)

| ISIS No | Week 0 | Week 6 | Week 8 | Week 10 |
|---|---|---|---|---|
| 516323 | 0 | 0 | 5.4 | 0.4 |
| 141923 | 0 | 8.28 | 8.6 | 5.6 |

TABLE 87
Proteinuria in NZB/W F1 mice measured
as total protein to creatinine ratio

| ISIS No | Week 0 | Week 6 | Week 8 | Week 10 |
|---|---|---|---|---|
| 516323 | 5.5 | 7.8 | 8.6 | 7.2 |
| 141923 | 6.9 | 10.0 | 13.5 | 7.2 |

### Survival

Survival of the mice was monitored by keeping count of the mice at the start of treatment and then again at week 12. The results are presented in the table below and demonstrate that treatment with antisense oligonucleotides targeting CFB increased survival in the mice compared to the PBS control and the control oligonucleotide treated mice.

TABLE 88
Number of surviving mice

| | Week 1 | Week 12 |
|---|---|---|
| PBS | 10 | 9 |
| ISIS 516323 | 10 | 10 |
| ISIS 141923 | 10 | 9 |

### Example 18: Effect of Antisense Inhibition of CFB in the MRL Mouse Model

The MRL/lpr lupus nephritis mouse model develops an SLE-like phenotype characterized by lymphadenopathy due to an accumulation of double negative (CD4⁻ CD8⁻) and B220⁺ T-cells. These mice display an accelerated mortality rate. In addition, the mice have high concentrations of circulating immunoglobulins, which included elevated levels of autoantibodies such as ANA, anti-ssDNA, anti-dsDNA, anti-Sm, and rheumatoid factors, resulting in large amounts of immune complexes (Andrews, B. et al., J. Exp. Med. 148: 1198-1215, 1978).

### Treatment

A study was conducted to investigate whether treatment with antisense oligonucleotides targeting CFB would reverse renal pathology in the mouse model. Female MRL/lpr mice, 14 weeks old, were purchased from Jackson Laboratories. A group of 10 mice received doses of 50 μg/kg/week of ISIS 516323 for 7 weeks. Another group of 10 mice received doses of 50 μg/kg/week of control oligonucleotide ISIS 141923 for 7 weeks. Another group of 10 mice received doses of PBS for 7 weeks and served as the control group to which all the other groups were compared. Terminal endpoints were collected 48 hours after the last dose was injected.

### CFB RNA Analysis

RNA was extracted from liver tissue for real-time PCR analysis of CFB, using primer probe set RTS3430. As shown in the Table below, ISIS 516323 reduced CFB over the PBS control. Results are presented as percent inhibition of CFB, relative to control.

TABLE 89
Percent inhibition of murine CFB mRNA in MRL/lpr mice

| ISIS No | % |
|---|---|
| 516323 | 68 |
| 141923 | 4 |

### Renal Pathology

Renal pathology was evaluated by two methods. Histological sections of the kidney were stained with Haematoxylin & Eosin. The PBS control demonstrated presence of multiglomerular crescents tubular casts, which is a symptom of glomerulosclerosis. In contrast, the sections from mice treated with ISIS 516323 showed absent crescents tubular casts with minimal bowman capsule fibrotic changes, moderate to severe segmental mesangial cell expansion and glomerular basement membrane thickening.

Accumulation of C3 in the kidney was also assessed by immunohistochemistry with anti-C3 antibodies. The whole kidney C3 immunohistochemistry intensity score was calculated by intensity scoring system, which was computed by capturing 10 glomeruli per kidney and calculation the intensity of positive C3 staining. The results are presented in the table below and demonstrate that treatment with ISIS 516323 reduced renal C3 accumulation compared to the control groups.

TABLE 90
Renal C3 accumulation in MRL/lpr mice

| | Whole | C3 quantification |
|---|---|---|
| | kidney C3 | (area/total area) % |
| | intensity score | of average PBS |
| PBS | 2.5 | 100 |
| ISIS 516323 | 1.6 | 68 |
| ISIS 141923 | 2.2 | 99 |

### Plasma C3 Levels

Reduction of CFB inhibits activation of the alternative complement pathway, preventing C₃ consumption and leading to an apparent elevation of plasma C3 levels. Plasma C3 levels from terminal bleed were measured by clinical analyzer. The results are presented in the table below and demonstrate that treatment with ISIS 516323 increased C3 levels (p<0.001) in the plasma compared to the control groups.

TABLE 91
Plasma C3 levels (mg/dL) in MRL/lpr mice

| ISIS No | C3 |
|---|---|
| 516323 | 28 |
| 141923 | 16 |

The results indicate that treatment with antisense oligonucleotides targeting CFB reverses renal pathology in the lupus mouse model.

### Example 19: Effect of Antisense Inhibition of CFB in the CFH Het Mouse Model

CFH heterozygous (CFH Het, CFH^{+/-}) mouse model expresses a mutant Factor H protein in combination with the full-length mouse protein (Pickering, M. C. et al., J. Exp. Med. 2007, 204: 1249-56). Renal histology remains normal in these mice up to six months old.

### Study 1

Groups of 8 CFH^{+/-} mice, 6 weeks old, each received doses of 75 mg/kg/week of ISIS 516323 or ISIS 516341 for 6 weeks. Another group of 8 mice received doses of 75 mg/kg/week of control oligonucleotide ISIS 141923 for 6 weeks. Another group of 8 mice received doses of PBS for 6 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. As shown in the Table below, the antisense oligonucleotides reduced CFB over the PBS control. Results are presented as percent inhibition of CFB, relative to control.

TABLE 92
Percent inhibition of murine CFB mRNA in CFH^{+/-} mice

| ISIS No | Liver | Kidney |
|---|---|---|
| 516323 | 80 | 38 |
| 516341 | 90 | 44 |
| 141923 | 0 | 17 |

Plasma C₃ levels

Reduction of CFB inhibits activation of the alternative complement pathway, preventing C₃ consumption and leading to an apparent elevation of plasma C₃ levels. Plasma C₃ levels from terminal plasma collection were measured by clinical analyzer. The results are presented in the table below and demonstrate that treatment with ISIS 516323 increased C₃ to normal levels in the plasma.

TABLE 93
Plasma C3 levels (mg/dL) in CFH^{+/-} mice

| ISIS No | C3 |
|---|---|
| 516323 | 15 |
| 516341 | 17 |
| 141923 | 8 |

### Study 2

Groups of 5 CFH^{+/-} mice each received doses of 12.5 mg/kg/week, 25 mg/kg/week, 50 mg/kg/week, 75 mg/kg/week, or 100 mg/kg/week of ISIS 516323 or ISIS 516341 for 6 weeks. Another group of 5 mice received doses of 75 μg/kg/week of control oligonucleotide ISIS 141923 for 6 weeks. Another group of 5 mice received doses of PBS for 6 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. As shown in the Table below, the antisense oligonucleotides reduced CFB over the PBS control in a dose dependent manner. Results are presented as percent inhibition of CFB, relative to control.

TABLE 94
Percent inhibition of murine CFB mRNA in
the liver of CFH^{+/-} mice

| ISIS No | Dose (mg/kg/week) | % |
|---|---|---|
| 516323 | 12.5 | 34 |
| | 25 | 51 |
| | 50 | 72 |
| | 75 | 79 |
| | 100 | 92 |
| 516341 | 12.5 | 38 |
| | 25 | 57 |
| | 50 | 89 |
| | 75 | 92 |
| | 100 | 90 |
| 141923 | 75 | 13 |

Plasma C₃ levels

Reduction of CFB inhibits activation of the alternative complement pathway, preventing C₃ consumption and leading to an apparent elevation of plasma C₃ levels. Plasma C₃ levels from terminal plasma collection were measured by clinical analyzer. The results are presented in the table below and demonstrate that treatment with ISIS oligonucleotides targeting CFB increased C₃ levels in the plasma.

TABLE 95
Plasma C3 levels (mg/dL) in CFH^{+/-} mice

| | Dose (mg/kg/week) | C3 |
|---|---|---|
| PBS | — | 10.1 |
| 516323 | 12.5 | 11.4 |
| | 25 | 15.5 |
| | 50 | 17.0 |
| | 75 | 18.3 |
| | 100 | 18.8 |
| 516341 | 12.5 | 12.1 |
| | 25 | 16.3 |
| | 50 | 18.6 |
| | 75 | 22.1 |
| | 100 | 19.1 |
| 141923 | 75 | 8.9 |

### Example 20: Effect of ISIS Antisense Oligonucleotides Targeting Human CFB in Cynomolgus Monkeys

Cynomolgus monkeys were treated with ISIS antisense oligonucleotides selected from studies described in the Examples above. Antisense oligonucleotide efficacy and tolerability, as well as their pharmacokinetic profile in the liver and kidney, were evaluated.

At the time this study was undertaken, the cynomolgus monkey genomic sequence was not available in the National Center for Biotechnology Information (NCBI) database; therefore cross-reactivity with the cynomolgus monkey gene sequence could not be confirmed. Instead, the sequences of the ISIS antisense oligonucleotides used in the cynomolgus monkeys was compared to a rhesus monkey sequence for homology. The human antisense oligonucleotides tested below are cross-reactive (with 0 or 1 mismatches) with the rhesus genomic sequence (GENBANK Accession No. NW_001116486.1 truncated from nucleotides 536000 to 545000, designated herein as SEQ ID NO: 3). The start and stop sites of each oligonucleotide targeted to SEQ ID NO: 3 is presented in the Table below. “Start site” indicates the 5′-most nucleotide to which the gapmer is targeted in the rhesus monkey gene sequence. ‘Mismatches’ indicates the number of nucleobases in the human oligonucleotide that are mismatched with the rhesus genomic sequence.

TABLE 96
Antisense oligonucleotides complementary to the
rhesus CFB genomic sequence (SEQ ID NO: 3)

| | Target | | | SEQ |
|---|---|---|---|---|
| ISIS No | Start Site | Mismatches | Chemistry | ID NO |
| 532770 | 6788 | 0 | 5-10-5 MOE | 198 |
| 532800 | 7500 | 0 | 5-10-5 MOE | 228 |
| 532809 | 7614 | 0 | 5-10-5 MOE | 237 |
| 588540 | 7627 | 1 | 5-10-5 MOE | 440 |
| 588544 | 7631 | 1 | 5-10-5 MOE | 444 |
| 588548 | 7635 | 1 | 5-10-5 MOE | 448 |
| 588550 | 7637 | 1 | 5-10-5 MOE | 450 |
| 588553 | 7640 | 1 | 5-10-5 MOE | 453 |
| 588555 | 7643 | 0 | 5-10-5 MOE | 455 |
| 588848 | 7639 | 1 | Deoxy, MOE and cEt | 598 |
| 594430 | 6790 | 0 | 3-10-3 cEt | 549 |

### Treatment

Prior to the study, the monkeys were kept in quarantine for at least a 30 day period, during which the animals were observed daily for general health. The monkeys were 2-4 years old and weighed between 2 and 4 kg. Eleven groups of 4-6 randomly assigned male cynomolgus monkeys each were injected subcutaneously with ISIS oligonucleotide or PBS at four sites on the back in a clockwise rotation (i.e. left, top, right, and bottom), one site per dose. The monkeys were given four loading doses of PBS or 40 mg/kg of ISIS 532800, ISIS 532809, ISIS 588540, ISIS 588544, ISIS 588548, ISIS 588550, ISIS 588553, ISIS 588555, ISIS 588848, or ISIS 594430 for the first week (days 1, 3, 5, and 7), and were subsequently dosed once a week for 12 weeks (days 14, 21, 28, 35, 42, 49, 56, 63, 70, 77, and 84) with PBS or 40 mg/kg of ISIS oligonucleotide. ISIS 532770 was tested in a separate study with similar conditions with two male and two female cynomolgus monkeys in the group.

### Hepatic Target Reduction

### RNA Analysis

On day 86, liver and kidney samples were collected in duplicate (approximately 250 mg each) for CFB mRNA analysis. The samples were flash frozen in liquid nitrogen at necropsy within approximately 10 minutes of sacrifice.

RNA was extracted from liver and kidney for real-time PCR analysis of measurement of mRNA expression of CFB. Results are presented as percent change of mRNA, relative to PBS control, normalized with RIBOGREEN®. RNA levels were also normalized with the house-keeping gene, Cyclophilin A. RNA levels were measured with the primer probe sets RTS3459, described above, or RTS4445_MGB (forward sequence CGAAGAAGCTCAGTGAAATCAA, designated herein as SEQ ID NO: 819; reverse sequence TGCCTGGAGGGCCCTCTT, designated herein as SEQ ID NO: 820; probe sequence AGACCACAAGTTGAAGTC, designated herein as SEQ ID NO: 815).

As shown in the Tables below, treatment with ISIS antisense oligonucleotides resulted in reduction of CFB mRNA in comparison to the PBS control. Analysis of CFB mRNA levels revealed that several of the ISIS oligonucleotides reduced CFB levels in liver and/or kidney. Here ‘0’ indicates that the expression levels were not inhibited. ‘*’ indicates that the oligonucleotide was tested in a separate study with similar conditions.

TABLE 97
Percent inhibition of CFB mRNA in the cynomolgus
monkey liver relative to the PBS control

| | RTS3459/ | RTS3459/ | RTS445_MGB/ | RTS445_MGB/ |
|---|---|---|---|---|
| ISIS No | Cyclophilin A | RIBOGREEN | Cyclophilin A | RIBOGREEN |
| 532770* | 12 | 37 | 24 | 45 |
| 532800 | 54 | 45 | 56 | 46 |
| 588540 | 31 | 27 | 28 | 24 |
| 588548 | 68 | 67 | 68 | 67 |
| 588550 | 53 | 39 | 51 | 37 |
| 588553 | 74 | 59 | 74 | 59 |
| 588555 | 73 | 71 | 71 | 69 |
| 588848 | 9 | 0 | 6 | 0 |
| 594430 | 24 | 26 | 23 | 25 |

TABLE 98
Percent inhibition of CFB mRNA in the cynomolgus
monkey kidney relative to the PBS control

| | RTS3459/ | RTS3459/ | RTS445_MGB/ | RTS445_MGB/ |
|---|---|---|---|---|
| ISIS No | Cyclophilin A | RIBOGREEN | Cyclophilin A | RIBOGREEN |
| 532770* | 34 | 56 | 2 | 31 |
| 532800 | 36 | 30 | 43 | 37 |
| 588540 | 70 | 71 | 67 | 69 |
| 588548 | 83 | 84 | 82 | 83 |
| 588550 | 81 | 77 | 78 | 74 |
| 588553 | 86 | 84 | 86 | 85 |
| 588555 | 32 | 34 | 48 | 50 |
| 588848 | 89 | 91 | 87 | 90 |
| 594430 | 33 | 37 | 19 | 23 |

### Protein Analysis

Approximately 1 mL of blood was collected from all available animals at day 85 and placed in tubes containing the potassium salt of EDTA. The blood samples were placed in wet-ice or Kryorack immediately, and centrifuged (3000 rpm for 10 min at 4° C.) to obtain plasma (approximately 0.4 mL) within 60 minutes of collection. Plasma levels of CFB were measured in the plasma by radial immunodiffusion (RID), using a polyclonal anti-Factor B antibody. The results are presented in the Table below. ISIS 532770 was tested in a separate study and plasma protein levels were measured on day 91 or 92 in that group.

Analysis of plasma CFB revealed that several ISIS oligonucleotides reduced protein levels in a sustained manner. ISIS 532770, which was tested in a separate study, reduced CFB protein levels on day 91/92 by 50% compared to baseline values. The reduction in plasma CFB protein levels correlates well with liver CFB mRNA level reduction in the corresponding groups of animals.

TABLE 99
Plasma protein levels (% baseline
values) in the cynomolgus monkey

| | Day 1 | Day 30 | Day 58 | Day 72 | Day 86 |
|---|---|---|---|---|---|
| PBS | 113 | 115 | 95 | 83 | 86 |
| ISIS 532800 | 117 | 68 | 52 | 39 | 34 |
| ISIS 532809 | 104 | 121 | 100 | 80 | 71 |
| ISIS 588540 | 108 | 72 | 61 | 40 | 38 |
| ISIS 588544 | 118 | 74 | 53 | 33 | 29 |
| ISIS 588548 | 110 | 41 | 28 | 20 | 16 |
| ISIS 588550 | 104 | 64 | 54 | 38 | 37 |
| ISIS 588553 | 97 | 42 | 35 | 18 | 16 |
| ISIS 588555 | 107 | 35 | 37 | 18 | 18 |
| ISIS 588848 | 116 | 95 | 92 | 69 | 71 |
| ISIS 594430 | 104 | 64 | 59 | 45 | 46 |

### Tolerability Studies

### Body Weight Measurements

To evaluate the effect of ISIS oligonucleotides on the overall health of the animals, body and organ weights were measured and are presented in the Table below. ‘*’ indicates that the oligonucleotide was tested in a separate study with similar conditions and is the average of the measurements from male and female monkeys. The results indicate that effect of treatment with antisense oligonucleotides on body and organ weights was within the expected range for antisense oligonucleotides.

TABLE 100
Final body weights (g) in cynomolgus monkey

| | Day | Day | Day | Day | Day | Day | Day |
|---|---|---|---|---|---|---|---|
| | 1 | 14 | 28 | 42 | 56 | 70 | 84 |
| PBS | 2887 | 2953 | 3028 | 3094 | 3125 | 3143 | 3193 |
| ISIS 532770* | 2963 | 2947 | 2966 | 3050 | 3097 | 3138 | 3160 |
| ISIS 532800 | 2886 | 2976 | 3072 | 3149 | 3220 | 3269 | 3265 |
| ISIS 532809 | 2755 | 2836 | 2927 | 2983 | 3019 | 3071 | 3098 |
| ISIS 588540 | 2779 | 2834 | 2907 | 2934 | 2981 | 3034 | 3057 |
| ISIS 588544 | 2837 | 2896 | 3009 | 3064 | 3132 | 3163 | 3199 |
| ISIS 588548 | 2694 | 2816 | 2882 | 2990 | 3073 | 3149 | 3161 |
| ISIS 588550 | 2855 | 2988 | 3062 | 3188 | 3219 | 3282 | 3323 |
| ISIS 588553 | 3033 | 3156 | 3256 | 3335 | 3379 | 3372 | 3442 |
| ISIS 588555 | 2757 | 2863 | 2965 | 3022 | 3075 | 3088 | 3158 |
| ISIS 588848 | 2850 | 3018 | 3032 | 3187 | 3230 | 3212 | 3291 |
| ISIS 594430 | 2884 | 2963 | 2953 | 3149 | 3187 | 3204 | 3256 |

TABLE 101
Final organ weights (g) in cynomolgus monkey

| | Spleen | Heart | Kidney | Liver |
|---|---|---|---|---|
| PBS | 2.8 | 11.6 | 11.9 | 55.8 |
| ISIS 532770* | 5.0 | 11.3 | 20.6 | 77.9 |
| ISIS 532800 | 6.2 | 11.9 | 18.6 | 94.4 |
| ISIS 588540 | 4.0 | 11.4 | 13.5 | 67.1 |
| ISIS 588548 | 4.1 | 11.7 | 17.3 | 72.0 |
| ISIS 588550 | 5.8 | 10.9 | 18.5 | 81.8 |
| ISIS 588553 | 5.0 | 12.7 | 17.2 | 85.9 |
| ISIS 588555 | 4.7 | 11.8 | 15.9 | 88.3 |
| ISIS 588848 | 5.0 | 12.7 | 14.4 | 75.7 |
| ISIS 594430 | 3.9 | 11.9 | 14.8 | 69.9 |

### Liver Function

To evaluate the effect of ISIS oligonucleotides on hepatic function, blood samples were collected from all the study groups. The blood samples were collected from the cephalic, saphenous, or femoral veins, 48 hours post-dosing. The monkeys were fasted overnight prior to blood collection. Blood (1.5 mL) was collected in tubes without anticoagulant for serum separation. The tubes were kept at room temperature for a minimum of 90 minutes and then centrifuged (approximately 3,000 rpm for 10 min) to obtain serum. Levels of various liver function markers were measured using a Toshiba 200FR NEO chemistry analyzer (Toshiba Co., Japan).

Plasma levels of ALT and AST were measured and the results are presented in the Table below, expressed in IU/L. Bilirubin, a liver function marker, was similarly measured and is presented in the Table below expressed in mg/dL. ‘*’ indicates that the oligonucleotide was tested in a separate study with similar conditions and is the average of the measurements from male and female monkeys. The results indicate that most of the antisense oligonucleotides had no effect on liver function outside the expected range for antisense oligonucleotides.

TABLE 102
Liver chemistry marker levels in cynomolgus
monkey plasma on day 86

| | ALT (IU/L) | AST (IU/L) | Bilirubin (mg/dL) |
|---|---|---|---|
| PBS | 71 | 57 | 0.3 |
| ISIS 532770* | 59 | 58 | 0.1 |
| ISIS 532800 | 65 | 86 | 0.1 |
| ISIS 532809 | 35 | 58 | 0.1 |
| ISIS 588540 | 70 | 88 | 0.2 |
| ISIS 588544 | 55 | 97 | 0.2 |
| ISIS 588548 | 61 | 85 | 0.2 |
| ISIS 588550 | 94 | 84 | 0.2 |
| ISIS 588553 | 44 | 65 | 0.2 |
| ISIS 588555 | 63 | 84 | 0.2 |
| ISIS 588848 | 69 | 65 | 0.2 |
| ISIS 594430 | 86 | 53 | 0.2 |

### Kidney Function

To evaluate the effect of ISIS oligonucleotides on kidney function, blood samples were collected from all the study groups. The blood samples were collected from the cephalic, saphenous, or femoral veins, 48 hours post-dosing. The monkeys were fasted overnight prior to blood collection. Blood was collected in tubes without anticoagulant for serum separation. The tubes were kept at room temperature for a minimum of 90 minutes and then centrifuged (approximately 3,000 rpm for 10 min) to obtain serum. Levels of BUN and creatinine were measured using a Toshiba 200FR NEO chemistry analyzer (Toshiba Co., Japan). Results are presented in the Table below, expressed in mg/dL. “*” indicates that the oligonucleotide was tested in a separate study with similar conditions and is the average of the measurements from male and female monkeys.

For urinalysis, fresh urine from all the animals was collected in the morning using a clean cage pan on wet ice. Food was removed overnight the day before urine collection but water was supplied. Urine samples (approximately 1 mL) were analyzed for protein to creatinine (P/C) ratio using a Toshiba 200FR NEO automated chemistry analyzer (Toshiba Co., Japan). ‘n.d.’ indicates that the urine protein level was under the detection limit of the analyzer.

The plasma and urine chemistry data indicate that most of the ISIS oligonucleotides did not have any effect on the kidney function outside the expected range for antisense oligonucleotides.

TABLE 103
Renal chemistry marker levels (mg/dL)
in cynomolgus monkey plasma on day 86

| | BUN | Creatinine | Total protein |
|---|---|---|---|
| PBS | 28 | 0.9 | 8.0 |
| ISIS 532770* | 20 | 0.9 | 6.9 |
| ISIS 532800 | 25 | 0.9 | 7.5 |
| ISIS 532809 | 23 | 0.8 | 7.4 |
| ISIS 588540 | 30 | 0.8 | 7.5 |
| ISIS 588544 | 26 | 0.9 | 7.4 |
| ISIS 588548 | 25 | 0.9 | 7.6 |
| ISIS 588550 | 24 | 0.9 | 7.2 |
| ISIS 588553 | 25 | 0.8 | 7.2 |
| ISIS 588555 | 25 | 0.8 | 7.6 |
| ISIS 588848 | 24 | 0.9 | 7.5 |
| ISIS 594430 | 25 | 0.8 | 7.2 |

TABLE 104
Total Protein/Creatinine ratio in cynomolgus monkey urine

| | Day 44 | Day 86 |
|---|---|---|
| PBS | 0.03 | n.d. |
| ISIS 532800 | 0.01 | n.d. |
| ISIS 532809 | 0.01 | n.d. |
| ISIS 588540 | 0.03 | n.d. |
| ISIS 588544 | 0.01 | 0.09 |
| ISIS 588548 | 0.01 | 0.01 |
| ISIS 588550 | 0.04 | 0.01 |
| ISIS 588553 | 0.05 | n.d. |
| ISIS 588555 | 0.03 | 0.03 |
| ISIS 588848 | 0.09 | n.d. |
| ISIS 594430 | 0.03 | n.d. |

### Hematology

To evaluate any effect of ISIS oligonucleotides in cynomolgus monkeys on hematologic parameters, blood samples of approximately 0.5 mL of blood was collected from each of the available study animals in tubes containing K₂-EDTA. Samples were analyzed for red blood cell (RBC) count, white blood cells (WBC) count, individual white blood cell counts, such as that of monocytes, neutrophils, lymphocytes, as well as for platelet count, hemoglobin content and hematocrit, using an ADVIA120 hematology analyzer (Bayer, USA). The data is presented in the Tables below. ‘*’ indicates that the oligonucleotide was tested in a separate study with similar conditions and is the average of the measurements from male and female monkeys.

The data indicate the oligonucleotides did not cause any changes in hematologic parameters outside the expected range for antisense oligonucleotides at this dose.

TABLE 105
Blood cell counts in cynomolgus monkeys

| | RBC | Platelets | WBC | Neutrophils | Lymphocytes | Monocytes |
|---|---|---|---|---|---|---|
| | (×10⁶/μL) | (×10³/μL) | (×10³/μL) | (% WBC) | (% total) | (% total) |
| PBS | 5.8 | 347 | 9.4 | 42.7 | 53.1 | 3.0 |
| ISIS 532770* | 5.4 | 386 | 10.8 | 22.3 | 71.7 | 3.3 |
| ISIS 532800 | 5.6 | 360 | 13.1 | 29.5 | 61.1 | 6.5 |
| ISIS 532809 | 5.2 | 400 | 11.5 | 56.6 | 38.2 | 2.5 |
| ISIS 588540 | 5.5 | 367 | 11.7 | 50.9 | 42.7 | 2.1 |
| ISIS 588544 | 5.2 | 373 | 14.3 | 56.6 | 37.6 | 4.3 |
| ISIS 588548 | 5.1 | 373 | 9.7 | 40.4 | 54.3 | 3.9 |
| ISIS 588550 | 6.1 | 343 | 9.9 | 32.1 | 61.7 | 4.6 |
| ISIS 588553 | 5.2 | 424 | 9.3 | 41.7 | 53.2 | 3.6 |
| ISIS 588555 | 5.1 | 411 | 9.6 | 45.1 | 49.7 | 3.5 |
| ISIS 588848 | 5.7 | 370 | 10.0 | 39.8 | 55.8 | 3.1 |
| ISIS 594430 | 5.7 | 477 | 10.6 | 47.3 | 47.8 | 3.6 |

TABLE 106
Hematologic parameters in cynomolgus monkeys

| | Hemoglobin | HCT |
|---|---|---|
| | (g/dL) | (%) |
| PBS | 14.1 | 46.6 |
| ISIS 532770* | 12.4 | 40.9 |
| ISIS 532800 | 12.3 | 40.5 |
| ISIS 532809 | 12.2 | 40.4 |
| ISIS 588540 | 12.5 | 41.5 |
| ISIS 588544 | 11.9 | 38.1 |
| ISIS 588548 | 12.3 | 39.6 |
| ISIS 588550 | 13.4 | 45.0 |
| ISIS 588553 | 12.6 | 39.8 |
| ISIS 588555 | 11.6 | 38.1 |
| ISIS 588848 | 13.2 | 42.7 |
| ISIS 594430 | 13.4 | 43.1 |

### Measurement of Oligonucleotide Concentration

The concentration of the full-length oligonucleotide was measured in the kidney and liver tissues. The method used is a modification of previously published methods (Leeds et al., 1996; Geary et al., 1999) which consist of a phenol-chloroform (liquid-liquid) extraction followed by a solid phase extraction. Tissue sample concentrations were calculated using calibration curves, with a lower limit of quantitation (LLOQ) of approximately 1.14 μg/g. The results are presented in the Table below, expressed as μg/g liver or kidney tissue.

TABLE 107
Antisense oligonucleotide distribution

| | Kidney (μg/g) | Liver (μg/g) | Kidney/Liver ratio |
|---|---|---|---|
| ISIS 532800 | 3881 | 1633 | 2.4 |
| ISIS 588540 | 3074 | 1410 | 2.2 |
| ISIS 588548 | 3703 | 1233 | 3.0 |
| ISIS 588550 | 4242 | 860 | 4.9 |
| ISIS 588553 | 3096 | 736 | 4.2 |
| ISIS 588555 | 4147 | 1860 | 2.2 |
| ISIS 588848 | 2235 | 738 | 3.0 |
| ISIS 594430 | 1548 | 752 | 2.1 |

1-94. (canceled)

95. A compound comprising a modified oligonucleotide consisting of 20 linked nucleosides having a nucleobase sequence consisting of the sequence recited in SEQ ID NO: 440 or SEQ ID NO: 455, or consisting of 16 linked nucleosides having a nucleobase sequence consisting of the sequence recited in SEQ ID NO: 598, wherein the modified oligonucleotide comprises:
a gap segment consisting of ten linked deoxynucleosides;
a 5′ wing segment consisting of five linked nucleosides for SEQ ID NO: 440 and SEQ ID NO: 455, or three linked nucleosides for SEQ ID NO: 598; and
a 3′ wing segment consisting of five linked nucleosides for SEQ ID NO: 440 and SEQ ID NO: 455, or three linked nucleosides for SEQ ID NO: 598;
wherein the gap segment is positioned between the 5′ wing segment and the 3′ wing segment, wherein
each nucleoside of each wing segment of SEQ ID NO: 440 and SEQ ID NO: 455 comprises a 2′—O-methoxyethyl sugar; wherein each internucleoside linkage is a phosphorothioate linkage and wherein each cytosine is a 5-methylcytosine; and
for SEQ ID NO: 598, the 5′ wing segment comprises a 2′—O-methoxyethyl sugar, 2′—O-methoxyethyl sugar, and cEt sugar in the 5′ to 3′ direction; wherein the 3′ wing segment comprises a cEt sugar, cEt sugar, and 2′—O-methoxyethyl sugar in the 5′ to 3′ direction; wherein each internucleoside linkage is a phosphorothioate linkage; and wherein each cytosine is a 5-methylcytosine.

96. The compound of claim 95, wherein the modified oligonucleotide is SEQ ID NO: 440.

97. The compound of claim 95, wherein the modified oligonucleotide is SEQ ID NO: 455.

98. The compound of claim 95, wherein the modified oligonucleotide is SEQ ID NO: 598.

99. The compound of claim 95, wherein the compound consists of the modified oligonucleotide.

100. The compound of claim 95, wherein the modified oligonucleotide further comprises a conjugate group.

101. A composition comprising the compound of claim 95 or salt thereof and a pharmaceutically acceptable carrier.

102. A method of treating or ameliorating a disease associated with dysregulation of the complement alternative pathway in a subject comprising administering to the subject the compound of claim 95, thereby treating, preventing, or ameliorating the disease.

103. The method of claim 102, wherein the complement alternative pathway is activated greater than normal.

104. The method of claim 102, wherein the disease is macular degeneration.

105. The method of claim 104, wherein the macular degeneration is wet macular degeneration (AMD).

106. The method of claim 102, wherein the disease is a kidney disease.


SEQUENCE LISTING (820 sequences)

SEQ ID NO: 1 - DNA, 2646 bp, Homo sapiens
  source 1..2646: mol_type = genomic DNA
gacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacaaaaaaaaaaaaaaaaaaaa

SEQ ID NO: 2 - DNA, 9001 bp, Homo sapiens
  source 1..9001: mol_type = genomic DNA
ggaggtgagggtctcaggttggggatgctgggatccccctgtgacagctcccagaatgtctctcttccttctccaggtctggctgctttctctctctgacgcgggtcacccctcctcccaagcctcacaaacctgctaggtgtccctgggtctgcttattctttttttgttgttattgagatggagtcttgctctgtctcccaggctggagtgcagtggcacgacctcagctcactgcaacttctgcctcctgggttcaagcgattctcctacttcagcctcccgagtagctgagattacaggtgcccaccaccacaccagctaatttttgtatttttagtagagacgggatttcgccatgttggccaggatggtcttgaactcctgacctcaagtgatctgcctgcctcaacctcccaaagtgctgagattacaggcgtgagccactgcacccacccgggtctgcttattctacccttctctctggttccacccctgctgcagtggacaagctgtgccgaggttgtctcccaagaaaaaaccatgttccccaacttgacagatgtcagggaggtggtgacagaccagttcctatgcagtgggacccaggaggatgagagtccctgcaagggtgagtccctcaccatgcctggattcccaaggggaaggccacctgtgtctctgtggccagcatgcatgccagaacaccagtccactgccctagatgacactgtctcctgtcaccctttgctggcaggagaatctgggggagcagttttccttgagcggagattcaggttttttcaggtgagaaggtagaagcttgcaggacccaggggttacaggatctcagccttgttggggggatgagggaggcctttgagggatctagggaggttggggcttacagttggggctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccacatctctcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtaagcgagggtaaccttcccttcctgctgtctccagcatccctccttggccttttggggccaggcttcatcagcctttctcttcaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagaggtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggaggtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaaggtgacctttgacctgtacccccaggtcagatcctggtcttccatcctactgtcttctctccccacctcaaccctgctctttcctcactttgtttaaacctccctgtacaactatctcacttctgagccttttataccctggaaacccatgatcccccgtctctttggtcactgtatccctgacactcccagacatttgacctcatttctgactctcccagactccttcatgtacgacacccctcaagaggtggccgaagctttcctgtcttccctgacagagaccatagaaggagtcgatgctgaggatgggcacggcccaggtttgaagacagagaagggaggcagggcagggaactgggggaaaatggagaagggacagaactgttaatgctggagcctgagccactctcctggcacccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtcctattctgtcctccttcccttttacttgaagcagtttcttgactggtaattcctccatgaacctcagcccttgagcctcttactgagagcctccctgtcccagcaaagtcgctgaaatctcccaatcacagtattctattttcaatgccatggcgccttgttctcctcacccacaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaaggtcagaggttagggaatggtgggaggttcactttggggtcaggaggttcagggtggagggggtcatgagactaccttgagggcgacagggaggaccactttgtagtcaaaagttgaacagcaggatcgttgggcaatggaggttagtgggaacctgttgggggctggaagggccactttgtggtcaaagggaagtccgtgtaatgatgattaacttaaaaagttgaaagatgtgggatttcagttgcagattggtctctggggttaaaagatggcttggaagaccaggtgaggtgatggtctcttccctctccacagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctgggtgagtaacctgcctaggacccagcaccccacttcctcagggcttggaccctcatccttcctttttatccctcagatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctcctcaggttcccctgaagtaattcattcttcctctacacctgaagctctagttgcctggaaagccttcttcattcctccttctctacctcagtgtcactattcttgtttcctggcactgttcacttaaccttagaatcacagagctctgagcacttcagagatctttctatagtcctacatttgacacgtggaaacagaagccaaaggaggtcaagggacagcaagttagcaacaagggtgggcttgaaaacagccaggcctctgacagcttgatcccaagttctttcccttttcagtccaccatagcagttttctcctaacacgaggaaacaaatacccgtggtctttccctttctccttttgggcctttgctccccatagactcctacccaaaaggctgctgccatttgggaatgaagtgttccgagttttcagcacattctccttctctgccagatgaaagccagtctctgagtctctgtggcatggtttgggaacacaggaagggtaccgattaccacaagcaaccatggcaggccaagatctcagtcattgtaagcacagaatcccagtagtggggacttgggggaggtgaggtcaaggtgaaatgggagtaggggaaggaaaaaatggccataagagatggtggtttgtgaaagttgagctttccctctctactgttgtgtccccagcgcccttcaaagggacacgagagctgtatgggggctgtggtgtctgagtactttgtgctgacagcagcacattgtttcactgtggatgacaaggaacactcaatcaaggtcagcgtaggtaaggatgcaactgaaggtcctgggctgcacctatgctctccaggcaacacctcccactttctacagatcctacactccacccatcctcaatgcagccccattccttgcaccccagaccagtcagggatgggggaagacgtgaagttaggaatgacacggggccagaggcaggaagctgcccacaaagaggtggtacctactctcctacttcaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggtgagagcgtccagatccctgaggaaaggctgggaaaggctggaggactggggtgaggagcaggcctggtttgctgttctccttgtcctttataggcccatttgtctcccctgcaccgagggaacaactcgagctttgaggcttcctccaactaccacttgccagcaacaaagtaagacatacttggcaagaggataaggatgagatcccaagagacaagtggggcatgagagggaggtgcaataggaagagatgatgcctggcccagaacctagctctagaagggcttaggggacatctactgagtgacaaaggcaatggggagatgacagtggtgggagcagctgaagtgacgcagtctattcgtccagaggaagagctgctccctgcacaggatatcaaagctctgtttgtgtctgaggaggagaaaaagctgactcggaaggaggtctacatcaagaatggggataaggtgagaaacgggcatcctaaggaggcactctaggccccaatccttcctaagccacttctgttcattacttctccatgcttcccacctcccctacagaaaggcagctgtgagagagatgctcaatatgccccaggctatgacaaagtcaaggacatctcagaggtggtcacccctcggttcctttgtactggaggagtgagtccctatgctgaccccaatacttgcagaggtgagagaatgctctttggttgtgctacaagtgcccaaggcccaacagtccttttctctacagcttctcctctccttgcaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacacaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacacctgtgttccagatccttttggggcaagggagtggggaacaggcactggccatgttgttacactgagatcaaacctgacagccgtttttaaaggtttaaccccaatcccaagtgctgaaaaaccagaggctgagggagatgtgtaagcttccacctcagtgttttactgagaccagcattggggcatatgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagaacagaccctgaggctgactcctgaccacctcactcctggccactggcccctggaagcccagtttccacgctgccctctggtggccaggatggcctgtcttccttagctcctttgtgccaacccatggccaagaaaagtataagtggacattttgatgaatgttttgttcttagaaaaatcccaaatgtcattgttgagacacgtgaatgatattaacccactacttacagtcagtatgtcagaagctaaaaactagaaaacctctgtagcccttttttgacatgctggtcaattctagttcctttcttttgcctgaagggccactgtagctgagcccttctttctgctcactcctttcccaggaaaatctactttcagggaaaatggattattcacactaagaaatgctactagctccaccagaactcattcagggtgtagctttggccctcaccattctctctcaagcctctagctgtttcttccccttcctctttcctccctccaccagacatgttactctcttcaccccatccaatggttccatccccaccacccttgagctacagagaatctctctcacccactcccatcctgtgatctctgtgcctcaacactgctggctactccctctttctcaaagtgtgtgtccttttgcttcagtggcccaggcccctgcggtgctgctcccagccctccgacccctcctcctgtctcctttgctaacgttaggctcaacgttagcctaacatgtcaggacagctggggacatgtgggg

SEQ ID NO: 3 - DNA, 9001 bp, Macaca mulatta
  source 1..9001: mol_type = genomic DNA
gatggaatcttgctctgtctcccacactggagtgcagtggcacgatcttggctcactgcgacttctgcctcccagatttaagtgattctcctacctcggcctcccaagtagctgggattataggtgcttgccaccacatgcagctaatttttgtatttttagtagagacaggattccgccatgttggccaggatggtcttgaactcctgacctcaagtgattcgcccacctcagcctcccaaactgctgggattacaggcgtgagccattgcacccagtcaggtctgcttattcttcccttctctctggttccacccctacggcagtggacaagctgtgccgaggtcgtctcccaagaaaaaaccatgttccccaacttgacagatgtcagggaggtggtgacagaccagtttctatgcagtgggacccaggaggatgagagtccctgcaagggtgagtccctcaccatgcctggattcccaaaggggaaggccacctgtgtctctgtggccagggtgcatgccagaacaccagtccactgccctgtatgacgctgtctcctgtcaccctttgctggcaggagaatctgggggagcagttttccttgagcggagattcaggttttttcaggtgagaaggtggaagcttgcaggacccaggggttacaggatgtcagccttgttggggggatgagggaggcctttgagggatctagggaggttggggcttacagctggagctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctttagtcatggccactgagccctctgctgtcctgttagaatccgccccccctccatcttctacctctgaatgcccacccttagactctgtgacccatgctgtctcctagttgagtaaatctgggtctctaggatgccaggggcagcgcacacaagctgggaaatcctcagggctcctaccagcgggactgcctcgctgccccacctcccgctccttggcctgtccccaaattcctcccctggttgacttgactcatgctcatttcactttcatatggaatttcccagttatgaaattaataaaaatcagtggtttccacatctgtctgtgactctatctggaggccaggtagggctggcctggggggaaggggaggccagaatgactccaagagccacaggaaggcaggtcagagaccccactggacaaacagtggctggactctgtaccataacacacaagcaacaggggagtgagctggatccttatttctggtccctaagtgggtggtctgggcttgctggggaggagctgaggccagaaggaggtactgaaggggagagtcctggaccttgggcagcaaagggtgggacttctgcagtttctgcttccttgactggcagctcagcggggccctcccgcttggatgttccgggaaagtgatgagggtaggacaggcggggcaagctgcaggtgccagaacacagattgcataaaaggccgggagctggtggggggcaggggaagggaatgtgaccaggtctaggtctggagtttcagcttggacactgagctaagtagacaagcaaaacaagccaggacacgccatcctgccccaggcccagcttctctcctgccttctaacgccatggggagcagtctcagcccccagctctacctgatgcccttcatcttgggcctcttatctggaggtaagtgagggtaaccttcccttcctgctgtccccagcatccctccttggccttttggggccaggcttcatcagcctttctcttcaggtgtgaccaccactccattgtcttcggcccagcctcaaggatcctgctctctggagggggtagagatcaaaggtggctccttccgacttctccaagagggccaggcactggaatacgtgtgtccttctggcttctacccgtaccctgtgcagacacgtacctgcagatccacggggtcctggagcaccctgcagactcaagatcgaaaaactgtcaagaaggcagagtgcagaggtttgagggcaatgagtgtgggcagtggcctaagggagaaacagggcagatggcagcaaggtcaggactaggatgagactaggcagggtgacaaggtgggctgaccaggagtaggagcagttttagggttgtagagggaaaggaagggaaaaaaaaaggggagttaacctttagtaagcatttaccctgggcttccacgcagccctggaagtcaagagaacactcagcaatggggagggaggagcagcggaaacccctatgggttgaagggtaggtaagatgcagcttctgcaggactgggaatgctctgtttctcagtgacctggtctctgagaccaggagggaaacacctaaggcagcctttccctcttaatgacttctacttctcccctcttctcaaagcaatccgctgtccacgaccacaggacttcgagaacggggaataccggccccggtctccctactacaatgtgagtgatgagatctctttccactgctatgacggttacactctccggggctctgccaatcgcacctgccaagtgaatggccggtggagtgggcagacagcgatctgtgacaacggaggtgagaagcatcctctccccccacattgctgtctccctgacagcgcctagcctgaggagtgggcatttgcccccggacactgtaactcttgctctctaccttgccctcggggcctcaggcttcagcgcttacctccatgtctcatgcctctgcagcggggtactgctccaacccaggcatccccattggcacaaggaaggtgggcagccggtaccgccttgaagacagcgtcacctaccactgcagccgggggcttaccctgcgtggctcccagcggcgaacatgtcaggaaggtggctcttggagcgggacggagccttcctgccaaggtgacccttgacctgtacccccaggtcagatcctgatcttgcatcctactgtcttctctccccacctcaaccctgctctttcctcacttcttttaaaccttcctctagaactgtctcacttctgagccttttctaccctggaaacccacaatcccctgtctctttggtcactgtgtccctgacactcccagacatttgacctcatttctgactctcccagactccttcatgtacgacacccctcaagaggtggccgaagctttcctgtcttccctgacggagaccatagaaggagtcgatgccgaggatgggcacagcccaggtttgaaggcagagaggggaggcaaggcagggaactgggggaaaatggagaagggacaagataatcgttcatgctggagcctgagtcactctcctggcacccaggggaacaacagaagcggaggatcatcctagacccttcaggctccatgaacatctacctggtgctagatggatcagacagcattggggccggcaacttcacaggagccaaaaagtgtctagtcaacttaattgagaaggtggagtcctcctatccctgaacttgggggaatggaatcttgctgatcttccaggactagctccctgatcattccagcccctctgaaccgcagggccccaggaaagtctccaggtcctattctgtcctccttcccttgtacttgattcctccatgaacctgtgcttgagcctcttcctaagagcctccctgtcccagcaacgttgctgaagtctcccaatcacagtattctactttcaatgccatggcgccttgttctcctcacccacaggtggcaagttatggtgtgaagccaagatatgctctagtgacatatgccacataccccagaatttgggtcaaagtgtctgaccaagagagcagcaatgcagactgggtcacgaagaagctcagtgaaatcaattatgaaggtcagaggttagggaatggtgggaggttcactttggggtcaggaggttcaggagtgttgtgtggagggggtcatgagactaccttgagggcaacagggggaccactttgtagtcaaaggttgaacagcaggatcattgggcaatggaggttagtgggaacctgctgagggctggaagggccactttgtggtcaaagggaagtccatatgatgattaacttaaaaagttgaagatgtgagatttcagttgcagattggtctctggggttaaaagatggcttggaagaccaggtgaggcgatgctctcttccctccccacagaccacaagttgaagtcagggactaacaccaagagggccctccaggcagtgtacagcatgatgagttggccagaggacatccctcctgaaggctggaaccgcacccgccatgtcatcatcctcatgaccgatggtcagaagggacctctctcctgtcccagcctccccaccttctcagaccagcatgtggcccttaagtccacttgtaacactatacccatggttggggccctgaatgtgactcgtaactggctgttcatctctcctgtgacccttcataaagaattattcctaaagccctgtgatcaactacctctaacccttcctcaacttactcaccctgccacgtgtatcactgcctctagccaatttatcttatctcctaccctcatggtcccgtctcttctgcaggattgcacaacatgggcggggacccaattactgtcattgatgagatccgggacttgttatacatcggcaaggatcgtaaaaacccgagggaggattatctgggtgagtaacctgcctaggacccagcaccctacttcctcagggcttggaccgtcatccttcctttttctccctcagatgtctatgtgtttggggttggacctttggtggaccaagtgaacatcaatgctttggcttccaagaaagacaatgagcaacatgtgttcaaagtcaaggatatggaaaacctggaagacgttttcttccaaatgattggtaggcagacacaagggaatcaagaacgcaactctcctcagcttcccctgaaataattcattcttcctctacccctgaagctctagttgcctggaaagccttcttcattcctccttctctacctcagtatcactattcttgtttcctggcactgtttgcttcttaaccttagaatcacagagctctaggcacttcagagatctttctattgtcctacatttgacacatgtggaaacaaaggccaaaggaggtcaaggggcagcaagctagcaacagggctgggcttgaaaacagccaggcctctgatagcttgatcccaagttctttcccttttcactccaccacagcagttttctcctaacacgaggaaacaaatacctgtggcctttccctttctccttttgggcctctgccccccacagacttctacccaaaggctgctgccgtttgggaatgaagtgttccaagttttcagcacattctccttctctgccagatgaaagccagtctctgagtctctgtggcatggtttgggaacacagcaagggtaccgattaccacaagcaaccatggcaggccaagatctcagtcactgtaagcacagaatcccagtagtgaggacttgggggaggtgaggtcaaggtgaaatgggagtaggggaagggcaaaatggccgtaagagatggtggtttgtgaaagttgagttttccctttctactgttctgttcccagcgcccttcgaagggacatgagagctgtatgggggctgtggtgtctgagtactttgtgctgacagcagcacattgttttactgtggacgacaaggaacactcaatcaaggtcagcgtgggtaaggatgcaactgaaggtcccgggctgcacctacgccctccaggcaacacctcccactttctacagatcccacactccactcatctgcaatgcagccccatcccttgcaccccagaccagtcagggatggggaagacttgaagttaggaatgacatggggccagaggcaagaagctgcccacaaagaggtggtacctattctcctacttcaagggaagaagcgggacctggagatagaaaaagtcctatttcaccccgactacaacattagcgagaaaaaagaagcaggaattcctgaattttatgactatgacgttgccctgatcaagctcaagaataagttgaattatgacccgactatcaggtgagagcatccagatccctgaggaaaggctgggaaaggctggaggactggggtgaggagcaggcctagtttgctgttctttctccatcctttataggcccatttgtctcccctgcaccgagggaacaactcgagctttgaggcttcctccaactaccacttgccagcaacagagtaagacatactaggggggaggataaggatgagatcccgagacaagtgaggcatgagagggagatgcaataggaagagacgatgcctggcccagaacctagcactaggaagggcttaggggacatctgctgagtgacaaagtcaatagggagatgacagtggtgggagcagctgaagtgatgcagtctatttgtccagaggaagagctgctccctgcacaggatatcaaagctctgtttgtgtctgaggaggagaagaagctgactcggaaggaggtctacatcaagaatggggataaggtgagaaatgggcatcctaaggaggcactctaggccctaatccttcctaagccacctctgttcattacctttctccatgcttcccacctcccctacagaaaggcagctgtgagagagatgctcaatatgccccaggctatgacaaagtcaaggacatctcggaggtggtcacccctcggttcctttgtactggaggagtgagtccctatgctgaccccaatacttgcagaggtgagagaacgctctctggttgtgctccaagtgcccgagggccaagagtccttttccctacagcttctcctctccttgcaggtgattctggcggccccttgatagttcacaagagaagtcgtttcattcaagtgagtcctccctttcctatctggggagatgccaagtggtcagcatgggccccaaagcaggaaagcacaatgcatgtggctagtaattcgaggtgggcagagcctgcctcactttaggactgcatgtctggcctgtgtgtgtcaagaatgaggctgagctgggtccctagcctgattcctttaggtcagctaagacacaatcaggaacagtcatgcttccaggattaggaattctatgaatgatccatggcaccccactgcctctgcaggttggtgtcatcagctggggagtagtggatgtctgcaaaaaccagaagcggcaaaagcaggtacctgctcacgcccgagactttcacgtcaacctcttccaagtgctgccctggctgaaggagaaactccaagatgaggatttgggttttctctaaggggtttcctgctggacaggggcgcgggattgaattaaaacagctgcgacaacacttgtgttccagatccttttggggcaagggagtggggaacgggcactggccatgttgttacactgagatcaaacctgacacccatttttaaaggcttaaccccaatcccaagtgctgaaaaaccagaggctgagggagatatgtaagcttccacctcagtgttttactgagaccagcattggggcatttgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagatcagaccctgaggctgactcctgaccacctcagtcctggctgctggcccctggaaacccagtttccacgctgccctctggtggccaggatggcctgtcttccttagctcctttgtgccaacccatggccaaggagagtgtaagtggacattttgatgaatgttttgttcttagaaaaatcccaaatgtcattgttgagatatatgaatgatattaacccactacttatagtcagtatgtcagaagctaaaaactagaaaacctctgtagccctttattgacatgctggtcaactctagttcctttcttttgcctgaaaggccactgttgctctgagtccttctttctgctcactcctttcccaggaaaatctactttcaggtaaatgggttactcatactaaggaatgctactagctccaccagaactcatccagcatgtagctttggccctcaccattctctctcaagcctctagctgtttcttccccttcctcttttcctccctccaccagacatgttactctcttcaccccatccaaagattccatccccaccacccttgacctagagagaatctctcccacccacttctcatcctgtgatctctgtaccttgacactgctggctactccctctttctcaaagcatgtgtcctttcgcttcagtggcccaggcccctctggtgctgctcccagccctctgacccctcctcctgtctcctttgctaacgttaggctcaacgttagcctaacgtgtcaggagagctggagacacgtggggcgtaaggtggacagtcctgtttcctaacatagtccctgagtattcctcaagtctagtcctgggtcgttttttttctccgaaatcagtctccctcatgatcggggagccaccctgtgatgcagatgacttaatctatgttttcattccttacctcacacctgagttccagacccctaatttcaaatactt

SEQ ID NO: 4 - DNA, 4086 bp, Macaca mulatta
  source 1..4086: mol_type = genomic DNA
atagatatattagcatcagggagacagggcaaaggttccacccttcagctcagtccccagtccctgcttattatttccctaacagaagaccatcccccttgccactccctgggttttcttctctggcagcaatgaagcagctgctgagccagctctggttttcgggaagtcagatgaccttttccctcccgcggctctctgcctctcgctgtccctagggaggacaccatggacccactgatggttcttttttgcctgctgttcctgtacccaggtccggcagactcggctacctcctgccctcagaacgtgaatatctctggtggcaccttcaccctcagccatggctgggcccctgggagccttctcatctactcctgtccccagggcctgtacccatccccagcgtcacggctgtgcaagagcagcggacagtggcagaccccaagagccacccggtctctgactaaggcggtctgcaaacctggccactgccccaaccccggcatttcgctgggcgcggtgcggacaggctcccgctttggtcatggggacaaggtccgctatcgctgctcctcgaatcttgtgctcacggggtctgcggagcgggagtgccagggcaacggggtctggagtggaacggagcccatctgccgccagccctactcttatgacttccctgaggacgtggcccctgccctgggcacctccttctcccacatgcttggggccaccaatcccacccagaggacaaaggatcatgaaaatggaactgggactaacacctatgcagccctaaacagtgtctatctcatgatgaacaatcaaatgcaactccttggcatgaaaacgatggcctggcaggaaatccgacatgccatcatccttctgacagatggaaagtccaatatgggtggctctcccaaaacagctgttgaccaaatcagagagatcttgaatatcaaccagaagaggaatgactatctggacatctatgccatcggggtgggcaagctggatgtggactggagagaactgaatgagctggggtccaagaaggatggcgagaggcatgccttcattctgcaggacacaaaggctctgcaccaggtctttgaacatatgctggatgtctccaagctcacagacaccatctgcggggtggggaacatgtcagcaaacgcctctgaccaagagaggacaccctggcatgtcactattaagcccaagagccaagagacctgccggggagccctcatctccgaccaatgggtcctgacagcggctcactgcttccgcgatggcaacgaccactccctatggagggtcaatgtgggagaccccaaatcccagtggggcaaagaattccttattgagaaggcagtgatttccccaggatttgatgtctttgccaaaaagaaccagggaatcctggagttctatggtgatgacatcgccctgctgaagctggcccagaaagtaaagatgtccacccatgccaggcccatctgccttccctgcaccatggaggccaatctggctctgcggagacctcaaggcagcacctgtagggaccatgagaatgaactgctgaacaaacagagtgttcctgctcattttgtcgccttgaatgggagcaaactgaacattaaccttaagatgggagtggagtggacaagctgtgccgaggtcgtctcccaagaaaaaaccatgttccccaacttgacagatgtcagggaggtggtgacagaccagtttctatgcagtgggacccaggaggatgagagtccctgcaagggtgtgaccaccactccattgtcttcggcccagcctcaaggatcctgctctctggagggggtagagatcaaaggtggctccttccgacttctccaagagggccaggcactggaatacgtgtgtccttctggcttctacccgtaccctgtgcagacacgtacctgcagatccacggggtcctggagcaccctgcagactcaagatcgaaaaactgtcaagaaggcagagtgcagagcaatccgctgtccacgaccacaggacttcgagaacggggaataccggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccaggcatccccattggcacaaggaaggtgggcagccggtaccgccttgaagacagcgtcacctaccactgcagccgggggcttaccctgcgtggctcccagcggcgaacatgtcaggaaggtggctcttggagcgggacggagccttcctgccaagactccttcatgtacgacacccctcaagaggtggccgaagctttcctgtcttccctgacggagaccatagaaggagtcgatgccgaggatgggcacagcccaggggaacaacagaagcggaggatcatcctagacccttcaggctccatgaacatctacctggtgctagatggatcagacagcattggggccggcaacttcacaggagccaaaaagtgtctagtcaacttaattgagaaggtggcaagttatggtgtgaagccaagatatgctctagtgacatatgccacataccccagaatttgggtcaaagtgtctgaccaagagagcagcaatgcagactgggtcacgaagaagctcagtgaaatcaattatgaagaccacaagttgaagtcagggactaacaccaagagggccctccaggcagtgtacagcatgatgagttggccagaggacatccctcctgaaggctggaaccgcacccgccatgtcatcatcctcatgaccgatggattgcacaacatgggcggggacccaattactgtcattgatgagatccgggacttgttatacatcggcaaggatcgtaaaaacccgagggaggattatctggatgtctatgtgtttggggttggacctttggtggaccaagtgaacatcaatgctttggcttccaagaaagacaatgagcaacatgtgttcaaagtcaaggatatggaaaacctggaagacgttttcttccaaatgattgatgaaagccagtctctgagtctctgtggcatggtttgggaacacagcaagggtaccgattaccacaagcaaccatggcaggccaagatctcagtcactcgcccttcgaagggacatgagagctgtatgggggctgtggtgtctgagtactttgtgctgacagcagcacattgttttactgtggacgacaaggaacactcaatcaaggtcagcgtgggagggaagaagcgggacctggagatagaaaaagtcctatttcaccccgactacaacattagcgagaaaaaagaagcaggaattcctgaattttatgactatgacgttgccctgatcaagctcaagaataagttgaattatgacccgactatcaggcccatttgtctcccctgcaccgagggaacaactcgagctttgaggcttcctccaactaccacttgccagcaacagaaggaagagctgctccctgcacaggatatcaaagctctgtttgtgtctgaggaggagaagaagctgactcggaaggaggtctacatcaagaatggggataagaaaggcagctgtgagagagatgctcaatatgccccaggctatgacaaagtcaaggacatctcggaggtggtcacccctcggttcctttgtactggaggagtgagtccctatgctgaccccaatacttgcagaggtgattctggcggccccttgatagttcacaagagaagtcgtttcattcaagttggtgtcatcagctggggagtagtggatgtctgcaaaaaccagaagcggcaaaagcaggtacctgctcacgcccgagactttcacgtcaacctcttccaagtgctgccctggctgaaggagaaactccaagatgaggatttgggttttctctaaggggtttcctgctggacaggggcgcgggattgaattaaaacagctgcgacaacactt

SEQ ID NO: 5 - DNA, 2767 bp, Mus musculus
  source 1..2767: mol_type = genomic DNA
gctccatcacacagtccatggaaagactgatcttttaaattgggggtagtggaggtggtggtctgtgcttgttaggaggggtctgggggctaagagggagctttgaaagggaagttctggcccttggtcagtcaagggtggggctcacatagtttctgtttcctcagttggcagttcagctggggccctccttcatgaatgttccgggaagcagtggctgcgtgcgcagggtaggctggccaggctgcagatgccagagcagattgcataaaaggttaggggacagtgggaaaggggtgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctataaagagcttcctgcagggagagtgtgaggacagattaaagcagttacaataacaaaaaaaaaaaaaaaaaaaaaa

SEQ ID NO: 6 - DNA, 20 bp, synthetic construct
  misc_feature 1..20: note = Synthetic oligonucleotide
  source 1..20: mol_type = other DNA
gctgagctgccagtcaagga

SEQ ID NO: 7 - DNA, 20 bp, synthetic construct
  misc_feature 1..20: note = Synthetic oligonucleotide
  source 1..20: mol_type = other DNA
ggccccgctgagctgccagt

SEQ ID NO: 8 - DNA, 20 bp, synthetic construct
  misc_feature 1..20: note = Synthetic oligonucleotide
  source 1..20: mol_type = other DNA
cggaacatccaagcgggagg

SEQ ID NO: 9 - DNA, 20 bp, synthetic construct
  misc_feature 1..20: note = Synthetic oligonucleotide
  source 1..20: mol_type = other DNA
ctttcccggaacatccaagc

SEQ ID NO: 10 - DNA, 20 bp, synthetic construct
  misc_feature 1..20: note = Synthetic oligonucleotide
  source 1..20: mol_type = other DNA
atctgtgttctggcacctgc

SEQ ID NO: 11 - DNA, 20 bp, synthetic construct
  misc_feature 1..20: note = Synthetic oligonucleotide
  source 1..20: mol_type = other DNA
gtcacattcccttcccctgc

SEQ ID NO: 12 - DNA, 20 bp, synthetic construct
  misc_feature 1..20: note = Synthetic oligonucleotide
  source 1..20: mol_type = other DNA
gacctggtcacattcccttc

SEQ ID NO: 13 - DNA, 20 bp, synthetic construct
  misc_feature 1..20: note = Synthetic oligonucleotide
  source 1..20: mol_type = other DNA
gacctagacctggtcacatt

SEQ ID NO: 14 - DNA, 20 bp, synthetic construct
  misc_feature 1..20: note = Synthetic oligonucleotide
  source 1..20: mol_type = other DNA
actccagacctagacctggt

SEQ ID NO: 15 - DNA, 20 bp, synthetic construct
  misc_feature 1..20: note = Synthetic oligonucleotide
  source 1..20: mol_type = other DNA
gctgaaactccagacctaga

SEQ ID NO: 16 - DNA, 20 bp, synthetic construct
  misc_feature 1..20: note = Synthetic oligonucleotide
  source 1..20: mol_type = other DNA
gtccaagctgaaactccaga

SEQ ID NO: 17 - DNA, 20 bp, synthetic construct
  misc_feature 1..20: note = Synthetic oligonucleotide
  source 1..20: mol_type = other DNA
ctcagtgtccaagctgaaac

SEQ ID NO: 18 - DNA, 20 bp, synthetic construct
  misc_feature 1..20: note = Synthetic oligonucleotide
  source 1..20: mol_type = other DNA
aggagagaagctgggcctgg

SEQ ID NO: 19 - DNA, 20 bp, synthetic construct
  misc_feature 1..20: note = Synthetic oligonucleotide
  source 1..20: mol_type = other DNA
gaaggcaggagagaagctgg

SEQ ID NO: 20 - DNA, 20 bp, synthetic construct
  misc_feature 1..20: note = Synthetic oligonucleotide
  source 1..20: mol_type = other DNA
gtggtggtcacacctccaga

SEQ ID NO: 21 - DNA, 20 bp, synthetic construct
  misc_feature 1..20: note = Synthetic oligonucleotide
  source 1..20: mol_type = other DNA
ccctccagagagcaggatcc

SEQ ID NO: 22 - DNA, 20 bp, synthetic construct
  misc_feature 1..20: note = Synthetic oligonucleotide
  source 1..20: mol_type = other DNA
tctaccccctccagagagca

SEQ ID NO: 23 - DNA, 20 bp, synthetic construct
  misc_feature 1..20: note = Synthetic oligonucleotide
  source 1..20: mol_type = other DNA
ttgatctctaccccctccag

SEQ ID NO: 24 - DNA, 20 bp, synthetic construct
  misc_feature 1..20: note = Synthetic oligonucleotide
  source 1..20: mol_type = other DNA
tggagaagtcggaaggagcc

SEQ ID NO: 25 - DNA, 20 bp, synthetic construct
  misc_feature 1..20: note = Synthetic oligonucleotide
  source 1..20: mol_type = other DNA
ccctcttggagaagtcggaa

SEQ ID NO: 26 - DNA, 20 bp, synthetic construct
  misc_feature 1..20: note = Synthetic oligonucleotide
  source 1..20: mol_type = other DNA
gcctggccctcttggagaag

SEQ ID NO: 27 - DNA, 20 bp, synthetic construct
  misc_feature 1..20: note = Synthetic oligonucleotide
  source 1..20: mol_type = other DNA
tccagtgcctggccctcttg

SEQ ID NO: 28 - DNA, 20 bp, synthetic construct
  misc_feature 1..20: note = Synthetic oligonucleotide
  source 1..20: mol_type = other DNA
agaagccagaaggacacacg

SEQ ID NO: 29 - DNA, 20 bp, synthetic construct
  misc_feature 1..20: note = Synthetic oligonucleotide
  source 1..20: mol_type = other DNA
acgggtagaagccagaagga

SEQ ID NO: 30 - DNA, 20 bp, synthetic construct
  misc_feature 1..20: note = Synthetic oligonucleotide
  source 1..20: mol_type = other DNA
cgtgtctgcacagggtacgg

SEQ ID NO: 31 - DNA, 20 bp, synthetic construct
  misc_feature 1..20: note = Synthetic oligonucleotide
  source 1..20: mol_type = other DNA
agggtgctccaggaccccgt

SEQ ID NO: 32 - DNA, 20 bp, synthetic construct
  misc_feature 1..20: note = Synthetic oligonucleotide
  source 1..20: mol_type = other DNA
ttgctctgcactctgccttc

SEQ ID NO: 33 - DNA, 20 bp, synthetic construct
  misc_feature 1..20: note = Synthetic oligonucleotide
  source 1..20: mol_type = other DNA
tattccccgttctcgaagtc

SEQ ID NO: 34 - DNA, 20 bp, synthetic construct
  misc_feature 1..20: note = Synthetic oligonucleotide
  source 1..20: mol_type = other DNA
cattgtagtagggagaccgg

SEQ ID NO: 35 - DNA, 20 bp, synthetic construct
  misc_feature 1..20: note = Synthetic oligonucleotide
  source 1..20: mol_type = other DNA
cactcacattgtagtaggga

SEQ ID NO: 36 - DNA, 20 bp, synthetic construct
  misc_feature 1..20: note = Synthetic oligonucleotide
  source 1..20: mol_type = other DNA
tctcatcactcacattgtag

SEQ ID NO: 37 - DNA, 20 bp, synthetic construct
  misc_feature 1..20: note = Synthetic oligonucleotide
  source 1..20: mol_type = other DNA
aagagatctcatcactcaca

SEQ ID NO: 38 - DNA, 20 bp, synthetic construct
  misc_feature 1..20: note = Synthetic oligonucleotide
  source 1..20: mol_type = other DNA
agtggaaagagatctcatca

SEQ ID NO: 39 - DNA, 20 bp, synthetic construct
  misc_feature 1..20: note = Synthetic oligonucleotide
  source 1..20: mol_type = other DNA
catagcagtggaaagagatc

SEQ ID NO: 40 - DNA, 20 bp, synthetic construct
  misc_feature 1..20: note = Synthetic oligonucleotide
  source 1..20: mol_type = other DNA
aaccgtcatagcagtggaaa

SEQ ID NO: 41 - DNA, 20 bp, synthetic construct
  misc_feature 1..20: note = Synthetic oligonucleotide
  source 1..20: mol_type = other DNA
gagtgtaaccgtcatagcag

SEQ ID NO: 42 - DNA, 20 bp, synthetic construct
  misc_feature 1..20: note = Synthetic oligonucleotide
  source 1..20: mol_type = other DNA
cccggagagtgtaaccgtca

SEQ ID NO: 43 - DNA, 20 bp, synthetic construct
  misc_feature 1..20: note = Synthetic oligonucleotide
  source 1..20: mol_type = other DNA
cagagccccggagagtgtaa

SEQ ID NO: 44 - DNA, 20 bp, synthetic construct
  misc_feature 1..20: note = Synthetic oligonucleotide
  source 1..20: mol_type = other DNA
gattggcagagccccggaga

SEQ ID NO: 45 - DNA, 20 bp, synthetic construct
  misc_feature 1..20: note = Synthetic oligonucleotide
  source 1..20: mol_type = other DNA
aggtgcgattggcagagccc

SEQ ID NO: 46 - DNA, 20 bp, synthetic construct
  misc_feature 1..20: note = Synthetic oligonucleotide
  source 1..20: mol_type = other DNA
cttggcaggtgcgattggca

SEQ ID NO: 47 - DNA, 20 bp, synthetic construct
  misc_feature 1..20: note = Synthetic oligonucleotide
  source 1..20: mol_type = other DNA
cattcacttggcaggtgcga

SEQ ID NO: 48 - DNA, 20 bp, synthetic construct
  misc_feature 1..20: note = Synthetic oligonucleotide
  source 1..20: mol_type = other DNA
atcgctgtctgcccactcca

SEQ ID NO: 49 - DNA, 20 bp, synthetic construct
  misc_feature 1..20: note = Synthetic oligonucleotide
  source 1..20: mol_type = other DNA
tcacagatcgctgtctgccc

SEQ ID NO: 50 - DNA, 20 bp, synthetic construct
  misc_feature 1..20: note = Synthetic oligonucleotide
  source 1..20: mol_type = other DNA
ccgttgtcacagatcgctgt

SEQ ID NO: 51 - DNA, 20 bp, synthetic construct
  misc_feature 1..20: note = Synthetic oligonucleotide
  source 1..20: mol_type = other DNA
cccgctccgttgtcacagat

SEQ ID NO: 52 - DNA, 20 bp, synthetic construct
  misc_feature 1..20: note = Synthetic oligonucleotide
  source 1..20: mol_type = other DNA
cagtaccccgctccgttgtc

SEQ ID NO: 53 - DNA, 20 bp, synthetic construct
  misc_feature 1..20: note = Synthetic oligonucleotide
  source 1..20: mol_type = other DNA
ttggagcagtaccccgctcc

SEQ ID NO: 54 - DNA, 20 bp, synthetic construct
  misc_feature 1..20: note = Synthetic oligonucleotide
  source 1..20: mol_type = other DNA
accttccttgtgccaatggg

SEQ ID NO: 55 - DNA, 20 bp, synthetic construct
  misc_feature 1..20: note = Synthetic oligonucleotide
  source 1..20: mol_type = other DNA
ctgcccaccttccttgtgcc

SEQ ID NO: 56 - DNA, 20 bp, synthetic construct
  misc_feature 1..20: note = Synthetic oligonucleotide
  source 1..20: mol_type = other DNA
cgctgtcttcaaggcggtac

SEQ ID NO: 57 - DNA, 20 bp, synthetic construct
  misc_feature 1..20: note = Synthetic oligonucleotide
  source 1..20: mol_type = other DNA
gctgcagtggtaggtgacgc

SEQ ID NO: 58 - DNA, 20 bp, synthetic construct
  misc_feature 1..20: note = Synthetic oligonucleotide
  source 1..20: mol_type = other DNA
cccccggctgcagtggtagg

SEQ ID NO: 59 - DNA, 20 bp, synthetic construct
  misc_feature 1..20: note = Synthetic oligonucleotide
  source 1..20: mol_type = other DNA
ggtaagcccccggctgcagt

SEQ ID NO: 60 - DNA, 20 bp, synthetic construct
  misc_feature 1..20: note = Synthetic oligonucleotide
  source 1..20: mol_type = other DNA
acgcagggtaagcccccggc

SEQ ID NO: 61 - DNA, 20 bp, synthetic construct
  misc_feature 1..20: note = Synthetic oligonucleotide
  source 1..20: mol_type = other DNA
ggagccacgcagggtaagcc

SEQ ID NO: 62 - DNA, 20 bp, synthetic construct
  misc_feature 1..20: note = Synthetic oligonucleotide
  source 1..20: mol_type = other DNA
gccgctgggagccacgcagg

SEQ ID NO: 63 - DNA, 20 bp, synthetic construct
  misc_feature 1..20: note = Synthetic oligonucleotide
  source 1..20: mol_type = other DNA
caagagccaccttcctgaca

SEQ ID NO: 64 - DNA, 20 bp, synthetic construct
  misc_feature 1..20: note = Synthetic oligonucleotide
  source 1..20: mol_type = other DNA
ccgctccaagagccaccttc

SEQ ID NO: 65 - DNA, 20 bp, synthetic construct
  misc_feature 1..20: note = Synthetic oligonucleotide
  source 1..20: mol_type = other DNA
tccgtcccgctccaagagcc

SEQ ID NO: 66 - DNA, 20 bp, synthetic construct
  misc_feature 1..20: note = Synthetic oligonucleotide
  source 1..20: mol_type = other DNA
gaaggctccgtcccgctcca

SEQ ID NO: 67 - DNA, 20 bp, synthetic construct
  misc_feature 1..20: note = Synthetic oligonucleotide
  source 1..20: mol_type = other DNA
tggcaggaaggctccgtccc

SEQ ID NO: 68 - DNA, 20 bp, synthetic construct
  misc_feature 1..20: note = Synthetic oligonucleotide
  source 1..20: mol_type = other DNA
gagtcttggcaggaaggctc

SEQ ID NO: 69 - DNA, 20 bp, synthetic construct
  misc_feature 1..20: note = Synthetic oligonucleotide
  source 1..20: mol_type = other DNA
atgaaggagtcttggcagga

SEQ ID NO: 70 - DNA, 20 bp, synthetic construct
  misc_feature 1..20: note = Synthetic oligonucleotide
  source 1..20: mol_type = other DNA
cttcggccacctcttgaggg

SEQ ID NO: 71 - DNA, 20 bp, synthetic construct
  misc_feature 1..20: note = Synthetic oligonucleotide
  source 1..20: mol_type = other DNA
ggaaagcttcggccacctct

SEQ ID NO: 72 - DNA, 20 bp, synthetic construct
  misc_feature 1..20: note = Synthetic oligonucleotide
  source 1..20: mol_type = other DNA
aagacaggaaagcttcggcc

SEQ ID NO: 73 - DNA, 20 bp, synthetic construct
  misc_feature 1..20: note = Synthetic oligonucleotide
  source 1..20: mol_type = other DNA
tcagggaagacaggaaagct

SEQ ID NO: 74 - DNA, 20 bp, synthetic construct
  misc_feature 1..20: note = Synthetic oligonucleotide
  source 1..20: mol_type = other DNA
tcgactccttctatggtctc

SEQ ID NO: 75 - DNA, 20 bp, synthetic construct
  misc_feature 1..20: note = Synthetic oligonucleotide
  source 1..20: mol_type = other DNA
cttctgttgttcccctgggc

SEQ ID NO: 76 - DNA, 20 bp, synthetic construct
  misc_feature 1..20: note = Synthetic oligonucleotide
  source 1..20: mol_type = other DNA
ttcatggagcctgaagggtc

SEQ ID NO: 77 - DNA, 20 bp, synthetic construct
  misc_feature 1..20: note = Synthetic oligonucleotide
  source 1..20: mol_type = other DNA
tagatgttcatggagcctga

SEQ ID NO: 78 - DNA, 20 bp, synthetic construct
  misc_feature 1..20: note = Synthetic oligonucleotide
  source 1..20: mol_type = other DNA
accaggtagatgttcatgga

SEQ ID NO: 79 - DNA, 20 bp, synthetic construct
  misc_feature 1..20: note = Synthetic oligonucleotide
  source 1..20: mol_type = other DNA
tctagcaccaggtagatgtt

SEQ ID NO: 80 - DNA, 20 bp, synthetic construct
  misc_feature 1..20: note = Synthetic oligonucleotide
  source 1..20: mol_type = other DNA
gatccatctagcaccaggta

SEQ ID NO: 81 - DNA, 20 bp, synthetic construct
  misc_feature 1..20: note = Synthetic oligonucleotide
  source 1..20: mol_type = other DNA
ctgtctgatccatctagcac

SEQ ID NO: 82 - DNA, 20 bp, synthetic construct
  misc_feature 1..20: note = Synthetic oligonucleotide
  source 1..20: mol_type = other DNA
ccaatgctgtctgatccatc

SEQ ID NO: 83 - DNA, 20 bp, synthetic construct
  misc_feature 1..20: note = Synthetic oligonucleotide
  source 1..20: mol_type = other DNA
tttggctcctgtgaagttgc

SEQ ID NO: 84 - DNA, 20 bp, synthetic construct
  misc_feature 1..20: note = Synthetic oligonucleotide
  source 1..20: mol_type = other DNA
acactttttggctcctgtga

SEQ ID NO: 85 - DNA, 20 bp, synthetic construct
  misc_feature 1..20: note = Synthetic oligonucleotide
  source 1..20: mol_type = other DNA
gactagacactttttggctc

SEQ ID NO: 86 - DNA, 20 bp, synthetic construct
  misc_feature 1..20: note = Synthetic oligonucleotide
  source 1..20: mol_type = other DNA
taagttgactagacactttt

SEQ ID NO: 87 - DNA, 20 bp, synthetic construct
  misc_feature 1..20: note = Synthetic oligonucleotide
  source 1..20: mol_type = other DNA
ctcaattaagttgactagac

SEQ ID NO: 88 - DNA, 20 bp, synthetic construct
  misc_feature 1..20: note = Synthetic oligonucleotide
  source 1..20: mol_type = other DNA
caccttctcaattaagttga

SEQ ID NO: 89 - DNA, 20 bp, synthetic construct
  misc_feature 1..20: note = Synthetic oligonucleotide
  source 1..20: mol_type = other DNA
acttgccaccttctcaatta

SEQ ID NO: 90 - DNA, 20 bp, synthetic construct
  misc_feature 1..20: note = Synthetic oligonucleotide
  source 1..20: mol_type = other DNA
accataacttgccaccttct

SEQ ID NO: 91 - DNA, 20 bp, synthetic construct
  misc_feature 1..20: note = Synthetic oligonucleotide
  source 1..20: mol_type = other DNA
cttcacaccataacttgcca

SEQ ID NO: 92 - DNA, 20 bp, synthetic construct
  misc_feature 1..20: note = Synthetic oligonucleotide
  source 1..20: mol_type = other DNA
tcttggcttcacaccataac

SEQ ID NO: 93 - DNA, 20 bp, synthetic construct
  misc_feature 1..20: note = Synthetic oligonucleotide
  source 1..20: mol_type = other DNA
atgtggcatatgtcactaga

SEQ ID NO: 94 - DNA, 20 bp, synthetic construct
  misc_feature 1..20: note = Synthetic oligonucleotide
  source 1..20: mol_type = other DNA
cagacactttgacccaaatt

SEQ ID NO: 95 - DNA, 20 bp, synthetic construct
  misc_feature 1..20: note = Synthetic oligonucleotide
  source 1..20: mol_type = other DNA
ggtcttcataattgatttca

SEQ ID NO: 96 - DNA, 20 bp, synthetic construct
  misc_feature 1..20: note = Synthetic oligonucleotide
  source 1..20: mol_type = other DNA
acttgtggtcttcataattg

SEQ ID NO: 97 - DNA, 20 bp, synthetic construct
  misc_feature 1..20: note = Synthetic oligonucleotide
  source 1..20: mol_type = other DNA
acttcaacttgtggtcttca

SEQ ID NO: 98 - DNA, 20 bp, synthetic construct
  misc_feature 1..20: note = Synthetic oligonucleotide
  source 1..20: mol_type = other DNA
tccctgacttcaacttgtgg

SEQ ID NO: 99 - DNA, 20 bp, synthetic construct
  misc_feature 1..20: note = Synthetic oligonucleotide
  source 1..20: mol_type = other DNA
tgttagtccctgacttcaac

SEQ ID NO: 100 - DNA, 20 bp, synthetic construct
  misc_feature 1..20: note = Synthetic oligonucleotide
  source 1..20: mol_type = other DNA
tcttggtgttagtccctgac

SEQ ID NO: 101 - DNA, 20 bp, synthetic construct
  misc_feature 1..20: note = Synthetic oligonucleotide
  source 1..20: mol_type = other DNA
tgtacactgcctggagggcc

SEQ ID NO: 102 - DNA, 20 bp, synthetic construct
  misc_feature 1..20: note = Synthetic oligonucleotide
  source 1..20: mol_type = other DNA
tcatgctgtacactgcctgg

SEQ ID NO: 103 - DNA, 20 bp, synthetic construct
  misc_feature 1..20: note = Synthetic oligonucleotide
  source 1..20: mol_type = other DNA
gttccagccttcaggaggga

SEQ ID NO: 104 - DNA, 20 bp, synthetic construct
  misc_feature 1..20: note = Synthetic oligonucleotide
  source 1..20: mol_type = other DNA
ggtgcggttccagccttcag

SEQ ID NO: 105 - DNA, 20 bp, synthetic construct
  misc_feature 1..20: note = Synthetic oligonucleotide
  source 1..20: mol_type = other DNA
atggcgggtgcggttccagc

SEQ ID NO: 106 - DNA, 20 bp, synthetic construct
  misc_feature 1..20: note = Synthetic oligonucleotide
  source 1..20: mol_type = other DNA
gatgacatggcgggtgcggt

SEQ ID NO: 107 - DNA, 20 bp, synthetic construct
  misc_feature 1..20: note = Synthetic oligonucleotide
  source 1..20: mol_type = other DNA
gaggatgatgacatggcggg

SEQ ID NO: 108 - DNA, 20 bp, synthetic construct
  misc_feature 1..20: note = Synthetic oligonucleotide
  source 1..20: mol_type = other DNA
cccatgttgtgcaatccatc

SEQ ID NO: 109 - DNA, 20 bp, synthetic construct
  misc_feature 1..20: note = Synthetic oligonucleotide
  source 1..20: mol_type = other DNA
tccccgcccatgttgtgcaa

SEQ ID NO: 110 - DNA, 20 bp, synthetic construct
  misc_feature 1..20: note = Synthetic oligonucleotide
  source 1..20: mol_type = other DNA
attgggtccccgcccatgtt

SEQ ID NO: 111 - DNA, 20 bp, synthetic construct
  misc_feature 1..20: note = Synthetic oligonucleotide
  source 1..20: mol_type = other DNA
acagtaattgggtccccgcc

SEQ ID NO: 112 - DNA, 20 bp, synthetic construct
  misc_feature 1..20: note = Synthetic oligonucleotide
  source 1..20: mol_type = other DNA
tcaatgacagtaattgggtc

SEQ ID NO: 113 - DNA, 20 bp, synthetic construct
  misc_feature 1..20: note = Synthetic oligonucleotide
  source 1..20: mol_type = other DNA
atctcatcaatgacagtaat

SEQ ID NO: 114 - DNA, 20 bp, synthetic construct
  misc_feature 1..20: note = Synthetic oligonucleotide
  source 1..20: mol_type = other DNA
tcccggatctcatcaatgac

SEQ ID NO: 115 - DNA, 20 bp, synthetic construct
  misc_feature 1..20: note = Synthetic oligonucleotide
  source 1..20: mol_type = other DNA
acatccagataatcctccct

SEQ ID NO: 116 - DNA, 20 bp, synthetic construct
  misc_feature 1..20: note = Synthetic oligonucleotide
  source 1..20: mol_type = other DNA
acatagacatccagataatc

SEQ ID NO: 117 - DNA, 20 bp, synthetic construct
  misc_feature 1..20: note = Synthetic oligonucleotide
  source 1..20: mol_type = other DNA
ccaaacacatagacatccag

SEQ ID NO: 118 - DNA, 20 bp, synthetic construct
  misc_feature 1..20: note = Synthetic oligonucleotide
  source 1..20: mol_type = other DNA
agcattgatgttcacttggt

SEQ ID NO: 119 - DNA, 20 bp, synthetic construct
  misc_feature 1..20: note = Synthetic oligonucleotide
  source 1..20: mol_type = other DNA
agccaaagcattgatgttca

SEQ ID NO: 120 - DNA, 20 bp, synthetic construct
  misc_feature 1..20: note = Synthetic oligonucleotide
  source 1..20: mol_type = other DNA
cttggaagccaaagcattga

SEQ ID NO: 121 - DNA, 20 bp, synthetic construct
  misc_feature 1..20: note = Synthetic oligonucleotide
  source 1..20: mol_type = other DNA
gtctttcttggaagccaaag

SEQ ID NO: 122 - DNA, 20 bp, synthetic construct
  misc_feature 1..20: note = Synthetic oligonucleotide
  source 1..20: mol_type = other DNA
ctcattgtctttcttggaag

SEQ ID NO: 123 - DNA, 20 bp, synthetic construct
  misc_feature 1..20: note = Synthetic oligonucleotide
  source 1..20: mol_type = other DNA
atgttgctcattgtctttct

SEQ ID NO: 124 - DNA, 20 bp, synthetic construct
  misc_feature 1..20: note = Synthetic oligonucleotide
  source 1..20: mol_type = other DNA
gaacacatgttgctcattgt

SEQ ID NO: 125 - DNA, 20 bp, synthetic construct
  misc_feature 1..20: note = Synthetic oligonucleotide
  source 1..20: mol_type = other DNA
gactttgaacacatgttgct

SEQ ID NO: 126 - DNA, 20 bp, synthetic construct
  misc_feature 1..20: note = Synthetic oligonucleotide
  source 1..20: mol_type = other DNA
atccttgactttgaacacat

SEQ ID NO: 127 - DNA, 20 bp, synthetic construct
  misc_feature 1..20: note = Synthetic oligonucleotide
  source 1..20: mol_type = other DNA
ttccatatccttgactttga

SEQ ID NO: 128 - DNA, 20 bp, synthetic construct
  misc_feature 1..20: note = Synthetic oligonucleotide
  source 1..20: mol_type = other DNA
caggttttccatatccttga

SEQ ID NO: 129 - DNA, 20 bp, synthetic construct
  misc_feature 1..20: note = Synthetic oligonucleotide
  source 1..20: mol_type = other DNA
ctcagagactggctttcatc

SEQ ID NO: 130 - DNA, 20 bp, synthetic construct
  misc_feature 1..20: note = Synthetic oligonucleotide
  source 1..20: mol_type = other DNA
cagagactcagagactggct

SEQ ID NO: 131 - DNA, 20 bp, synthetic construct
  misc_feature 1..20: note = Synthetic oligonucleotide
  source 1..20: mol_type = other DNA
atgccacagagactcagaga

SEQ ID NO: 132 - DNA, 20 bp, synthetic construct
  misc_feature 1..20: note = Synthetic oligonucleotide
  source 1..20: mol_type = other DNA
caaaccatgccacagagact

SEQ ID NO: 133 - DNA, 20 bp, synthetic construct
  misc_feature 1..20: note = Synthetic oligonucleotide
  source 1..20: mol_type = other DNA
tgttcccaaaccatgccaca

SEQ ID NO: 134 - DNA, 20 bp, synthetic construct
  misc_feature 1..20: note = Synthetic oligonucleotide
  source 1..20: mol_type = other DNA
ttgtggtaatcggtaccctt

SEQ ID NO: 135 - DNA, 20 bp, synthetic construct
  misc_feature 1..20: note = Synthetic oligonucleotide
  source 1..20: mol_type = other DNA
ggttgcttgtggtaatcggt

SEQ ID NO: 136 - DNA, 20 bp, synthetic construct
  misc_feature 1..20: note = Synthetic oligonucleotide
  source 1..20: mol_type = other DNA
tgccatggttgcttgtggta

SEQ ID NO: 137 - DNA, 20 bp, synthetic construct
  misc_feature 1..20: note = Synthetic oligonucleotide
  source 1..20: mol_type = other DNA
ttggcctgccatggttgctt

SEQ ID NO: 138 - DNA, 20 bp, synthetic construct
  misc_feature 1..20: note = Synthetic oligonucleotide
  source 1..20: mol_type = other DNA
gagatcttggcctgccatgg

SEQ ID NO: 139 - DNA, 20 bp, synthetic construct
  misc_feature 1..20: note = Synthetic oligonucleotide
  source 1..20: mol_type = other DNA
acagcccccatacagctctc

SEQ ID NO: 140 - DNA, 20 bp, synthetic construct
  misc_feature 1..20: note = Synthetic oligonucleotide
  source 1..20: mol_type = other DNA
gacaccacagcccccataca

SEQ ID NO: 141 - DNA, 20 bp, synthetic construct
  misc_feature 1..20: note = Synthetic oligonucleotide
  source 1..20: mol_type = other DNA
tactcagacaccacagcccc

SEQ ID NO: 142 - DNA, 20 bp, synthetic construct
  misc_feature 1..20: note = Synthetic oligonucleotide
  source 1..20: mol_type = other DNA
acaaagtactcagacaccac

SEQ ID NO: 143 - DNA, 20 bp, synthetic construct
  misc_feature 1..20: note = Synthetic oligonucleotide
  source 1..20: mol_type = other DNA
gtcagcacaaagtactcaga

SEQ ID NO: 144 - DNA, 20 bp, synthetic construct
  misc_feature 1..20: note = Synthetic oligonucleotide
  source 1..20: mol_type = other DNA
ttgattgagtgttccttgtc

SEQ ID NO: 145 - DNA, 20 bp, synthetic construct
  misc_feature 1..20: note = Synthetic oligonucleotide
  source 1..20: mol_type = other DNA
ctgaccttgattgagtgttc

SEQ ID NO: 146 - DNA, 20 bp, synthetic construct
  misc_feature 1..20: note = Synthetic oligonucleotide
  source 1..20: mol_type = other DNA
tatctccaggtcccgcttct

SEQ ID NO: 147 - DNA, 20 bp, synthetic construct
  misc_feature 1..20: note = Synthetic oligonucleotide
  source 1..20: mol_type = other DNA
gaattcctgcttcttttttc

SEQ ID NO: 148 - DNA, 20 bp, synthetic construct
  misc_feature 1..20: note = Synthetic oligonucleotide
  source 1..20: mol_type = other DNA
attcaggaattcctgcttct

SEQ ID NO: 149 - DNA, 20 bp, synthetic construct
  misc_feature 1..20: note = Synthetic oligonucleotide
  source 1..20: mol_type = other DNA
cataaaattcaggaattcct

SEQ ID NO: 150 - DNA, 20 bp, synthetic construct
  misc_feature 1..20: note = Synthetic oligonucleotide
  source 1..20: mol_type = other DNA
catagtcataaaattcagga

SEQ ID NO: 151 - DNA, 20 bp, synthetic construct
  misc_feature 1..20: note = Synthetic oligonucleotide
  source 1..20: mol_type = other DNA
tgagcttgatcagggcaacg

SEQ ID NO: 152 - DNA, 20 bp, synthetic construct
  misc_feature 1..20: note = Synthetic oligonucleotide
  source 1..20: mol_type = other DNA
tattcttgagcttgatcagg

SEQ ID NO: 153 - DNA, 20 bp, synthetic construct
  misc_feature 1..20: note = Synthetic oligonucleotide
  source 1..20: mol_type = other DNA
gacaaatgggcctgatagtc

SEQ ID NO: 154 - DNA, 20 bp, synthetic construct
  misc_feature 1..20: note = Synthetic oligonucleotide
  source 1..20: mol_type = other DNA
gttgttccctcggtgcaggg

SEQ ID NO: 155 - DNA, 20 bp, synthetic construct
  misc_feature 1..20: note = Synthetic oligonucleotide
  source 1..20: mol_type = other DNA
gctcgagttgttccctcggt

SEQ ID NO: 156 - DNA, 20 bp, synthetic construct
  misc_feature 1..20: note = Synthetic oligonucleotide
  source 1..20: mol_type = other DNA
ctcaaagctcgagttgttcc

SEQ ID NO: 157 - DNA, 20 bp, synthetic construct
  misc_feature 1..20: note = Synthetic oligonucleotide
  source 1..20: mol_type = other DNA
ggaagcctcaaagctcgagt

SEQ ID NO: 158 - DNA, 20 bp, synthetic construct
  misc_feature 1..20: note = Synthetic oligonucleotide
  source 1..20: mol_type = other DNA
gttggaggaagcctcaaagc

SEQ ID NO: 159 - DNA, 20 bp, synthetic construct
  misc_feature 1..20: note = Synthetic oligonucleotide
  source 1..20: mol_type = other DNA
gtggtagttggaggaagcct

SEQ ID NO: 160 - DNA, 20 bp, synthetic construct
  misc_feature 1..20: note = Synthetic oligonucleotide
  source 1..20: mol_type = other DNA
tggcaagtggtagttggagg

SEQ ID NO: 161 - DNA, 20 bp, synthetic construct
  misc_feature 1..20: note = Synthetic oligonucleotide
  source 1..20: mol_type = other DNA
tgttgctggcaagtggtagt

SEQ ID NO: 162 - DNA, 20 bp, synthetic construct
  misc_feature 1..20: note = Synthetic oligonucleotide
  source 1..20: mol_type = other DNA
tccagctcactcccctgttg

SEQ ID NO: 163 - DNA, 20 bp, synthetic construct
  misc_feature 1..20: note = Synthetic oligonucleotide
  source 1..20: mol_type = other DNA
taaggatccagctcactccc

SEQ ID NO: 164 - DNA, 20 bp, synthetic construct
  misc_feature 1..20: note = Synthetic oligonucleotide
  source 1..20: mol_type = other DNA
cagaaataaggatccagctc

SEQ ID NO: 165 - DNA, 20 bp, synthetic construct
  misc_feature 1..20: note = Synthetic oligonucleotide
  source 1..20: mol_type = other DNA
agggaccagaaataaggatc

SEQ ID NO: 166 - DNA, 20 bp, synthetic construct
  misc_feature 1..20: note = Synthetic oligonucleotide
  source 1..20: mol_type = other DNA
ccacttagggaccagaaata

SEQ ID NO: 167 - DNA, 20 bp, synthetic construct
  misc_feature 1..20: note = Synthetic oligonucleotide
  source 1..20: mol_type = other DNA
tccaggactctccccttcag

SEQ ID NO: 168 - DNA, 20 bp, synthetic construct
  misc_feature 1..20: note = Synthetic oligonucleotide
  source 1..20: mol_type = other DNA
aagtcccaccctttgctgcc

SEQ ID NO: 169 - DNA, 20 bp, synthetic construct
  misc_feature 1..20: note = Synthetic oligonucleotide
  source 1..20: mol_type = other DNA
ctgcagaagtcccacccttt

SEQ ID NO: 170 - DNA, 20 bp, synthetic construct
  misc_feature 1..20: note = Synthetic oligonucleotide
  source 1..20: mol_type = other DNA
cagaaactgcagaagtccca

SEQ ID NO: 171 - DNA, 20 bp, synthetic construct
  misc_feature 1..20: note = Synthetic oligonucleotide
  source 1..20: mol_type = other DNA
aacctctgcactctgccttc

SEQ ID NO: 172 - DNA, 20 bp, synthetic construct
  misc_feature 1..20: note = Synthetic oligonucleotide
  source 1..20: mol_type = other DNA
ccctcaaacctctgcactct

SEQ ID NO: 173 - DNA, 20 bp, synthetic construct
  misc_feature 1..20: note = Synthetic oligonucleotide
  source 1..20: mol_type = other DNA
tcattgccctcaaacctctg

SEQ ID NO: 174 - DNA, 20 bp, synthetic construct
  misc_feature 1..20: note = Synthetic oligonucleotide
  source 1..20: mol_type = other DNA
ccacactcattgccctcaaa

SEQ ID NO: 175 - DNA, 20 bp, synthetic construct
  misc_feature 1..20: note = Synthetic oligonucleotide
  source 1..20: mol_type = other DNA
cactgcccacactcattgcc

SEQ ID NO: 176 - DNA, 20 bp, synthetic construct
  misc_feature 1..20: note = Synthetic oligonucleotide
  source 1..20: mol_type = other DNA
ttaggccactgcccacactc

SEQ ID NO: 177 - DNA, 20 bp, synthetic construct
  misc_feature 1..20: note = Synthetic oligonucleotide
  source 1..20: mol_type = other DNA
ctagtcctgaccttgctgcc

SEQ ID NO: 178 - DNA, 20 bp, synthetic construct
  misc_feature 1..20: note = Synthetic oligonucleotide
  source 1..20: mol_type = other DNA
ctcatcctagtcctgacctt

SEQ ID NO: 179 - DNA, 20 bp, synthetic construct
  misc_feature 1..20: note = Synthetic oligonucleotide
  source 1..20: mol_type = other DNA
cctagtctcatcctagtcct

SEQ ID NO: 180 - DNA, 20 bp, synthetic construct
  misc_feature 1..20: note = Synthetic oligonucleotide
  source 1..20: mol_type = other DNA
accctgcctagtctcatcct

SEQ ID NO: 181 - DNA, 20 bp, synthetic construct
  misc_feature 1..20: note = Synthetic oligonucleotide
  source 1..20: mol_type = other DNA
cttgtcaccctgcctagtct

SEQ ID NO: 182 - DNA, 20 bp, synthetic construct
  misc_feature 1..20: note = Synthetic oligonucleotide
  source 1..20: mol_type = other DNA
gcccaccttgtcaccctgcc

SEQ ID NO: 183 - DNA, 20 bp, synthetic construct
  misc_feature 1..20: note = Synthetic oligonucleotide
  source 1..20: mol_type = other DNA
cctaaaactgctcctactcc

SEQ ID NO: 184 - DNA, 20 bp, synthetic construct
  misc_feature 1..20: note = Synthetic oligonucleotide
  source 1..20: mol_type = other DNA
gagtcagaaatgaggtcaaa

SEQ ID NO: 185 - DNA, 20 bp, synthetic construct
  misc_feature 1..20: note = Synthetic oligonucleotide
  source 1..20: mol_type = other DNA
ccctactcccatttcacctt

SEQ ID NO: 186 - DNA, 20 bp, synthetic construct
  misc_feature 1..20: note = Synthetic oligonucleotide
  source 1..20: mol_type = other DNA
tgttgtgcaatcctgcagaa

SEQ ID NO: 187 - DNA, 20 bp, synthetic construct
  misc_feature 1..20: note = Synthetic oligonucleotide
  source 1..20: mol_type = other DNA
aaaggctgatgaagcctggc

SEQ ID NO: 188 - DNA, 20 bp, synthetic construct
  misc_feature 1..20: note = Synthetic oligonucleotide
  source 1..20: mol_type = other DNA
cctttgaccacaaagtggcc

SEQ ID NO: 189 - DNA, 20 bp, synthetic construct
  misc_feature 1..20: note = Synthetic oligonucleotide
  source 1..20: mol_type = other DNA
aggtaccacctctttgtggg

SEQ ID NO: 190 - DNA, 20 bp, synthetic construct
  misc_feature 1..20: note = Synthetic oligonucleotide
  source 1..20: mol_type = other DNA
tggtggtcacacctgaagag

SEQ ID NO: 191 - DNA, 20 bp, synthetic construct
  misc_feature 1..20: note = Synthetic oligonucleotide
  source 1..20: mol_type = other DNA
gcagggagcagctcttcctt

SEQ ID NO: 192 - DNA, 20 bp, synthetic construct
  misc_feature 1..20: note = Synthetic oligonucleotide
  source 1..20: mol_type = other DNA
tcctgtgcagggagcagctc

SEQ ID NO: 193 - DNA, 20 bp, synthetic construct
  misc_feature 1..20: note = Synthetic oligonucleotide
  source 1..20: mol_type = other DNA
ttgatatcctgtgcagggag

SEQ ID NO: 194 - DNA, 20 bp, synthetic construct
  misc_feature 1..20: note = Synthetic oligonucleotide
  source 1..20: mol_type = other DNA
agagctttgatatcctgtgc

SEQ ID NO: 195 - DNA, 20 bp, synthetic construct
  misc_feature 1..20: note = Synthetic oligonucleotide
  source 1..20: mol_type = other DNA
acaaacagagctttgatatc

SEQ ID NO: 196 - DNA, 20 bp, synthetic construct
  misc_feature 1..20: note = Synthetic oligonucleotide
  source 1..20: mol_type = other DNA
tcagacacaaacagagcttt

SEQ ID NO: 197 - DNA, 20 bp, synthetic construct
  misc_feature 1..20: note = Synthetic oligonucleotide
  source 1..20: mol_type = other DNA
tcctcctcagacacaaacag

SEQ ID NO: 198 - DNA, 20 bp, synthetic construct
  misc_feature 1..20: note = Synthetic oligonucleotide
  source 1..20: mol_type = other DNA
acctccttccgagtcagctt

SEQ ID NO: 199 - DNA, 20 bp, synthetic construct
  misc_feature 1..20: note = Synthetic oligonucleotide
  source 1..20: mol_type = other DNA
atgtagacctccttccgagt

SEQ ID NO: 200 - DNA, 20 bp, synthetic construct
  misc_feature 1..20: note = Synthetic oligonucleotide
  source 1..20: mol_type = other DNA
ttcttgatgtagacctcctt

SEQ ID NO: 201 - DNA, 20 bp, synthetic construct
  misc_feature 1..20: note = Synthetic oligonucleotide
  source 1..20: mol_type = other DNA
tccccattcttgatgtagac

SEQ ID NO: 202 - DNA, 20 bp, synthetic construct
  misc_feature 1..20: note = Synthetic oligonucleotide
  source 1..20: mol_type = other DNA
ttcttatccccattcttgat

SEQ ID NO: 203 - DNA, 20 bp, synthetic construct
  misc_feature 1..20: note = Synthetic oligonucleotide
  source 1..20: mol_type = other DNA
ctgcctttcttatccccatt

SEQ ID NO: 204 - DNA, 20 bp, synthetic construct
  misc_feature 1..20: note = Synthetic oligonucleotide
  source 1..20: mol_type = other DNA
tcacagctgcctttcttatc

SEQ ID NO: 205 - DNA, 20 bp, synthetic construct
  misc_feature 1..20: note = Synthetic oligonucleotide
  source 1..20: mol_type = other DNA
tctctctcacagctgccttt

SEQ ID NO: 206 - DNA, 20 bp, synthetic construct
  misc_feature 1..20: note = Synthetic oligonucleotide
  source 1..20: mol_type = other DNA
tgagcatctctctcacagct

SEQ ID NO: 207 - DNA, 20 bp, synthetic construct
  misc_feature 1..20: note = Synthetic oligonucleotide
  source 1..20: mol_type = other DNA
gcatattgagcatctctctc

SEQ ID NO: 208 - DNA, 20 bp, synthetic construct
  misc_feature 1..20: note = Synthetic oligonucleotide
  source 1..20: mol_type = other DNA
tgactttgtcatagcctggg

SEQ ID NO: 209 - DNA, 20 bp, synthetic construct
  misc_feature 1..20: note = Synthetic oligonucleotide
  source 1..20: mol_type = other DNA
tgtccttgactttgtcatag

SEQ ID NO: 210 - DNA, 20 bp, synthetic construct
  misc_feature 1..20: note = Synthetic oligonucleotide
  source 1..20: mol_type = other DNA
cagtacaaaggaaccgaggg

SEQ ID NO: 211 - DNA, 20 bp, synthetic construct
  misc_feature 1..20: note = Synthetic oligonucleotide
  source 1..20: mol_type = other DNA
ctcctccagtacaaaggaac

SEQ ID NO: 212 - DNA, 20 bp, synthetic construct
  misc_feature 1..20: note = Synthetic oligonucleotide
  source 1..20: mol_type = other DNA
gactcactcctccagtacaa

SEQ ID NO: 213 - DNA, 20 bp, synthetic construct
  misc_feature 1..20: note = Synthetic oligonucleotide
  source 1..20: mol_type = other DNA
catagggactcactcctcca

SEQ ID NO: 214 - DNA, 20 bp, synthetic construct
  misc_feature 1..20: note = Synthetic oligonucleotide
  source 1..20: mol_type = other DNA
ggtcagcatagggactcact

SEQ ID NO: 215 - DNA, 20 bp, synthetic construct
  misc_feature 1..20: note = Synthetic oligonucleotide
  source 1..20: mol_type = other DNA
tcacctctgcaagtattggg

SEQ ID NO: 216 - DNA, 20 bp, synthetic construct
  misc_feature 1..20: note = Synthetic oligonucleotide
  source 1..20: mol_type = other DNA
ccagaatcacctctgcaagt

SEQ ID NO: 217 - DNA, 20 bp, synthetic construct
  misc_feature 1..20: note = Synthetic oligonucleotide
  source 1..20: mol_type = other DNA
gggccgccagaatcacctct

SEQ ID NO: 218 - DNA, 20 bp, synthetic construct
  misc_feature 1..20: note = Synthetic oligonucleotide
  source 1..20: mol_type = other DNA
ctcttgtgaactatcaaggg

SEQ ID NO: 219 - DNA, 20 bp, synthetic construct
  misc_feature 1..20: note = Synthetic oligonucleotide
  source 1..20: mol_type = other DNA
cgacttctcttgtgaactat

SEQ ID NO: 220 - DNA, 20 bp, synthetic construct
  misc_feature 1..20: note = Synthetic oligonucleotide
  source 1..20: mol_type = other DNA
atgaaacgacttctcttgtg

SEQ ID NO: 221 - DNA, 20 bp, synthetic construct
  misc_feature 1..20: note = Synthetic oligonucleotide
  source 1..20: mol_type = other DNA
acttgaatgaaacgacttct

SEQ ID NO: 222 - DNA, 20 bp, synthetic construct
  misc_feature 1..20: note = Synthetic oligonucleotide
  source 1..20: mol_type = other DNA
acaccaacttgaatgaaacg

SEQ ID NO: 223 - DNA, 20 bp, synthetic construct
  misc_feature 1..20: note = Synthetic oligonucleotide
  source 1..20: mol_type = other DNA
tccactactccccagctgat

SEQ ID NO: 224 - DNA, 20 bp, synthetic construct
  misc_feature 1..20: note = Synthetic oligonucleotide
  source 1..20: mol_type = other DNA
cagacatccactactcccca

SEQ ID NO: 225 - DNA, 20 bp, synthetic construct
  misc_feature 1..20: note = Synthetic oligonucleotide
  source 1..20: mol_type = other DNA
tttttgcagacatccactac

SEQ ID NO: 226 - DNA, 20 bp, synthetic construct
  misc_feature 1..20: note = Synthetic oligonucleotide
  source 1..20: mol_type = other DNA
ttctggtttttgcagacatc

SEQ ID NO: 227 - DNA, 20 bp, synthetic construct
  misc_feature 1..20: note = Synthetic oligonucleotide
  source 1..20: mol_type = other DNA
tgccgcttctggtttttgca

SEQ ID NO: 228 - DNA, 20 bp, synthetic construct
  misc_feature 1..20: note = Synthetic oligonucleotide
  source 1..20: mol_type = other DNA
tgcttttgccgcttctggtt

SEQ ID NO: 229 - DNA, 20 bp, synthetic construct
  misc_feature 1..20: note = Synthetic oligonucleotide
  source 1..20: mol_type = other DNA
ggtacctgcttttgccgctt

SEQ ID NO: 230 - DNA, 20 bp, synthetic construct
  misc_feature 1..20: note = Synthetic oligonucleotide
  source 1..20: mol_type = other DNA
tgagcaggtacctgcttttg

SEQ ID NO: 231 - DNA, 20 bp, synthetic construct
  misc_feature 1..20: note = Synthetic oligonucleotide
  source 1..20: mol_type = other DNA
ttcagccagggcagcacttg

SEQ ID NO: 232 - DNA, 20 bp, synthetic construct
  misc_feature 1..20: note = Synthetic oligonucleotide
  source 1..20: mol_type = other DNA
ttctccttcagccagggcag

SEQ ID NO: 233 - DNA, 20 bp, synthetic construct
  misc_feature 1..20: note = Synthetic oligonucleotide
  source 1..20: mol_type = other DNA
tggagtttctccttcagcca

SEQ ID NO: 234 - DNA, 20 bp, synthetic construct
  misc_feature 1..20: note = Synthetic oligonucleotide
  source 1..20: mol_type = other DNA
tcatcttggagtttctcctt

SEQ ID NO: 235 - DNA, 20 bp, synthetic construct
  misc_feature 1..20: note = Synthetic oligonucleotide
  source 1..20: mol_type = other DNA
aaatcctcatcttggagttt

SEQ ID NO: 236 - DNA, 20 bp, synthetic construct
  misc_feature 1..20: note = Synthetic oligonucleotide
  source 1..20: mol_type = other DNA
aaacccaaatcctcatcttg

SEQ ID NO: 237 - DNA, 20 bp, synthetic construct
  misc_feature 1..20: note = Synthetic oligonucleotide
  source 1..20: mol_type = other DNA
gtccagcaggaaacccctta

SEQ ID NO: 238 - DNA, 20 bp, synthetic construct
  misc_feature 1..20: note = Synthetic oligonucleotide
  source 1..20: mol_type = other DNA
gcccctgtccagcaggaaac

SEQ ID NO: 239 - DNA, 20 bp, synthetic construct
  misc_feature 1..20: note = Synthetic oligonucleotide
  source 1..20: mol_type = other DNA
agctgttttaattcaatccc

SEQ ID NO: 240 - DNA, 20 bp, synthetic construct
  misc_feature 1..20: note = Synthetic oligonucleotide
  source 1..20: mol_type = other DNA
aacttgccacctgtgggtga

SEQ ID NO: 241 - DNA, 20 bp, synthetic construct
  misc_feature 1..20: note = Synthetic oligonucleotide
  source 1..20: mol_type = other DNA
tcaccttatccccattcttg

SEQ ID NO: 242 - DNA, 20 bp, synthetic construct
  misc_feature 1..20: note = Synthetic oligonucleotide
  source 1..20: mol_type = other DNA
tcaactttcacaaaccacca

SEQ ID NO: 243 - DNA, 20 bp, synthetic construct
  misc_feature 1..20: note = Synthetic oligonucleotide
  source 1..20: mol_type = other DNA
ccgccagaatcacctgcaag

SEQ ID NO: 244 - DNA, 20 bp, synthetic construct
  misc_feature 1..20: note = Synthetic oligonucleotide
  source 1..20: mol_type = other DNA
aggaggaatgaagaaggctt

SEQ ID NO: 245 - DNA, 20 bp, synthetic construct
  misc_feature 1..20: note = Synthetic oligonucleotide
  source 1..20: mol_type = other DNA
gcctttcctcagggatctgg

SEQ ID NO: 246 - DNA, 20 bp, synthetic construct
  misc_feature 1..20: note = Synthetic oligonucleotide
  source 1..20: mol_type = other DNA
aaatgtctgggagtgtcagg

SEQ ID NO: 247 - DNA, 20 bp, synthetic construct
  misc_feature 1..20: note = Synthetic oligonucleotide
  source 1..20: mol_type = other DNA
gcctagagtgcctccttagg

SEQ ID NO: 248 - DNA, 20 bp, synthetic construct
  misc_feature 1..20: note = Synthetic oligonucleotide
  source 1..20: mol_type = other DNA
ggcatctccccagataggaa

SEQ ID NO: 249 - DNA, 20 bp, synthetic construct
  misc_feature 1..20: note = Synthetic oligonucleotide
  source 1..20: mol_type = other DNA
agggagctagtcctggaaga

SEQ ID NO: 250 - DNA, 20 bp, synthetic construct
  misc_feature 1..20: note = Synthetic oligonucleotide
  source 1..20: mol_type = other DNA
acacctgaagagaaaggctg

SEQ ID NO: 251 - DNA, 20 bp, synthetic construct
  misc_feature 1..20: note = Synthetic oligonucleotide
  source 1..20: mol_type = other DNA
ccctttgaccacaaagtggc

SEQ ID NO: 252 - DNA, 20 bp, synthetic construct
  misc_feature 1..20: note = Synthetic oligonucleotide
  source 1..20: mol_type = other DNA
gccctcaaggtagtctcatg

SEQ ID NO: 253 - DNA, 20 bp, synthetic construct
  misc_feature 1..20: note = Synthetic oligonucleotide
  source 1..20: mol_type = other DNA
aagggaaggaggacagaata

SEQ ID NO: 254 - DNA, 20 bp, synthetic construct
  misc_feature 1..20: note = Synthetic oligonucleotide
  source 1..20: mol_type = other DNA
aaaggccaaggagggatgct

SEQ ID NO: 255 - DNA, 20 bp, synthetic construct
  misc_feature 1..20: note = Synthetic oligonucleotide
  source 1..20: mol_type = other DNA
agaggtcccttctgaccatc

SEQ ID NO: 256 - DNA, 20 bp, synthetic construct
  misc_feature 1..20: note = Synthetic oligonucleotide
  source 1..20: mol_type = other DNA
gctgggacaggagagaggtc

SEQ ID NO: 257 - DNA, 20 bp, synthetic construct
  misc_feature 1..20: note = Synthetic oligonucleotide
  source 1..20: mol_type = other DNA
tcaaatgtctgggagtgtca

SEQ ID NO: 258 - DNA, 20 bp, synthetic construct
  misc_feature 1..20: note = Synthetic oligonucleotide
  source 1..20: mol_type = other DNA
agaaggagaatgtgctgaaa

SEQ ID NO: 259 - DNA, 20 bp, synthetic construct
  misc_feature 1..20: note = Synthetic oligonucleotide
  source 1..20: mol_type = other DNA
tgctgaccacttggcatctc

SEQ ID NO: 260 - DNA, 20 bp, synthetic construct
  misc_feature 1..20: note = Synthetic oligonucleotide
  source 1..20: mol_type = other DNA
caactttcacaaaccaccat

SEQ ID NO: 261 - DNA, 20 bp, synthetic construct
  misc_feature 1..20: note = Synthetic oligonucleotide
  source 1..20: mol_type = other DNA
agctctgtgattctaaggtt

SEQ ID NO: 262 - DNA, 20 bp, synthetic construct
  misc_feature 1..20: note = Synthetic oligonucleotide
  source 1..20: mol_type = other DNA
ccacctgtgggtgaggagaa

SEQ ID NO: 263 - DNA, 20 bp, synthetic construct
  misc_feature 1..20: note = Synthetic oligonucleotide
  source 1..20: mol_type = other DNA
gaggactcacttgaatgaaa

SEQ ID NO: 264 - DNA, 20 bp, synthetic construct
  misc_feature 1..20: note = Synthetic oligonucleotide
  source 1..20: mol_type = other DNA
tggaatgatcagggagctag

SEQ ID NO: 265 - DNA, 20 bp, synthetic construct
  misc_feature 1..20: note = Synthetic oligonucleotide
  source 1..20: mol_type = other DNA
gtcccttctccattttcccc

SEQ ID NO: 266 - DNA, 20 bp, synthetic construct
  misc_feature 1..20: note = Synthetic oligonucleotide
  source 1..20: mol_type = other DNA
tcaactttttaagttaatca

SEQ ID NO: 267 - DNA, 20 bp, synthetic construct
  misc_feature 1..20: note = Synthetic oligonucleotide
  source 1..20: mol_type = other DNA
gggtgaggagaacaaggcgc

SEQ ID NO: 268 - DNA, 20 bp, synthetic construct
  misc_feature 1..20: note = Synthetic oligonucleotide
  source 1..20: mol_type = other DNA
cttccaagccatcttttaac

SEQ ID NO: 269 - DNA, 20 bp, synthetic construct
  misc_feature 1..20: note = Synthetic oligonucleotide
  source 1..20: mol_type = other DNA
aggactcacttgaatgaaac

SEQ ID NO: 270 - DNA, 20 bp, synthetic construct
  misc_feature 1..20: note = Synthetic oligonucleotide
  source 1..20: mol_type = other DNA
ttccaggcaactagagcttc

SEQ ID NO: 271 - DNA, 20 bp, synthetic construct
  misc_feature 1..20: note = Synthetic oligonucleotide
  source 1..20: mol_type = other DNA
cagagtccagccactgtttg

SEQ ID NO: 272 - DNA, 20 bp, synthetic construct
  misc_feature 1..20: note = Synthetic oligonucleotide
  source 1..20: mol_type = other DNA
ccaacctgcagaggcagtgg

SEQ ID NO: 273 - DNA, 20 bp, synthetic construct
  misc_feature 1..20: note = Synthetic oligonucleotide
  source 1..20: mol_type = other DNA
tgcaaggagaggagaagctg

SEQ ID NO: 274 - DNA, 20 bp, synthetic construct
  misc_feature 1..20: note = Synthetic oligonucleotide
  source 1..20: mol_type = other DNA
ctaggcaggttactcaccca

SEQ ID NO: 275 - DNA, 20 bp, synthetic construct
  misc_feature 1..20: note = Synthetic oligonucleotide
  source 1..20: mol_type = other DNA
caccataacttgccacctgt

SEQ ID NO: 276 - DNA, 20 bp, synthetic construct
  misc_feature 1..20: note = Synthetic oligonucleotide
  source 1..20: mol_type = other DNA
taggtaccacctctttgtgg

SEQ ID NO: 277 - DNA, 20 bp, synthetic construct
  misc_feature 1..20: note = Synthetic oligonucleotide
  source 1..20: mol_type = other DNA
cttgacctcacctcccccaa

SEQ ID NO: 278 - DNA, 20 bp, synthetic construct
  misc_feature 1..20: note = Synthetic oligonucleotide
  source 1..20: mol_type = other DNA
ccacctctttgtgggcagct

SEQ ID NO: 279 - DNA, 20 bp, synthetic construct
  misc_feature 1..20: note = Synthetic oligonucleotide
  source 1..20: mol_type = other DNA
ttcacaaaccaccatctctt

SEQ ID NO: 280 - DNA, 20 bp, synthetic construct
  misc_feature 1..20: note = Synthetic oligonucleotide
  source 1..20: mol_type = other DNA
ttctcacctccgttgtcaca

SEQ ID NO: 281 - DNA, 20 bp, synthetic construct
  misc_feature 1..20: note = Synthetic oligonucleotide
  source 1..20: mol_type = other DNA
gaaagtgggaggtgttgcct

SEQ ID NO: 282 - DNA, 20 bp, synthetic construct
  misc_feature 1..20: note = Synthetic oligonucleotide
  source 1..20: mol_type = other DNA
acagcaggaagggaaggtta

SEQ ID NO: 283 - DNA, 20 bp, synthetic construct
  misc_feature 1..20: note = Synthetic oligonucleotide
  source 1..20: mol_type = other DNA
catgctgaccacttggcatc

SEQ ID NO: 284 - DNA, 20 bp, synthetic construct
  misc_feature 1..20: note = Synthetic oligonucleotide
  source 1..20: mol_type = other DNA
ggtcaccttggcaggaaggc

SEQ ID NO: 285 - DNA, 20 bp, synthetic construct
  misc_feature 1..20: note = Synthetic oligonucleotide
  source 1..20: mol_type = other DNA
gtatagtgttacaagtggac

SEQ ID NO: 286 - DNA, 20 bp, synthetic construct
  misc_feature 1..20: note = Synthetic oligonucleotide
  source 1..20: mol_type = other DNA
ggacttccctttgaccacaa

SEQ ID NO: 287 - DNA, 20 bp, synthetic construct
  misc_feature 1..20: note = Synthetic oligonucleotide
  source 1..20: mol_type = other DNA
tcaccttgacctcacctccc

SEQ ID NO: 288 - DNA, 20 bp, synthetic construct
  misc_feature 1..20: note = Synthetic oligonucleotide
  source 1..20: mol_type = other DNA
tagagtgcctccttaggatg

SEQ ID NO: 289 - DNA, 20 bp, synthetic construct
  misc_feature 1..20: note = Synthetic oligonucleotide
  source 1..20: mol_type = other DNA
tgacttcaacttgtggtctg

SEQ ID NO: 290 - DNA, 20 bp, synthetic construct
  misc_feature 1..20: note = Synthetic oligonucleotide
  source 1..20: mol_type = other DNA
cagagaaggagaatgtgctg

SEQ ID NO: 291 - DNA, 20 bp, synthetic construct
  misc_feature 1..20: note = Synthetic oligonucleotide
  source 1..20: mol_type = other DNA
agggagcagctcttcctctg

SEQ ID NO: 292 - DNA, 20 bp, synthetic construct
  misc_feature 1..20: note = Synthetic oligonucleotide
  source 1..20: mol_type = other DNA
tgttcccctgggtgccagga

SEQ ID NO: 293 - DNA, 20 bp, synthetic construct
  misc_feature 1..20: note = Synthetic oligonucleotide
  source 1..20: mol_type = other DNA
ggcctggctgttttcaagcc

SEQ ID NO: 294 - DNA, 20 bp, synthetic construct
  misc_feature 1..20: note = Synthetic oligonucleotide
  source 1..20: mol_type = other DNA
gactggctttcatctggcag

SEQ ID NO: 295 - DNA, 20 bp, synthetic construct
  misc_feature 1..20: note = Synthetic oligonucleotide
  source 1..20: mol_type = other DNA
gaaggctttccaggcaacta

SEQ ID NO: 296 - DNA, 20 bp, synthetic construct
  misc_feature 1..20: note = Synthetic oligonucleotide
  source 1..20: mol_type = other DNA
tcacttgaatgaaacgactt

SEQ ID NO: 297 - DNA, 20 bp, synthetic construct
  misc_feature 1..20: note = Synthetic oligonucleotide
  source 1..20: mol_type = other DNA
ggccccaaaaggccaaggag

SEQ ID NO: 298 - DNA, 20 bp, synthetic construct
  misc_feature 1..20: note = Synthetic oligonucleotide
  source 1..20: mol_type = other DNA
aatcacctgcaaggagagga

SEQ ID NO: 299 - DNA, 20 bp, synthetic construct
  misc_feature 1..20: note = Synthetic oligonucleotide
  source 1..20: mol_type = other DNA
gaccttcagttgcatcctta

SEQ ID NO: 300 - DNA, 20 bp, synthetic construct
  misc_feature 1..20: note = Synthetic oligonucleotide
  source 1..20: mol_type = other DNA
tgatgaagcctggccccaaa

SEQ ID NO: 301 - DNA, 20 bp, synthetic construct
  misc_feature 1..20: note = Synthetic oligonucleotide
  source 1..20: mol_type = other DNA
tagaaagtgggaggtgttgc

SEQ ID NO: 302 - DNA, 20 bp, synthetic construct
  misc_feature 1..20: note = Synthetic oligonucleotide
  source 1..20: mol_type = other DNA
cccatccctgactggtctgg

SEQ ID NO: 303 - DNA, 20 bp, synthetic construct
  misc_feature 1..20: note = Synthetic oligonucleotide
  source 1..20: mol_type = other DNA
ccatgggtatagtgttacaa

SEQ ID NO: 304 - DNA, 20 bp, synthetic construct
  misc_feature 1..20: note = Synthetic oligonucleotide
  source 1..20: mol_type = other DNA
gtgttctcttgacttccagg

SEQ ID NO: 305 - DNA, 20 bp, synthetic construct
  misc_feature 1..20: note = Synthetic oligonucleotide
  source 1..20: mol_type = other DNA
ggcctgctcctcaccccagt

SEQ ID NO: 306 - DNA, 20 bp, synthetic construct
  misc_feature 1..20: note = Synthetic oligonucleotide
  source 1..20: mol_type = other DNA
gaggcctggctgttttcaag

SEQ ID NO: 307 - DNA, 20 bp, synthetic construct
  misc_feature 1..20: note = Synthetic oligonucleotide
  source 1..20: mol_type = other DNA
gactctccccttcagtacct

SEQ ID NO: 308 - DNA, 20 bp, synthetic construct
  misc_feature 1..20: note = Synthetic oligonucleotide
  source 1..20: mol_type = other DNA
catgggtatagtgttacaag

SEQ ID NO: 309 - DNA, 20 bp, synthetic construct
  misc_feature 1..20: note = Synthetic oligonucleotide
  source 1..20: mol_type = other DNA
gaaggagaatgtgctgaaaa

SEQ ID NO: 310 - DNA, 20 bp, synthetic construct
  misc_feature 1..20: note = Synthetic oligonucleotide
  source 1..20: mol_type = other DNA
tcacctggtcttccaagcca

SEQ ID NO: 311 - DNA, 20 bp, synthetic construct
  misc_feature 1..20: note = Synthetic oligonucleotide
  source 1..20: mol_type = other DNA
ctccccagataggaaaggga

SEQ ID NO: 312 - DNA, 20 bp, synthetic construct
  misc_feature 1..20: note = Synthetic oligonucleotide
  source 1..20: mol_type = other DNA
ggactcacttgaatgaaacg

SEQ ID NO: 313 - DNA, 20 bp, synthetic construct
  misc_feature 1..20: note = Synthetic oligonucleotide
  source 1..20: mol_type = other DNA
ggccgccagaatcacctgca

SEQ ID NO: 314 - DNA, 20 bp, synthetic construct
  misc_feature 1..20: note = Synthetic oligonucleotide
  source 1..20: mol_type = other DNA
ctcacttgaatgaaacgact

SEQ ID NO: 315 - DNA, 20 bp, synthetic construct
  misc_feature 1..20: note = Synthetic oligonucleotide
  source 1..20: mol_type = other DNA
ctttcccagcctttcctcag

SEQ ID NO: 316 - DNA, 20 bp, synthetic construct
  misc_feature 1..20: note = Synthetic oligonucleotide
  source 1..20: mol_type = other DNA
agaaagtgggaggtgttgcc

SEQ ID NO: 317 - DNA, 20 bp, synthetic construct
  misc_feature 1..20: note = Synthetic oligonucleotide
  source 1..20: mol_type = other DNA
gtcgcagctgttttaattca

SEQ ID NO: 318 - DNA, 20 bp, synthetic construct
  misc_feature 1..20: note = Synthetic oligonucleotide
  source 1..20: mol_type = other DNA
ccaggactctccccttcagt

SEQ ID NO: 319 - DNA, 20 bp, synthetic construct
  misc_feature 1..20: note = Synthetic oligonucleotide
  source 1..20: mol_type = other DNA
agggaaggaggacagaatag

SEQ ID NO: 320 - DNA, 20 bp, synthetic construct
  misc_feature 1..20: note = Synthetic oligonucleotide
  source 1..20: mol_type = other DNA
gaaatgaggtcaaatgtctg

SEQ ID NO: 321 - DNA, 20 bp, synthetic construct
  misc_feature 1..20: note = Synthetic oligonucleotide
  source 1..20: mol_type = other DNA
ggagagtcagaaatgaggtc

SEQ ID NO: 322 - DNA, 20 bp, synthetic construct
  misc_feature 1..20: note = Synthetic oligonucleotide
  source 1..20: mol_type = other DNA
gtagaaagtgggaggtgttg

SEQ ID NO: 323 - DNA, 20 bp, synthetic construct
  misc_feature 1..20: note = Synthetic oligonucleotide
  source 1..20: mol_type = other DNA
tagaaagatctctgaagtgc

SEQ ID NO: 324 - DNA, 20 bp, synthetic construct
  misc_feature 1..20: note = Synthetic oligonucleotide
  source 1..20: mol_type = other DNA
ctgctcctcaccccagtcct

SEQ ID NO: 325 - DNA, 20 bp, synthetic construct
  misc_feature 1..20: note = Synthetic oligonucleotide
  source 1..20: mol_type = other DNA
ctactgggattctgtgctta

SEQ ID NO: 326 - DNA, 20 bp, synthetic construct
  misc_feature 1..20: note = Synthetic oligonucleotide
  source 1..20: mol_type = other DNA
cccaaaaggccaaggaggga

SEQ ID NO: 327 - DNA, 20 bp, synthetic construct
  misc_feature 1..20: note = Synthetic oligonucleotide
  source 1..20: mol_type = other DNA
tgaccacttggcatctcccc

SEQ ID NO: 328 - DNA, 20 bp, synthetic construct
  misc_feature 1..20: note = Synthetic oligonucleotide
  source 1..20: mol_type = other DNA
cctgcaaggagaggagaagc

SEQ ID NO: 329 - DNA, 20 bp, synthetic construct
  misc_feature 1..20: note = Synthetic oligonucleotide
  source 1..20: mol_type = other DNA
ctctcacctctgcaagtatt

SEQ ID NO: 330 - DNA, 20 bp, synthetic construct
  misc_feature 1..20: note = Synthetic oligonucleotide
  source 1..20: mol_type = other DNA
ccccaaaaggccaaggaggg

SEQ ID NO: 331 - DNA, 20 bp, synthetic construct
  misc_feature 1..20: note = Synthetic oligonucleotide
  source 1..20: mol_type = other DNA
gtcttccaagccatctttta

SEQ ID NO: 332 - DNA, 20 bp, synthetic construct
  misc_feature 1..20: note = Synthetic oligonucleotide
  source 1..20: mol_type = other DNA
gttacaagtggacttaaggg

SEQ ID NO: 333 - DNA, 20 bp, synthetic construct
  misc_feature 1..20: note = Synthetic oligonucleotide
  source 1..20: mol_type = other DNA
cccatgttgtgcaatcctgc

SEQ ID NO: 334 - DNA, 20 bp, synthetic construct
  misc_feature 1..20: note = Synthetic oligonucleotide
  source 1..20: mol_type = other DNA
gaggtgggaagcatggagaa

SEQ ID NO: 335 - DNA, 20 bp, synthetic construct
  misc_feature 1..20: note = Synthetic oligonucleotide
  source 1..20: mol_type = other DNA
tgctcccaccactgtcatct

SEQ ID NO: 336 - DNA, 20 bp, synthetic construct
  misc_feature 1..20: note = Synthetic oligonucleotide
  source 1..20: mol_type = other DNA
aggcaggttactcacccaga

SEQ ID NO: 337 - DNA, 20 bp, synthetic construct
  misc_feature 1..20: note = Synthetic oligonucleotide
  source 1..20: mol_type = other DNA
tactgggattctgtgcttac

SEQ ID NO: 338 - DNA, 20 bp, synthetic construct
  misc_feature 1..20: note = Synthetic oligonucleotide
  source 1..20: mol_type = other DNA
gcctttcccagcctttcctc

SEQ ID NO: 339 - DNA, 20 bp, synthetic construct
  misc_feature 1..20: note = Synthetic oligonucleotide
  source 1..20: mol_type = other DNA
gtgcaatcctgcagaagaga

SEQ ID NO: 340 - DNA, 20 bp, synthetic construct
  misc_feature 1..20: note = Synthetic oligonucleotide
  source 1..20: mol_type = other DNA
acaggagagaggtcccttct

SEQ ID NO: 341 - DNA, 20 bp, synthetic construct
  misc_feature 1..20: note = Synthetic oligonucleotide
  source 1..20: mol_type = other DNA
cccaaaaggagaaagggaaa

SEQ ID NO: 342 - DNA, 20 bp, synthetic construct
  misc_feature 1..20: note = Synthetic oligonucleotide
  source 1..20: mol_type = other DNA
aagcccagggtaaatgctta

SEQ ID NO: 343 - DNA, 20 bp, synthetic construct
  misc_feature 1..20: note = Synthetic oligonucleotide
  source 1..20: mol_type = other DNA
gatgaagcctggccccaaaa

SEQ ID NO: 344 - DNA, 20 bp, synthetic construct
  misc_feature 1..20: note = Synthetic oligonucleotide
  source 1..20: mol_type = other DNA
tggcagagaaggagaatgtg

SEQ ID NO: 345 - DNA, 20 bp, synthetic construct
  misc_feature 1..20: note = Synthetic oligonucleotide
  source 1..20: mol_type = other DNA
ttcccagcctttcctcaggg

SEQ ID NO: 346 - DNA, 20 bp, synthetic construct
  misc_feature 1..20: note = Synthetic oligonucleotide
  source 1..20: mol_type = other DNA
ggcagagaaggagaatgtgc

SEQ ID NO: 347 - DNA, 20 bp, synthetic construct
  misc_feature 1..20: note = Synthetic oligonucleotide
  source 1..20: mol_type = other DNA
acagtgccaggaaacaagaa

SEQ ID NO: 348 - DNA, 20 bp, synthetic construct
  misc_feature 1..20: note = Synthetic oligonucleotide
  source 1..20: mol_type = other DNA
taggcaggttactcacccag

SEQ ID NO: 349 - DNA, 20 bp, synthetic construct
  misc_feature 1..20: note = Synthetic oligonucleotide
  source 1..20: mol_type = other DNA
ttctcttgacttccagggct

SEQ ID NO: 350 - DNA, 20 bp, synthetic construct
  misc_feature 1..20: note = Synthetic oligonucleotide
  source 1..20: mol_type = other DNA
cctgctcctcaccccagtcc

SEQ ID NO: 351 - DNA, 20 bp, synthetic construct
  misc_feature 1..20: note = Synthetic oligonucleotide
  source 1..20: mol_type = other DNA
tcccactaacctccattgcc

SEQ ID NO: 352 - DNA, 20 bp, synthetic construct
  misc_feature 1..20: note = Synthetic oligonucleotide
  source 1..20: mol_type = other DNA
ttccctttgaccacaaagtg

SEQ ID NO: 353 - DNA, 20 bp, synthetic construct
  misc_feature 1..20: note = Synthetic oligonucleotide
  source 1..20: mol_type = other DNA
ctgggtcctaggcaggttac

SEQ ID NO: 354 - DNA, 20 bp, synthetic construct
  misc_feature 1..20: note = Synthetic oligonucleotide
  source 1..20: mol_type = other DNA
tccaggcaactagagcttca

SEQ ID NO: 355 - DNA, 20 bp, synthetic construct
  misc_feature 1..20: note = Synthetic oligonucleotide
  source 1..20: mol_type = other DNA
gcccatgttgtgcaatcctg

SEQ ID NO: 356 - DNA, 20 bp, synthetic construct
  misc_feature 1..20: note = Synthetic oligonucleotide
  source 1..20: mol_type = other DNA
ggttcccactaacctccatt

SEQ ID NO: 357 - DNA, 20 bp, synthetic construct
  misc_feature 1..20: note = Synthetic oligonucleotide
  source 1..20: mol_type = other DNA
aggtagagagcaagagttac

SEQ ID NO: 358 - DNA, 20 bp, synthetic construct
  misc_feature 1..20: note = Synthetic oligonucleotide
  source 1..20: mol_type = other DNA
ccactaacctccattgccca

SEQ ID NO: 359 - DNA, 20 bp, synthetic construct
  misc_feature 1..20: note = Synthetic oligonucleotide
  source 1..20: mol_type = other DNA
tcacaaaccaccatctctta

SEQ ID NO: 360 - DNA, 20 bp, synthetic construct
  misc_feature 1..20: note = Synthetic oligonucleotide
  source 1..20: mol_type = other DNA
tactcacccagataatcctc

SEQ ID NO: 361 - DNA, 20 bp, synthetic construct
  misc_feature 1..20: note = Synthetic oligonucleotide
  source 1..20: mol_type = other DNA
tgctcctcaccccagtcctc

SEQ ID NO: 362 - DNA, 20 bp, synthetic construct
  misc_feature 1..20: note = Synthetic oligonucleotide
  source 1..20: mol_type = other DNA
tctcacagctgcctttctgt

SEQ ID NO: 363 - DNA, 20 bp, synthetic construct
  misc_feature 1..20: note = Synthetic oligonucleotide
  source 1..20: mol_type = other DNA
gaaagggaggactcacttga

SEQ ID NO: 364 - DNA, 20 bp, synthetic construct
  misc_feature 1..20: note = Synthetic oligonucleotide
  source 1..20: mol_type = other DNA
ccatcttttaaccccagaga

SEQ ID NO: 365 - DNA, 20 bp, synthetic construct
  misc_feature 1..20: note = Synthetic oligonucleotide
  source 1..20: mol_type = other DNA
tcctcaccccagtcctccag

SEQ ID NO: 366 - DNA, 20 bp, synthetic construct
  misc_feature 1..20: note = Synthetic oligonucleotide
  source 1..20: mol_type = other DNA
ctggcagagaaggagaatgt

SEQ ID NO: 367 - DNA, 20 bp, synthetic construct
  misc_feature 1..20: note = Synthetic oligonucleotide
  source 1..20: mol_type = other DNA
tctccccagataggaaaggg

SEQ ID NO: 368 - DNA, 20 bp, synthetic construct
  misc_feature 1..20: note = Synthetic oligonucleotide
  source 1..20: mol_type = other DNA
acttcagctgctcccaccac

SEQ ID NO: 369 - DNA, 20 bp, synthetic construct
  misc_feature 1..20: note = Synthetic oligonucleotide
  source 1..20: mol_type = other DNA
gacagcaggaagggaaggtt

SEQ ID NO: 370 - DNA, 20 bp, synthetic construct
  misc_feature 1..20: note = Synthetic oligonucleotide
  source 1..20: mol_type = other DNA
ggagacaaatgggcctataa

SEQ ID NO: 371 - DNA, 20 bp, synthetic construct
  misc_feature 1..20: note = Synthetic oligonucleotide
  source 1..20: mol_type = other DNA
ctgctcccaccactgtcatc

SEQ ID NO: 372 - DNA, 20 bp, synthetic construct
  misc_feature 1..20: note = Synthetic oligonucleotide
  source 1..20: mol_type = other DNA
aggaatgaagaaggctttcc

SEQ ID NO: 373 - DNA, 20 bp, synthetic construct
  misc_feature 1..20: note = Synthetic oligonucleotide
  source 1..20: mol_type = other DNA
gggatctcatccttatcctc

SEQ ID NO: 374 - DNA, 20 bp, synthetic construct
  misc_feature 1..20: note = Synthetic oligonucleotide
  source 1..20: mol_type = other DNA
gtgctgggtcctaggcaggt

SEQ ID NO: 375 - DNA, 20 bp, synthetic construct
  misc_feature 1..20: note = Synthetic oligonucleotide
  source 1..20: mol_type = other DNA
caaaaggccaaggagggatg

SEQ ID NO: 376 - DNA, 20 bp, synthetic construct
  misc_feature 1..20: note = Synthetic oligonucleotide
  source 1..20: mol_type = other DNA
ccatgctgaccacttggcat

SEQ ID NO: 377 - DNA, 20 bp, synthetic construct
  misc_feature 1..20: note = Synthetic oligonucleotide
  source 1..20: mol_type = other DNA
ggaggctgggacaggagaga

SEQ ID NO: 378 - DNA, 20 bp, synthetic construct
  misc_feature 1..20: note = Synthetic oligonucleotide
  source 1..20: mol_type = other DNA
ggagcagctcttcctctgga

SEQ ID NO: 379 - DNA, 20 bp, synthetic construct
  misc_feature 1..20: note = Synthetic oligonucleotide
  source 1..20: mol_type = other DNA
tctcacctccgttgtcacag

SEQ ID NO: 380 - DNA, 20 bp, synthetic construct
  misc_feature 1..20: note = Synthetic oligonucleotide
  source 1..20: mol_type = other DNA
cagtcctccagcctttccca

SEQ ID NO: 381 - DNA, 20 bp, synthetic construct
  misc_feature 1..20: note = Synthetic oligonucleotide
  source 1..20: mol_type = other DNA
agtcctccagcctttcccag

SEQ ID NO: 382 - DNA, 20 bp, synthetic construct
  misc_feature 1..20: note = Synthetic oligonucleotide
  source 1..20: mol_type = other DNA
tgaaggagtctgggagagtc

SEQ ID NO: 383 - DNA, 20 bp, synthetic construct
  misc_feature 1..20: note = Synthetic oligonucleotide
  source 1..20: mol_type = other DNA
cagaatcacctgcaaggaga

SEQ ID NO: 384 - DNA, 20 bp, synthetic construct
  misc_feature 1..20: note = Synthetic oligonucleotide
  source 1..20: mol_type = other DNA
taggaaagggaggactcact

SEQ ID NO: 385 - DNA, 20 bp, synthetic construct
  misc_feature 1..20: note = Synthetic oligonucleotide
  source 1..20: mol_type = other DNA
accttggcaggaaggctccg

SEQ ID NO: 386 - DNA, 20 bp, synthetic construct
  misc_feature 1..20: note = Synthetic oligonucleotide
  source 1..20: mol_type = other DNA
gagacaaatgggcctataaa

SEQ ID NO: 387 - DNA, 20 bp, synthetic construct
  misc_feature 1..20: note = Synthetic oligonucleotide
  source 1..20: mol_type = other DNA
ctgaagagaaaggctgatga

SEQ ID NO: 388 - DNA, 20 bp, synthetic construct
  misc_feature 1..20: note = Synthetic oligonucleotide
  source 1..20: mol_type = other DNA
aatgatcagggagctagtcc

SEQ ID NO: 389 - DNA, 20 bp, synthetic construct
  misc_feature 1..20: note = Synthetic oligonucleotide
  source 1..20: mol_type = other DNA
cttagctgacctaaaggaat

SEQ ID NO: 390 - DNA, 20 bp, synthetic construct
  misc_feature 1..20: note = Synthetic oligonucleotide
  source 1..20: mol_type = other DNA
tgggtatagtgttacaagtg

SEQ ID NO: 391 - DNA, 20 bp, synthetic construct
  misc_feature 1..20: note = Synthetic oligonucleotide
  source 1..20: mol_type = other DNA
tgaagagaaaggctgatgaa

SEQ ID NO: 392 - DNA, 20 bp, synthetic construct
  misc_feature 1..20: note = Synthetic oligonucleotide
  source 1..20: mol_type = other DNA
gtgttacaagtggacttaag

SEQ ID NO: 393 - DNA, 20 bp, synthetic construct
  misc_feature 1..20: note = Synthetic oligonucleotide
  source 1..20: mol_type = other DNA
acctgtgggtgaggagaaca

SEQ ID NO: 394 - DNA, 20 bp, synthetic construct
  misc_feature 1..20: note = Synthetic oligonucleotide
  source 1..20: mol_type = other DNA
tcacccagataatcctccct

SEQ ID NO: 395 - DNA, 20 bp, synthetic construct
  misc_feature 1..20: note = Synthetic oligonucleotide
  source 1..20: mol_type = other DNA
tgttgtcgcagctgttttaa

SEQ ID NO: 396 - DNA, 20 bp, synthetic construct
  misc_feature 1..20: note = Synthetic oligonucleotide
  source 1..20: mol_type = other DNA
tggtcacattcccttcccct

SEQ ID NO: 397 - DNA, 20 bp, synthetic construct
  misc_feature 1..20: note = Synthetic oligonucleotide
  source 1..20: mol_type = other DNA
cctggtcacattcccttccc

SEQ ID NO: 398 - DNA, 20 bp, synthetic construct
  misc_feature 1..20: note = Synthetic oligonucleotide
  source 1..20: mol_type = other DNA
tagacctggtcacattccct

SEQ ID NO: 399 - DNA, 20 bp, synthetic construct
  misc_feature 1..20: note = Synthetic oligonucleotide
  source 1..20: mol_type = other DNA
cctagacctggtcacattcc

SEQ ID NO: 400 - DNA, 20 bp, synthetic construct
  misc_feature 1..20: note = Synthetic oligonucleotide
  source 1..20: mol_type = other DNA
ccttccgagtcagctttttc

SEQ ID NO: 401 - DNA, 20 bp, synthetic construct
  misc_feature 1..20: note = Synthetic oligonucleotide
  source 1..20: mol_type = other DNA
ctccttccgagtcagctttt

SEQ ID NO: 402 - DNA, 20 bp, synthetic construct
  misc_feature 1..20: note = Synthetic oligonucleotide
  source 1..20: mol_type = other DNA
agacctccttccgagtcagc

SEQ ID NO: 403 - DNA, 20 bp, synthetic construct
  misc_feature 1..20: note = Synthetic oligonucleotide
  source 1..20: mol_type = other DNA
gtagacctccttccgagtca

SEQ ID NO: 404 - DNA, 20 bp, synthetic construct
  misc_feature 1..20: note = Synthetic oligonucleotide
  source 1..20: mol_type = other DNA
tttgccgcttctggtttttg

SEQ ID NO: 405 - DNA, 20 bp, synthetic construct
  misc_feature 1..20: note = Synthetic oligonucleotide
  source 1..20: mol_type = other DNA
cttttgccgcttctggtttt

SEQ ID NO: 406 - DNA, 20 bp, synthetic construct
  misc_feature 1..20: note = Synthetic oligonucleotide
  source 1..20: mol_type = other DNA
cctgcttttgccgcttctgg

SEQ ID NO: 407 - DNA, 20 bp, synthetic construct
  misc_feature 1..20: note = Synthetic oligonucleotide
  source 1..20: mol_type = other DNA
tacctgcttttgccgcttct

SEQ ID NO: 408 - DNA, 20 bp, synthetic construct
  misc_feature 1..20: note = Synthetic oligonucleotide
  source 1..20: mol_type = other DNA
agaaaacccaaatcctcatc

SEQ ID NO: 409 - DNA, 20 bp, synthetic construct
  misc_feature 1..20: note = Synthetic oligonucleotide
  source 1..20: mol_type = other DNA
tagaaaacccaaatcctcat

SEQ ID NO: 410 - DNA, 20 bp, synthetic construct
  misc_feature 1..20: note = Synthetic oligonucleotide
  source 1..20: mol_type = other DNA
atagaaaacccaaatcctca

SEQ ID NO: 411 - DNA, 20 bp, synthetic construct
  misc_feature 1..20: note = Synthetic oligonucleotide
  source 1..20: mol_type = other DNA
tatagaaaacccaaatcctc

SEQ ID NO: 412 - DNA, 20 bp, synthetic construct
  misc_feature 1..20: note = Synthetic oligonucleotide
  source 1..20: mol_type = other DNA
ttatagaaaacccaaatcct

SEQ ID NO: 413 - DNA, 20 bp, synthetic construct
  misc_feature 1..20: note = Synthetic oligonucleotide
  source 1..20: mol_type = other DNA
cttatagaaaacccaaatcc

SEQ ID NO: 414 - DNA, 20 bp, synthetic construct
  misc_feature 1..20: note = Synthetic oligonucleotide
  source 1..20: mol_type = other DNA
ccttatagaaaacccaaatc

SEQ ID NO: 415 - DNA, 20 bp, synthetic construct
  misc_feature 1..20: note = Synthetic oligonucleotide
  source 1..20: mol_type = other DNA
cccttatagaaaacccaaat

SEQ ID NO: 416 - DNA, 20 bp, synthetic construct
  misc_feature 1..20: note = Synthetic oligonucleotide
  source 1..20: mol_type = other DNA
ccccttatagaaaacccaaa

SEQ ID NO: 417 - DNA, 20 bp, synthetic construct
  misc_feature 1..20: note = Synthetic oligonucleotide
  source 1..20: mol_type = other DNA
accccttatagaaaacccaa

SEQ ID NO: 418 - DNA, 20 bp, synthetic construct
  misc_feature 1..20: note = Synthetic oligonucleotide
  source 1..20: mol_type = other DNA
aaccccttatagaaaaccca

SEQ ID NO: 419 - DNA, 20 bp, synthetic construct
  misc_feature 1..20: note = Synthetic oligonucleotide
  source 1..20: mol_type = other DNA
aaaccccttatagaaaaccc

SEQ ID NO: 420 - DNA, 20 bp, synthetic construct
  misc_feature 1..20: note = Synthetic oligonucleotide
  source 1..20: mol_type = other DNA
gaaaccccttatagaaaacc

SEQ ID NO: 421 - DNA, 20 bp, synthetic construct
  misc_feature 1..20: note = Synthetic oligonucleotide
  source 1..20: mol_type = other DNA
ggaaaccccttatagaaaac

SEQ ID NO: 422 - DNA, 20 bp, synthetic construct
  misc_feature 1..20: note = Synthetic oligonucleotide
  source 1..20: mol_type = other DNA
aggaaaccccttatagaaaa

SEQ ID NO: 423 - DNA, 20 bp, synthetic construct
  misc_feature 1..20: note = Synthetic oligonucleotide
  source 1..20: mol_type = other DNA
caggaaaccccttatagaaa

SEQ ID NO: 424 - DNA, 20 bp, synthetic construct
  misc_feature 1..20: note = Synthetic oligonucleotide
  source 1..20: mol_type = other DNA
gcaggaaaccccttatagaa

SEQ ID NO: 425 - DNA, 20 bp, synthetic construct
  misc_feature 1..20: note = Synthetic oligonucleotide
  source 1..20: mol_type = other DNA
agcaggaaaccccttataga

SEQ ID NO: 426 - DNA, 20 bp, synthetic construct
  misc_feature 1..20: note = Synthetic oligonucleotide
  source 1..20: mol_type = other DNA
cagcaggaaaccccttatag

SEQ ID NO: 427 - DNA, 20 bp, synthetic construct
  misc_feature 1..20: note = Synthetic oligonucleotide
  source 1..20: mol_type = other DNA
ccagcaggaaaccccttata

SEQ ID NO: 428 - DNA, 20 bp, synthetic construct
  misc_feature 1..20: note = Synthetic oligonucleotide
  source 1..20: mol_type = other DNA
tccagcaggaaaccccttat

SEQ ID NO: 429 - DNA, 20 bp, synthetic construct
  misc_feature 1..20: note = Synthetic oligonucleotide
  source 1..20: mol_type = other DNA
tgtccagcaggaaacccctt

SEQ ID NO: 430 - DNA, 20 bp, synthetic construct
  misc_feature 1..20: note = Synthetic oligonucleotide
  source 1..20: mol_type = other DNA
ctgtccagcaggaaacccct

SEQ ID NO: 431 - DNA, 20 bp, synthetic construct
  misc_feature 1..20: note = Synthetic oligonucleotide
  source 1..20: mol_type = other DNA
cctgtccagcaggaaacccc

SEQ ID NO: 432 - DNA, 20 bp, synthetic construct
  misc_feature 1..20: note = Synthetic oligonucleotide
  source 1..20: mol_type = other DNA
ccctgtccagcaggaaaccc

SEQ ID NO: 433 - DNA, 20 bp, synthetic construct
  misc_feature 1..20: note = Synthetic oligonucleotide
  source 1..20: mol_type = other DNA
cccctgtccagcaggaaacc

SEQ ID NO: 434 - DNA, 20 bp, synthetic construct
  misc_feature 1..20: note = Synthetic oligonucleotide
  source 1..20: mol_type = other DNA
cgcccctgtccagcaggaaa

SEQ ID NO: 435 - DNA, 20 bp, synthetic construct
  misc_feature 1..20: note = Synthetic oligonucleotide
  source 1..20: mol_type = other DNA
acgcccctgtccagcaggaa

SEQ ID NO: 436 - DNA, 20 bp, synthetic construct
  misc_feature 1..20: note = Synthetic oligonucleotide
  source 1..20: mol_type = other DNA
cacgcccctgtccagcagga

SEQ ID NO: 437 - DNA, 20 bp, synthetic construct
  misc_feature 1..20: note = Synthetic oligonucleotide
  source 1..20: mol_type = other DNA
ccacgcccctgtccagcagg

SEQ ID NO: 438 - DNA, 20 bp, synthetic construct
  misc_feature 1..20: note = Synthetic oligonucleotide
  source 1..20: mol_type = other DNA
cccacgcccctgtccagcag

SEQ ID NO: 439 - DNA, 20 bp, synthetic construct
  misc_feature 1..20: note = Synthetic oligonucleotide
  source 1..20: mol_type = other DNA
tcccacgcccctgtccagca

SEQ ID NO: 440 - DNA, 20 bp, synthetic construct
  misc_feature 1..20: note = Synthetic oligonucleotide
  source 1..20: mol_type = other DNA
atcccacgcccctgtccagc

SEQ ID NO: 441 - DNA, 20 bp, synthetic construct
  misc_feature 1..20: note = Synthetic oligonucleotide
  source 1..20: mol_type = other DNA
aatcccacgcccctgtccag

SEQ ID NO: 442 - DNA, 20 bp, synthetic construct
  misc_feature 1..20: note = Synthetic oligonucleotide
  source 1..20: mol_type = other DNA
caatcccacgcccctgtcca

SEQ ID NO: 443 - DNA, 20 bp, synthetic construct
  misc_feature 1..20: note = Synthetic oligonucleotide
  source 1..20: mol_type = other DNA
tcaatcccacgcccctgtcc

SEQ ID NO: 444 - DNA, 20 bp, synthetic construct
  misc_feature 1..20: note = Synthetic oligonucleotide
  source 1..20: mol_type = other DNA
ttcaatcccacgcccctgtc

SEQ ID NO: 445 - DNA, 20 bp, synthetic construct
  misc_feature 1..20: note = Synthetic oligonucleotide
  source 1..20: mol_type = other DNA
attcaatcccacgcccctgt

SEQ ID NO: 446 - DNA, 20 bp, synthetic construct
  misc_feature 1..20: note = Synthetic oligonucleotide
  source 1..20: mol_type = other DNA
aattcaatcccacgcccctg

SEQ ID NO: 447 - DNA, 20 bp, synthetic construct
  misc_feature 1..20: note = Synthetic oligonucleotide
  source 1..20: mol_type = other DNA
taattcaatcccacgcccct

SEQ ID NO: 448 - DNA, 20 bp, synthetic construct
  misc_feature 1..20: note = Synthetic oligonucleotide
  source 1..20: mol_type = other DNA
ttaattcaatcccacgcccc

SEQ ID NO: 449 - DNA, 20 bp, synthetic construct
  misc_feature 1..20: note = Synthetic oligonucleotide
  source 1..20: mol_type = other DNA
tttaattcaatcccacgccc

SEQ ID NO: 450 - DNA, 20 bp, synthetic construct
  misc_feature 1..20: note = Synthetic oligonucleotide
  source 1..20: mol_type = other DNA
ttttaattcaatcccacgcc

SEQ ID NO: 451 - DNA, 20 bp, synthetic construct
  misc_feature 1..20: note = Synthetic oligonucleotide
  source 1..20: mol_type = other DNA
gttttaattcaatcccacgc

SEQ ID NO: 452 - DNA, 20 bp, synthetic construct
  misc_feature 1..20: note = Synthetic oligonucleotide
  source 1..20: mol_type = other DNA
tgttttaattcaatcccacg

SEQ ID NO: 453 - DNA, 20 bp, synthetic construct
  misc_feature 1..20: note = Synthetic oligonucleotide
  source 1..20: mol_type = other DNA
ctgttttaattcaatcccac

SEQ ID NO: 454 - DNA, 20 bp, synthetic construct
  misc_feature 1..20: note = Synthetic oligonucleotide
  source 1..20: mol_type = other DNA
gctgttttaattcaatccca

SEQ ID NO: 455 - DNA, 20 bp, synthetic construct
  misc_feature 1..20: note = Synthetic oligonucleotide
  source 1..20: mol_type = other DNA
cagctgttttaattcaatcc

SEQ ID NO: 456 - DNA, 20 bp, synthetic construct
  misc_feature 1..20: note = Synthetic oligonucleotide
  source 1..20: mol_type = other DNA
gcagctgttttaattcaatc

SEQ ID NO: 457 - DNA, 20 bp, synthetic construct
  misc_feature 1..20: note = Synthetic oligonucleotide
  source 1..20: mol_type = other DNA
cgcagctgttttaattcaat

SEQ ID NO: 458 - DNA, 20 bp, synthetic construct
  misc_feature 1..20: note = Synthetic oligonucleotide
  source 1..20: mol_type = other DNA
tcgcagctgttttaattcaa

SEQ ID NO: 459 - DNA, 20 bp, synthetic construct
  misc_feature 1..20: note = Synthetic oligonucleotide
  source 1..20: mol_type = other DNA
tgtcgcagctgttttaattc

SEQ ID NO: 460 - DNA, 20 bp, synthetic construct
  misc_feature 1..20: note = Synthetic oligonucleotide
  source 1..20: mol_type = other DNA
ttgtcgcagctgttttaatt

SEQ ID NO: 461 - DNA, 20 bp, synthetic construct
  misc_feature 1..20: note = Synthetic oligonucleotide
  source 1..20: mol_type = other DNA
gttgtcgcagctgttttaat

SEQ ID NO: 462 - DNA, 20 bp, synthetic construct
  misc_feature 1..20: note = Synthetic oligonucleotide
  source 1..20: mol_type = other DNA
ttgttgtcgcagctgtttta

SEQ ID NO: 463 - DNA, 20 bp, synthetic construct
  misc_feature 1..20: note = Synthetic oligonucleotide
  source 1..20: mol_type = other DNA
tttgttgtcgcagctgtttt

SEQ ID NO: 464 - DNA, 20 bp, synthetic construct
  misc_feature 1..20: note = Synthetic oligonucleotide
  source 1..20: mol_type = other DNA
ttttgttgtcgcagctgttt

SEQ ID NO: 465 - DNA, 20 bp, synthetic construct
  misc_feature 1..20: note = Synthetic oligonucleotide
  source 1..20: mol_type = other DNA
tttttgttgtcgcagctgtt

SEQ ID NO: 466 - DNA, 20 bp, synthetic construct
  misc_feature 1..20: note = Synthetic oligonucleotide
  source 1..20: mol_type = other DNA
ggatccagctcactcccctg

SEQ ID NO: 467 - DNA, 20 bp, synthetic construct
  misc_feature 1..20: note = Synthetic oligonucleotide
  source 1..20: mol_type = other DNA
aaataaggatccagctcact

SEQ ID NO: 468 - DNA, 20 bp, synthetic construct
  misc_feature 1..20: note = Synthetic oligonucleotide
  source 1..20: mol_type = other DNA
gaccagaaataaggatccag

SEQ ID NO: 469 - DNA, 20 bp, synthetic construct
  misc_feature 1..20: note = Synthetic oligonucleotide
  source 1..20: mol_type = other DNA
cttagggaccagaaataagg

SEQ ID NO: 470 - DNA, 20 bp, synthetic construct
  misc_feature 1..20: note = Synthetic oligonucleotide
  source 1..20: mol_type = other DNA
cacccacttagggaccagaa

SEQ ID NO: 471 - DNA, 20 bp, synthetic construct
  misc_feature 1..20: note = Synthetic oligonucleotide
  source 1..20: mol_type = other DNA
accacccacttagggaccag

SEQ ID NO: 472 - DNA, 20 bp, synthetic construct
  misc_feature 1..20: note = Synthetic oligonucleotide
  source 1..20: mol_type = other DNA
aggtccaggactctcccctt

SEQ ID NO: 473 - DNA, 20 bp, synthetic construct
  misc_feature 1..20: note = Synthetic oligonucleotide
  source 1..20: mol_type = other DNA
aaggtccaggactctcccct

SEQ ID NO: 474 - DNA, 20 bp, synthetic construct
  misc_feature 1..20: note = Synthetic oligonucleotide
  source 1..20: mol_type = other DNA
aaactgcagaagtcccaccc

SEQ ID NO: 475 - DNA, 20 bp, synthetic construct
  misc_feature 1..20: note = Synthetic oligonucleotide
  source 1..20: mol_type = other DNA
ggagggccccgctgagctgc

SEQ ID NO: 476 - DNA, 20 bp, synthetic construct
  misc_feature 1..20: note = Synthetic oligonucleotide
  source 1..20: mol_type = other DNA
tcccggaacatccaagcggg

SEQ ID NO: 477 - DNA, 20 bp, synthetic construct
  misc_feature 1..20: note = Synthetic oligonucleotide
  source 1..20: mol_type = other DNA
catcactttcccggaacatc

SEQ ID NO: 478 - DNA, 20 bp, synthetic construct
  misc_feature 1..20: note = Synthetic oligonucleotide
  source 1..20: mol_type = other DNA
ctggtcacattcccttcccc

SEQ ID NO: 479 - DNA, 20 bp, synthetic construct
  misc_feature 1..20: note = Synthetic oligonucleotide
  source 1..20: mol_type = other DNA
ctagacctggtcacattccc

SEQ ID NO: 480 - DNA, 20 bp, synthetic construct
  misc_feature 1..20: note = Synthetic oligonucleotide
  source 1..20: mol_type = other DNA
ggagtggtggtcacacctcc

SEQ ID NO: 481 - DNA, 20 bp, synthetic construct
  misc_feature 1..20: note = Synthetic oligonucleotide
  source 1..20: mol_type = other DNA
accccctccagagagcagga

SEQ ID NO: 482 - DNA, 20 bp, synthetic construct
  misc_feature 1..20: note = Synthetic oligonucleotide
  source 1..20: mol_type = other DNA
atctctaccccctccagaga

SEQ ID NO: 483 - DNA, 20 bp, synthetic construct
  misc_feature 1..20: note = Synthetic oligonucleotide
  source 1..20: mol_type = other DNA
ggtacgggtagaagccagaa

SEQ ID NO: 484 - DNA, 20 bp, synthetic construct
  misc_feature 1..20: note = Synthetic oligonucleotide
  source 1..20: mol_type = other DNA
ggagagtgtaaccgtcatag

SEQ ID NO: 485 - DNA, 20 bp, synthetic construct
  misc_feature 1..20: note = Synthetic oligonucleotide
  source 1..20: mol_type = other DNA
tgcgattggcagagccccgg

SEQ ID NO: 486 - DNA, 20 bp, synthetic construct
  misc_feature 1..20: note = Synthetic oligonucleotide
  source 1..20: mol_type = other DNA
ggcaggtgcgattggcagag

SEQ ID NO: 487 - DNA, 20 bp, synthetic construct
  misc_feature 1..20: note = Synthetic oligonucleotide
  source 1..20: mol_type = other DNA
ggccattcacttggcaggtg

SEQ ID NO: 488 - DNA, 20 bp, synthetic construct
  misc_feature 1..20: note = Synthetic oligonucleotide
  source 1..20: mol_type = other DNA
ttgtcacagatcgctgtctg

SEQ ID NO: 489 - DNA, 20 bp, synthetic construct
  misc_feature 1..20: note = Synthetic oligonucleotide
  source 1..20: mol_type = other DNA
aaggagtcttggcaggaagg

SEQ ID NO: 490 - DNA, 20 bp, synthetic construct
  misc_feature 1..20: note = Synthetic oligonucleotide
  source 1..20: mol_type = other DNA
gtacatgaaggagtcttggc

SEQ ID NO: 491 - DNA, 20 bp, synthetic construct
  misc_feature 1..20: note = Synthetic oligonucleotide
  source 1..20: mol_type = other DNA
aagcttcggccacctcttga

SEQ ID NO: 492 - DNA, 20 bp, synthetic construct
  misc_feature 1..20: note = Synthetic oligonucleotide
  source 1..20: mol_type = other DNA
ccatctagcaccaggtagat

SEQ ID NO: 493 - DNA, 20 bp, synthetic construct
  misc_feature 1..20: note = Synthetic oligonucleotide
  source 1..20: mol_type = other DNA
ggccccaatgctgtctgatc

SEQ ID NO: 494 - DNA, 20 bp, synthetic construct
  misc_feature 1..20: note = Synthetic oligonucleotide
  source 1..20: mol_type = other DNA
aattaagttgactagacact

SEQ ID NO: 495 - DNA, 20 bp, synthetic construct
  misc_feature 1..20: note = Synthetic oligonucleotide
  source 1..20: mol_type = other DNA
tgccaccttctcaattaagt

SEQ ID NO: 496 - DNA, 20 bp, synthetic construct
  misc_feature 1..20: note = Synthetic oligonucleotide
  source 1..20: mol_type = other DNA
taacttgccaccttctcaat

SEQ ID NO: 497 - DNA, 20 bp, synthetic construct
  misc_feature 1..20: note = Synthetic oligonucleotide
  source 1..20: mol_type = other DNA
cataacttgccaccttctca

SEQ ID NO: 498 - DNA, 20 bp, synthetic construct
  misc_feature 1..20: note = Synthetic oligonucleotide
  source 1..20: mol_type = other DNA
acaccataacttgccacctt

SEQ ID NO: 499 - DNA, 20 bp, synthetic construct
  misc_feature 1..20: note = Synthetic oligonucleotide
  source 1..20: mol_type = other DNA
tcacaccataacttgccacc

SEQ ID NO: 500 - DNA, 20 bp, synthetic construct
  misc_feature 1..20: note = Synthetic oligonucleotide
  source 1..20: mol_type = other DNA
tagtccctgacttcaacttg

SEQ ID NO: 501 - DNA, 20 bp, synthetic construct
  misc_feature 1..20: note = Synthetic oligonucleotide
  source 1..20: mol_type = other DNA
tggtgttagtccctgacttc

SEQ ID NO: 502 - DNA, 20 bp, synthetic construct
  misc_feature 1..20: note = Synthetic oligonucleotide
  source 1..20: mol_type = other DNA
gcggttccagccttcaggag

SEQ ID NO: 503 - DNA, 20 bp, synthetic construct
  misc_feature 1..20: note = Synthetic oligonucleotide
  source 1..20: mol_type = other DNA
tcatgaggatgatgacatgg

SEQ ID NO: 504 - DNA, 20 bp, synthetic construct
  misc_feature 1..20: note = Synthetic oligonucleotide
  source 1..20: mol_type = other DNA
ccgcccatgttgtgcaatcc

SEQ ID NO: 505 - DNA, 20 bp, synthetic construct
  misc_feature 1..20: note = Synthetic oligonucleotide
  source 1..20: mol_type = other DNA
gtaattgggtccccgcccat

SEQ ID NO: 506 - DNA, 20 bp, synthetic construct
  misc_feature 1..20: note = Synthetic oligonucleotide
  source 1..20: mol_type = other DNA
aagtcccggatctcatcaat

SEQ ID NO: 507 - DNA, 20 bp, synthetic construct
  misc_feature 1..20: note = Synthetic oligonucleotide
  source 1..20: mol_type = other DNA
aacacatagacatccagata

SEQ ID NO: 508 - DNA, 20 bp, synthetic construct
  misc_feature 1..20: note = Synthetic oligonucleotide
  source 1..20: mol_type = other DNA
caaagcattgatgttcactt

SEQ ID NO: 509 - DNA, 20 bp, synthetic construct
  misc_feature 1..20: note = Synthetic oligonucleotide
  source 1..20: mol_type = other DNA
tttgaacacatgttgctcat

SEQ ID NO: 510 - DNA, 20 bp, synthetic construct
  misc_feature 1..20: note = Synthetic oligonucleotide
  source 1..20: mol_type = other DNA
cttccaggttttccatatcc

SEQ ID NO: 511 - DNA, 20 bp, synthetic construct
  misc_feature 1..20: note = Synthetic oligonucleotide
  source 1..20: mol_type = other DNA
tcttccaggttttccatatc

SEQ ID NO: 512 - DNA, 20 bp, synthetic construct
  misc_feature 1..20: note = Synthetic oligonucleotide
  source 1..20: mol_type = other DNA
agactcagagactggctttc

SEQ ID NO: 513 - DNA, 20 bp, synthetic construct
  misc_feature 1..20: note = Synthetic oligonucleotide
  source 1..20: mol_type = other DNA
gcctgccatggttgcttgtg

SEQ ID NO: 514 - DNA, 20 bp, synthetic construct
  misc_feature 1..20: note = Synthetic oligonucleotide
  source 1..20: mol_type = other DNA
tgactgagatcttggcctgc

SEQ ID NO: 515 - DNA, 20 bp, synthetic construct
  misc_feature 1..20: note = Synthetic oligonucleotide
  source 1..20: mol_type = other DNA
ttctatctccaggtcccgct

SEQ ID NO: 516 - DNA, 20 bp, synthetic construct
  misc_feature 1..20: note = Synthetic oligonucleotide
  source 1..20: mol_type = other DNA
agtcataaaattcaggaatt

SEQ ID NO: 517 - DNA, 20 bp, synthetic construct
  misc_feature 1..20: note = Synthetic oligonucleotide
  source 1..20: mol_type = other DNA
cgagttgttccctcggtgca

SEQ ID NO: 518 - DNA, 20 bp, synthetic construct
  misc_feature 1..20: note = Synthetic oligonucleotide
  source 1..20: mol_type = other DNA
agcctcaaagctcgagttgt

SEQ ID NO: 519 - DNA, 20 bp, synthetic construct
  misc_feature 1..20: note = Synthetic oligonucleotide
  source 1..20: mol_type = other DNA
ggaggaagcctcaaagctcg

SEQ ID NO: 520 - DNA, 20 bp, synthetic construct
  misc_feature 1..20: note = Synthetic oligonucleotide
  source 1..20: mol_type = other DNA
gtagttggaggaagcctcaa

SEQ ID NO: 521 - DNA, 20 bp, synthetic construct
  misc_feature 1..20: note = Synthetic oligonucleotide
  source 1..20: mol_type = other DNA
caagtggtagttggaggaag

SEQ ID NO: 522 - DNA, 20 bp, synthetic construct
  misc_feature 1..20: note = Synthetic oligonucleotide
  source 1..20: mol_type = other DNA
tcctcagacacaaacagagc

SEQ ID NO: 523 - DNA, 20 bp, synthetic construct
  misc_feature 1..20: note = Synthetic oligonucleotide
  source 1..20: mol_type = other DNA
ttctcctcctcagacacaaa

SEQ ID NO: 524 - DNA, 20 bp, synthetic construct
  misc_feature 1..20: note = Synthetic oligonucleotide
  source 1..20: mol_type = other DNA
tagacctccttccgagtcag

SEQ ID NO: 525 - DNA, 20 bp, synthetic construct
  misc_feature 1..20: note = Synthetic oligonucleotide
  source 1..20: mol_type = other DNA
ttgatgtagacctccttccg

SEQ ID NO: 526 - DNA, 20 bp, synthetic construct
  misc_feature 1..20: note = Synthetic oligonucleotide
  source 1..20: mol_type = other DNA
ctttcttatccccattcttg

SEQ ID NO: 527 - DNA, 20 bp, synthetic construct
  misc_feature 1..20: note = Synthetic oligonucleotide
  source 1..20: mol_type = other DNA
gcctttcttatccccattct

SEQ ID NO: 528 - DNA, 20 bp, synthetic construct
  misc_feature 1..20: note = Synthetic oligonucleotide
  source 1..20: mol_type = other DNA
agctgcctttcttatcccca

SEQ ID NO: 529 - DNA, 20 bp, synthetic construct
  misc_feature 1..20: note = Synthetic oligonucleotide
  source 1..20: mol_type = other DNA
cagctgcctttcttatcccc

SEQ ID NO: 530 - DNA, 20 bp, synthetic construct
  misc_feature 1..20: note = Synthetic oligonucleotide
  source 1..20: mol_type = other DNA
acagctgcctttcttatccc

SEQ ID NO: 531 - DNA, 20 bp, synthetic construct
  misc_feature 1..20: note = Synthetic oligonucleotide
  source 1..20: mol_type = other DNA
gcatctctctcacagctgcc

SEQ ID NO: 532 - DNA, 20 bp, synthetic construct
  misc_feature 1..20: note = Synthetic oligonucleotide
  source 1..20: mol_type = other DNA
agatgtccttgactttgtca

SEQ ID NO: 533 - DNA, 20 bp, synthetic construct
  misc_feature 1..20: note = Synthetic oligonucleotide
  source 1..20: mol_type = other DNA
cagcatagggactcactcct

SEQ ID NO: 534 - DNA, 20 bp, synthetic construct
  misc_feature 1..20: note = Synthetic oligonucleotide
  source 1..20: mol_type = other DNA
ccgccagaatcacctctgca

SEQ ID NO: 535 - DNA, 20 bp, synthetic construct
  misc_feature 1..20: note = Synthetic oligonucleotide
  source 1..20: mol_type = other DNA
tgaatgaaacgacttctctt

SEQ ID NO: 536 - DNA, 20 bp, synthetic construct
  misc_feature 1..20: note = Synthetic oligonucleotide
  source 1..20: mol_type = other DNA
acatccactactccccagct

SEQ ID NO: 537 - DNA, 20 bp, synthetic construct
  misc_feature 1..20: note = Synthetic oligonucleotide
  source 1..20: mol_type = other DNA
cgcttctggtttttgcagac

SEQ ID NO: 538 - DNA, 20 bp, synthetic construct
  misc_feature 1..20: note = Synthetic oligonucleotide
  source 1..20: mol_type = other DNA
ttttgccgcttctggttttt

SEQ ID NO: 539 - DNA, 20 bp, synthetic construct
  misc_feature 1..20: note = Synthetic oligonucleotide
  source 1..20: mol_type = other DNA
gcaggtacctgcttttgccg

SEQ ID NO: 540 - DNA, 20 bp, synthetic construct
  misc_feature 1..20: note = Synthetic oligonucleotide
  source 1..20: mol_type = other DNA
tcttggagtttctccttcag

SEQ ID NO: 541 - DNA, 16 bp, synthetic construct
  misc_feature 1..16: note = Synthetic oligonucleotide
  source 1..16: mol_type = other DNA
ggaacatccaagcggg

SEQ ID NO: 542 - DNA, 16 bp, synthetic construct
  misc_feature 1..16: note = Synthetic oligonucleotide
  source 1..16: mol_type = other DNA
tggtcacattcccttc

SEQ ID NO: 543 - DNA, 16 bp, synthetic construct
  misc_feature 1..16: note = Synthetic oligonucleotide
  source 1..16: mol_type = other DNA
cctggtcacattccct

SEQ ID NO: 544 - DNA, 16 bp, synthetic construct
  misc_feature 1..16: note = Synthetic oligonucleotide
  source 1..16: mol_type = other DNA
gacctggtcacattcc

SEQ ID NO: 545 - DNA, 16 bp, synthetic construct
  misc_feature 1..16: note = Synthetic oligonucleotide
  source 1..16: mol_type = other DNA
taacttgccaccttct

SEQ ID NO: 546 - DNA, 16 bp, synthetic construct
  misc_feature 1..16: note = Synthetic oligonucleotide
  source 1..16: mol_type = other DNA
cataacttgccacctt

SEQ ID NO: 547 - DNA, 16 bp, synthetic construct
  misc_feature 1..16: note = Synthetic oligonucleotide
  source 1..16: mol_type = other DNA
accataacttgccacc

SEQ ID NO: 548 - DNA, 16 bp, synthetic construct
  misc_feature 1..16: note = Synthetic oligonucleotide
  source 1..16: mol_type = other DNA
ccttccgagtcagctt

SEQ ID NO: 549 - DNA, 16 bp, synthetic construct
  misc_feature 1..16: note = Synthetic oligonucleotide
  source 1..16: mol_type = other DNA
ctccttccgagtcagc

SEQ ID NO: 550 - DNA, 16 bp, synthetic construct
  misc_feature 1..16: note = Synthetic oligonucleotide
  source 1..16: mol_type = other DNA
acctccttccgagtca

SEQ ID NO: 551 - DNA, 16 bp, synthetic construct
  misc_feature 1..16: note = Synthetic oligonucleotide
  source 1..16: mol_type = other DNA
ctttcttatccccatt

SEQ ID NO: 552 - DNA, 16 bp, synthetic construct
  misc_feature 1..16: note = Synthetic oligonucleotide
  source 1..16: mol_type = other DNA
gcctttcttatcccca

SEQ ID NO: 553 - DNA, 16 bp, synthetic construct
  misc_feature 1..16: note = Synthetic oligonucleotide
  source 1..16: mol_type = other DNA
ctgcctttcttatccc

SEQ ID NO: 554 - DNA, 16 bp, synthetic construct
  misc_feature 1..16: note = Synthetic oligonucleotide
  source 1..16: mol_type = other DNA
tttgccgcttctggtt

SEQ ID NO: 555 - DNA, 16 bp, synthetic construct
  misc_feature 1..16: note = Synthetic oligonucleotide
  source 1..16: mol_type = other DNA
cttttgccgcttctgg

SEQ ID NO: 556 - DNA, 16 bp, synthetic construct
  misc_feature 1..16: note = Synthetic oligonucleotide
  source 1..16: mol_type = other DNA
tgcttttgccgcttct

SEQ ID NO: 557 - DNA, 16 bp, synthetic construct
  misc_feature 1..16: note = Synthetic oligonucleotide
  source 1..16: mol_type = other DNA
aaacccaaatcctcat

SEQ ID NO: 558 - DNA, 16 bp, synthetic construct
  misc_feature 1..16: note = Synthetic oligonucleotide
  source 1..16: mol_type = other DNA
gaaaacccaaatcctc

SEQ ID NO: 559 - DNA, 16 bp, synthetic construct
  misc_feature 1..16: note = Synthetic oligonucleotide
  source 1..16: mol_type = other DNA
tagaaaacccaaatcc

SEQ ID NO: 560 - DNA, 16 bp, synthetic construct
  misc_feature 1..16: note = Synthetic oligonucleotide
  source 1..16: mol_type = other DNA
atagaaaacccaaatc

SEQ ID NO: 561 - DNA, 16 bp, synthetic construct
  misc_feature 1..16: note = Synthetic oligonucleotide
  source 1..16: mol_type = other DNA
cttatagaaaacccaa

SEQ ID NO: 562 - DNA, 16 bp, synthetic construct
  misc_feature 1..16: note = Synthetic oligonucleotide
  source 1..16: mol_type = other DNA
ccttatagaaaaccca

SEQ ID NO: 563 - DNA, 16 bp, synthetic construct
  misc_feature 1..16: note = Synthetic oligonucleotide
  source 1..16: mol_type = other DNA
cccttatagaaaaccc

SEQ ID NO: 564 - DNA, 16 bp, synthetic construct
  misc_feature 1..16: note = Synthetic oligonucleotide
  source 1..16: mol_type = other DNA
ccccttatagaaaacc

SEQ ID NO: 565 - DNA, 16 bp, synthetic construct
  misc_feature 1..16: note = Synthetic oligonucleotide
  source 1..16: mol_type = other DNA
accccttatagaaaac

SEQ ID NO: 566 - DNA, 16 bp, synthetic construct
  misc_feature 1..16: note = Synthetic oligonucleotide
  source 1..16: mol_type = other DNA
aaccccttatagaaaa

SEQ ID NO: 567 - DNA, 16 bp, synthetic construct
  misc_feature 1..16: note = Synthetic oligonucleotide
  source 1..16: mol_type = other DNA
aaaccccttatagaaa

SEQ ID NO: 568 - DNA, 16 bp, synthetic construct
  misc_feature 1..16: note = Synthetic oligonucleotide
  source 1..16: mol_type = other DNA
gaaaccccttatagaa

SEQ ID NO: 569 - DNA, 16 bp, synthetic construct
  misc_feature 1..16: note = Synthetic oligonucleotide
  source 1..16: mol_type = other DNA
ggaaaccccttataga

SEQ ID NO: 570 - DNA, 16 bp, synthetic construct
  misc_feature 1..16: note = Synthetic oligonucleotide
  source 1..16: mol_type = other DNA
aggaaaccccttatag

SEQ ID NO: 571 - DNA, 16 bp, synthetic construct
  misc_feature 1..16: note = Synthetic oligonucleotide
  source 1..16: mol_type = other DNA
caggaaaccccttata

SEQ ID NO: 572 - DNA, 16 bp, synthetic construct
  misc_feature 1..16: note = Synthetic oligonucleotide
  source 1..16: mol_type = other DNA
gcaggaaaccccttat

SEQ ID NO: 573 - DNA, 16 bp, synthetic construct
  misc_feature 1..16: note = Synthetic oligonucleotide
  source 1..16: mol_type = other DNA
agcaggaaacccctta

SEQ ID NO: 574 - DNA, 16 bp, synthetic construct
  misc_feature 1..16: note = Synthetic oligonucleotide
  source 1..16: mol_type = other DNA
cagcaggaaacccctt

SEQ ID NO: 575 - DNA, 16 bp, synthetic construct
  misc_feature 1..16: note = Synthetic oligonucleotide
  source 1..16: mol_type = other DNA
ccagcaggaaacccct

SEQ ID NO: 576 - DNA, 16 bp, synthetic construct
  misc_feature 1..16: note = Synthetic oligonucleotide
  source 1..16: mol_type = other DNA
tccagcaggaaacccc

SEQ ID NO: 577 - DNA, 16 bp, synthetic construct
  misc_feature 1..16: note = Synthetic oligonucleotide
  source 1..16: mol_type = other DNA
gtccagcaggaaaccc

SEQ ID NO: 578 - DNA, 16 bp, synthetic construct
  misc_feature 1..16: note = Synthetic oligonucleotide
  source 1..16: mol_type = other DNA
tgtccagcaggaaacc

SEQ ID NO: 579 - DNA, 16 bp, synthetic construct
  misc_feature 1..16: note = Synthetic oligonucleotide
  source 1..16: mol_type = other DNA
ctgtccagcaggaaac

SEQ ID NO: 580 - DNA, 16 bp, synthetic construct
  misc_feature 1..16: note = Synthetic oligonucleotide
  source 1..16: mol_type = other DNA
cctgtccagcaggaaa

SEQ ID NO: 581 - DNA, 16 bp, synthetic construct
  misc_feature 1..16: note = Synthetic oligonucleotide
  source 1..16: mol_type = other DNA
ccctgtccagcaggaa

SEQ ID NO: 582 - DNA, 16 bp, synthetic construct
  misc_feature 1..16: note = Synthetic oligonucleotide
  source 1..16: mol_type = other DNA
cccctgtccagcagga

SEQ ID NO: 583 - DNA, 16 bp, synthetic construct
  misc_feature 1..16: note = Synthetic oligonucleotide
  source 1..16: mol_type = other DNA
gcccctgtccagcagg

SEQ ID NO: 584 - DNA, 16 bp, synthetic construct
  misc_feature 1..16: note = Synthetic oligonucleotide
  source 1..16: mol_type = other DNA
cgcccctgtccagcag

SEQ ID NO: 585 - DNA, 16 bp, synthetic construct
  misc_feature 1..16: note = Synthetic oligonucleotide
  source 1..16: mol_type = other DNA
acgcccctgtccagca

SEQ ID NO: 586 - DNA, 16 bp, synthetic construct
  misc_feature 1..16: note = Synthetic oligonucleotide
  source 1..16: mol_type = other DNA
cacgcccctgtccagc

SEQ ID NO: 587 - DNA, 16 bp, synthetic construct
  misc_feature 1..16: note = Synthetic oligonucleotide
  source 1..16: mol_type = other DNA
ccacgcccctgtccag

SEQ ID NO: 588 - DNA, 16 bp, synthetic construct
  misc_feature 1..16: note = Synthetic oligonucleotide
  source 1..16: mol_type = other DNA
cccacgcccctgtcca

SEQ ID NO: 589 - DNA, 16 bp, synthetic construct
  misc_feature 1..16: note = Synthetic oligonucleotide
  source 1..16: mol_type = other DNA
tcccacgcccctgtcc

SEQ ID NO: 590 - DNA, 16 bp, synthetic construct
  misc_feature 1..16: note = Synthetic oligonucleotide
  source 1..16: mol_type = other DNA
atcccacgcccctgtc

SEQ ID NO: 591 - DNA, 16 bp, synthetic construct
  misc_feature 1..16: note = Synthetic oligonucleotide
  source 1..16: mol_type = other DNA
aatcccacgcccctgt

SEQ ID NO: 592 - DNA, 16 bp, synthetic construct
  misc_feature 1..16: note = Synthetic oligonucleotide
  source 1..16: mol_type = other DNA
caatcccacgcccctg

SEQ ID NO: 593 - DNA, 16 bp, synthetic construct
  misc_feature 1..16: note = Synthetic oligonucleotide
  source 1..16: mol_type = other DNA
tcaatcccacgcccct

SEQ ID NO: 594 - DNA, 16 bp, synthetic construct
  misc_feature 1..16: note = Synthetic oligonucleotide
  source 1..16: mol_type = other DNA
ttcaatcccacgcccc

SEQ ID NO: 595 - DNA, 16 bp, synthetic construct
  misc_feature 1..16: note = Synthetic oligonucleotide
  source 1..16: mol_type = other DNA
attcaatcccacgccc

SEQ ID NO: 596 - DNA, 16 bp, synthetic construct
  misc_feature 1..16: note = Synthetic oligonucleotide
  source 1..16: mol_type = other DNA
aattcaatcccacgcc

SEQ ID NO: 597 - DNA, 16 bp, synthetic construct
  misc_feature 1..16: note = Synthetic oligonucleotide
  source 1..16: mol_type = other DNA
taattcaatcccacgc

SEQ ID NO: 598 - DNA, 16 bp, synthetic construct
  misc_feature 1..16: note = Synthetic oligonucleotide
  source 1..16: mol_type = other DNA
ttaattcaatcccacg

SEQ ID NO: 599 - DNA, 16 bp, synthetic construct
  misc_feature 1..16: note = Synthetic oligonucleotide
  source 1..16: mol_type = other DNA
tttaattcaatcccac

SEQ ID NO: 600 - DNA, 16 bp, synthetic construct
  misc_feature 1..16: note = Synthetic oligonucleotide
  source 1..16: mol_type = other DNA
ttttaattcaatccca

SEQ ID NO: 601 - DNA, 16 bp, synthetic construct
  misc_feature 1..16: note = Synthetic oligonucleotide
  source 1..16: mol_type = other DNA
gttttaattcaatccc

SEQ ID NO: 602 - DNA, 16 bp, synthetic construct
  misc_feature 1..16: note = Synthetic oligonucleotide
  source 1..16: mol_type = other DNA
tgttttaattcaatcc

SEQ ID NO: 603 - DNA, 16 bp, synthetic construct
  misc_feature 1..16: note = Synthetic oligonucleotide
  source 1..16: mol_type = other DNA
ctgttttaattcaatc

SEQ ID NO: 604 - DNA, 16 bp, synthetic construct
  misc_feature 1..16: note = Synthetic oligonucleotide
  source 1..16: mol_type = other DNA
gctgttttaattcaat

SEQ ID NO: 605 - DNA, 16 bp, synthetic construct
  misc_feature 1..16: note = Synthetic oligonucleotide
  source 1..16: mol_type = other DNA
agctgttttaattcaa

SEQ ID NO: 606 - DNA, 16 bp, synthetic construct
  misc_feature 1..16: note = Synthetic oligonucleotide
  source 1..16: mol_type = other DNA
cagctgttttaattca

SEQ ID NO: 607 - DNA, 16 bp, synthetic construct
  misc_feature 1..16: note = Synthetic oligonucleotide
  source 1..16: mol_type = other DNA
gcagctgttttaattc

SEQ ID NO: 608 - DNA, 16 bp, synthetic construct
  misc_feature 1..16: note = Synthetic oligonucleotide
  source 1..16: mol_type = other DNA
cgcagctgttttaatt

SEQ ID NO: 609 - DNA, 16 bp, synthetic construct
  misc_feature 1..16: note = Synthetic oligonucleotide
  source 1..16: mol_type = other DNA
tcgcagctgttttaat

SEQ ID NO: 610 - DNA, 16 bp, synthetic construct
  misc_feature 1..16: note = Synthetic oligonucleotide
  source 1..16: mol_type = other DNA
gtcgcagctgttttaa

SEQ ID NO: 611 - DNA, 16 bp, synthetic construct
  misc_feature 1..16: note = Synthetic oligonucleotide
  source 1..16: mol_type = other DNA
tgtcgcagctgtttta

SEQ ID NO: 612 - DNA, 16 bp, synthetic construct
  misc_feature 1..16: note = Synthetic oligonucleotide
  source 1..16: mol_type = other DNA
ttgtcgcagctgtttt

SEQ ID NO: 613 - DNA, 16 bp, synthetic construct
  misc_feature 1..16: note = Synthetic oligonucleotide
  source 1..16: mol_type = other DNA
gttgtcgcagctgttt

SEQ ID NO: 614 - DNA, 16 bp, synthetic construct
  misc_feature 1..16: note = Synthetic oligonucleotide
  source 1..16: mol_type = other DNA
tgttgtcgcagctgtt

SEQ ID NO: 615 - DNA, 16 bp, synthetic construct
  misc_feature 1..16: note = Synthetic oligonucleotide
  source 1..16: mol_type = other DNA
ttgttgtcgcagctgt

SEQ ID NO: 616 - DNA, 16 bp, synthetic construct
  misc_feature 1..16: note = Synthetic oligonucleotide
  source 1..16: mol_type = other DNA
tttgttgtcgcagctg

SEQ ID NO: 617 - DNA, 16 bp, synthetic construct
  misc_feature 1..16: note = Synthetic oligonucleotide
  source 1..16: mol_type = other DNA
ttttgttgtcgcagct

SEQ ID NO: 618 - DNA, 16 bp, synthetic construct
  misc_feature 1..16: note = Synthetic oligonucleotide
  source 1..16: mol_type = other DNA
tttttgttgtcgcagc

SEQ ID NO: 619 - DNA, 17 bp, synthetic construct
  misc_feature 1..17: note = Synthetic oligonucleotide
  source 1..17: mol_type = other DNA
gaaaacccaaatcctca

SEQ ID NO: 620 - DNA, 17 bp, synthetic construct
  misc_feature 1..17: note = Synthetic oligonucleotide
  source 1..17: mol_type = other DNA
agaaaacccaaatcctc

SEQ ID NO: 621 - DNA, 17 bp, synthetic construct
  misc_feature 1..17: note = Synthetic oligonucleotide
  source 1..17: mol_type = other DNA
tagaaaacccaaatcct

SEQ ID NO: 622 - DNA, 17 bp, synthetic construct
  misc_feature 1..17: note = Synthetic oligonucleotide
  source 1..17: mol_type = other DNA
atagaaaacccaaatcc

SEQ ID NO: 623 - DNA, 17 bp, synthetic construct
  misc_feature 1..17: note = Synthetic oligonucleotide
  source 1..17: mol_type = other DNA
ttatagaaaacccaaat

SEQ ID NO: 624 - DNA, 17 bp, synthetic construct
  misc_feature 1..17: note = Synthetic oligonucleotide
  source 1..17: mol_type = other DNA
cttatagaaaacccaaa

SEQ ID NO: 625 - DNA, 17 bp, synthetic construct
  misc_feature 1..17: note = Synthetic oligonucleotide
  source 1..17: mol_type = other DNA
ccttatagaaaacccaa

SEQ ID NO: 626 - DNA, 17 bp, synthetic construct
  misc_feature 1..17: note = Synthetic oligonucleotide
  source 1..17: mol_type = other DNA
cccttatagaaaaccca

SEQ ID NO: 627 - DNA, 17 bp, synthetic construct
  misc_feature 1..17: note = Synthetic oligonucleotide
  source 1..17: mol_type = other DNA
ccccttatagaaaaccc

SEQ ID NO: 628 - DNA, 17 bp, synthetic construct
  misc_feature 1..17: note = Synthetic oligonucleotide
  source 1..17: mol_type = other DNA
accccttatagaaaacc

SEQ ID NO: 629 - DNA, 17 bp, synthetic construct
  misc_feature 1..17: note = Synthetic oligonucleotide
  source 1..17: mol_type = other DNA
aaccccttatagaaaac

SEQ ID NO: 630 - DNA, 17 bp, synthetic construct
  misc_feature 1..17: note = Synthetic oligonucleotide
  source 1..17: mol_type = other DNA
aaaccccttatagaaaa

SEQ ID NO: 631 - DNA, 17 bp, synthetic construct
  misc_feature 1..17: note = Synthetic oligonucleotide
  source 1..17: mol_type = other DNA
gaaaccccttatagaaa

SEQ ID NO: 632 - DNA, 17 bp, synthetic construct
  misc_feature 1..17: note = Synthetic oligonucleotide
  source 1..17: mol_type = other DNA
ggaaaccccttatagaa

SEQ ID NO: 633 - DNA, 17 bp, synthetic construct
  misc_feature 1..17: note = Synthetic oligonucleotide
  source 1..17: mol_type = other DNA
aggaaaccccttataga

SEQ ID NO: 634 - DNA, 17 bp, synthetic construct
  misc_feature 1..17: note = Synthetic oligonucleotide
  source 1..17: mol_type = other DNA
caggaaaccccttatag

SEQ ID NO: 635 - DNA, 17 bp, synthetic construct
  misc_feature 1..17: note = Synthetic oligonucleotide
  source 1..17: mol_type = other DNA
gcaggaaaccccttata

SEQ ID NO: 636 - DNA, 17 bp, synthetic construct
  misc_feature 1..17: note = Synthetic oligonucleotide
  source 1..17: mol_type = other DNA
agcaggaaaccccttat

SEQ ID NO: 637 - DNA, 17 bp, synthetic construct
  misc_feature 1..17: note = Synthetic oligonucleotide
  source 1..17: mol_type = other DNA
cagcaggaaacccctta

SEQ ID NO: 638 - DNA, 17 bp, synthetic construct
  misc_feature 1..17: note = Synthetic oligonucleotide
  source 1..17: mol_type = other DNA
ccagcaggaaacccctt

SEQ ID NO: 639 - DNA, 17 bp, synthetic construct
  misc_feature 1..17: note = Synthetic oligonucleotide
  source 1..17: mol_type = other DNA
tccagcaggaaacccct

SEQ ID NO: 640 - DNA, 17 bp, synthetic construct
  misc_feature 1..17: note = Synthetic oligonucleotide
  source 1..17: mol_type = other DNA
gtccagcaggaaacccc

SEQ ID NO: 641 - DNA, 17 bp, synthetic construct
  misc_feature 1..17: note = Synthetic oligonucleotide
  source 1..17: mol_type = other DNA
tgtccagcaggaaaccc

SEQ ID NO: 642 - DNA, 17 bp, synthetic construct
  misc_feature 1..17: note = Synthetic oligonucleotide
  source 1..17: mol_type = other DNA
ctgtccagcaggaaacc

SEQ ID NO: 643 - DNA, 17 bp, synthetic construct
  misc_feature 1..17: note = Synthetic oligonucleotide
  source 1..17: mol_type = other DNA
cctgtccagcaggaaac

SEQ ID NO: 644 - DNA, 17 bp, synthetic construct
  misc_feature 1..17: note = Synthetic oligonucleotide
  source 1..17: mol_type = other DNA
ccctgtccagcaggaaa

SEQ ID NO: 645 - DNA, 17 bp, synthetic construct
  misc_feature 1..17: note = Synthetic oligonucleotide
  source 1..17: mol_type = other DNA
gcccctgtccagcagga

SEQ ID NO: 646 - DNA, 17 bp, synthetic construct
  misc_feature 1..17: note = Synthetic oligonucleotide
  source 1..17: mol_type = other DNA
cgcccctgtccagcagg

SEQ ID NO: 647 - DNA, 17 bp, synthetic construct
  misc_feature 1..17: note = Synthetic oligonucleotide
  source 1..17: mol_type = other DNA
acgcccctgtccagcag

SEQ ID NO: 648 - DNA, 17 bp, synthetic construct
  misc_feature 1..17: note = Synthetic oligonucleotide
  source 1..17: mol_type = other DNA
cacgcccctgtccagca

SEQ ID NO: 649 - DNA, 17 bp, synthetic construct
  misc_feature 1..17: note = Synthetic oligonucleotide
  source 1..17: mol_type = other DNA
ccacgcccctgtccagc

SEQ ID NO: 650 - DNA, 17 bp, synthetic construct
  misc_feature 1..17: note = Synthetic oligonucleotide
  source 1..17: mol_type = other DNA
cccacgcccctgtccag

SEQ ID NO: 651 - DNA, 17 bp, synthetic construct
  misc_feature 1..17: note = Synthetic oligonucleotide
  source 1..17: mol_type = other DNA
tcccacgcccctgtcca

SEQ ID NO: 652 - DNA, 17 bp, synthetic construct
  misc_feature 1..17: note = Synthetic oligonucleotide
  source 1..17: mol_type = other DNA
atcccacgcccctgtcc

SEQ ID NO: 653 - DNA, 17 bp, synthetic construct
  misc_feature 1..17: note = Synthetic oligonucleotide
  source 1..17: mol_type = other DNA
aatcccacgcccctgtc

SEQ ID NO: 654 - DNA, 17 bp, synthetic construct
  misc_feature 1..17: note = Synthetic oligonucleotide
  source 1..17: mol_type = other DNA
caatcccacgcccctgt

SEQ ID NO: 655 - DNA, 17 bp, synthetic construct
  misc_feature 1..17: note = Synthetic oligonucleotide
  source 1..17: mol_type = other DNA
tcaatcccacgcccctg

SEQ ID NO: 656 - DNA, 17 bp, synthetic construct
  misc_feature 1..17: note = Synthetic oligonucleotide
  source 1..17: mol_type = other DNA
ttcaatcccacgcccct

SEQ ID NO: 657 - DNA, 17 bp, synthetic construct
  misc_feature 1..17: note = Synthetic oligonucleotide
  source 1..17: mol_type = other DNA
attcaatcccacgcccc

SEQ ID NO: 658 - DNA, 17 bp, synthetic construct
  misc_feature 1..17: note = Synthetic oligonucleotide
  source 1..17: mol_type = other DNA
aattcaatcccacgccc

SEQ ID NO: 659 - DNA, 17 bp, synthetic construct
  misc_feature 1..17: note = Synthetic oligonucleotide
  source 1..17: mol_type = other DNA
taattcaatcccacgcc

SEQ ID NO: 660 - DNA, 17 bp, synthetic construct
  misc_feature 1..17: note = Synthetic oligonucleotide
  source 1..17: mol_type = other DNA
ttaattcaatcccacgc

SEQ ID NO: 661 - DNA, 17 bp, synthetic construct
  misc_feature 1..17: note = Synthetic oligonucleotide
  source 1..17: mol_type = other DNA
tttaattcaatcccacg

SEQ ID NO: 662 - DNA, 17 bp, synthetic construct
  misc_feature 1..17: note = Synthetic oligonucleotide
  source 1..17: mol_type = other DNA
ttttaattcaatcccac

SEQ ID NO: 663 - DNA, 17 bp, synthetic construct
  misc_feature 1..17: note = Synthetic oligonucleotide
  source 1..17: mol_type = other DNA
gttttaattcaatccca

SEQ ID NO: 664 - DNA, 17 bp, synthetic construct
  misc_feature 1..17: note = Synthetic oligonucleotide
  source 1..17: mol_type = other DNA
tgttttaattcaatccc

SEQ ID NO: 665 - DNA, 17 bp, synthetic construct
  misc_feature 1..17: note = Synthetic oligonucleotide
  source 1..17: mol_type = other DNA
ctgttttaattcaatcc

SEQ ID NO: 666 - DNA, 17 bp, synthetic construct
  misc_feature 1..17: note = Synthetic oligonucleotide
  source 1..17: mol_type = other DNA
gctgttttaattcaatc

SEQ ID NO: 667 - DNA, 17 bp, synthetic construct
  misc_feature 1..17: note = Synthetic oligonucleotide
  source 1..17: mol_type = other DNA
agctgttttaattcaat

SEQ ID NO: 668 - DNA, 17 bp, synthetic construct
  misc_feature 1..17: note = Synthetic oligonucleotide
  source 1..17: mol_type = other DNA
cagctgttttaattcaa

SEQ ID NO: 669 - DNA, 17 bp, synthetic construct
  misc_feature 1..17: note = Synthetic oligonucleotide
  source 1..17: mol_type = other DNA
gcagctgttttaattca

SEQ ID NO: 670 - DNA, 17 bp, synthetic construct
  misc_feature 1..17: note = Synthetic oligonucleotide
  source 1..17: mol_type = other DNA
cgcagctgttttaattc

SEQ ID NO: 671 - DNA, 17 bp, synthetic construct
  misc_feature 1..17: note = Synthetic oligonucleotide
  source 1..17: mol_type = other DNA
tcgcagctgttttaatt

SEQ ID NO: 672 - DNA, 17 bp, synthetic construct
  misc_feature 1..17: note = Synthetic oligonucleotide
  source 1..17: mol_type = other DNA
gtcgcagctgttttaat

SEQ ID NO: 673 - DNA, 17 bp, synthetic construct
  misc_feature 1..17: note = Synthetic oligonucleotide
  source 1..17: mol_type = other DNA
tgtcgcagctgttttaa

SEQ ID NO: 674 - DNA, 17 bp, synthetic construct
  misc_feature 1..17: note = Synthetic oligonucleotide
  source 1..17: mol_type = other DNA
ttgtcgcagctgtttta

SEQ ID NO: 675 - DNA, 17 bp, synthetic construct
  misc_feature 1..17: note = Synthetic oligonucleotide
  source 1..17: mol_type = other DNA
gttgtcgcagctgtttt

SEQ ID NO: 676 - DNA, 17 bp, synthetic construct
  misc_feature 1..17: note = Synthetic oligonucleotide
  source 1..17: mol_type = other DNA
tgttgtcgcagctgttt

SEQ ID NO: 677 - DNA, 17 bp, synthetic construct
  misc_feature 1..17: note = Synthetic oligonucleotide
  source 1..17: mol_type = other DNA
ttgttgtcgcagctgtt

SEQ ID NO: 678 - DNA, 17 bp, synthetic construct
  misc_feature 1..17: note = Synthetic oligonucleotide
  source 1..17: mol_type = other DNA
tttgttgtcgcagctgt

SEQ ID NO: 679 - DNA, 17 bp, synthetic construct
  misc_feature 1..17: note = Synthetic oligonucleotide
  source 1..17: mol_type = other DNA
ttttgttgtcgcagctg

SEQ ID NO: 680 - DNA, 17 bp, synthetic construct
  misc_feature 1..17: note = Synthetic oligonucleotide
  source 1..17: mol_type = other DNA
tttttgttgtcgcagct

SEQ ID NO: 681 - DNA, 19 bp, synthetic construct
  misc_feature 1..19: note = Synthetic oligonucleotide
  source 1..19: mol_type = other DNA
tagaaaacccaaatcctca

SEQ ID NO: 682 - DNA, 19 bp, synthetic construct
  misc_feature 1..19: note = Synthetic oligonucleotide
  source 1..19: mol_type = other DNA
atagaaaacccaaatcctc

SEQ ID NO: 683 - DNA, 19 bp, synthetic construct
  misc_feature 1..19: note = Synthetic oligonucleotide
  source 1..19: mol_type = other DNA
tatagaaaacccaaatcct

SEQ ID NO: 684 - DNA, 19 bp, synthetic construct
  misc_feature 1..19: note = Synthetic oligonucleotide
  source 1..19: mol_type = other DNA
cttatagaaaacccaaatc

SEQ ID NO: 685 - DNA, 19 bp, synthetic construct
  misc_feature 1..19: note = Synthetic oligonucleotide
  source 1..19: mol_type = other DNA
ccttatagaaaacccaaat

SEQ ID NO: 686 - DNA, 19 bp, synthetic construct
  misc_feature 1..19: note = Synthetic oligonucleotide
  source 1..19: mol_type = other DNA
cccttatagaaaacccaaa

SEQ ID NO: 687 - DNA, 19 bp, synthetic construct
  misc_feature 1..19: note = Synthetic oligonucleotide
  source 1..19: mol_type = other DNA
ccccttatagaaaacccaa

SEQ ID NO: 688 - DNA, 19 bp, synthetic construct
  misc_feature 1..19: note = Synthetic oligonucleotide
  source 1..19: mol_type = other DNA
accccttatagaaaaccca

SEQ ID NO: 689 - DNA, 19 bp, synthetic construct
  misc_feature 1..19: note = Synthetic oligonucleotide
  source 1..19: mol_type = other DNA
aaccccttatagaaaaccc

SEQ ID NO: 690 - DNA, 19 bp, synthetic construct
  misc_feature 1..19: note = Synthetic oligonucleotide
  source 1..19: mol_type = other DNA
aaaccccttatagaaaacc

SEQ ID NO: 691 - DNA, 19 bp, synthetic construct
  misc_feature 1..19: note = Synthetic oligonucleotide
  source 1..19: mol_type = other DNA
gaaaccccttatagaaaac

SEQ ID NO: 692 - DNA, 19 bp, synthetic construct
  misc_feature 1..19: note = Synthetic oligonucleotide
  source 1..19: mol_type = other DNA
ggaaaccccttatagaaaa

SEQ ID NO: 693 - DNA, 19 bp, synthetic construct
  misc_feature 1..19: note = Synthetic oligonucleotide
  source 1..19: mol_type = other DNA
aggaaaccccttatagaaa

SEQ ID NO: 694 - DNA, 19 bp, synthetic construct
  misc_feature 1..19: note = Synthetic oligonucleotide
  source 1..19: mol_type = other DNA
caggaaaccccttatagaa

SEQ ID NO: 695 - DNA, 19 bp, synthetic construct
  misc_feature 1..19: note = Synthetic oligonucleotide
  source 1..19: mol_type = other DNA
gcaggaaaccccttataga

SEQ ID NO: 696 - DNA, 19 bp, synthetic construct
  misc_feature 1..19: note = Synthetic oligonucleotide
  source 1..19: mol_type = other DNA
agcaggaaaccccttatag

SEQ ID NO: 697 - DNA, 19 bp, synthetic construct
  misc_feature 1..19: note = Synthetic oligonucleotide
  source 1..19: mol_type = other DNA
cagcaggaaaccccttata

SEQ ID NO: 698 - DNA, 19 bp, synthetic construct
  misc_feature 1..19: note = Synthetic oligonucleotide
  source 1..19: mol_type = other DNA
ccagcaggaaaccccttat

SEQ ID NO: 699 - DNA, 19 bp, synthetic construct
  misc_feature 1..19: note = Synthetic oligonucleotide
  source 1..19: mol_type = other DNA
tccagcaggaaacccctta

SEQ ID NO: 700 - DNA, 19 bp, synthetic construct
  misc_feature 1..19: note = Synthetic oligonucleotide
  source 1..19: mol_type = other DNA
gtccagcaggaaacccctt

SEQ ID NO: 701 - DNA, 19 bp, synthetic construct
  misc_feature 1..19: note = Synthetic oligonucleotide
  source 1..19: mol_type = other DNA
tgtccagcaggaaacccct

SEQ ID NO: 702 - DNA, 19 bp, synthetic construct
  misc_feature 1..19: note = Synthetic oligonucleotide
  source 1..19: mol_type = other DNA
cctgtccagcaggaaaccc

SEQ ID NO: 703 - DNA, 19 bp, synthetic construct
  misc_feature 1..19: note = Synthetic oligonucleotide
  source 1..19: mol_type = other DNA
ccctgtccagcaggaaacc

SEQ ID NO: 704 - DNA, 19 bp, synthetic construct
  misc_feature 1..19: note = Synthetic oligonucleotide
  source 1..19: mol_type = other DNA
cccctgtccagcaggaaac

SEQ ID NO: 705 - DNA, 19 bp, synthetic construct
  misc_feature 1..19: note = Synthetic oligonucleotide
  source 1..19: mol_type = other DNA
gcccctgtccagcaggaaa

SEQ ID NO: 706 - DNA, 19 bp, synthetic construct
  misc_feature 1..19: note = Synthetic oligonucleotide
  source 1..19: mol_type = other DNA
acgcccctgtccagcagga

SEQ ID NO: 707 - DNA, 19 bp, synthetic construct
  misc_feature 1..19: note = Synthetic oligonucleotide
  source 1..19: mol_type = other DNA
cacgcccctgtccagcagg

SEQ ID NO: 708 - DNA, 19 bp, synthetic construct
  misc_feature 1..19: note = Synthetic oligonucleotide
  source 1..19: mol_type = other DNA
ccacgcccctgtccagcag

SEQ ID NO: 709 - DNA, 19 bp, synthetic construct
  misc_feature 1..19: note = Synthetic oligonucleotide
  source 1..19: mol_type = other DNA
cccacgcccctgtccagca

SEQ ID NO: 710 - DNA, 19 bp, synthetic construct
  misc_feature 1..19: note = Synthetic oligonucleotide
  source 1..19: mol_type = other DNA
tcccacgcccctgtccagc

SEQ ID NO: 711 - DNA, 19 bp, synthetic construct
  misc_feature 1..19: note = Synthetic oligonucleotide
  source 1..19: mol_type = other DNA
atcccacgcccctgtccag

SEQ ID NO: 712 - DNA, 19 bp, synthetic construct
  misc_feature 1..19: note = Synthetic oligonucleotide
  source 1..19: mol_type = other DNA
aatcccacgcccctgtcca

SEQ ID NO: 713 - DNA, 19 bp, synthetic construct
  misc_feature 1..19: note = Synthetic oligonucleotide
  source 1..19: mol_type = other DNA
caatcccacgcccctgtcc

SEQ ID NO: 714 - DNA, 19 bp, synthetic construct
  misc_feature 1..19: note = Synthetic oligonucleotide
  source 1..19: mol_type = other DNA
tcaatcccacgcccctgtc

SEQ ID NO: 715 - DNA, 19 bp, synthetic construct
  misc_feature 1..19: note = Synthetic oligonucleotide
  source 1..19: mol_type = other DNA
ttcaatcccacgcccctgt

SEQ ID NO: 716 - DNA, 19 bp, synthetic construct
  misc_feature 1..19: note = Synthetic oligonucleotide
  source 1..19: mol_type = other DNA
attcaatcccacgcccctg

SEQ ID NO: 717 - DNA, 19 bp, synthetic construct
  misc_feature 1..19: note = Synthetic oligonucleotide
  source 1..19: mol_type = other DNA
aattcaatcccacgcccct

SEQ ID NO: 718 - DNA, 19 bp, synthetic construct
  misc_feature 1..19: note = Synthetic oligonucleotide
  source 1..19: mol_type = other DNA
taattcaatcccacgcccc

SEQ ID NO: 719 - DNA, 19 bp, synthetic construct
  misc_feature 1..19: note = Synthetic oligonucleotide
  source 1..19: mol_type = other DNA
ttaattcaatcccacgccc

SEQ ID NO: 720 - DNA, 19 bp, synthetic construct
  misc_feature 1..19: note = Synthetic oligonucleotide
  source 1..19: mol_type = other DNA
tttaattcaatcccacgcc

SEQ ID NO: 721 - DNA, 19 bp, synthetic construct
  misc_feature 1..19: note = Synthetic oligonucleotide
  source 1..19: mol_type = other DNA
ttttaattcaatcccacgc

SEQ ID NO: 722 - DNA, 19 bp, synthetic construct
  misc_feature 1..19: note = Synthetic oligonucleotide
  source 1..19: mol_type = other DNA
gttttaattcaatcccacg

SEQ ID NO: 723 - DNA, 19 bp, synthetic construct
  misc_feature 1..19: note = Synthetic oligonucleotide
  source 1..19: mol_type = other DNA
tgttttaattcaatcccac

SEQ ID NO: 724 - DNA, 19 bp, synthetic construct
  misc_feature 1..19: note = Synthetic oligonucleotide
  source 1..19: mol_type = other DNA
ctgttttaattcaatccca

SEQ ID NO: 725 - DNA, 19 bp, synthetic construct
  misc_feature 1..19: note = Synthetic oligonucleotide
  source 1..19: mol_type = other DNA
gctgttttaattcaatccc

SEQ ID NO: 726 - DNA, 19 bp, synthetic construct
  misc_feature 1..19: note = Synthetic oligonucleotide
  source 1..19: mol_type = other DNA
agctgttttaattcaatcc

SEQ ID NO: 727 - DNA, 19 bp, synthetic construct
  misc_feature 1..19: note = Synthetic oligonucleotide
  source 1..19: mol_type = other DNA
cagctgttttaattcaatc

SEQ ID NO: 728 - DNA, 19 bp, synthetic construct
  misc_feature 1..19: note = Synthetic oligonucleotide
  source 1..19: mol_type = other DNA
gcagctgttttaattcaat

SEQ ID NO: 729 - DNA, 19 bp, synthetic construct
  misc_feature 1..19: note = Synthetic oligonucleotide
  source 1..19: mol_type = other DNA
cgcagctgttttaattcaa

SEQ ID NO: 730 - DNA, 19 bp, synthetic construct
  misc_feature 1..19: note = Synthetic oligonucleotide
  source 1..19: mol_type = other DNA
tcgcagctgttttaattca

SEQ ID NO: 731 - DNA, 19 bp, synthetic construct
  misc_feature 1..19: note = Synthetic oligonucleotide
  source 1..19: mol_type = other DNA
gtcgcagctgttttaattc

SEQ ID NO: 732 - DNA, 19 bp, synthetic construct
  misc_feature 1..19: note = Synthetic oligonucleotide
  source 1..19: mol_type = other DNA
tgtcgcagctgttttaatt

SEQ ID NO: 733 - DNA, 19 bp, synthetic construct
  misc_feature 1..19: note = Synthetic oligonucleotide
  source 1..19: mol_type = other DNA
ttgtcgcagctgttttaat

SEQ ID NO: 734 - DNA, 19 bp, synthetic construct
  misc_feature 1..19: note = Synthetic oligonucleotide
  source 1..19: mol_type = other DNA
gttgtcgcagctgttttaa

SEQ ID NO: 735 - DNA, 19 bp, synthetic construct
  misc_feature 1..19: note = Synthetic oligonucleotide
  source 1..19: mol_type = other DNA
tgttgtcgcagctgtttta

SEQ ID NO: 736 - DNA, 19 bp, synthetic construct
  misc_feature 1..19: note = Synthetic oligonucleotide
  source 1..19: mol_type = other DNA
ttgttgtcgcagctgtttt

SEQ ID NO: 737 - DNA, 19 bp, synthetic construct
  misc_feature 1..19: note = Synthetic oligonucleotide
  source 1..19: mol_type = other DNA
tttgttgtcgcagctgttt

SEQ ID NO: 738 - DNA, 19 bp, synthetic construct
  misc_feature 1..19: note = Synthetic oligonucleotide
  source 1..19: mol_type = other DNA
ttttgttgtcgcagctgtt

SEQ ID NO: 739 - DNA, 19 bp, synthetic construct
  misc_feature 1..19: note = Synthetic oligonucleotide
  source 1..19: mol_type = other DNA
tttttgttgtcgcagctgt

SEQ ID NO: 740 - DNA, 18 bp, synthetic construct
  misc_feature 1..18: note = Synthetic oligonucleotide
  source 1..18: mol_type = other DNA
ccccttatagaaaaccca

SEQ ID NO: 741 - DNA, 18 bp, synthetic construct
  misc_feature 1..18: note = Synthetic oligonucleotide
  source 1..18: mol_type = other DNA
accccttatagaaaaccc

SEQ ID NO: 742 - DNA, 18 bp, synthetic construct
  misc_feature 1..18: note = Synthetic oligonucleotide
  source 1..18: mol_type = other DNA
aaccccttatagaaaacc

SEQ ID NO: 743 - DNA, 18 bp, synthetic construct
  misc_feature 1..18: note = Synthetic oligonucleotide
  source 1..18: mol_type = other DNA
aaaccccttatagaaaac

SEQ ID NO: 744 - DNA, 18 bp, synthetic construct
  misc_feature 1..18: note = Synthetic oligonucleotide
  source 1..18: mol_type = other DNA
gaaaccccttatagaaaa

SEQ ID NO: 745 - DNA, 18 bp, synthetic construct
  misc_feature 1..18: note = Synthetic oligonucleotide
  source 1..18: mol_type = other DNA
aggaaaccccttatagaa

SEQ ID NO: 746 - DNA, 18 bp, synthetic construct
  misc_feature 1..18: note = Synthetic oligonucleotide
  source 1..18: mol_type = other DNA
caggaaaccccttataga

SEQ ID NO: 747 - DNA, 18 bp, synthetic construct
  misc_feature 1..18: note = Synthetic oligonucleotide
  source 1..18: mol_type = other DNA
gcaggaaaccccttatag

SEQ ID NO: 748 - DNA, 18 bp, synthetic construct
  misc_feature 1..18: note = Synthetic oligonucleotide
  source 1..18: mol_type = other DNA
agcaggaaaccccttata

SEQ ID NO: 749 - DNA, 18 bp, synthetic construct
  misc_feature 1..18: note = Synthetic oligonucleotide
  source 1..18: mol_type = other DNA
cagcaggaaaccccttat

SEQ ID NO: 750 - DNA, 18 bp, synthetic construct
  misc_feature 1..18: note = Synthetic oligonucleotide
  source 1..18: mol_type = other DNA
ccagcaggaaacccctta

SEQ ID NO: 751 - DNA, 18 bp, synthetic construct
  misc_feature 1..18: note = Synthetic oligonucleotide
  source 1..18: mol_type = other DNA
tccagcaggaaacccctt

SEQ ID NO: 752 - DNA, 18 bp, synthetic construct
  misc_feature 1..18: note = Synthetic oligonucleotide
  source 1..18: mol_type = other DNA
gtccagcaggaaacccct

SEQ ID NO: 753 - DNA, 18 bp, synthetic construct
  misc_feature 1..18: note = Synthetic oligonucleotide
  source 1..18: mol_type = other DNA
tgtccagcaggaaacccc

SEQ ID NO: 754 - DNA, 18 bp, synthetic construct
  misc_feature 1..18: note = Synthetic oligonucleotide
  source 1..18: mol_type = other DNA
ctgtccagcaggaaaccc

SEQ ID NO: 755 - DNA, 18 bp, synthetic construct
  misc_feature 1..18: note = Synthetic oligonucleotide
  source 1..18: mol_type = other DNA
cctgtccagcaggaaacc

SEQ ID NO: 756 - DNA, 18 bp, synthetic construct
  misc_feature 1..18: note = Synthetic oligonucleotide
  source 1..18: mol_type = other DNA
ccctgtccagcaggaaac

SEQ ID NO: 757 - DNA, 18 bp, synthetic construct
  misc_feature 1..18: note = Synthetic oligonucleotide
  source 1..18: mol_type = other DNA
cccctgtccagcaggaaa

SEQ ID NO: 758 - DNA, 18 bp, synthetic construct
  misc_feature 1..18: note = Synthetic oligonucleotide
  source 1..18: mol_type = other DNA
cgcccctgtccagcagga

SEQ ID NO: 759 - DNA, 18 bp, synthetic construct
  misc_feature 1..18: note = Synthetic oligonucleotide
  source 1..18: mol_type = other DNA
acgcccctgtccagcagg

SEQ ID NO: 760 - DNA, 18 bp, synthetic construct
  misc_feature 1..18: note = Synthetic oligonucleotide
  source 1..18: mol_type = other DNA
cacgcccctgtccagcag

SEQ ID NO: 761 - DNA, 18 bp, synthetic construct
  misc_feature 1..18: note = Synthetic oligonucleotide
  source 1..18: mol_type = other DNA
ccacgcccctgtccagca

SEQ ID NO: 762 - DNA, 18 bp, synthetic construct
  misc_feature 1..18: note = Synthetic oligonucleotide
  source 1..18: mol_type = other DNA
cccacgcccctgtccagc

SEQ ID NO: 763 - DNA, 18 bp, synthetic construct
  misc_feature 1..18: note = Synthetic oligonucleotide
  source 1..18: mol_type = other DNA
tcccacgcccctgtccag

SEQ ID NO: 764 - DNA, 18 bp, synthetic construct
  misc_feature 1..18: note = Synthetic oligonucleotide
  source 1..18: mol_type = other DNA
atcccacgcccctgtcca

SEQ ID NO: 765 - DNA, 18 bp, synthetic construct
  misc_feature 1..18: note = Synthetic oligonucleotide
  source 1..18: mol_type = other DNA
aatcccacgcccctgtcc

SEQ ID NO: 766 - DNA, 18 bp, synthetic construct
  misc_feature 1..18: note = Synthetic oligonucleotide
  source 1..18: mol_type = other DNA
caatcccacgcccctgtc

SEQ ID NO: 767 - DNA, 18 bp, synthetic construct
  misc_feature 1..18: note = Synthetic oligonucleotide
  source 1..18: mol_type = other DNA
tcaatcccacgcccctgt

SEQ ID NO: 768 - DNA, 18 bp, synthetic construct
  misc_feature 1..18: note = Synthetic oligonucleotide
  source 1..18: mol_type = other DNA
ttcaatcccacgcccctg

SEQ ID NO: 769 - DNA, 18 bp, synthetic construct
  misc_feature 1..18: note = Synthetic oligonucleotide
  source 1..18: mol_type = other DNA
attcaatcccacgcccct

SEQ ID NO: 770 - DNA, 18 bp, synthetic construct
  misc_feature 1..18: note = Synthetic oligonucleotide
  source 1..18: mol_type = other DNA
aattcaatcccacgcccc

SEQ ID NO: 771 - DNA, 18 bp, synthetic construct
  misc_feature 1..18: note = Synthetic oligonucleotide
  source 1..18: mol_type = other DNA
taattcaatcccacgccc

SEQ ID NO: 772 - DNA, 18 bp, synthetic construct
  misc_feature 1..18: note = Synthetic oligonucleotide
  source 1..18: mol_type = other DNA
ttaattcaatcccacgcc

SEQ ID NO: 773 - DNA, 18 bp, synthetic construct
  misc_feature 1..18: note = Synthetic oligonucleotide
  source 1..18: mol_type = other DNA
tttaattcaatcccacgc

SEQ ID NO: 774 - DNA, 18 bp, synthetic construct
  misc_feature 1..18: note = Synthetic oligonucleotide
  source 1..18: mol_type = other DNA
ttttaattcaatcccacg

SEQ ID NO: 775 - DNA, 18 bp, synthetic construct
  misc_feature 1..18: note = Synthetic oligonucleotide
  source 1..18: mol_type = other DNA
gttttaattcaatcccac

SEQ ID NO: 776 - DNA, 18 bp, synthetic construct
  misc_feature 1..18: note = Synthetic oligonucleotide
  source 1..18: mol_type = other DNA
tgttttaattcaatccca

SEQ ID NO: 777 - DNA, 18 bp, synthetic construct
  misc_feature 1..18: note = Synthetic oligonucleotide
  source 1..18: mol_type = other DNA
ctgttttaattcaatccc

SEQ ID NO: 778 - DNA, 18 bp, synthetic construct
  misc_feature 1..18: note = Synthetic oligonucleotide
  source 1..18: mol_type = other DNA
gctgttttaattcaatcc

SEQ ID NO: 779 - DNA, 18 bp, synthetic construct
  misc_feature 1..18: note = Synthetic oligonucleotide
  source 1..18: mol_type = other DNA
agctgttttaattcaatc

SEQ ID NO: 780 - DNA, 18 bp, synthetic construct
  misc_feature 1..18: note = Synthetic oligonucleotide
  source 1..18: mol_type = other DNA
cagctgttttaattcaat

SEQ ID NO: 781 - DNA, 18 bp, synthetic construct
  misc_feature 1..18: note = Synthetic oligonucleotide
  source 1..18: mol_type = other DNA
gcagctgttttaattcaa

SEQ ID NO: 782 - DNA, 18 bp, synthetic construct
  misc_feature 1..18: note = Synthetic oligonucleotide
  source 1..18: mol_type = other DNA
cgcagctgttttaattca

SEQ ID NO: 783 - DNA, 18 bp, synthetic construct
  misc_feature 1..18: note = Synthetic oligonucleotide
  source 1..18: mol_type = other DNA
tcgcagctgttttaattc

SEQ ID NO: 784 - DNA, 18 bp, synthetic construct
  misc_feature 1..18: note = Synthetic oligonucleotide
  source 1..18: mol_type = other DNA
gtcgcagctgttttaatt

SEQ ID NO: 785 - DNA, 18 bp, synthetic construct
  misc_feature 1..18: note = Synthetic oligonucleotide
  source 1..18: mol_type = other DNA
tagaaaacccaaatcctc

SEQ ID NO: 786 - DNA, 18 bp, synthetic construct
  misc_feature 1..18: note = Synthetic oligonucleotide
  source 1..18: mol_type = other DNA
atagaaaacccaaatcct

SEQ ID NO: 787 - DNA, 18 bp, synthetic construct
  misc_feature 1..18: note = Synthetic oligonucleotide
  source 1..18: mol_type = other DNA
tatagaaaacccaaatcc

SEQ ID NO: 788 - DNA, 18 bp, synthetic construct
  misc_feature 1..18: note = Synthetic oligonucleotide
  source 1..18: mol_type = other DNA
ttatagaaaacccaaatc

SEQ ID NO: 789 - DNA, 18 bp, synthetic construct
  misc_feature 1..18: note = Synthetic oligonucleotide
  source 1..18: mol_type = other DNA
cttatagaaaacccaaat

SEQ ID NO: 790 - DNA, 18 bp, synthetic construct
  misc_feature 1..18: note = Synthetic oligonucleotide
  source 1..18: mol_type = other DNA
ccttatagaaaacccaaa

SEQ ID NO: 791 - DNA, 18 bp, synthetic construct
  misc_feature 1..18: note = Synthetic oligonucleotide
  source 1..18: mol_type = other DNA
cccttatagaaaacccaa

SEQ ID NO: 792 - DNA, 18 bp, synthetic construct
  misc_feature 1..18: note = Synthetic oligonucleotide
  source 1..18: mol_type = other DNA
tgtcgcagctgttttaat

SEQ ID NO: 793 - DNA, 18 bp, synthetic construct
  misc_feature 1..18: note = Synthetic oligonucleotide
  source 1..18: mol_type = other DNA
ttgtcgcagctgttttaa

SEQ ID NO: 794 - DNA, 18 bp, synthetic construct
  misc_feature 1..18: note = Synthetic oligonucleotide
  source 1..18: mol_type = other DNA
gttgtcgcagctgtttta

SEQ ID NO: 795 - DNA, 18 bp, synthetic construct
  misc_feature 1..18: note = Synthetic oligonucleotide
  source 1..18: mol_type = other DNA
tgttgtcgcagctgtttt

SEQ ID NO: 796 - DNA, 18 bp, synthetic construct
  misc_feature 1..18: note = Synthetic oligonucleotide
  source 1..18: mol_type = other DNA
ttgttgtcgcagctgttt

SEQ ID NO: 797 - DNA, 18 bp, synthetic construct
  misc_feature 1..18: note = Synthetic oligonucleotide
  source 1..18: mol_type = other DNA
tttgttgtcgcagctgtt

SEQ ID NO: 798 - DNA, 18 bp, synthetic construct
  misc_feature 1..18: note = Synthetic oligonucleotide
  source 1..18: mol_type = other DNA
ttttgttgtcgcagctgt

SEQ ID NO: 799 - DNA, 18 bp, synthetic construct
  misc_feature 1..18: note = Synthetic oligonucleotide
  source 1..18: mol_type = other DNA
tttttgttgtcgcagctg

SEQ ID NO: 800 - DNA, 16 bp, synthetic construct
  misc_feature 1..16: note = Synthetic oligonucleotide
  source 1..16: mol_type = other DNA
aaaacccaaatcctca

SEQ ID NO: 801 - DNA, 16 bp, synthetic construct
  misc_feature 1..16: note = Synthetic oligonucleotide
  source 1..16: mol_type = other DNA
agaaaacccaaatcct

SEQ ID NO: 802 - DNA, 16 bp, synthetic construct
  misc_feature 1..16: note = Synthetic oligonucleotide
  source 1..16: mol_type = other DNA
tatagaaaacccaaat

SEQ ID NO: 803 - DNA, 16 bp, synthetic construct
  misc_feature 1..16: note = Synthetic oligonucleotide
  source 1..16: mol_type = other DNA
ttatagaaaacccaaa

SEQ ID NO: 804 - DNA, 20 bp, synthetic construct
  misc_feature 1..20: note = Synthetic oligonucleotide
  source 1..20: mol_type = other DNA
gcataagagggtaccagctg

SEQ ID NO: 805 - DNA, 20 bp, synthetic construct
  misc_feature 1..20: note = Synthetic oligonucleotide
  source 1..20: mol_type = other DNA
gtcctttagccagggcagca

SEQ ID NO: 806 - DNA, 20 bp, synthetic construct
  misc_feature 1..20: note = Synthetic oligonucleotide
  source 1..20: mol_type = other DNA
tccacccatgttgtgcaagc

SEQ ID NO: 807 - DNA, 20 bp, synthetic construct
  misc_feature 1..20: note = Synthetic oligonucleotide
  source 1..20: mol_type = other DNA
ccacaccatgccacagagac

SEQ ID NO: 808 - DNA, 20 bp, synthetic construct
  misc_feature 1..20: note = Synthetic oligonucleotide
  source 1..20: mol_type = other DNA
ttccgagtcaggctcttccc

SEQ ID NO: 809 - DNA, 20 bp, synthetic construct
  misc_feature 1..20: note = Synthetic oligonucleotide
  source 1..20: mol_type = other DNA
ccttccctgaaggttcctcc

SEQ ID NO: 810 - DNA, 21 bp, synthetic construct
  misc_feature 1..21: note = Primer
  source 1..21: mol_type = other DNA
agtctctgtggcatggtttgg

SEQ ID NO: 811 - DNA, 21 bp, synthetic construct
  misc_feature 1..21: note = Primer
  source 1..21: mol_type = other DNA
gggcgaatgactgagatcttg

SEQ ID NO: 812 - DNA, 27 bp, synthetic construct
  misc_feature 1..27: note = Probe
  source 1..27: mol_type = other DNA
taccgattaccacaagcaaccatggca

SEQ ID NO: 813 - DNA, 22 bp, synthetic construct
  misc_feature 1..22: note = Primer
  source 1..22: mol_type = other DNA
cgaagcagctcaatgaaatcaa

SEQ ID NO: 814 - DNA, 18 bp, synthetic construct
  misc_feature 1..18: note = Primer
  source 1..18: mol_type = other DNA
tgcctggagggccttctt

SEQ ID NO: 815 - DNA, 18 bp, synthetic construct
  misc_feature 1..18: note = Probe
  source 1..18: mol_type = other DNA
agaccacaagttgaagtc

SEQ ID NO: 816 - DNA, 20 bp, synthetic construct
  misc_feature 1..20: note = Primer
  source 1..20: mol_type = other DNA
gggcaaacagcaatttgtga

SEQ ID NO: 817 - DNA, 20 bp, synthetic construct
  misc_feature 1..20: note = Primer
  source 1..20: mol_type = other DNA
tggctacccaccttccttgt

SEQ ID NO: 818 - DNA, 28 bp, synthetic construct
  misc_feature 1..28: note = Probe
  source 1..28: mol_type = other DNA
ctggatactgtcccaatcccggtattcc

SEQ ID NO: 819 - DNA, 22 bp, synthetic construct
  misc_feature 1..22: note = Synthetic oligonucleotide
  source 1..22: mol_type = other DNA
cgaagaagctcagtgaaatcaa

SEQ ID NO: 820 - DNA, 18 bp, synthetic construct
  misc_feature 1..18: note = Synthetic oligonucleotide
  source 1..18: mol_type = other DNA
tgcctggagggccctctt